(19) United States
(12) Patent Application Publication
(10) Pub. No.: US 2024/0041547 A1
(43) Pub. Date: Feb. 8, 2024

(54) ROBOTIC & NAVIGATION ASSISTED TOTAL SPINAL JOINT REPLACEMENT

(71) Applicant: 3Spine, Inc., Chattanooga, TN (US)

(72) Inventors: Christian Davis, Cambridge, MA (US); Marc M. Peterman, Duxbury, MA (US); Steven C. Humphreys, Soldotna, AK (US); Scott Hodges, Chattanooga, TN (US); Ron Yarbrough, Round Rock, TX (US)

(21) Appl. No.: 18/226,979

(22) Filed: Jul. 27, 2023

Related U.S. Application Data
(63) Continuation of application No. PCT/US22/74635, filed on Aug. 5, 2022.
(60) Provisional application No. 63/229,989, filed on Aug. 5, 2021.
(60) Provisional application No. 63/351,568, filed on Jun. 13, 2022.
(60) Provisional application No. 63/345,560, filed on May 25, 2022.

(51) Int. Cl. A61B 34/37; A61F 2/46

(52) U.S. Cl. CPC A61B 34/37; A61F 2/4611; A61B 2034/107

(57) ABSTRACT

Disclosed are systems, methods and devices for a computing device, robot and/or navigational assistance system to increase the accuracy of surgical access to the targeted vertebral segment, vertebral bony preparation, selecting the proper implant size and/or implant positioning, which will optimize the restoration of spinal alignment and/or range of motion to a desirable degree.

### CROSS-REFERENCE TO RELATED APPLICATIONS

This application is a continuation application of Patent Cooperation Treaty Application No. PCT/US22/74635 entitled “ROBOTIC & NAVIGATION ASSISTED TOTAL SPINAL JOINT METHODS” filed Aug. 5, 2022, which claims the benefit of U.S. Provisional Application No. 63/229,989 entitled “Spine System Improvements” filed Aug. 5, 2021; U.S. Provisional Application No. 63/351,568 entitled “Robotic & Navigation Assisted Total Spinal Joint Replacement Methods” filed Jun. 13, 2022 and U.S. Provisional Application No. 63/345,560 entitled “Total Spinal Joint Replacement Methods & Instrumentation” filed May 25, 2022, the disclosures of which are each incorporated by reference herein in their entireties.

### TECHNICAL FIELD

The disclosure relates to robotic assisted and/or navigational assisted spinal procedures. More specifically, the disclosure relates methods for assisting the surgeon to carryout different spinal procedures using a robotic system and/or navigational assistance to increase the increase the accuracy of vertebral preparation and/or implant positioning, thereby improving spinal alignment and range of motion.

### BACKGROUND OF THE INVENTION

Traditionally, surgeons use manual methods for spine surgeries. Spine surgeries often use a variety of instrumentation to prepare the vertebral surfaces to deploy implants or other fixations into the targeted vertebrae. The surgeon must develop a surgical technique and approach based on the pre-operative image scans. All access, bony preparations and deployment are judged visually during the operative procedure using fluoroscopic images and confirmed with the preoperative plan. However, such manual methods strongly rely on the surgeon's experience and can be subject to his changing predisposition, coordination, and hand stability. Furthermore, fluoroscopic images are only represented in 2D and in single views resulting in decreased accuracy for preparation and implantation. Some surgical procedures may be technically difficult because the tools are held in hand and performed manually. Due to the disadvantages described, the surgery may result in several inaccurate methods, including access, preparation and implant positioning.

### BRIEF DESCRIPTION OF THE SEVERAL VIEWS OF THE DRAWINGS

FIG. 1 graphically illustrates one embodiment of a preoperative method for a total spinal joint replacement method;

FIGS. 2A-2B graphically illustrates one embodiment of an imaging protocol of FIG. 1;

FIG. 2C depicts one embodiment of different static and/or dynamic patient positions for the imaging protocol;

FIGS. 3A-3B graphically illustrates different embodiments of a method to generate and manage a preoperative surgical plan using the surgical planning software;

FIG. 3C graphically illustrates one embodiment of a preoperative surgery planning software log-in, new user and/or authentication;

FIGS. 4A-4G depicts one embodiment of the one or more user interfaces of the preoperative surgery planning software;

FIGS. 5A-5K depicts various embodiments of code used for different modules within the preoperative surgery planning software;

FIGS. 6A-6C depicts different embodiments of database and storage layouts;

FIG. 7 graphically illustrates one embodiment of the UML diagram for the preoperative surgery planning software;

FIGS. 8A-8B graphically illustrates one embodiment of a preoperative surgery planning software image processing and output data;

FIG. 8C displays a table of the different selected surgical measurements (SSMs);

FIGS. 9A-9E depicts a portion of the 2D image processing of the preoperative surgery planning software;

FIGS. 10A-10D depicts a portion of 3D image processing of the preoperative surgery planning software;

FIGS. 11A-11O depicts the one or more embodiments using images for image analysis and computing SSMs using the preoperative surgery planning software;

FIGS. 12A-12F depicts the one or more embodiments using images for image analysis and computing SSMs using the preoperative surgery planning software;

FIG. 13 depicts the one or more embodiments using images for image analysis and computing SSMs using the preoperative surgery planning software;

FIGS. 14A-14C illustrates one embodiment of improving the accuracy of osteotomy coronal angle or trajectory using robotic and/or navigation assisted methods;

FIGS. 15A-15B illustrates one embodiment of improving the accuracy of Anterior/Poster (A/P) distance and Center of Rotation (COR) of one or more deployed spinal implants using robotic and/or navigation assisted methods;

FIGS. 16A-16C illustrates one embodiment of improving the accuracy of convergence angle or transverse pedicle angle using robotic and/or navigation assisted methods;

FIGS. 17A-17C illustrates one embodiment of improving the accuracy of the neutral alignment of one or more deployed spinal implants using robotic and/or navigation assisted methods;

FIGS. 18A-18B illustrates one embodiment of the navigation and/or robotic assisted operating room (OR) layout;

FIGS. 19A-19B graphically illustrates different embodiments of a standard intraoperative method for a total spinal joint method to improve spinal alignment and motion restoration;

FIGS. 20A-20B graphically illustrates different embodiments of robotic and/or navigation assisted intraoperative method for a total spinal joint method to improve spinal alignment and motion restoration;

FIGS. 21A-21D graphically illustrates different embodiments of robotic and/or navigation assisted methods that improve the accuracy of the different intraoperative methods;

FIGS. 22A-22B depicts different views of available surgical approaches for deployment a total joint replacement spinal implant system;

FIG. 23 graphically illustrates one embodiment of a method of preparing equipment for navigation and/or robotic assisted intraoperative method;

FIG. 24 graphically illustrates one embodiment of a method of identifying or re-registering one or more surgical instruments method;

FIG. 25A-25D depicts different embodiments of surgical instruments comprising a portion of reference frame for registration and tracking;

FIG. 26 graphically illustrates one embodiment of a method of synchronizing one or more preoperative images with one or more intraoperative images;

FIGS. 27A-27C depicts different embodiments of the surgical measurements transformed and/or superimposed onto one or more intraoperative images;

FIG. 28 graphically illustrates one least one embodiment for a method of accessing the localized spine segment in a spine region;

FIGS. 29A-29B graphically illustrates different embodiments for one or more decompression techniques for the total joint replacement spinal implant system;

FIGS. 30A-30B depicts different embodiments of surgical access windows or zones;

FIGS. 31A-31B graphically illustrates one embodiment for a method of selecting the proper spinal implant size;

FIGS. 32A-32B graphically illustrates different embodiments of a method of determining the proper spinal implant length using length trials;

FIGS. 33A-33B depict a top view of one embodiment of the surgical instrumentation for a total spinal joint replacement procedure;

FIGS. 34A-34C depict different views of one embodiment of a length trial;

FIG. 35 depicts one embodiment of the transformed and/or superimposed one objects comprising a convergence angle or transverse pedicle angle onto one or more intraoperative images;

FIGS. 36A-36D depicts different embodiments of the transformed and/or superimposed one or more objects comprising center of rotation (COR) within different vertebral segments onto one or more intraoperative images;

FIGS. 37A-37B depict different views of the length trial within an intravertebral space within a spine region with one embodiment of a method to measure COR;

FIG. 37C depicts a top view of a length trial and its angle of convergence or transverse pedicle angle in different spine segments in different spine regions;

FIGS. 38A-38B graphically illustrates different embodiments of a method of determining the proper spinal implant height using height trials;

FIGS. 39A-39C depict different views of one embodiment of a height trial;

FIG. 40 depict a sagittal view of the height trial within an intravertebral space within a spine region;

FIGS. 41A-41C graphically illustrates different embodiments of a method of preparing an intravertebral space within a localized spine segment for alignment restoration;

FIGS. 42A-42B graphically illustrates different embodiments of a method of preparing the caudal vertebral body;

FIGS. 43A-43B depict a sagittal view of one embodiment of a preparing a caudal vertebral body with an osteotomy sagittal angle;

FIG. 44A-44B depicts an anterior view of one embodiment of a prepared caudal vertebral body with an osteotomy coronal angle;

FIGS. 45A-45B graphically illustrates different embodiments of a method of preparing the cranial vertebral body;

FIGS. 46A-46C depicts a sagittal and posterior view of the prepared intravertebral space to acquire neutral alignment;

FIGS. 47A-47E depicts different isometric views of a powered reciprocating system;

FIGS. 48A-48C graphically illustrates different embodiments of a method of completing at least one keel cut on the caudal vertebral body;

FIGS. 49A-49B graphically illustrates different embodiments of a method of completing at least one keel channel on the cranial vertebral body;

FIGS. 50A-50B depict an isometric sagittal view and a top view of prepared keel channel on the caudal vertebral body;

FIGS. 51A-51D depict different views of one embodiment of a keel alignment tool for the method of completing at least one keel channel on the cranial vertebral body;

FIGS. 52A-52E depicts different views of a method to assemble a keel alignment tool;

FIGS. 53A-53D depicts sagittal views and magnified views of the keel alignment tool within the prepared intravertebral space to create at least one cranial keel channel;

FIG. 53E depicts a magnified view of one embodiment of an aligned caudal keel channel relative to a cranial keel channel;

FIGS. 54A-54B graphically illustrates different embodiments of a method of implanting a total joint replacement spinal implant;

FIGS. 55A-55C depicts different views of one embodiment of a deployment tool;

FIGS. 56A-56D depicts different views of preparing a spinal implant for deployment;

FIGS. 57A-57E depicts cross-sectional and magnified views of FIGS. 56A-56D of preparing a spinal implant for deployment;

FIGS. 58A-58F depicts different sagittal and exploded views of a method of deploying a spinal implant within the prepared intravertebral space;

FIGS. 59A-59F depicts different embodiments of one or more spinal implant characteristics required for confirmation of a deployed spinal implant into the prepared intervertebral space;

FIG. 60 graphically illustrates one embodiment of a method of removing at least one spinal implant from the prepared intervertebral space;

FIGS. 61A-61B depicts a side view of one embodiment of a removal tool and its insertion location on the spinal implant; and

FIGS. 62A-62C depicts a side and isometric view of one embodiment of spinal implant removal tool and its insertion location on the spinal implant.

### BRIEF DESCRIPTION OF THE INVENTION

There is a need to provide an improved method to improve the accuracy of one or more operative steps, including surgical access, surgical bony preparation and/or spinal implant positioning to further improve spinal alignment and range of motion by allowing surgeons to use a robotic assisted and/or navigational assisted spinal surgeries.

In one embodiment, a computer-assisted method to improve the accuracy of spinal alignment and motion restoration comprises the steps of: accessing a preoperative surgical plan from a storage device, the preoperative surgical plan including one or more preoperative virtual models, the one or more preoperative virtual models including a targeted anatomy having defined anatomical landmarks; acquiring one or more operative images of the targeted anatomy using one or more imaging techniques to create one or more operative virtual models and an operative plan; generating a first one or more spatial coordinates to create a first surgical trajectory for one or more operative methods; projecting a first one or more objects onto the display that substantially matches the first surgical trajectory to position a user to execute one or more operative methods; generating a second one or more spatial coordinates to create a second surgical trajectory and projecting a second one or more objects onto the display; comparing the first surgical trajectory relative to the second surgical trajectory; and correcting the user to return to the first surgical trajectory after a determination that the second surgical trajectory is substantially different than the first surgical trajectory by providing feedback.

In another embodiment, a computer-assisted method to improve the accuracy of spinal alignment and motion restoration comprises the steps of: accessing one or more preoperative images from a storage device comprising of a portion of a spine of a patient within a spine region collected using an imaging protocol and one or more imaging techniques; generating one or more preoperative virtual models, the at least one preoperative virtual model includes defined anatomical landmarks; creating a preoperative plan by analyzing the one or more preoperative virtual models to compute one or more desired selected surgical measurements for one or more operative methods; acquiring one or more operative images of the at least a portion of the spine of the patient within the spine region using one or more imaging techniques to create one or more operative virtual models; superimposing one or more preoperative virtual models onto the one or more operative virtual models onto a display to create one or more synchronized virtual models to confirm the preoperative surgical plan; generating a first one or more spatial coordinates to create a planned surgical trajectory for the one or more operative methods; projecting one or more objects onto the display that substantially matches the first surgical trajectory to help guide or position a user; generating a second one more spatial coordinates to create a second surgical trajectory to be projected as a second one or more objects onto the display; comparing the first surgical trajectory relative to the second surgical trajectory; and providing feedback to the user to return to the first surgical trajectory if the second surgical trajectory is substantially different than the first surgical trajectory.

In another embodiment, a computer-assisted method to improve the accuracy of spinal alignment and motion restoration comprises the steps of: accessing a preoperative plan from a storage device, the preoperative surgical plan including one or more preoperative virtual models, the one or more preoperative virtual models including a targeted anatomy having defined anatomical landmarks; acquiring one or more operative images of the targeted anatomy using one or more imaging techniques to create one or more operative virtual models and an operative plan; generating a first one or more spatial coordinates to create a first surgical trajectory for one or more operative methods; projecting one or more objects onto a display that substantially matches the first surgical trajectory to help position a user to execute the one or more operative methods; generating a second one or more spatial coordinates to create a second surgical trajectory to be projected as a second one or more objects onto the display; comparing the first surgical trajectory relative to the second surgical trajectory; and correcting the user to return to the first trajectory if the second surgical trajectory is substantially different than the first surgical trajectory. The computer-assisted method to improve the accuracy of spinal alignment and motion restoration further comprises the step of generating an outcome study report, the outcome study report including a comparison of the preoperative surgical plan relative to the operative surgical plan.

In another embodiment, a computer-assisted method to improve the accuracy of spinal alignment and motion restoration comprises the steps of: acquiring one or more operative images of the at least a portion of a targeted anatomy using one or more imaging techniques to create one or more operative virtual models; generating a first one or more spatial coordinates to create a first surgical trajectory from the one or more operative virtual models; projecting one or more objects onto the one or more operative virtual models that substantially matches the first surgical trajectory to help guide or position the user to execute the one or more operative methods; displaying a second one or more spatial coordinates that creates a second surgical trajectory onto a display; comparing the first surgical trajectory relative to the second surgical trajectory; and providing feedback to the user to return to the first trajectory after a determination that the second surgical trajectory is substantially different than the first surgical trajectory.

In another embodiment, a non-transitory computer-readable medium with instructions stored thereon, that when executed by a processor, perform the steps comprising: accessing one or more preoperative images of a target anatomy of a patient; generating a first data set, the first data set comprises one or more preoperative virtual models of the target anatomy, the one or more preoperative virtual models including identification of anatomical landmarks; calculating one or more selected surgical measurements from the one or more preoperative virtual models of the target anatomy to create a second data set; and creating a preoperative plan by analyzing the one or more preoperative virtual models and the one or more selected surgical measurements.

### DETAILED DESCRIPTION OF THE INVENTION

Traditionally, spinal surgeries are performed manually. The surgeon acquires a set of images preoperatively and uses the images to prepare a surgical plan. During the operative procedure, the surgeon relies on fluoroscopy guided free-hand surgeries and the developed preoperative plan. Long and narrow paths to the spine are difficult to navigate by free-hand leading to various complications (e.g., vascular injury, neurologic injury, improper implant positioning, etc.). However, using navigation assisted spine surgery and/or robotic assisted spine surgery may accurately identify and/or maintain bone access or bone entry position, bone access or bone entry trajectory, bone preparation trajectories, and/or implant placement or positioning. Accordingly, navigation assisted spine surgery and/or robotic assisted spine surgery may further decrease complication rates, reduce radiation exposure, reduce surgery time, reducing or eliminating surgeon individual characteristics (e.g., hand tremors, surgeon fatigue, etc.) and/or reducing incision size. Furthermore, the navigation and/or robotic systems allow the surgeon to access 2D and 3D visualizations in various planes of the patient's imaging, rather than the limited 2D single plane image of fluoroscopy.

In another embodiment, a method to improve the accuracy implant positioning may comprise the steps of: generating a preoperative surgical plan by using a preoperative planning using a software application; and completing one or more operative methods using the preoperative surgical plan. In another embodiment, the method to improve the accuracy of bony preparation of removing at least one deployed spinal implant. The preoperative planning software application will output and/or display a variety of surgical measurements or parameters and enhanced virtual models that may improve the accuracy accessing the spine, bony preparation, selecting the proper implant size and implant positioning after deployment, which will ultimately optimize spinal alignment and motion restoration of a patient.

Preoperative Protocol or Preoperative Method

The method to restore alignment and motion, to improve bony preparation accuracy and improve implant positioning will comprise a preoperative protocol and/or a pre-operative method. The ultimate goals of preoperative protocol are to reduce the patient's surgical and morbidity or mortality intraoperatively and postoperatively, and reduce the overall risk to the patient. More specifically, the detailed goals of the preoperative procedure includes the documentation of the condition(s) for which surgery is required; assessing the patient's overall health status to uncover hidden conditions that could increase perioperative and postoperative risk; development of an appropriate surgical or intraoperative plan; educate the patient about the upcoming surgery to reduce anxiety; and/or reduce costs by shortening hospital stay and increase satisfaction.

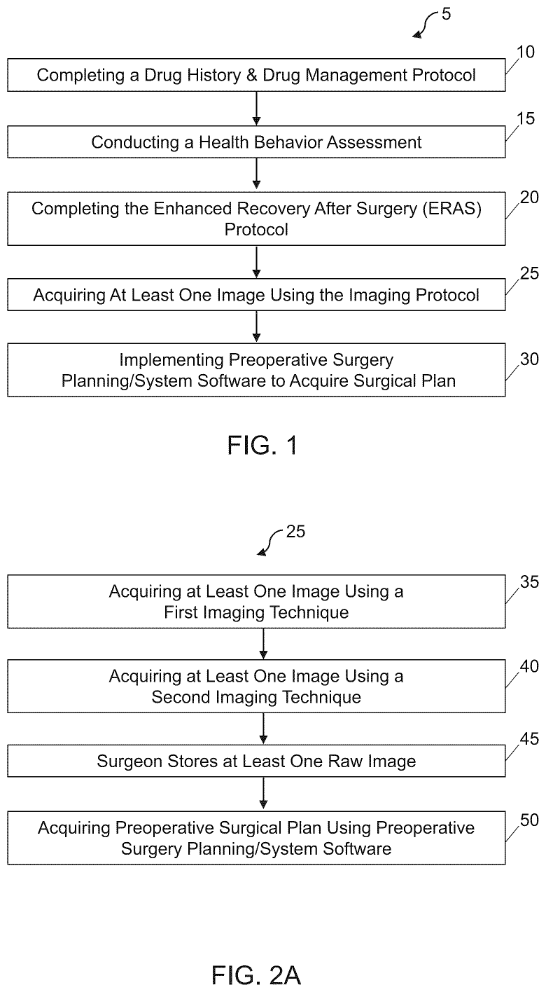

With reference to FIG. 1, the preoperative protocol or method 5 comprises the steps of: completing a drug history and a drug management protocol 10; conducting a health behavior assessment and health behavior protocol 15; assessing perioperative anesthesia risk and completing perioperative anesthesia protocol for Enhanced Recovery After Surgery (ERAS) 20; completing an imaging protocol 25; and/or implementing preoperative planning software application to acquire desired or projected surgical measurements (PSM) or selected surgical measurements (SSM) 30. The preoperative protocol 5 may further comprise the step of creating or developing a perioperative surgical plan and/or surgical approach using the projected surgical measurements (PSM) from the preoperative software. Furthermore, the preoperative protocol or method 5 may further comprise the step of pre-registration, tracking or identification of the various surgical instruments with the selected or desired navigation and/or robotic system.

The steps of completing a drug history and a drug management protocol 10; conducting a health behavior assessment and health behavior protocol and assessing perioperative anesthesia risk and completing perioperative anesthesia protocol for Enhanced Recovery After Surgery (ERAS) 20 may be completed to procedures known in the art for spine surgeries.

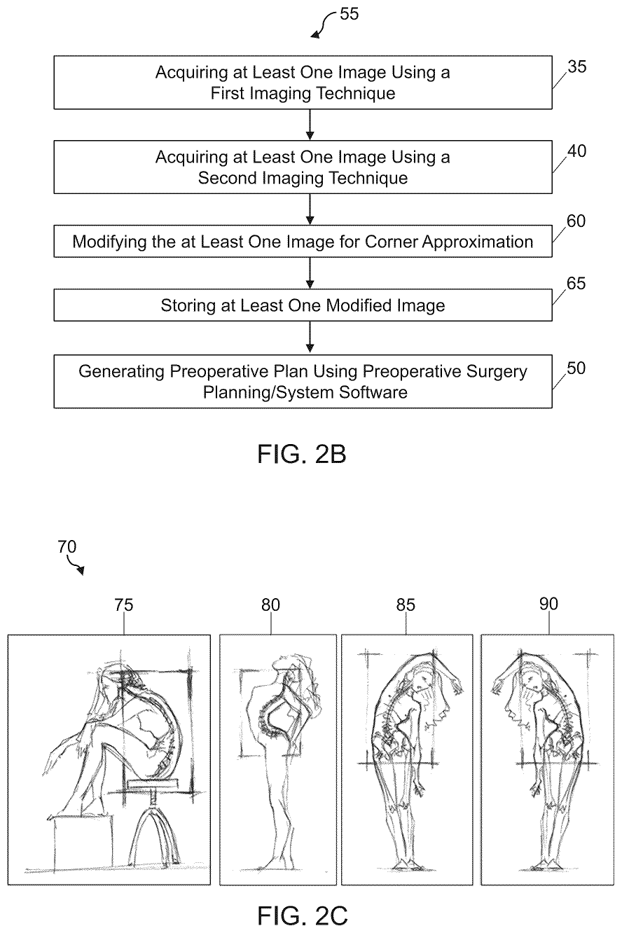

With reference to FIGS. 2A-2B, the preoperative protocol or method 5 comprises an imaging protocol 25,55. The preoperative imaging protocol 25,55 is essential for providing a current picture and understanding of a patient's condition, spinal biomechanics and morphological bony structures. Acquiring one or more images provides surgeons with more information and the surgeons can exploit the anatomical and functional data to help develop an intraoperative or surgical approach to restore alignment and/or restore mobility.

The spinal biomechanics may be evaluated during the lordotic changes between different positions as shown in FIG. 2C. Positions may include static or dynamic positions. Static positions comprise standing, sitting, or lying down (in supine or prone position). The standing neutral and lateral sitting, which may be indicative guarding, rigidity and pathology. The lateral sitting view may also be used to evaluate degree of spondylolisthesis and angulatory changes due to the translational force of this position. Furthermore, the bone morphology may evaluate the facet joints, spondylolisthesis, shape of the endplate and Schmorl's nodes. If necessary, the amount of spondylolisthesis should be measured, and it should be obtained or measured down the midline. Each of the static or dynamic positions comprises different posture 8-s, the postures include one or more of the following: flexion, extension, lateral flexion, lateral extension, neutral, rotating, bending, and/or any combination thereof.

In one embodiment, the imaging protocol 25,55 comprises the steps of: completing at least one MRI imaging scan to evaluate the soft-tissue related pathology, including disc degeneration grade, facet joint cartilage, and nerve compression; obtaining at least one or more radiographs (e.g., X-rays) at different static and dynamic positions, the positions including one or more postures 70, the postures 70 includes a lateral slump sitting views 75, at least a standing anterior/posterior 80, flexion/extension 85,90, and/or a lateral neutral to evaluate biomechanics and/or any combination thereof as shown in FIG. 2C. The imaging protocol 25,55 may further include the step of obtaining at least one CT scan.

In another embodiment, the imaging protocol 25 comprises the steps of acquiring at least one image using a first imaging technique 35; acquiring at least one image using a second imaging technique 40; storing at least one raw image from the first and/or second imaging techniques 45; and/or acquiring one or more projected surgical measurements (PSM) and/or processed images from the preoperative surgery planning software to create an operative plan 50. The imaging protocol 25,55 may further comprise a third imaging technique.

In another embodiment, the imaging protocol 55 comprises the steps of acquiring at least one image using a first imaging technique 35; acquiring at least one image using a second imaging technique 40; modifying at least one raw image to highlight relevant bony structures 60; storing at least one modified image from the first and/or second imaging techniques 65; and/or acquiring one or more projected surgical measurements (PSM) from the preoperative surgery planning software to create an operative plan 50. The imaging protocol 55 may further comprise a third imaging technique.

The first imaging technique may be the same as the second imaging technique. The first imaging technique may be different than the second imaging technique. Furthermore, each of the first imaging technique, the second imaging technique and/or the third imaging technique may comprise different imaging techniques. Each of the first imaging techniques, the second imaging techniques and/or the third imaging technique may comprise a same imaging technique. The imaging techniques include an MRI, a radiograph or X-ray, a CT scan, an ultrasound, and/or any combination thereof. Each of the first imaging techniques, the second imaging technique and/or the third imaging technique may comprise a single image and/or multiple images. Each of the first imaging technique, the second imaging technique and/or the third imaging technique comprises static or dynamic positions.

In one embodiment, acquiring at least one image using a CT scan technique may desirably help the surgeon by highlighting different types of structures in the body. A CT scan is an imaging tool that can provide detailed cross-sectional images of the structures, including tissues, bony structures, and organs, to understand the size, shape and position of the relevant structures. It can help identify the damaged disc(s) or disc degeneration, vertebral fractures, as well as identify other sources of pain. In another embodiment, acquiring at least one image using radiographs (e.g., X-rays) technique may desirably help the surgeon to investigate the cause of the patients' symptoms at a targeted spinal level, bone morphology, and/or identify presence of abnormal movement. The at least one image may comprise static images, dynamic images, full spine, portions of the spine and/or any combination thereof. In another embodiment, the acquiring at least one image using magnetic resonance imaging (MRI) technique may desirably help the surgeon to assess chronic low back pain by highlighting the disc and/or abnormal vertebral endplates. Such images may provide evidence for the pain being discogenic. It also provides information on the dimensions of the spinal canal and the appearance of the articular facets.

In another embodiment, the at least one radiograph image may comprise at least one static image. The at least one static image may comprise a posterior-anterior and a lateral image of the whole of the spine and pelvis under load and/or a portion of the spine and/or pelvis under load. The aim is to eliminate a non-discogenic etiology wholly or partly responsible for the patient's symptoms (non-degenerative lumbar causes: fractures, infection, neoplasia or extra-spinal causes) and to investigate the level concerned (disc degeneration, spondylolisthesis, joint abnormality, etc.). The at least one static images also provide useful information for preparing the surgical procedure, including the quality of the bone, the shape of the vertebral endplates, the orientation of the intervertebral spaces relative to the pubis to help anticipate surgical approach difficulties, on whether the disc is collapsed, identification of bony foraminal stenosis (which cannot be treated via the anterior route in isolation); on the state of adjacent discs and the lumbopelvic-femoral complex, and/or any combination thereof. The at least one static image comprises static positions, the static positions comprise standing, sitting, or lying down (in supine or prone position). Each of the static or dynamic positions comprises different postures, the postures include one or more of the following: flexion, extension, lateral flexion, lateral extension, neutral, rotating, bending, and/or any combination thereof.

In one embodiment, the at least one radiograph image may comprise at least one dynamic image. The at least one dynamic image comprises a lateral flexion, contralateral flexion, extension, flexion while lying down, sitting and/or standing. Using the at least one dynamic image may assist the surgeon to assess the mobility of the healthy discs and the presumed pathological disc (as decreased, normal or exaggerated mobility suggests intervertebral mechanical instability). The aim of dynamic images is to identify abnormal movement between two vertebrae. Each of the at least one dynamic image comprises dynamic positions, the dynamic positions comprise standing, sitting, or lying down (in supine or prone position). Each of the static or dynamic positions comprises different postures, the postures include one or more of the following: flexion, extension, lateral flexion, lateral extension, neutral, rotating, bending, and/or any combination thereof.

In another embodiment, the imaging techniques may comprise a targeted anatomy. The targeted anatomy comprises at least one full spine image or a portion of a spine image. The targeted anatomy may comprise a spine segment in a spine region. The targeted anatomy may further comprise a plurality of spine segments in a plurality of spine regions. The targeted anatomy may further comprise a first spine segment in a first spine region and a second spine segment in a second spine region. The at least on full spine image includes a full spine PA and/or a lateral static radiograph. The at least one full spine image helps the surgeon to assess the sagittal balance (e.g., lordosis), and/or scoliosis.

In another embodiment, the step of storing at least one image 45,65 comprises storing at least one raw or modified image. A raw image includes images that are minimally processed images that are directly acquired from the imaging technique. The raw image may be converted to a modified image by adjusting, annotating, highlighting, manipulating, and/or processing the raw image. The steps of modifying the at least one image 60 requires the surgeon to use at least one raw image for modification. The at least one raw image will be manipulated to highlight and predict the four corners of a vertebral body in one or more vertebral segments. The modified images may comprise annotations, the annotations include text or markers, that highlight the four corners of the one or more vertebral body for one or more targeted spine segments in a spine region. This results and/or creates at least one modified image.

The step of storing the at least one image 45,65 requires the surgeon to ensure that the one or more images collected using one or more imaging techniques are primarily stored into one or more storage devices and/or storage mediums. The one or more images may comprise CT scout images, raw images (e.g., unmodified images) and/or the modified images. The one or more storage devices may include internal (part of computer's hardware), external (installed outside of computer), virtual and/or portable. Virtual storage devices may include online or cloud-based storage. The portable computer external storage system or drive may comprise a flash storage, a USB storage, an external hard drive, external CD-ROM or DVD-ROM drives. Alternatively, the surgeon may require downloading the raw and/or modified images into a 2^{nd} storage. The second storage may comprise a portable computer external storage system or drive.

In another embodiment, the preoperative method may further comprise the step of pre-registration, tracking or identification of the various surgical instruments with the selected or desired navigation and/or robotic system. The navigation and/or robotic systems should be able to identify and track multiple surgical instruments to meet the requirements of applying multiple surgical instruments during a surgical procedure. At least one or more of the surgical instrumentations may be configured and/or adapted to receive at least one navigation and/or robotic equipment. The at least one navigation and/or robotic equipment may comprise one or more light emitting diodes (LEDs), markers, reference arrays and/or fiducials. The identification and/or tracking of the surgical instruments would allow the navigation and/or robotic systems to identify one or more surgical instruments based on the marker's geometrical arrangement in space in relation to the body, the instruments' stability, the space distance and/or rotation of instruments, as well as understand the instruments coordinates in a 2D and/or 3D coordinate system.

The identification and/or tracking may comprise a plurality of methods. The plurality of methods may include: tracking or identification of one or more surgical instruments based on the unique geometries formed by markers coupled to the surgical instrument; tracking or identifying one or more surgical instruments based on a motion vector; and/or a stereo matching algorithm using two intersecting lines and surgical instrument codes. See Mengshi Zhang, et al., “Multiple Instruments Motion Trajectory Tracking in Optical Surgical Navigation,” Opt. Express 27, 15827-15845 (2019) and Cai K, et al. Tracking multiple surgical instruments in A Near-Infrared Optical System, Comput. Assist. Surg. (Abingdon) (December 2016), 21(1):46-55. In another embodiment, the identification and/or tracking of one or more surgical instruments may further comprise the use of matching or identifying unique 2D or 3D bar codes disposed on each of the surgical instruments. In another embodiment, the identification and/or tacking of one or more surgical instruments further comprise the use of stereotactic principles and an optical tracking system. The stereotactic optical tracking system includes at least three optical LEDs, a Bumblee2 binocular camera, and/or a near-infrared filter. The at least three optical LEDs are used as the markers that are installed or disposed on the surgical instrument in one or more different positions. The BUMBLEBEE2 binocular camera is used to capture the images of luminescence of the markers. To eliminate the interference of ambient light, the near-infrared filter is disposed in front of the binocular camera lens. To extract the pixel coordinate of a marker's center, the region growing (RG) method is used to eliminate the singular point and then the gray centroid (GC) method is adopted to obtain the pixel coordinate of the marker's center. This image processing method greatly enhances the antijamming capability and improves the accuracy of the system. Next, the algorithm matches the markers in the left and right camera images based on the sequence of its spatial positions. As a last step, it derives the coordinates of the surgical instrument tip by 3-D coordinates reconstruction of markers and realizes the tracking system. See Zhentian Zhou, et al., “Optical Surgical Instrument Tracking System Based on the Principle of Stereo Vision, J. of Biomedical Optics, Vol. 22, Issue 6 (Jun. 28, 2017).

In another embodiment, the preoperative method 5 comprises the step of acquiring, generating or computing one or more selected surgical measurements (SMM) from the preoperative surgery planning software application 50. The preoperative surgical planning software is a preoperative software used to pre-visualize a surgical intervention using computer generated surgical measurements and/or processed 2D or 3D images, in order to predefine the preoperative and/or operative steps and navigation in the context of computer-assisted surgery. More specifically, the software application allows precise planning of a surgical operation using a real anatomical image or real anatomical virtual model of the targeted anatomy that provides a precise spatial visualization enabling the surgeon or user to “practice,” “engage” or visualize the patient specific procedure before the actual procedure takes place. The preoperative plan can be prepared for surgeon or other medical user review, and can include the planning of various bone resections, sizes and types of implants, and various geometric requirements including relevant dimensions, such as height, width, orientation of particular features, etc. The preoperative surgical plan can further include a recommendation of particular implants and associated instrumentation and/or guides to be used in the surgical procedure.

The surgical planning software further integrates patients anatomic and medical information with interactive participation by a surgeon or user, various hospital and/or imaging center personnel, and a service provider or original equipment manufacturer to plan and manage a surgery from initial consultation with a surgeon through postoperative reporting and archiving. In one exemplary embodiment, the planning process includes a partially automated process utilizing a centralized user interface or web portal where the surgeon or other user and/or patient can interact. The web portal may comprise various levels of user (e.g., Surgeon, service provider and patient) access to various tools for case management, preoperative planning, communicating/sharing, manufacturing, surgical execution, and postoperative planning and data archiving.

The web portal can provide a single source of access and information sharing thereby reducing complexity and increasing efficiency for the surgeon or other user. As will be explained in more detail below, the web portal can facilitate a single source of access to an integrated workflow of tools and solutions guiding users through the preoperative planning, surgical execution and/or postoperative aspects of a surgery.

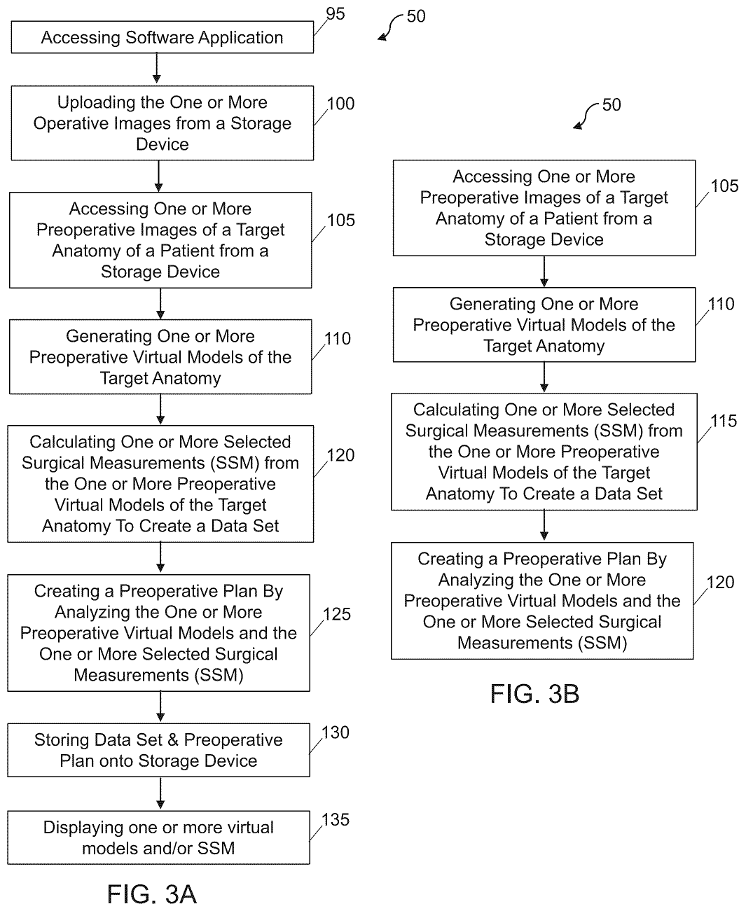

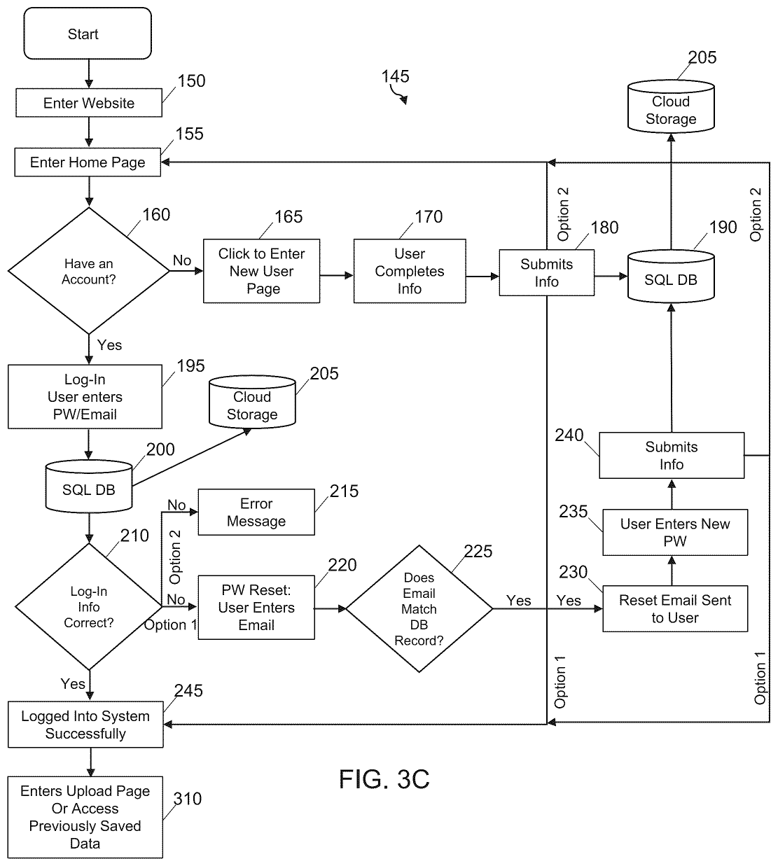

With reference to FIGS. 3A-3B, the step of acquiring, generating or creating a preoperative plan using the preoperative surgery planning software 50 may comprise the steps of: accessing one or more preoperative images of a target anatomy of a patient from a storage device 105; generating one or more preoperative virtual models of the target anatomy, the one or more preoperative virtual models including identification of anatomical landmarks 110; calculating one or more selected surgical measurements (SSM) from the one or more preoperative virtual models of the target anatomy to create a data set 115; and creating or generating a preoperative plan by analyzing the one or more preoperative virtual models and the one or more selected surgical measurements 120.

The step of acquiring, generating or creating a preoperative plan using the preoperative surgery planning software 50 may further comprise the step of accessing the software application 95. The step of accessing the software application may comprise a number of steps that requires the surgeon and/or other user to access a web portal to log-in and/or create a new-user sign-up (see FIG. 3C).

The step of acquiring, generating or creating a preoperative plan using the preoperative surgery planning software 50 may further comprise the step of uploading the one or more operative images into the software from a storage device 100. As described herein, the storage devices may include internal (part of computer's hardware), external (installed outside of computer), virtual and/or portable storage. The one or more operative images may comprise raw or modified images.

The step of acquiring, generating or creating a preoperative plan using the preoperative surgery planning software 50 may further comprise the step of storing the data set and/or preoperative plan onto a storage device 130. As described herein, the storage devices may include internal (part of computer's hardware), external (installed outside of computer), virtual and/or portable storage. The one or more operative images may comprise raw or modified images.

The step of acquiring, generating, or creating a preoperative plan using the preoperative surgery planning software 50 may further comprise the step of displaying the data set onto a display and/or a graphical user interface (GUI) 135. The data set comprises computed one more SSM and/or one or more preoperative virtual models. The preoperative virtual models may comprise one or more 2D virtual models and/or one or more 3D virtual models. The one or more preoperative virtual models may further comprise defined anatomical landmarks. The anatomical landmarks may include vertebral body edges, borders, width, height, length, surface texture, surface changes, axes or axis, and/or any combination thereof. The anatomical landmarks may be disposed onto a plurality of spinal segments in a spine region. The anatomical landmarks may be disposed onto a plurality of spinal segments in a plurality of spine regions. Such anatomical landmarks assist the software to compute the one or more SSMs and the virtual models.

In the step of generating one or more preoperative virtual models of the target anatomy, the one or more operative images may be exhibited to a variety of processing techniques to analyze the content of the images for the production or computation of SMM or PSM. The image processing may involve numerous procedures including formatting and correcting of the data, digital enhancement to facilitate better visual interpretation, or even automated classification of targets and features entirely by computer to produce a set a set of results or data extracted from the one or more preoperative images. Image processing functions may include, but not limited to preprocessing, image enhancements, image transformations, image classification, and/or image analysis. The results or data may comprise 2D processed images, 3D processed images, and/or selected surgical measurements (SMM). The results or data will be used by the surgeon to facilitate operative surgical planning.

Preoperative Planning Software Application System Architecture

With reference to FIGS. 3-13, the one or more stored raw and/or modified preoperative images are available to proceed to the preoperative planning software application system. The preoperative planning software application system is a way to interact remotely with a user or surgeon along with simple and easy-to-understand guidance messages. It may be used in conjunction with traditional manual surgery, navigation assisted surgery and/or robotic assisted spinal surgery. The software app stores an email and password combination in a secured database for logging-in purposes. It uses a unique algorithm called Secure Hash Algorithm 2 (SHA-2) to add extra security to user passwords. Upon successful account creation, users and/or surgeons will be able to store image files and other numeric data in their unique folder in the secure cloud storage.

The software system architecture for providing one or more methods of improving accuracy of bony preparation, implant positioning, spinal alignment and/or spinal motion. The software system architecture comprises a computer processing unit (CPU), a memory, a graphics adaptor, a network adapter, a storage device, a system bus and/or a transceiver. The transceiver includes a display and a keyboard. The transceiver may further include a pointing device or a mouse. In some embodiments, the display may include a touch screen display.

The memory holds instructions and data used by the processor. The graphics adapter displays images and/or other information on the display on the transceiver. The network adapter couples the transceiver to a network. The storage device comprises any device or system capable of holding data. The storage device may be fixed or portable. The portable storage may include a flash storage, a USB storage, an external hard drive, external CD-ROM or DVD-ROM drives, a memory stick, a secure digital storage (SD) card, and/or any combination thereof. The fixed storage may include a solid-state memory device or a hard-drive, cloud-storage, and/or any combination of.

The network enables communication between a client and a server coupled to a database and/or a client to a database. The network can include links using technologies such as Wi-Fi, Wi-Max, 2G, 3G, 4G, 5G, Universal Mobile Telecommunications System (UMTS), Ethernet, integrated services digital network (ISDN), digital subscriber line (DSL), asynchronous transfer mode (ATM), InfiniBand, PCI Express Advanced Switching, and/or any combination thereof.

The network may comprise network protocols. The network protocols may include transmission control protocol/Internet protocol (TCP/IP), a multi-protocol label switching (MPLS), a user datagram protocol (UDP), a multi-protocol label switching (MPLS), a user datagram protocol (UDP), a hypertext transport protocol (HTTP), a simple mail transfer protocol (SMTP), a file transfer protocol (FTP), a lightweight directory access protocol (LDAP), a code division multiple access (CDMA), wideband code division multiple access (WCDMA), global system for mobile communications (GSM), high-speed downlink packet access (HSDPA, and/or any combination thereof.

The data exchanged over the network may be represented using different formats. The formats including the hypertext markup language (HTML), the extensible markup language (XML). In addition, all or a portion of the data can be encrypted using conventional encryption technologies, such as secure sockets layer (SSL), secure HTTP, and/or virtual private networks (VPNs), Internet Protocol Security (IPsec), and/or any combination thereof.

In one embodiment, the client (e.g., a piece of computer hardware or software) or transceiver, executes a browser which may connect to a database within a cloud storage via network. The network may include the Internet. The network may further include at least one or more of a LAN, a MAN, a WAN, a mobile, wired or wireless network, a private network, a virtual network, and/or any combination thereof. While only a single client is shown, it is understood that very large numbers, from 0 to millions) of clients may be supported and can be in communication with the database at any time. The client may be a desktop computer or a portable computer. The portable computers may include a laptop, a notebook, a netbook, a tablet, a smartphone, and/or any combination thereof.

The surgeon and/or user should enter the designated website or mobile app for access to the preoperative planning software. In one embodiment, the software application may comprise a web-based software application or cloud-based software, which a user may access through a web browser such as Internet Explorer, Chrome, Firefox or Safari. The software application is housed in a server that displays the information through a native web environment as if the user was browsing a website. No additional software or hardware may be purchased or software downloaded to a user's computer. Alternatively, the software application may comprise a mobile software application, which is a software application designed to operate on a mobile device such as a phone, tablet or watch. Such mobile applications are native applications and may require software for different operating systems (e.g., Android, iOS, etc.).

The browser may be coupled to the database within a storage system. The database includes records. The records include user credentials for authentication. The user credentials may comprise first name, last name, email address, password, and/or any combination thereof. The user credentials may be placed into a folder. The folder may include other records, further including uploaded images, processed images, measurements, and/or any combination thereof.

In one embodiment, the transceiver and/or client is adapted to execute computer program modules for providing the functionality for the home, log-in and/or sign-up. The term “module” refers to a computer program utilized to provide a specified functionality. A module may be implemented in hardware, firmware, and/or software. In one embodiment, the program modules are stored on the storage device, loaded into the memory, and executed by the processor.

Accessing the Preoperative Planning Software Application (Home, Log-In & Sign-Up)

The step of acquiring, generating or creating a preoperative plan using the preoperative surgery planning software 50 may further comprise the step of accessing the software application 95. The step of accessing the software application 95 may comprise the steps of creating a new user profile using a new-user sign-up graphic user interface; and/or accessing current user profile using a current user log-in graphic user interface. The step of accessing the software application 95 may further include the step of accessing or entering the web page, web portal or website 150. The step of accessing the software application may further include the step of uploading one or more operative images from a storage device 100.

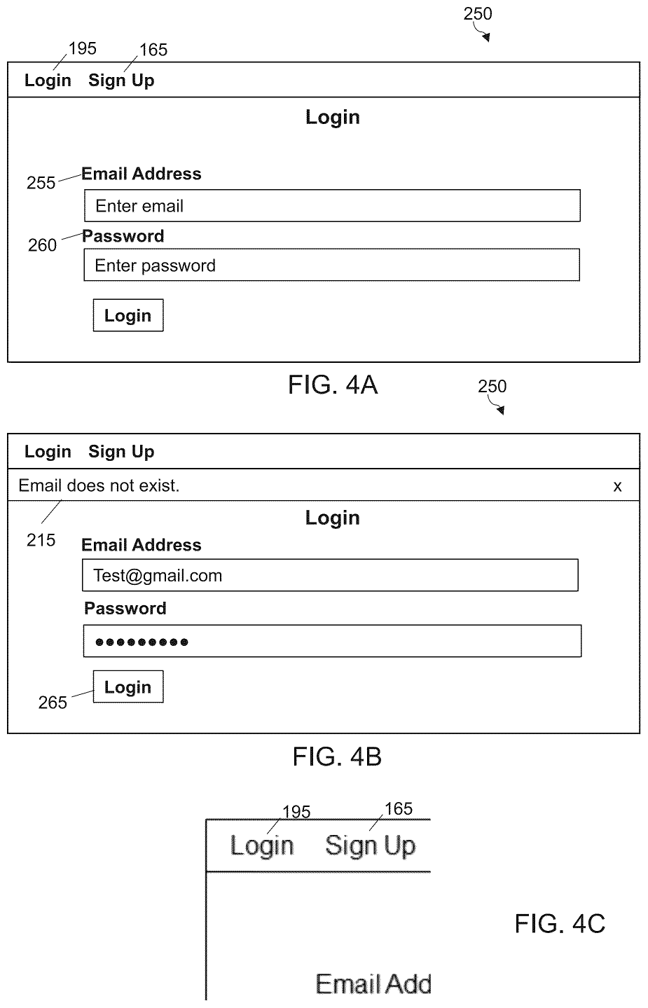

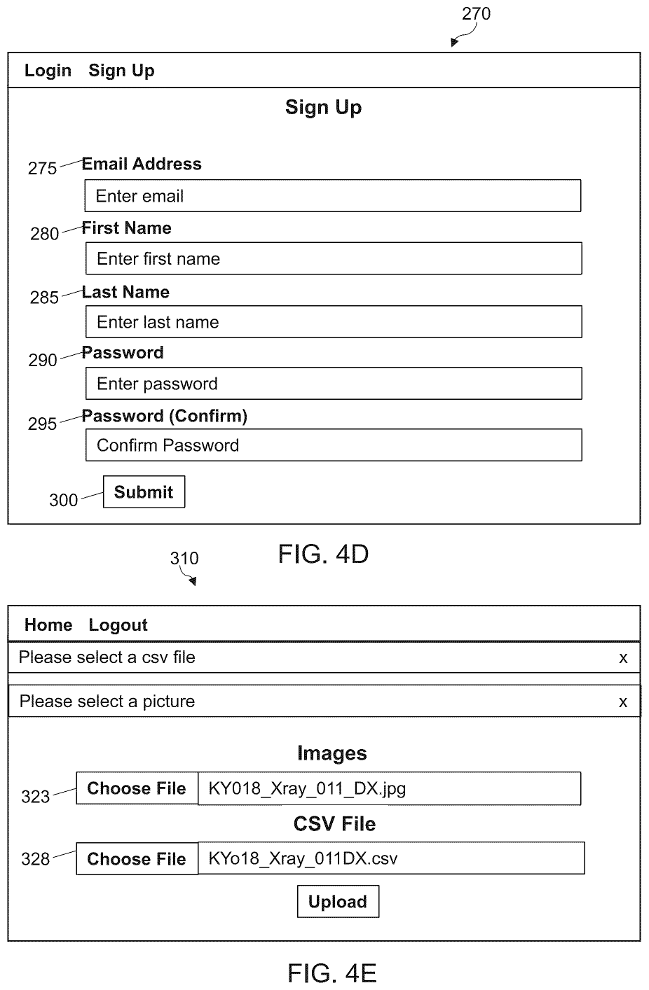

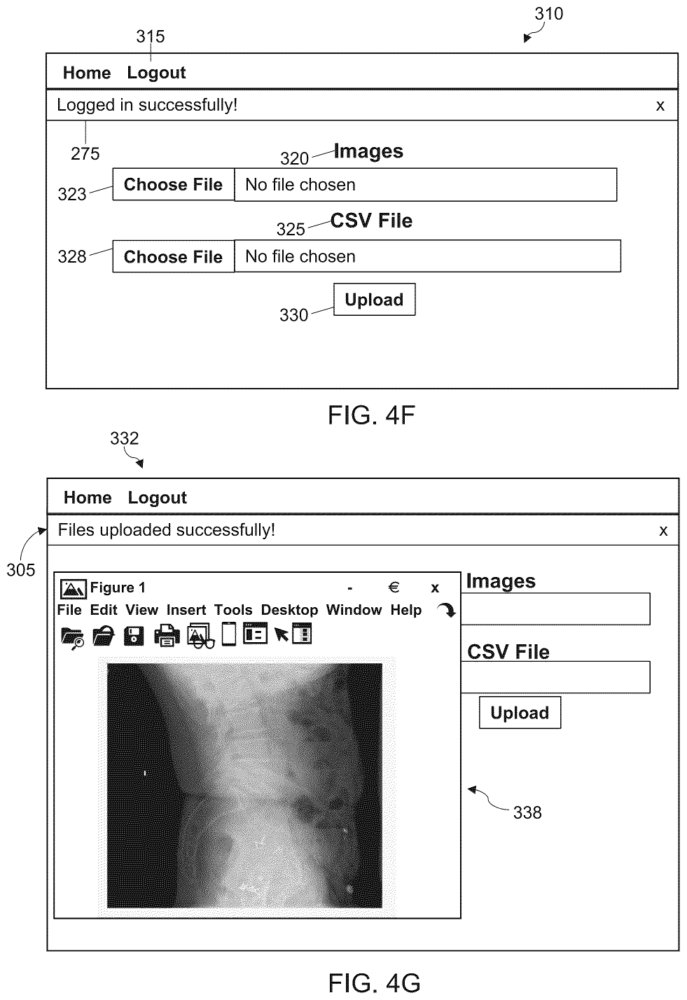

FIG. 3C is a flowchart 145 illustrating the step of accessing the software 95. The user and/or surgeon should enter the software application, the website or web portal via the client (e.g., computer) 150. By entering the software application, the website or web portal via the client (e.g., computer) 150, the surgeon and/or other user enters or views 155 a first graphic user interface (GUI) or first web page, the first GUI comprises a home page 250 as shown in FIG. 4A-4B. The home page 250 displays two options that requires the user to question whether they have an existing account to the software application 160.

With reference to FIGS. 3C and 4C, if the surgeon and/or other user does not have an existing account and/or log-in credentials, the surgeon and/or user should click or press the sign-up button 165 in the top left corner of the home page 250. The “sign-up” button 165 will redirect the website to a second graphic user interface or web page 270. The second GUI 270 comprises the “sign-up” page. The second GUI or webpage 270 asks for new users or surgeons to create a new account by entering personal information 170 before proceeding to use the software application. The personal information 170 comprises a unique email address 275, a first name 280, a last name 285, a unique password 290, and re-entry of the new password 295. A unique email address 275 should be entered, not an existing email address from an existing account. The user and/or surgeon submits the new account information 180, the information is validated and a new profile with a new account will be created onto the database 190 and stored on a storage device 205. Once validated, the second GUI 270 will be redirected to the home page 250 to enter their log-in credentials 170. A “successful” pop-up message 175 to tell the user and/or surgeon that the account was created successfully may be displayed on the home page 250.

With reference to FIGS. 3C and 4A-4B, if the user and/or surgeon has their log-in credentials, the user and/or surgeon's must enter the login credentials to access their account 195. As we can see in FIG. 4A-4B, the home page 250 comprises fields to allow the users or surgeons to enter their log-in credentials, the log-in credentials include a unique email address 255 and password 260 to access their account. The surgeon or other user can submit the email address 255 and password 260 by pressing the “log-in” button 265. The software application will confirm the log-in credentials 210, and once the log-in credentials 255, 260 are validated and/or authenticated, the software can access the database 190 that is stored in a storage device 205 to retrieve the data stored on the surgeon's profile and indicating that the surgeon or other user logged-in successfully 245. The software may provide a “successful” pop-up message 275 for successfully logging-in to the software as shown in FIG. 4F. The “successful” pop up message 275 may comprise a first color, the color may include a green color. However, any other color can be contemplated. As described herein, the storage devices 205 may include internal (part of computer's hardware), external (installed outside of computer), virtual (cloud based) and/or portable storage.

If the log-in credentials are not validated or authenticated, the home page 250 may display an “error” pop-up message 215 to tell the user that the log-in credentials 195 provided are invalid as shown in FIG. 4E. The “error” pop-up message 215 may comprise a second color, the second color may include a red color. However, any other color may be contemplated. Alternatively, If the log-in credentials are not validated or authenticated, the website may provide a password reset option by entering the email address 220 that is attached or tied to the surgeon's or other user's profile. The software application must confirm whether the email address 220 matches the current profile 225. If the software validates the entered email address, a new reset password email will be sent 230 to the confirmed email address. The surgeon or other user will enter another unique reset password 235 and submit the reset password 240. Once the reset password is accepted, the new log-in credentials 195 is stored within the database 190 that may be stored on a storage device 205.

With reference to FIGS. 4E-4F, once log-in-credentials are successful, the website will be redirected to a third graphic user interface or web page 310 or the “upload data set” page. The “upload data set” page 310 asks for the user and/or surgeon to upload one or more data sets 320, 325. The one or more data sets 320, 325 may comprise at least one or more operative images 320. The user and/or surgeon may press on the “choose file” button 323 to select the patient-specific one or more operative images 320 from a storage device. The one or more operative images 320 may comprise raw images or modified images. The one or more operative images 320 comprises a portion of a spine in a spine region. The portion of a spine in a spine region may comprise a single spinal segment within a single spinal region. The portion of a spine in a spine region may comprise a plurality of spinal segments in a plurality of spinal regions. The portion of a spine a spine region may comprise a plurality of spinal segments in a single spinal region. The spinal region may comprise a cervical region, a thoracic region, a lumbar region, a sacral region, and/or any combination thereof. The one or more data sets 320, 325 may comprise numerical data 325. The numerical data 325 may be disposed into a spreadsheet or a CSV file.

Similarly, users and/or surgeons can also select the patient specific digitized, modified images, the modified images. The modified images comprise one or more operative images that are modified by a surgeon with desired anatomical landmarks. The desired anatomical landmarks may include highlighting perimeter or borders of one or more spinal segments with markers or indicia to indicate his initial guess of vertebrae(s) of interest. The one or more operative images may further comprise static or dynamic images. The one or more operative images may comprise a portion of a patient's spine in a spine region. The spine region comprises a cervical region, a thoracic region, a lumbar region, a sacral region, and/or any combination thereof. The user and/or surgeon may press or click the second “Choose File” button 328 under the file typically stored under a “CSV” format 325 and/or any other desired format (please see FIG. 4E-4F). Finally, the surgeon and/or user can click on ‘Upload’ button 330 to transmit the data set 320, 325 to the database 190 and to store the data set 320, 325 onto a storage device 205. In one exemplary embodiment, the storage device 205 comprises virtual storage, the virtual storage may include a Box cloud database.

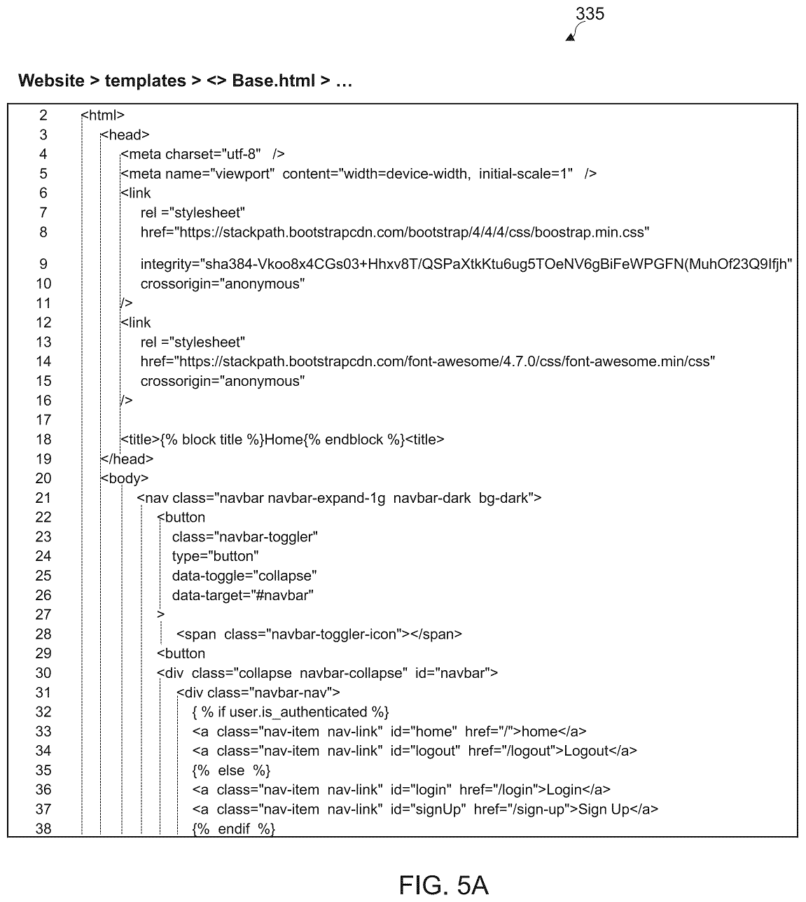

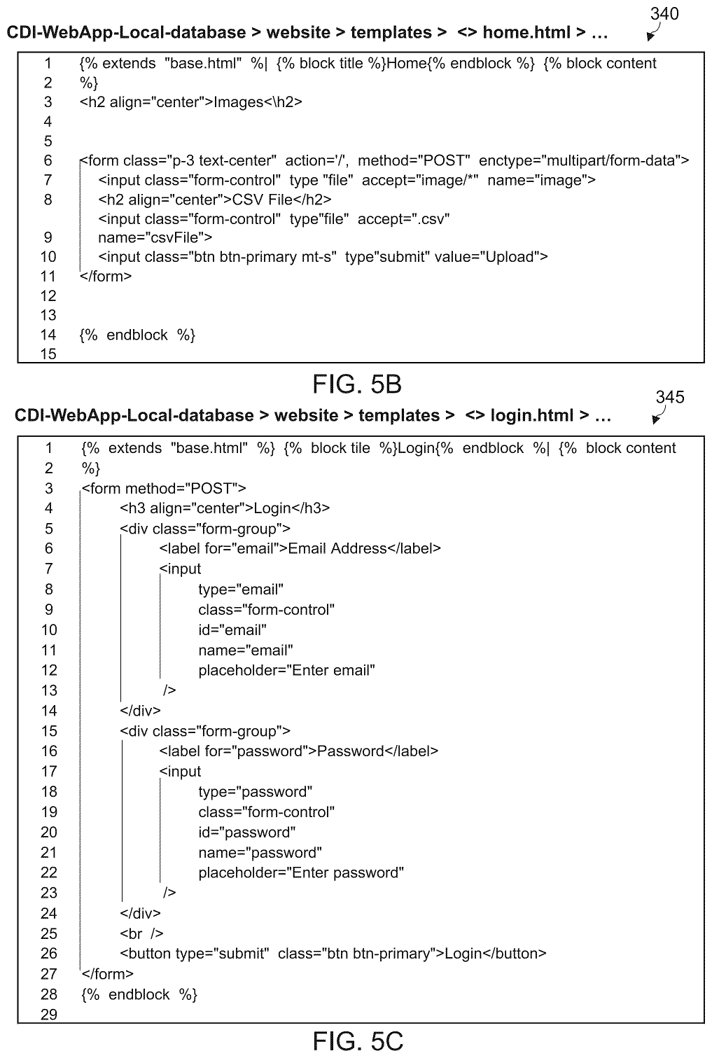

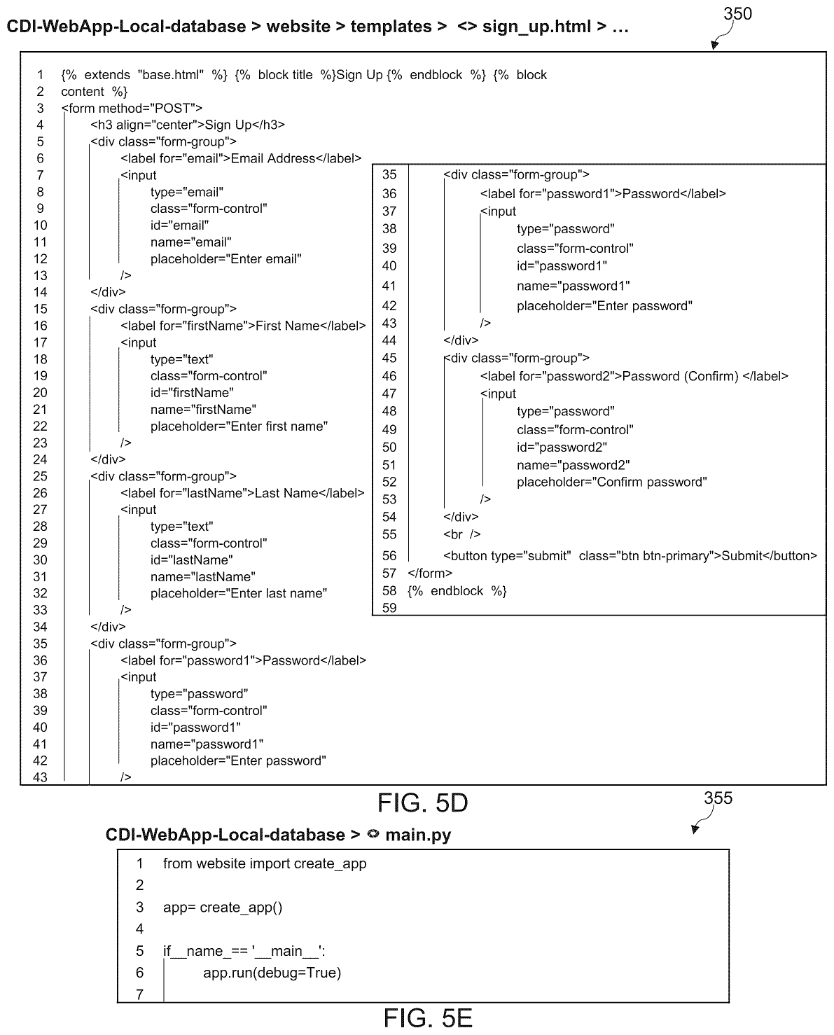

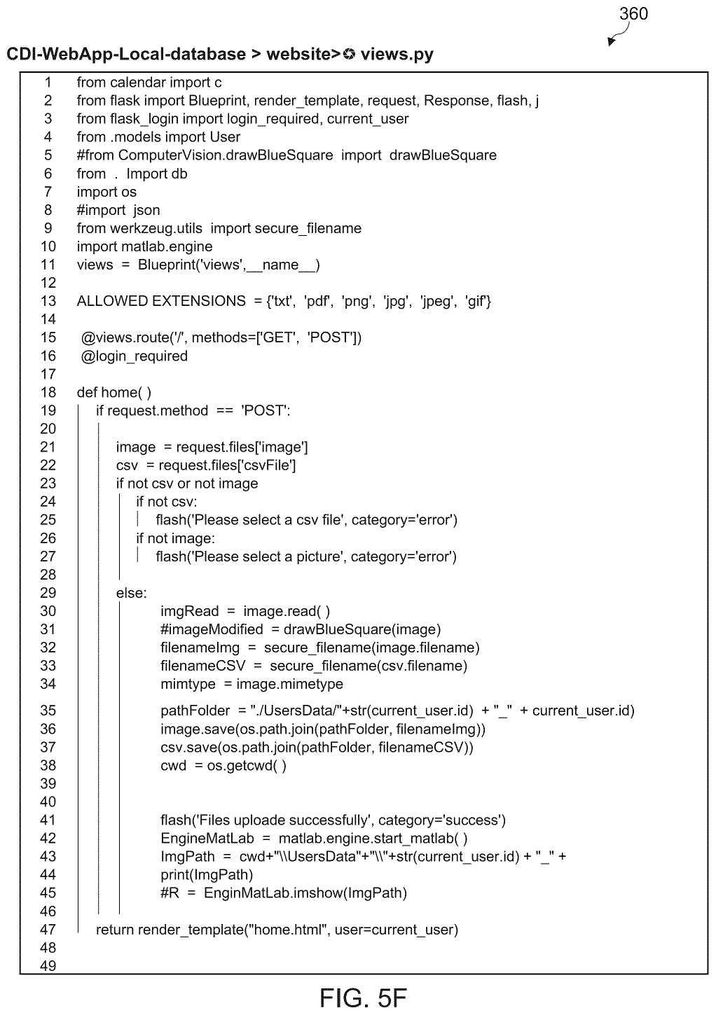

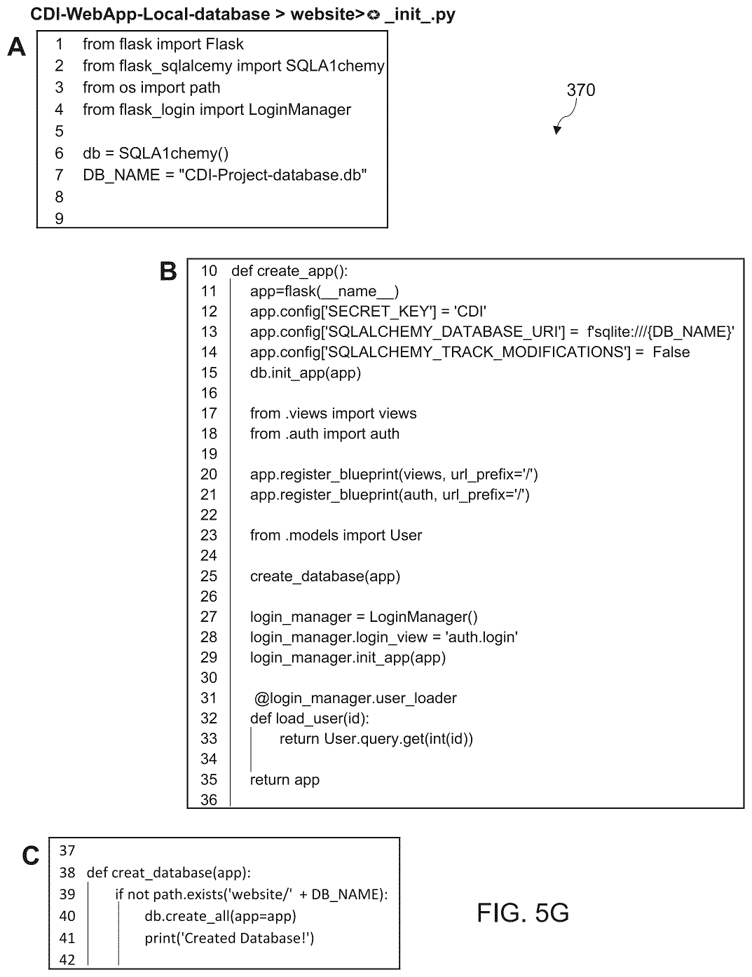

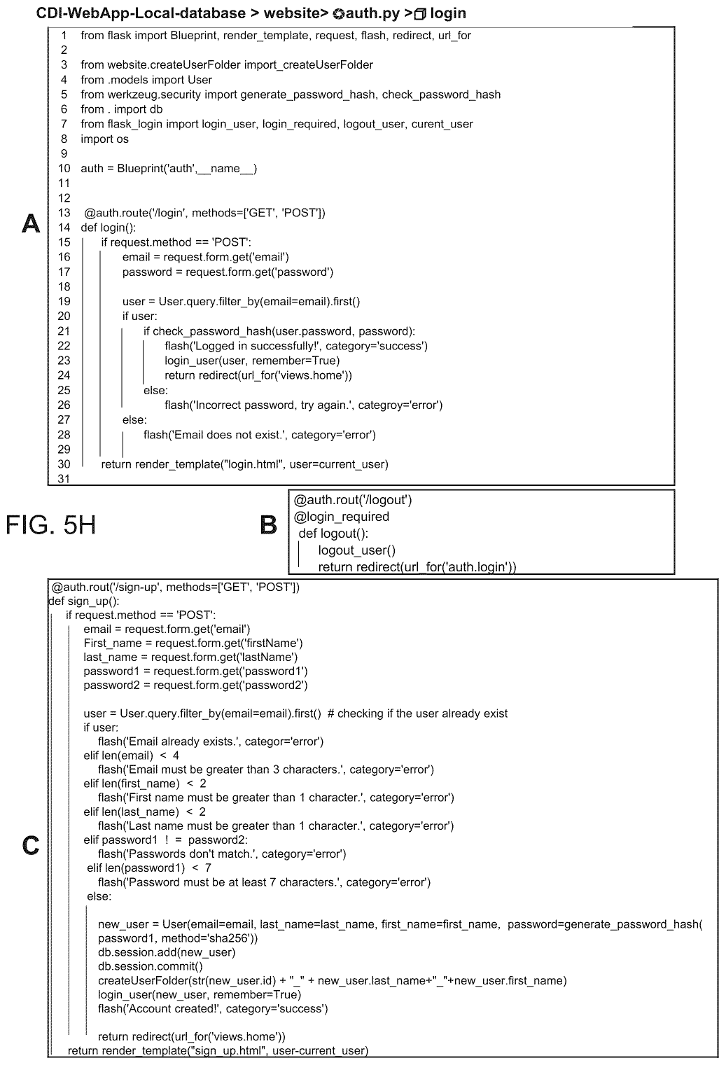

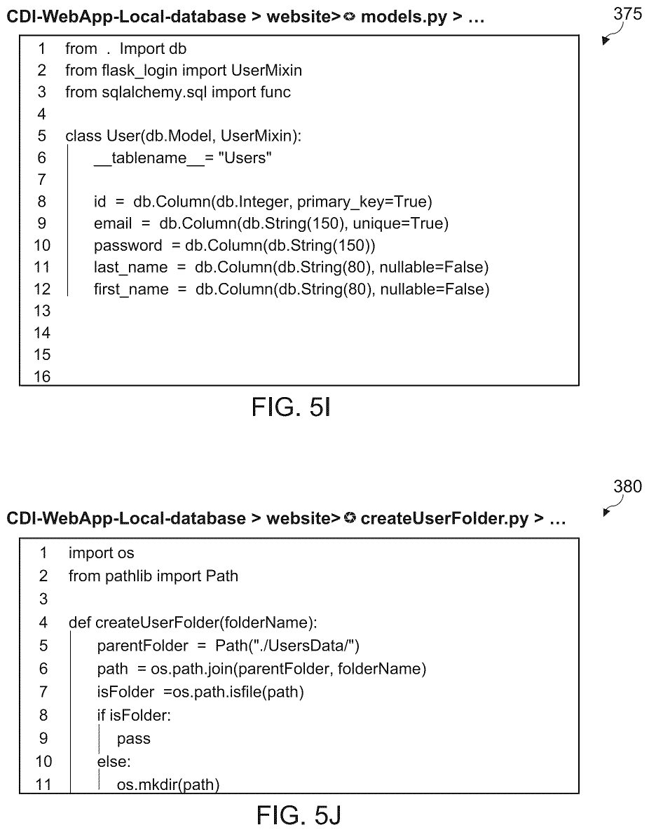

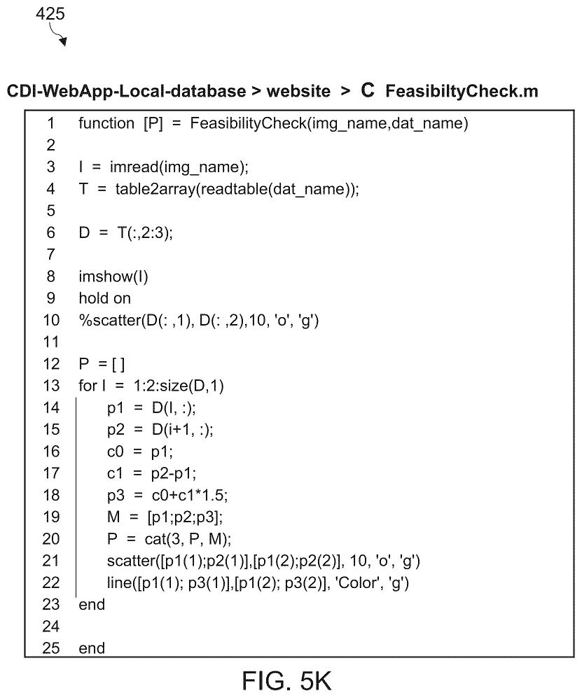

With reference to FIG. 4G, the uploaded one or more operative images initiates the views module 360 to display the data set results onto a fourth graphic user interface or web page 332. If uploading is completed and successful, the software will request further input from the views module 360 (see FIG. 5F) to initiate a MATLAB engine and/or run the MATLAB function called Feasibility Check module or feasibilitycheck.m module 425 as shown in FIG. 5K. If uploading is not successful or not successful, a pop-up message 305 may be displayed onto the fourth web page or GUI 332. Accordingly, the step of generating one or more virtual models of the target anatomy 110; and/or the calculating one or more SSM from the one or more virtual models of the target anatomy to create a data set 115 are engaged. The feasibility Check module or feasibilitycheck.m module 425 may output or display at least one sample of the one or more virtual models 338 (e.g., manipulated and/or processed images) onto the fourth graphic user interface or web page 332.

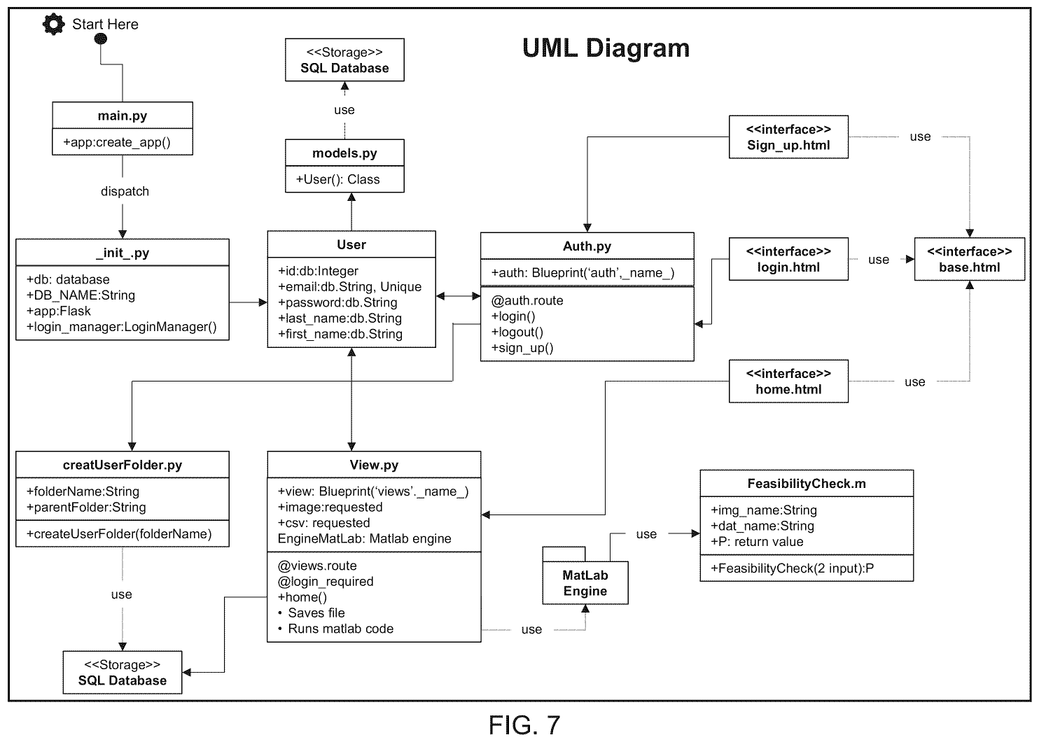

With reference to FIGS. 5A-5K and 7, the step of accessing the software application 95 comprises one or more program modules, the one or more program modules include a base module 335, a home module 340, a log-in module 345 and/or a sign-up module 350. Each of the base module 335, a home module 340, a log-in module 345 and/or a sign-up module 350 provides the structure for the webpages or the graphic user interface (GUIs). The program modules may further include a main module 355, a views module 360, an initializer module 365, an authentication module 370, a models module 375, a createuserfolder module 380 and/or any combination thereof. The program modules may be loaded, activated or initiated as part of the software application and/or the web-based software application. One or more of the program modules comprises HTML and/or python code, the HTML code is used to structure the web pages, e.g., the graphic user interfaces

In one embodiment, the software application comprises a base HTML module or a base module 335 as shown in FIG. 5A-5B. The base module 335 specifies the base URL information, font types, and other specific design for all the HTML files. Instead of specifying the style of the pages in every module, they are written in base.html. base.html is imported in home module, the log-in module 340 and/or the sign-up module 345.

In another embodiment, the software application comprises a home HTML module or a home module 340 as shown in FIG. 5B. The Home module 340 displays the home webpage or interface 250 we saw in FIG. 4A-4B above. It extends from base.html or base module 335 that uses a form class type of p-3 text-center with a post method. It creates two inputs (first input type image and second input type CSV file) as indicated in code lines 7 and 9 respectively in FIG. 5A and a third input type of ‘submit’ to create an event to upload the files. It passes the three inputs to views.py. or views module 360.

In another embodiment, the software application comprises a log-in HTML module or a log-in module 345 as shown in FIG. 5C. The Log-in HTML module or the log-in module 345 is represented by interfaces on the home page 250 within FIGS. 4A-4B above. It extends and/or interacts from base.html or the base module 335 and is a POST method that requests at least three types of inputs. The inputs may include: an email 255 which is a type ‘email’, a password 260 which is a type ‘password’, and a button type of ‘submit’. Once the ‘login’ event occurs, the request for email 255 and password 260 will be passed to auth.py or the authentication module 370, then depending on the outcome of home.html and/or the home module 340 will receive a command from auth.py and/or the authentication module 370 to display a message through the flask command.

In another embodiment, the software application comprises a sign-up HTML module or a sign-up module 350 as shown in FIG. 5D. The sign-up module 350 is represented by the interfaces on the “sign-up” GUI 270 shown in FIG. 4D. It extends from base.html or base module 335 and is a POST method that requests a plurality of inputs. The plurality of inputs includes an email which is a type ‘email’, two passwords which are type ‘password’ to check if they are identical, first and last name which is type ‘text’, and a button type of ‘submit’ 300. Once the ‘submit’ event occurs, the request for email 275, first name 280, last name 285, first password 290, and second password 295 will be passed to auth.py or the authentication module 370, then depending on the outcome sign_up.html or sign-up module 350 will receive a command from auth.py or the authentication module 370 to display the message through the flask command.

In another embodiment, the software application comprises a main.py and/or a main app module 355 as shown in FIG. 5E. The main app module 355 deploys the whole application. It creates a variable called ‘app’ using create_app( ) that is imported from the _init_.py or the initializer module 365 directory folder as shown in FIG. 5G. After the app is created a debugger will be turned on to notify the developer of any error or crash of the website. The code may include HTML or python.

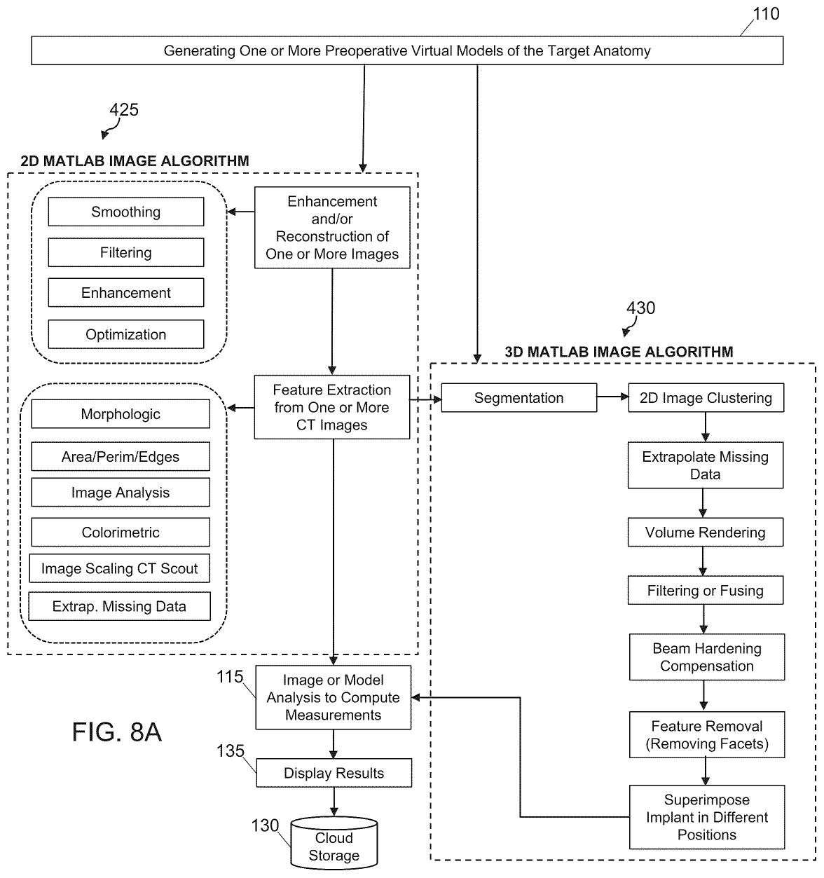

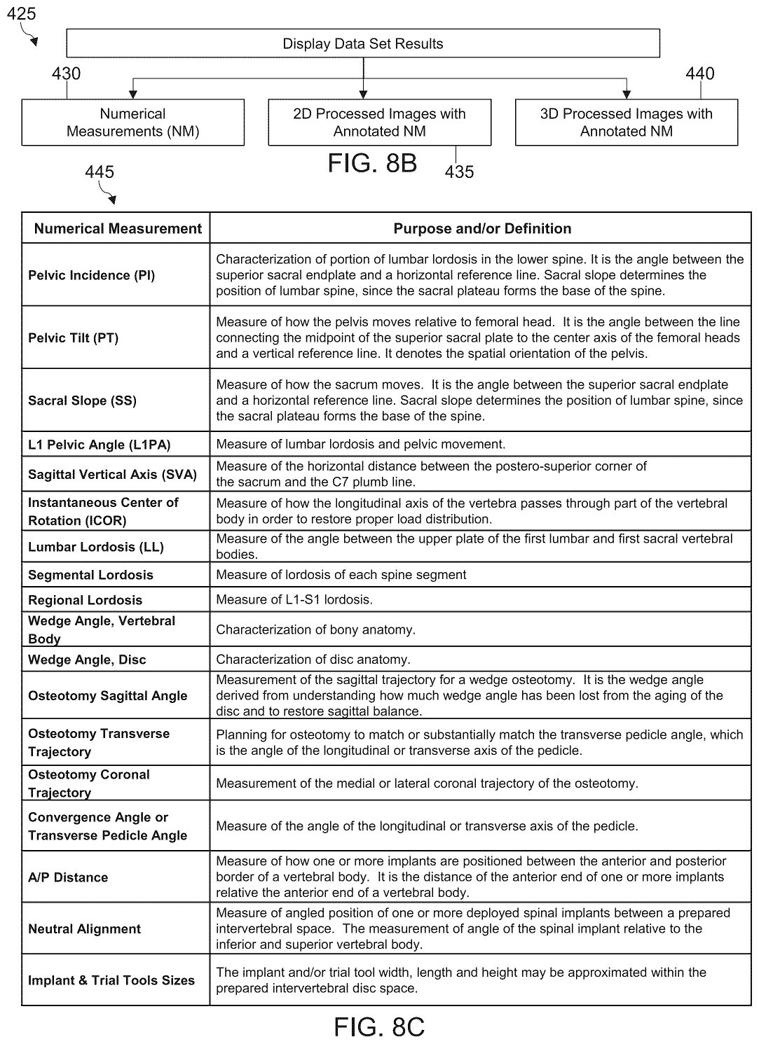

In another embodiment, the software application comprises a views.py and/or a views module 360 as shown in FIG. 5F. The views module 360 contains the code necessary for the upload page or GUI 310 when the user successfully logged in. Code line 16 in FIG. 5F requests a successful login before proceeding to upload an image or CSV file. A new function called home( ) is defined in this file that requests input from home.html and/or the home module 340 (see FIG. 5B). It will check for the right input and will start a MATLAB engine to run a MATLAB function called Feasibility Check (see FIG. 8A). The code may include python or HTML.

In another embodiment, the software application comprises an init_.py and/or an initializer module 365 as shown in FIG. 5G(A)-(C). This initializer module 365 defines the function called create_app and create_database. Create_app is a function that does not take any arguments but returns a Flask variable called “app.” In FIG. 5G(A), the function comprises the different libraries used. In FIG. 5G(B), illustrates the creation of a new function called create_app. When the create_app function is called, it will create a secret key called CDI, it will create a SQL database to store user credentials (code line 25), and it will load the current user to start the process. In FIG. 5G(C), illustrates another function that is called “create_database” that takes “app” as an argument. More specifically, the create_database is a void function that takes a Flask app as an argument. It checks if there is an existing database called “CDI-Project_database.db” then if there is not an existing file, it will create a new SQL database called “CDI-Project_database.db”, please refer to FIG. 5G(C) to see the details. The code may include python or HTML.

In another embodiment, the software application comprises an auth.py and/or an authentication module 370 as shown in FIG. 5H(A)-(C). The authentication module 370 comprises at least three functions, the functions include creates credential information for a user, retrieves data from the user database for secured log-in, and lets the user log out successfully. Log-in is one of the three functions in the auth.py, please refer to FIG. 5H(A). It requests inputs from the login.html file such as email and password entered. Login checks if the password and email exist in the database. If the user credentials aren't in the database, an error message will be displayed on the login.html and/or the log-in module 345 (see FIG. 4B); otherwise, the page will be redirected to home.html or the home module 350 (code line 24 FIG. 5H(A)). Logout is another function in the authentication module 370 that securely signs out the current user. It will require a successful login to log out a user. After successful logout, the page will be redirected to login.html or the log-in module 345. Please refer to FIG. 5H(B) for code details. The code may include python or HTML.

Sign-up is the last function that is created in the authentication module 370. This function will be called when a new user is detected. First, the user will need to press the button ‘Sign up’ in login.html or log-in module 345 and/or signup.html or sign-up module 350 (FIGS. 4A-4D). The function will request 5 inputs from signup.html or the sign-up module 350. The code will check whether the email exists in the database because emails are the one unique input from the plurality of inputs. Users must have a unique email address, but they can have the same names or passwords. When at least one or more inputs are valid, a new user will be added to the database and the page will be redirected to login.html or the log-in module 345. Alternatively, when at least five inputs are valid, a new user will be added to the database and the page will be redirected to login.html and/or the log-in module 345. Please refer to FIG. 5H(C) for the detailed code of the signup function.

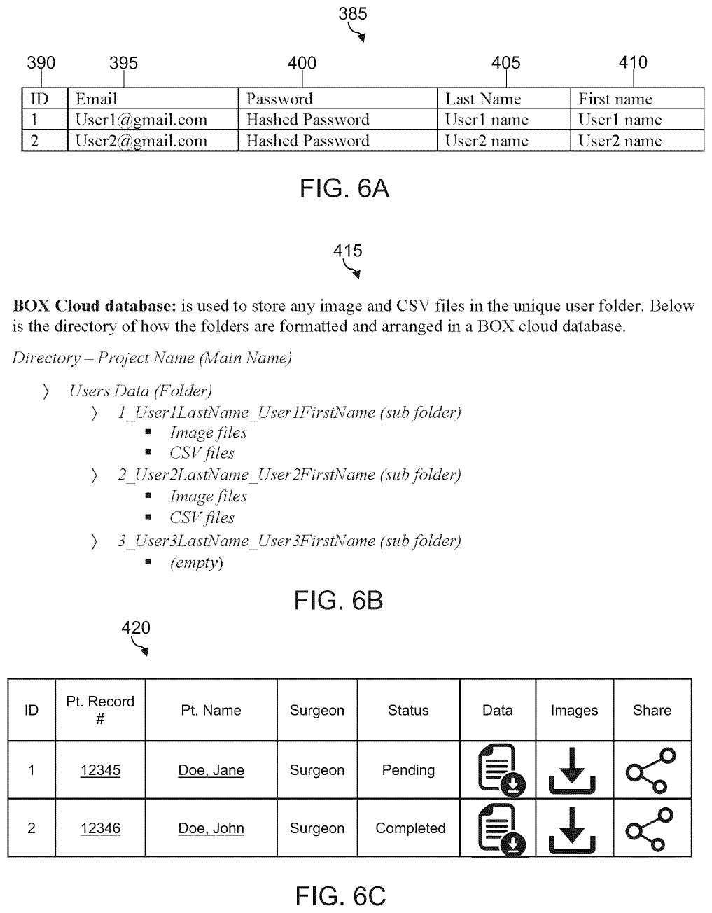

In another embodiment, the software application comprises a models module 375 as shown in FIG. 5I. The models module 375 has the main purpose of creating classes. In this file, a user is the only class that creates the table 385 name called “Users”. It creates a class type of database that comprises a table 385 with multiple columns that includes new user information 170, the new user information comprises a unique ID number 390, email 395, password 400, last name 405, and first name 410 (see FIG. 6A). Email 395 is a unique variable while ID number 390 is automatically assigned to the user or surgeon when creating an account.

In another embodiment, the software application comprises a user.py and/or createuserfolder module 380 as shown in FIG. 5J. The createuserfolder module 380 contains at least one function, the at least one function includes the creation of a new user folder when an account is created. During the sign-up phase, we call this function to create an empty folder for the user. It takes one argument called folder name and returns nothing. The folder name is specified to the user that user their unique ID number 390, first name 410, and last name 405 (for example, the folder name for a user with an ID of 1 is 1_firstName_lastName). Furthermore, each folder for each user may include one or more data sets from one or more patients.

In another embodiment, the software application comprises a store module 415, 420 as shown in FIG. 6B-6C. The store module 415 may comprise at least two functions that stores one or more folders for each user into a storage device. The store module 415 may refer to the createuserfolder module 380 to store the folder for each user and/or surgeon. Each of the one or more folders may comprise one or more data sets from one or more patients under each unique user or surgeon. A second function may comprise how the directory of each folder is formatted and arranged within a storage device as shown in FIGS. 6B and 6C. The arrangement may comprise a basic list format 415 (see FIG. 6B) and/or a table format 420 (see FIG. 6C).

Preoperative Planning Software Application Image Processing

Once the users and/or surgeons have successfully uploaded one or more operative images to the software application, the MATLAB engine is initiated to proceed to the step of generating one or more virtual models of the target anatomy 110; and/or the calculating one or more SSM from the one or more virtual models of the target anatomy to create a data set 115 are engaged (see FIGS. 3A-3B). As previously disclosed herein, the surgeon and/or user can upload patient specific one or more operative images, the one or more operative images may include raw or modified images. The modified one or more operative images may include the surgeon's or user's initial guess of the targeted anatomy. The one or more operative images may further comprise static or dynamic images. The one or more operative images may comprise a portion of a patient's spine in a spine region. The spine region comprises a cervical region, a thoracic region, a lumbar region, a sacral region, and/or any combination thereof.

With reference to FIGS. 8A-8B, the flowchart and table illustrate at least one embodiments of one or more steps, functions or modules comprising the step of: generating one or more virtual models of the target anatomy 110; and/or the calculating one or more SSM from the one or more virtual models of the target anatomy to create a data set 115. The flowchart may further illustrate the steps of: displaying results 135 and/or storing data set(s) onto a storage device 130. Alternatively, the steps may comprise generating one or more virtual models of the target anatomy 110 to create a first data set and the calculating one or more SSM from the one or more virtual models of the target anatomy to create a second data set 115.

With reference to FIGS. 8A, the step of generating one or more virtual models of the target anatomy 110 may comprise one or more program modules. The one or more program modules may comprise a feasibility check module or feasibilitycheck.m module 425 (see FIG. 5K). The feasibility module 425 comprises one or more functions, the functions include running the MATLAB engine; confirming target anatomy; and/or displaying the feasibility check results. The feasibility module 425 is activated after it communicates or engages with the views module 360. A library called matlab.engine is used to communicate to python (the library works for the python versions of 2.9 or below). The matlab.engine may comprise a 2D matlab file 425 to create one or more 2D virtual models 435 and/or a 3D matlab file 425 to create one or more 3D virtual models 440.

The feasibility module 425 runs the MATLAB file that takes two inputs as an argument (at least one operative image, the at least one operative image may comprise a raw or modified image) and returns a variable P. In one embodiment, the feasibility module 425 will read and manipulate the one or more operative images and converts them into a CSV file to an array, and draws points from the dat_name to the img_name to confirm that the one or more images comprises the target anatomy. If the target anatomy is improper or invalid, a pop-up message 305 will be displayed (see FIG. 4G). However, the target anatomy is confirmed, the feasibility module 425 may display feasibility images 338 onto a fourth webpage or GUI 820 on FIG. 4G. The one or more operative images that were feasibility checked may comprise one or more defined anatomical landmarks. In another embodiment, the feasibility check module 425 may comprise the function of image processing and/or the image processing module.

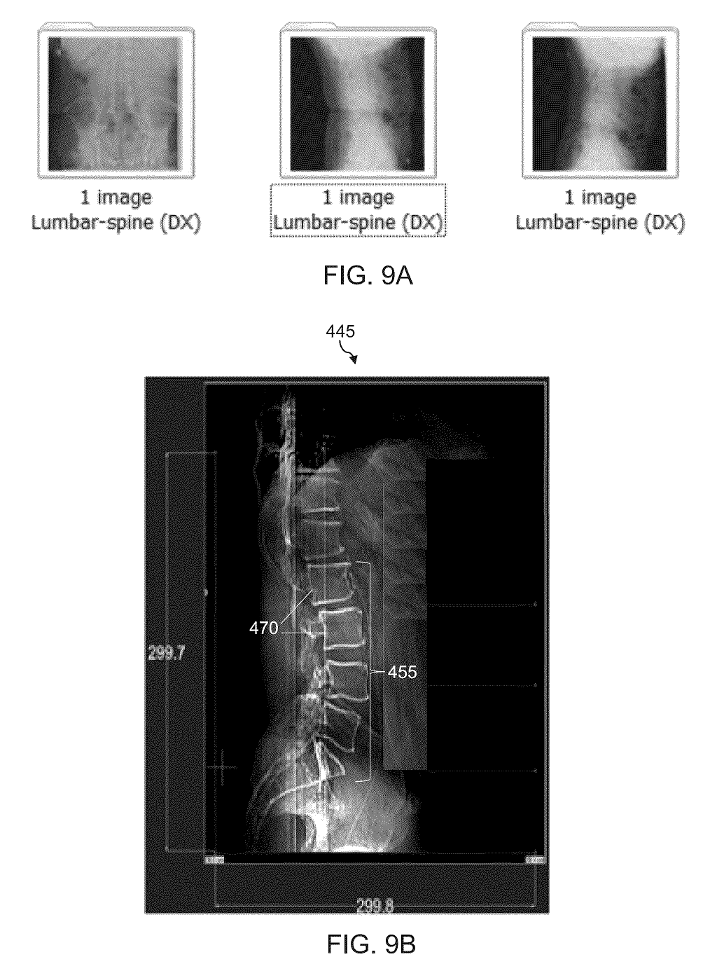

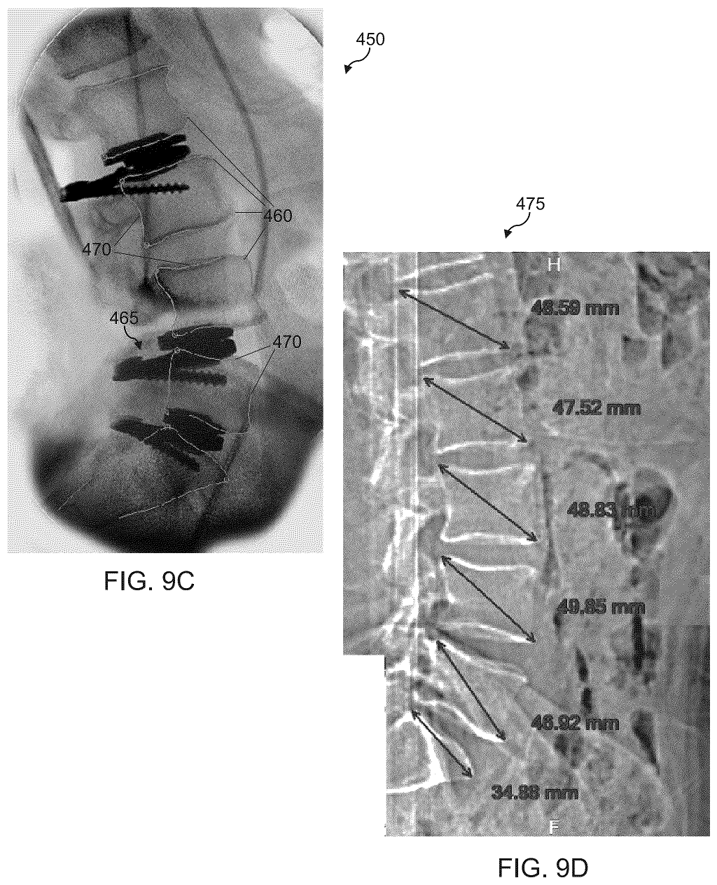

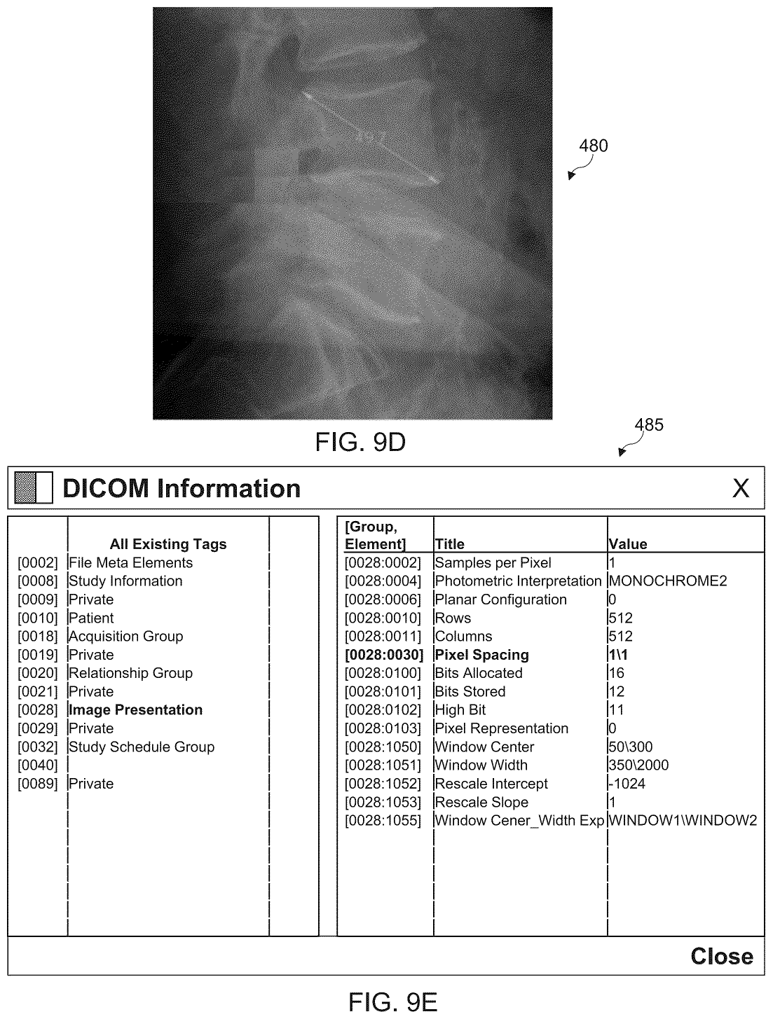

With reference to FIGS. 8A-8C, 9A-9E, and/or 10A-10D, the software application and/or the step of generating one or more virtual models of the target anatomy 110 may comprise an image processing module. Image processing and/or the image processing module is defined as the set of manipulation and/or computational techniques, functions or modules using the original or native one or more operative images to improve or enhance the image quality to develop one or more virtual models for acquiring selected surgical measurements (e.g., quantitative analysis) in preparation for operative surgery.

With reference to FIGS. 9B-9E, the image processing module may comprise a scaling module or a scaling function. The scaling module may be used to scale at least one raw and/or modified operative image uploaded by the user and/or surgeon. The scaling module may perform one or more functions, including a geometric transformation which can be used to shrink or zoom the size of an image (or part of an image); to change the visual appearance of an image; to alter the quantity of information stored in a scene representation, and/or or as a low-level preprocessor in multi-stage image processing chain which operates on features of a particular scale. The size of the image array may be determined by the number of sampling points measured. The information at each sampling position in the array represents the average irradiance over the small sampling area and is quantized into a finite number of bits. The resolution of the image increases as we increase the number of sampling and quantization levels.

In another embodiment, the scaling module or scaling function may comprise the manipulation of at least one or more images using CT-scout as shown in FIGS. 9B-9D. CT-scout images may be known as a scanogram, topogram, localizer, scan projection radiographs, surview, or pilot scans. Scout images may comprise large slice thickness and poor spatial resolution images aimed at providing “rudimentary” images in three orthogonal, axial, sagittal, and coronal planes. In general, the scout images are projectional overviews used as reference images for acquiring an axial CT series. The scout images may be used to localize and further interrogate different anatomic structures with detailed images. Even with reduced spatial resolution, the quality of these scout images can provide “sufficient” detail of incidental pathologies which can be investigated further with appropriate (clinical, biochemical, and imaging) investigations. Furthermore, parameters can be altered to improve the spatial resolution of the localizer images to improve diagnostic yield on a DICOM or CT-scout interface 485 as shown in FIG. 9E.

Scaling CT-scout images 445, 450, 475, 480 may be used as a base for scaling all subsequent one or more operative images in static or dynamic positions; allowing markers or indicia 470 that highlight anatomical landmarks 455, including vertebra corner points 460 that enable the mapping of the patient's targeted anatomy or a portion of a patient's spine; performing rigid body transformations using the markers or indicia highlighting anatomical landmarks; extrapolating and “fill-in” missing information of the targeted anatomy; and/or conducting post-operative analysis by matching post-operative images with the one or more preoperative images, including position of implant 465; and/or generate a report comparing the post-operative images from the analysis. The 2D virtual models 435 and/or 3D virtual models 440 may further comprise defined anatomical landmarks, indicia or markers, and/or annotations. The annotations may comprise numerical, alphabetical, and/or alphanumerical. The anatomical landmarks may include vertebral body or bony edges, borders, width, height, length, surface texture, surface changes, axes or axis, and/or any combination thereof. The anatomical landmarks may be disposed onto a plurality of spinal segments in a spine region. The anatomical landmarks may be disposed onto a plurality of spinal segments in a plurality of spine regions. Such anatomical landmarks assist the software to compute the one or more SSMs and the virtual models.

The indicia or markers 470 may comprise scout or reference lines, arrows, points, etc. The reference lines may be used to indicate the position of each cross-sectional image and the true size of the targeted anatomy. The markers or indicia 470 may be “burned into” the image by replacing actual image pixels, stored as a separate overlay, or, ideally, generated dynamically by a computer and/or a computer workstation software. In the last case, clinicians may be able to display specific cross-sectional images by clicking the appropriate scout or reference line in the localizer image. To dynamically generate scout lines, the workstation software must use orientation and slice location information in each cross-sectional image, as well as reference information in the localizer. The markers or indicia 470 may be added by a surgeon and/or user to create one or more modified operative images. The markers or indicia 470 may be added by the software application and/or the scaling module or function.

The scaling module or function may recognize the markers or indicia 470 as movingpoints, fixedpoints, and/or control points. The scale module or function will call upon tform, which tform will take the pairs of control points, movingpoints, and fixedpoints and uses them to infer the geometric transformation specified by the transformation type. Such function is expressed by tform=fitgeotrans*(movingpoints, fixedpoints, transformationtype). Subsequently, a forward geometric transformation will be applied to the entire targeted anatomy. Such function is expressed by [x,y]=transformpointsforward*(tform, u, v). True sizes, including width, depth, height, length, may be approximated. True sizes may also be displayed onto the scaled images, including numerical annotations.

In another embodiment, the software application and/or the image processing module may further comprise a transformation module or transformation function. A transformation is a geometric transformation of a Euclidean space that preserves the Euclidean distance between every pair of points. Any object will keep the same shape and size after a proper rigid transformation. The transformation function may comprise 2D or 3D transformations. The transformations may further comprise a rigid body transformation (e.g., preserves distance and angles), a conformal transformation (e.g., preserves angles), an affine transformation (e.g., preserves parallelism), rotation and/or scaling. In one exemplary embodiment, the transformation module or function comprises a rigid body transformation to preserve shape and size of an object. The properties preserved under a rigid-body transformation includes distance (lengths of segments remain the same), angle measures (angles remain the same), parallelism (parallel lines remain parallel), collinearity (points remain on the same lines), and/or orientation (order remains the same).

In another embodiment, the software application and/or the image processing module may further comprise an extrapolate module and/or function. The image extrapolation module or function aims to fill the surrounding region of a sub-image, e.g., completing the object appearing in the image or predicting the unseen view from the scene picture. The extrapolated missing information is used to enable mapping of the pre-operative vertebra models into subsequent post-operative spatial positions. Such a function may further improve one or more images brightness, contrast, sharpness, color, saturation, and/or any combination thereof for easier computing and evaluation.

In another embodiment, the software application and/or image processing module may further comprise an image reconstruction module and/or function. Image reconstruction is a mathematical process of forming a virtual model using the acquired raw data. A combination of multiple data sets captured at different angles or different time steps may be used. This procedure is repeated using multiple iteration steps that maps the estimated and true values or sizes, resulting in a convergence of the reconstruction process to the final image or virtual model. There is a large variety of iterative methods including maximum likelihood expectation maximization (MLEM), maximum a posteriori (MAP), algebraic reconstruction (ARC) technique, and/or any other standard methods known in the art.

In another embodiment, the software application and/or the image processing module may further comprise an enhancement module and/or function. The image enhancement module may conduct high-level image processing or low-level image enhancement of an image to improve interpretability of the contained information. Utilization of all these techniques allow for noise and inhomogeneity reduction, contrast optimization, enhancement of edges, elimination of artifacts, and improvement of other relevant properties that are crucial for the subsequent image analysis and its accurate interpretation.

In another embodiment, the software application and/or the processing module may further comprise a visualization and/or feature extraction module or function. The visualization and/or feature extraction module or function renders the image data to visually represent anatomical and physiological imaging information in a specific form over defined dimensions, resulting in optimized output or optimized images. Through direct interaction with data, the visualization can be performed both at the initial and intermediate phases of imaging analysis—for instance, to assist segmentation and registration processes, and at the final stage to display quantitative or imaging results. Other processes or functions may be available, including illumination, surface reconstruction, shading, and/or display of quantitative and/or imaging results.

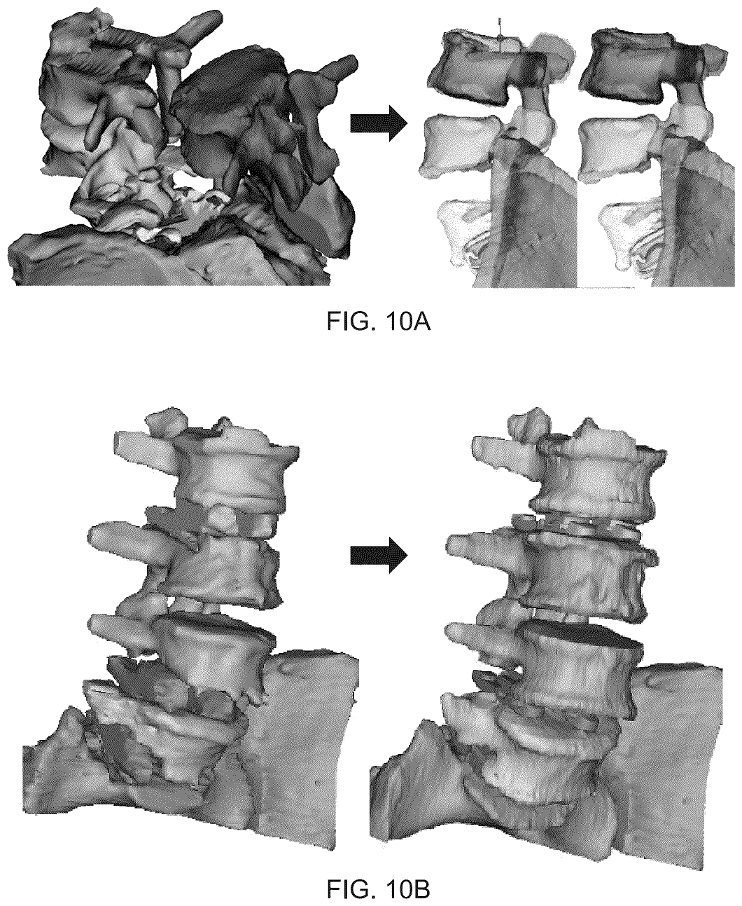

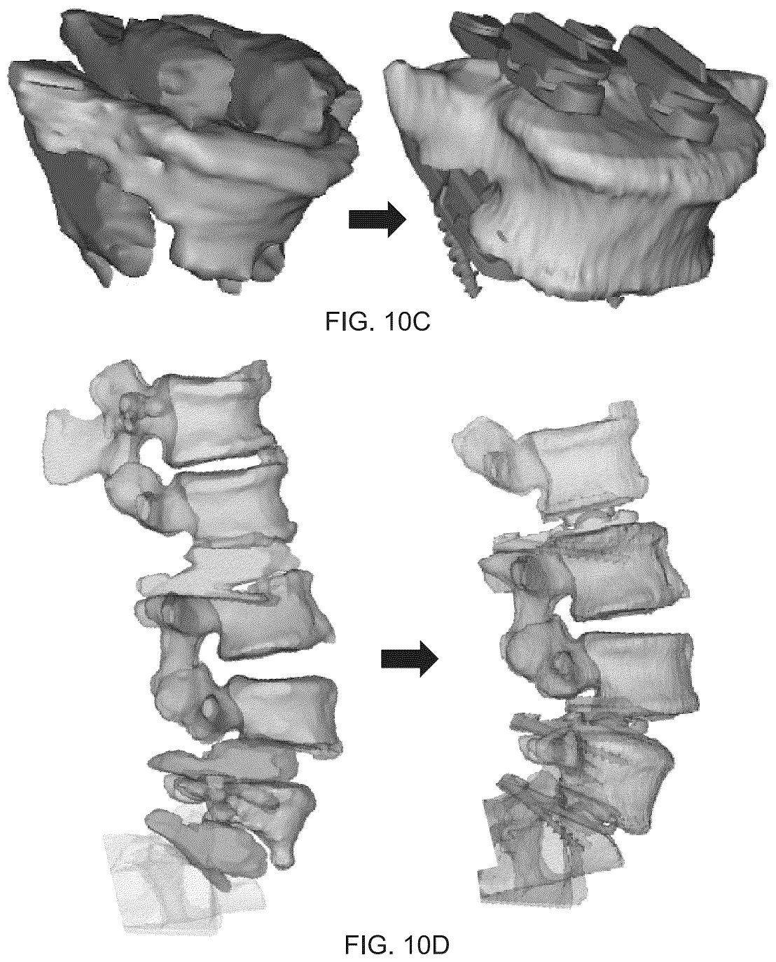

In another embodiment, the software application and/or the image processing module may further comprise a beam hardening correction module and/or function as shown in FIGS. 10A-10D. Beam hardening is a well-known effect in X-ray CT scanning that is caused by the interaction of a broad polychromatic X-ray spectrum and the energy-dependent material attenuation coefficient of materials cause beam hardening artifacts. These artifacts appear as cupping and streaking depending on the material composition and the geometry of the imaged object. The beam hardening correction module may comprise steps to correct and/or reduce the beam hardening artifacts from the at least one 2D and/or 3D image. The beam hardening function may compensate the “beam hardening effect” observed on the one or more operative images. Beam hardening may be resolved by employing a numerical optimization of 6 or more variables (at least 3 translation and at least 3 rotations). The beam hardening function may further move the targeted anatomy in space to best fit the unaffected portion of the targeted anatomy post-operatively. The beam hardening function may provide an optional removal of a portion of the target anatomy (e.g., removing facets).

In another embodiment, the software application and/or the image processing module may further comprise an image analysis module or function. The image analysis module may interact with one or more of the modules disclosed herein. The image analysis module may be used for quantitative measurements, and interpretations of medical images. The process of quantification interprets, identifies and determines properties of various structures such as volume, diameter, composition, and other relevant anatomical or physiological information. The process or function of image measurement may facilitate the ability to display results of quantitative measurements, including surgical measurements.

Preoperative Planning Software Application Computation of Data Set Results

With reference to FIG. 8A-8C and FIGS. 11A-110, 12A-12F and 13, describes the step of calculating one or more selected surgical measurements (SSM) from the one or more virtual models of the target anatomy to create a data set 115. The one or more selected surgical measurements (SSMs) are highlighted within FIG. 8C.

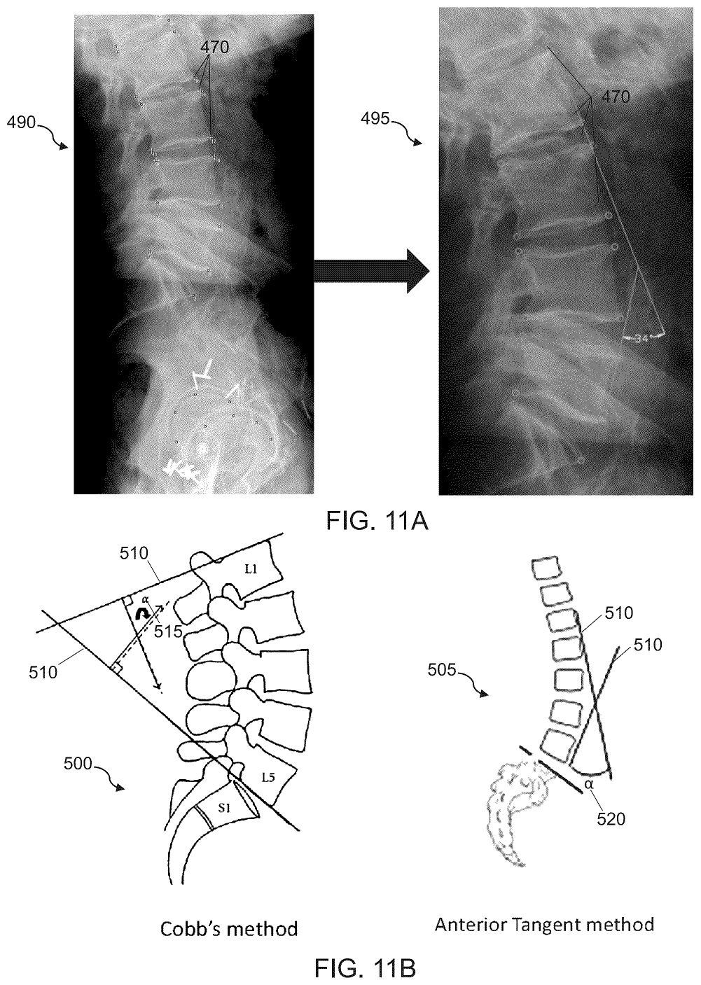

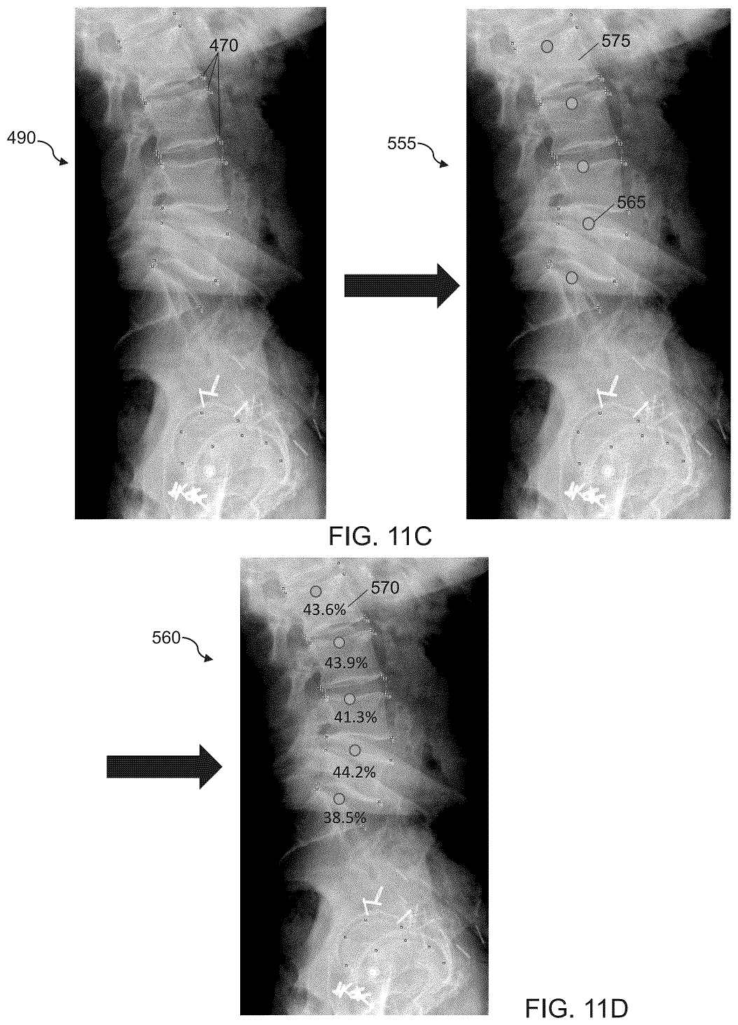

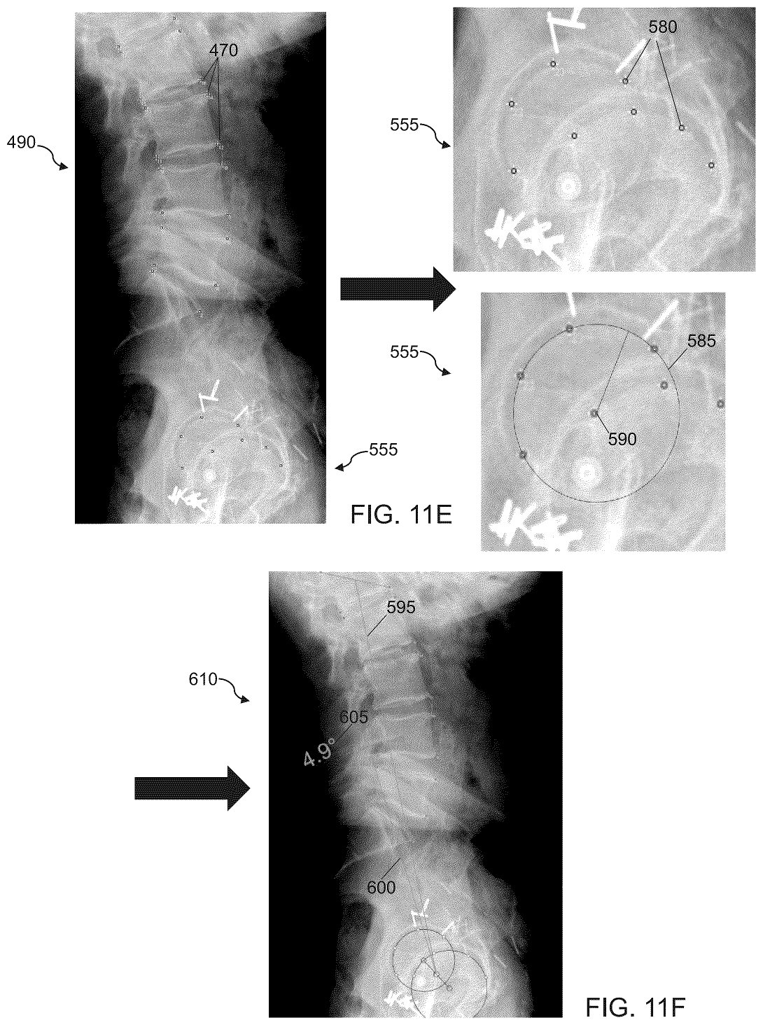

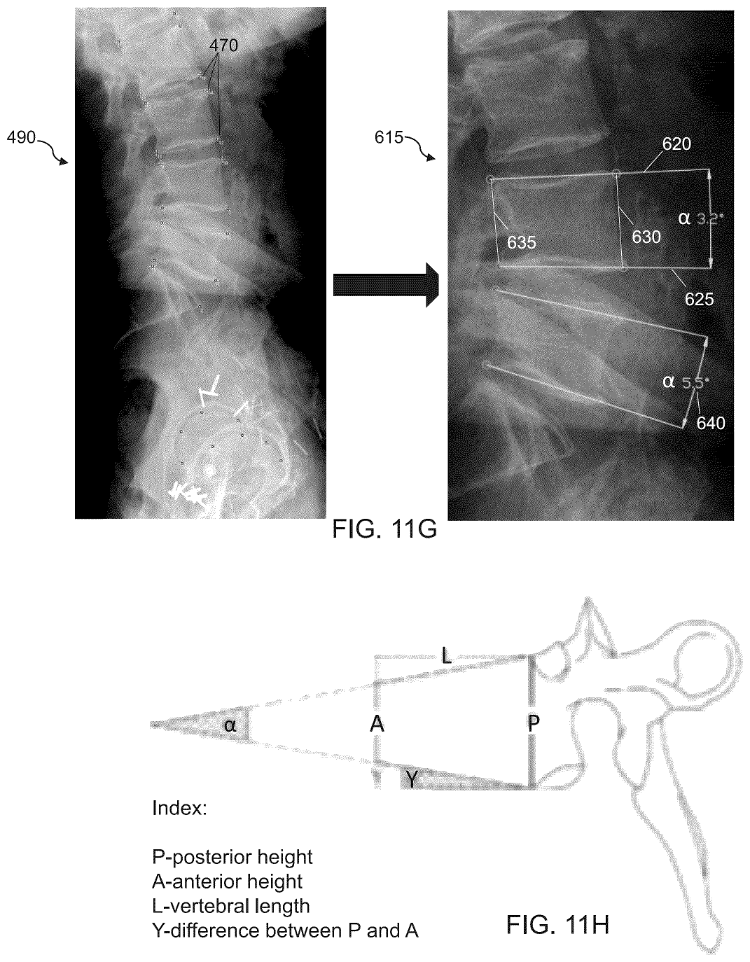

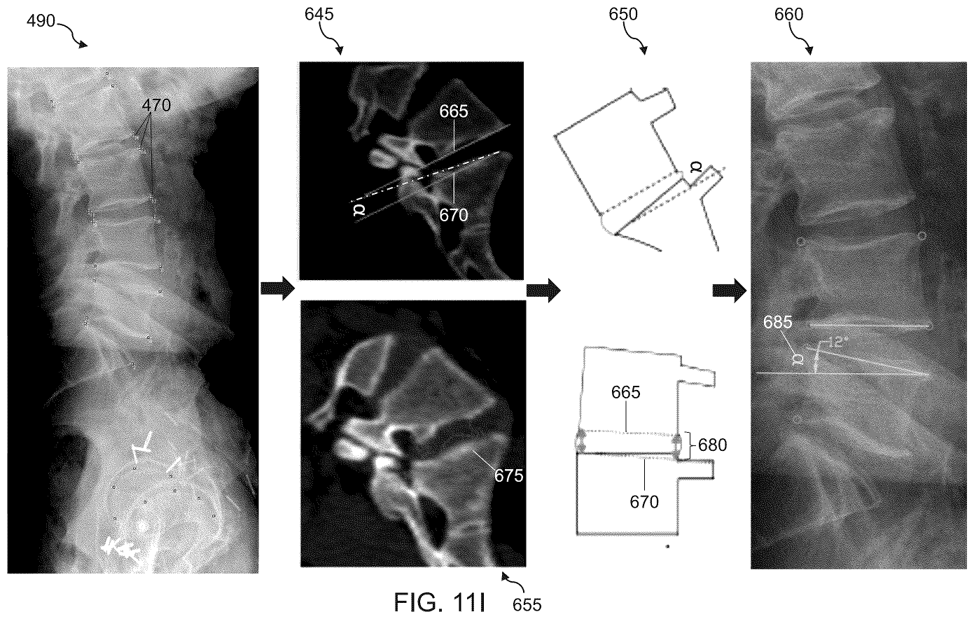

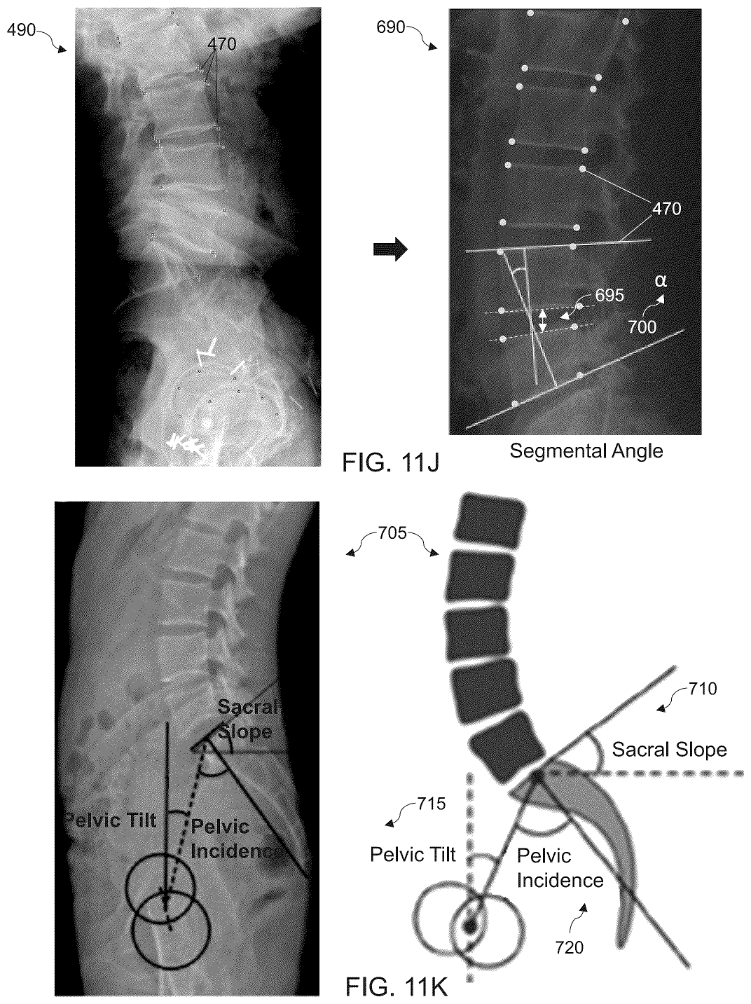

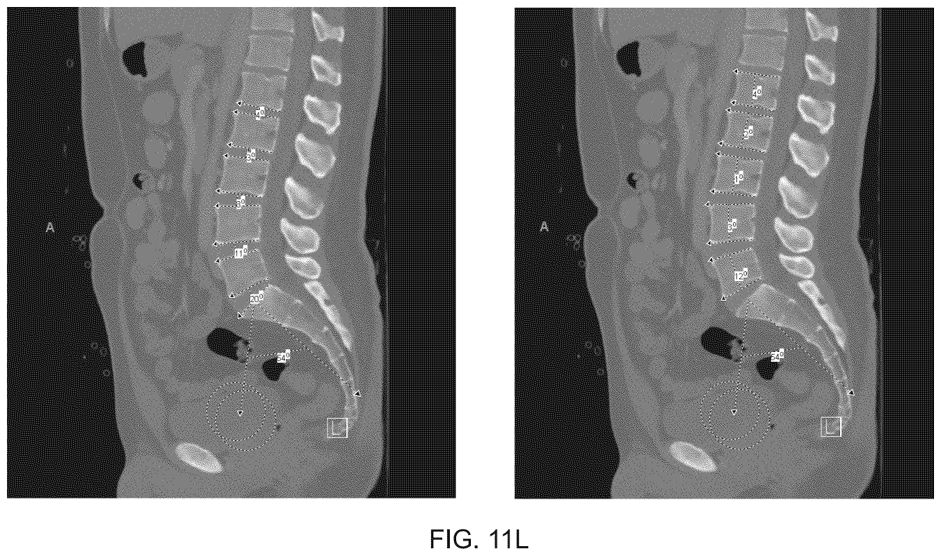

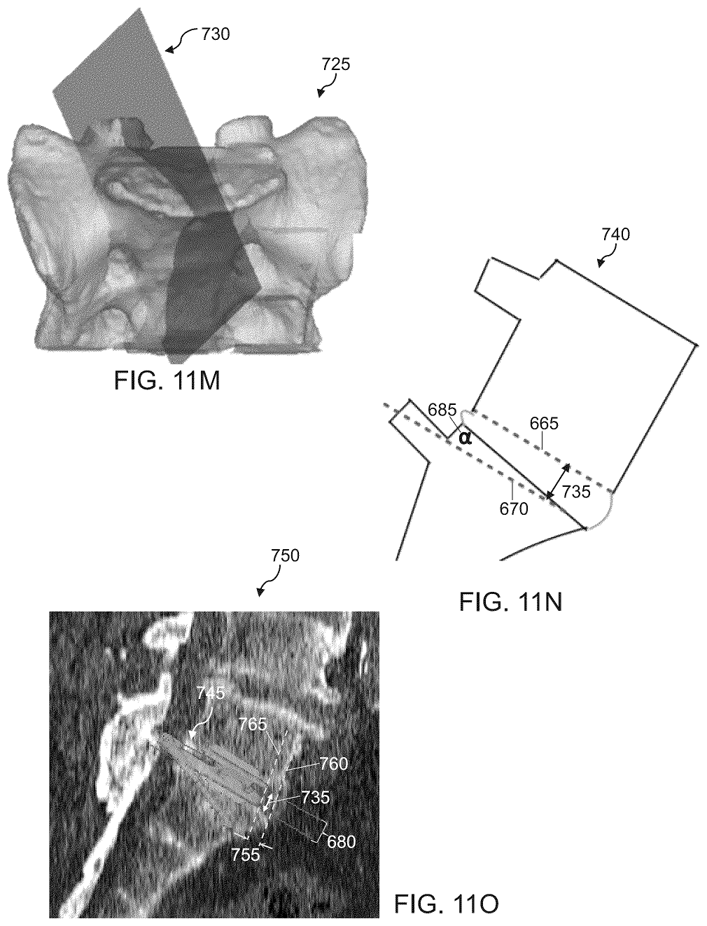

With reference to FIG. 8C and FIGS. 11A-11B, the step of calculating one or more selected surgical measurements (SSM) from the one or more virtual models of the target anatomy to create a data set 115 may comprise the calculation of vertebral lumbar lordosis (LL) 500, 505. The LL may use any methods known in the art. This includes using Cobb's method 500 or Anterior Tangent Method 505. The angle of the inward curve of the lumbar spine (just above the buttocks) is expressed as the average value between the angles obtained by Cobb's method 500 and the Anterior Tangent Method 495, 505. The one or more operative images 490 uploaded to the software and stored, may be accessed to draw patient-specific indicia or markers. Indicia or markers may further include tangent lines. Using Cobb's method 500, tangent lines 510 may be drawn along the superior end of L1 and S1. Perpendicular to each of the lines, an angle 515, α, is formed. Also, the Anterior Tangent Method 495, 505 is calculated as the angle 520 between tangent lines 510 drawn through the anterior aspect of L5 and L1. Tangent lines as described above may be derived from the user, surgeon or software indicated vertebral corner points 470 as seen in FIG. 9C.

With reference to FIG. 8C and FIGS. 11C-11D, the step of calculating one or more selected surgical measurements (SSM) from the one or more virtual models of the target anatomy to create a data set 115 may comprise the calculation of instantaneous center of rotation (ICOR) or center of rotation (COR) 570. The one or more operative images 490 uploaded to the software and stored, may be accessed to draw patient-specific indicia or markers. Indicia or markers may further include points or circles that highlight the center of rotation 555 within a portion of a spine within a spine region. COR points or circles 565 may be derived or calculated by using the vertebral corner points 470 within each vertebra 575 and displayed onto the processed image or virtual model. The vertebral corner points 470 may be user or software defined. The ICOR and/or the COR 570 may be different or the same within each vertebra 575 and/or spinal segment of a spinal region. The COR 570 may be calculated by known methods in art. For example, the COR 570 within the lumbar region for each spinal segment may be calculated using the Table 1 below. The COR 570 and the COR points 565 may be displayed onto the virtual model 560.

TABLE 1
COR Calculation

| Lumbar Level | Horizontal | Vertical |
|---|---|---|
| L51 | 38.5% of S1 corner points | 50.0% of L5S1 at 38.5% |
| L45 | 44.2% of L5 corners | 00.0% of L45 at 44.2% |
| L34 | 41.3% of L4 corners | 00.0% of L34 at 41.3% |
| L23 | 43.9% of L3 corners | 00.0% of L23 at 43.9% |
| L12 | 43.6% of L2 corners | 00.0% of L12 at 43.6% |

With reference to FIG. 8C and FIGS. 11E-11F, the step of calculating one or more selected surgical measurements (SSM) from the one or more virtual models of the target anatomy to create a data set 115 may comprise the calculation of L1 Pelvic Angle (L1 PA). The one or more operative images 490 uploaded to the software and stored. The one or more operative images 490 may further comprise a portion of the hip 555, including the femoral head 555. The one or more operative images 490 may be accessed to draw patient-specific indicia or markers. Indicia or markers may further include points or circles 580 that highlight the perimeter of the femoral head 555. A plurality of points or circles 580 will be positioned along the perimeter of the femoral head using “best fit” to create a circle 585 and obtain the best approximation for the center 590 of the femoral head. Also, a point refinement via the steepest gradient may be used. Once the center 590 of the femoral head is obtained, a first reference line 595 may be projected or displayed onto the processed image or virtual model that extends from the center 590 point of the femoral head axis to the L1 vertebral body. A second reference line 600 may be projected or displayed onto the processed image or virtual model 610 that extends from the center 590 point of the femoral head axis to the center of the S1 endplate. The first reference line 595 and the second reference line 605 forms an angle 605.

With reference to FIG. 8C and FIGS. 11G-11H, the step of calculating one or more selected surgical measurements (SSM) from the one or more virtual models of the target anatomy to create a data set 115 may comprise the calculation of a vertebral body wedge angle 640. The one or more operative images 490 may comprise patient-specific indicia or markers 470. Indicia or markers 470 may further include tangent lines, reference lines and/or points or angles. The indicia or makers may be disposed via user and/or by the software. The virtual model 615 may comprise a plurality of reference lines 620,625,630,635 on the vertebral body to calculate the vertebral body wedge angle 640, α. The vertebral wedge angle is defined as α=2 TAN⁻¹(Y/L) when Y=(P−A)/2. The vertebral wedge angle 640, α, may also be defined as the angle formed by two lines connecting upper endplate and lower endplate of a vertebral body.

With reference to FIG. 8C and FIG. 11I, the step of calculating one or more selected surgical measurements (SSM) from the one or more virtual models of the target anatomy to create a data set 115 may comprise the calculation of an osteotomy sagittal angle or degree of osteotomy in the sagittal plane and/or neutral alignment. The one or more operative images 490 may comprise patient-specific indicia or markers 470. Indicia or markers 470 may further include tangent lines, reference lines and/or points or angles. The indicia or makers may be disposed via user and/or by the software. The spinal implant is intended to be deployed between an intervertebral space of an upper and lower vertebra and the spinal implant should be parallel relative the lower endplate of the upper vertebra and the upper endplate of the lower vertebra. This allows the spinal implant to be disposed into a neutral position and/or neutral alignment allowing full range of motion and improve overall spinal alignment. A plurality of reference lines 665,670 may be projected or disposed onto a first virtual model 645, 665 that extend across the lower endplate of the upper vertebra and the upper endplate of the lower vertebra. The plurality of reference lines 665,670 should be parallel or neutrally aligned 680 to each other. A second virtual model 655 may comprise an object 675, the object 675 may include a virtual spinal implant. The object 675 may be disposed or projected into the second virtual model 655 to confirm that the object 675 contains a neutral alignment 680 within the intervertebral disc space.

Neutral alignment 680 is defined as the reference line 665 of the lower endplate of the upper vertebra and the reference line 670 of upper endplate of the lower vertebra resulting in a portion of the endplate reference lines 665 are parallel. Alternatively, neutral alignment 680 is defined as the reference line 665 of the lower endplate of the upper vertebra and the reference line 670 of upper endplate of the lower vertebra resulting in the entirety of the endplate reference lines 665 are parallel. The final calculation of the osteotomy sagittal angle or degree of osteotomy within the sagittal plane 685, α, is disposed onto a third virtual model 660. Furthermore, the degree of osteotomy within the coronal plane or the osteotomy coronal angle may also be calculated. The first, second, and/or third virtual model may be displayed and/or stored within the database.

With reference to FIG. 8C and FIG. 11J, the step of calculating one or more selected surgical measurements (SSM) from the one or more virtual models of the target anatomy to create a data set 115 may comprise the calculation of segmental angle 700. The one or more operative images 490, 690 may comprise patient-specific indicia or markers 470. Indicia or markers 470 may further include tangent lines, reference lines and/or points or angles. The technique for measuring segmental disc angles 700 may require measurement of the disc height 695 to produce the segmental angle 700. The segmental angle 700 is the angle between the upper and lower endplates at the targeted intervertebral disc space where surgery may take place. The disc height 695 is the distance between the upper and lower endplates at the center of the targeted intervertebral disc space at the surgical site from the sagittal view. Alternatively, the disc height 695 is the distance between the upper and lower endplates of the targeted intervertebral disc space at the right side and left side of the intervertebral space divided by two to be reflected as disc height 695=(a+b)/2.

With reference to FIG. 8C and FIGS. 11K-11L, the step of calculating one or more selected surgical measurements (SSM) from the one or more virtual models of the target anatomy to create a data set 115 may comprise the calculation of sacral slope (SS) 710, pelvic tilt (PT) 715 and pelvic incidence (PI) 720. The one or more operative images 490, 705 may comprise patient-specific indicia or markers 470. Indicia or markers 470 may further include tangent lines, reference lines and/or points or angles. The SS 710 and the PT 715 are variable dependent on the version of the pelvis around the hip axis. The SS 710 is defined as the angle between the endplate of S1 and a horizontal line. The PT 715 is defined as the angle between a vertical line and the line joining the middle of the sacral plate and the axis of the femoral heads. The PI 720 is constant for each person and defined as an angle between a line joining the center of the upper endplate of S1 to the axis of the femoral heads and a line perpendicular to the upper endplate of S1. The technique for measuring the PI 720 was measured by identifying the center of the femoral heads on the respective sagittal slices, then identifying the bicoxofemoral axis in the midsagittal plane. In the midsagittal plane, the endplates were identified of each vertebral body from L1 to L5 and the sacrum allowing measurement of the vertebral body and disc angles as shown in FIG. 11L. A negative value indicated a kyphotic value while a positive value was lordotic.

With reference to FIG. 8C and FIGS. 11M-110, the step of calculating one or more selected surgical measurements (SSM) from the one or more virtual models of the target anatomy to create a data set 115 may comprise the calculation or determination of implant sizing. Implant sizing may include implant length, implant height, and/or implant width. A sagittal plane view 730 may be taken from a first virtual model 725. One or more selected surgical measurements (SSM) may be determined or calculated on a second virtual model 740, including the osteotomy sagittal angle or degree of osteotomy within the sagittal plane 685 (see FIG. 11I) and a neutral alignment 680 (see FIG. 11I). As previously disclosed, neutral alignment 680 is defined as the reference line 665 of the lower endplate of the upper vertebra and the reference line 670 of upper endplate of the lower vertebra resulting in a portion of the endplate reference lines 665 are parallel and/or an entirety of the endplate reference lines 665 are parallel (along the entire endplate surfaces). A portion of parallel endplate surfaces or the entirety of parallel endplate surfaces can result in a neutral intervertebral disc height 735. An approximated implant 745 may be disposed onto a third virtual model 750. One or more implants 745 of different sizes may be inserted within the neutrally aligned intervertebral disc space 680 until the desired one or more implants 745 obtains a desired implant positioning, implant height, implant width, and/or implant length. The virtual models 725, 740, 750 may be rotated in different axis and plane views to confirm proper or desired implant sizing and/or implant positioning. The first 725, second 740, and/or third virtual model 750 may comprise the same models and/or different models. The first 725, second 740, and/or third virtual model 750 may comprise a 2D or 3D model.

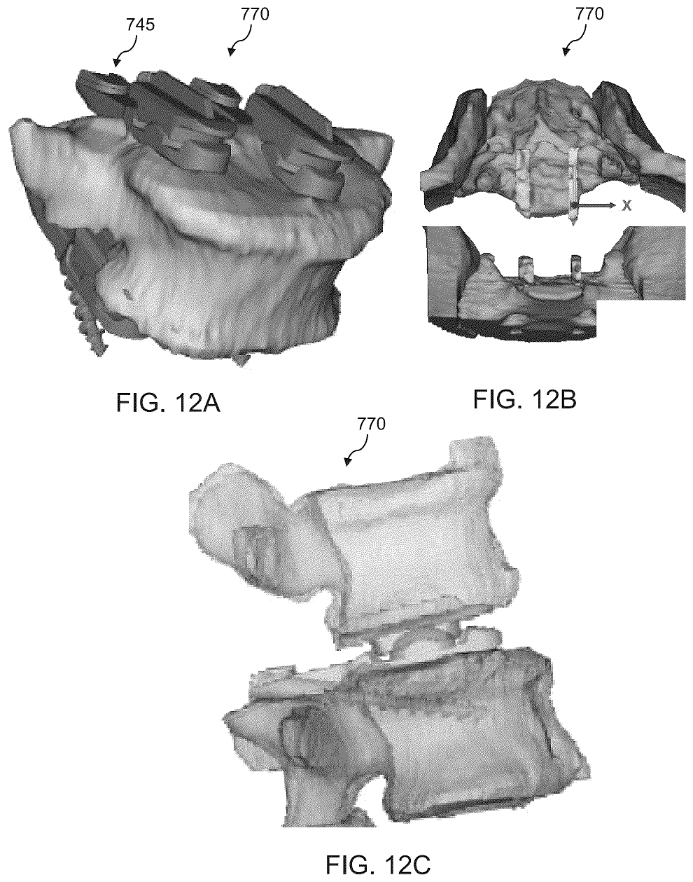

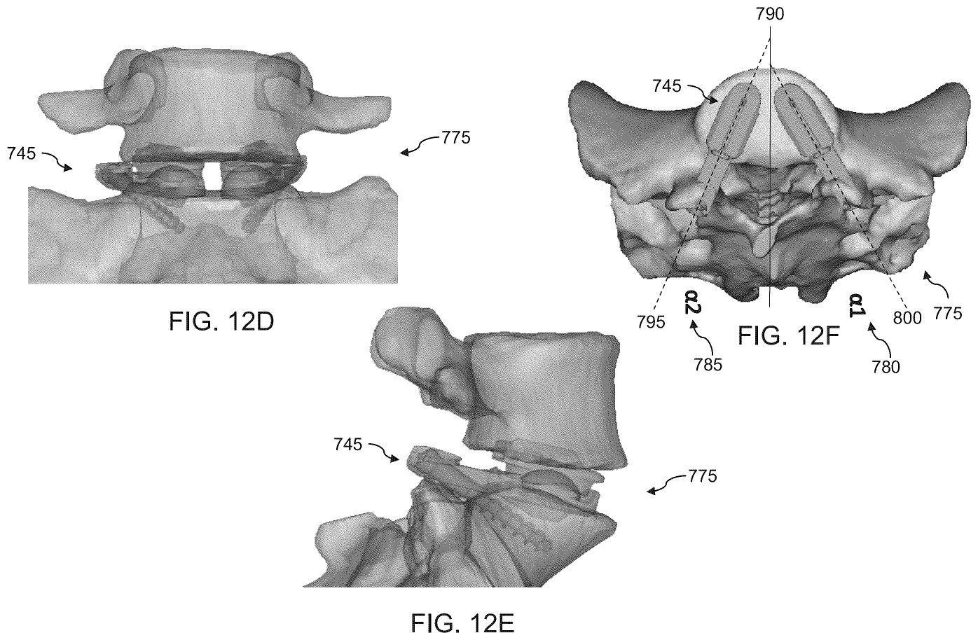

With reference to FIG. 8C and FIGS. 12A-12F, the step of calculating one or more selected surgical measurements (SSM) from the one or more virtual models of the target anatomy to create a data set 115 may comprise the calculation or determination of implant positioning. Implant positioning may comprise one or more of the following: (1) COR (see FIGS. 11C-11D); (2) A/P distance 755 that the anterior most portion 765 of the one or more implants 745 from the anterior surface 760 of the upper and/or lower vertebral body (see FIG. 11O); and/or (3) convergence angle or transverse pedicle angle, and/or any combination thereof (see FIGS. 12A-12F). Convergence angle or transverse pedicle angle 780, 785 is defined by creating a central axis 790 measurement from spinous process to the anterior vertebral body then measuring one or more angles 780, 785 from the central axis 790 to the mid-axis of at least one pedicle 795,800. Alternatively, the convergence angle or transverse pedicle angle 780, 785 is defined by creating a central axis 790 measurement from spinous process to the anterior vertebral body then measuring a first angles 780 from the central axis 790 to the mid-axis of the first pedicle 795 and measuring a second angle 785 from the central axis 795 to the mid-axis of the second pedicle 800.

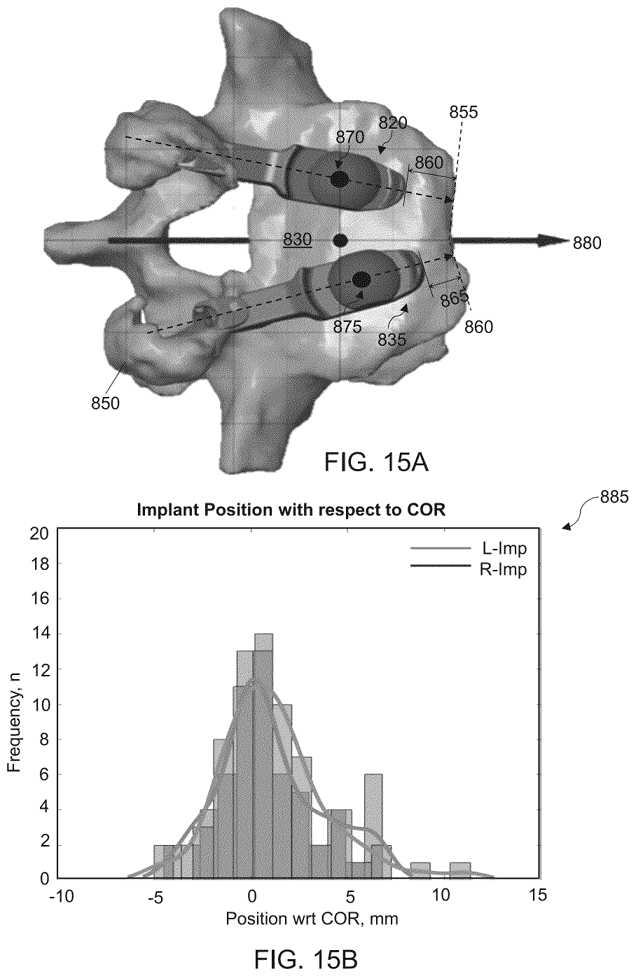

Implant positioning may help determine implant sizing. Accordingly, a fourth virtual model 775 with one or more implants 745 may be rotated in different planes—isometric, sagittal, coronal, A/P and/or any combination thereof—to confirm implant sizing and implant position. The first 725, second 740, third 750 and/or fourth virtual model 770 may comprise the same models and/or different models. The first 725, second 740, third 750 and/or fourth virtual model 770 may comprise a 2D or 3D model. Implant positioning may further comprise (4) sagittal osteotomy angle (see FIG. 11I); (5) coronal osteotomy angle (see FIGS. 15A-15C); and/or (6) neutral alignment.

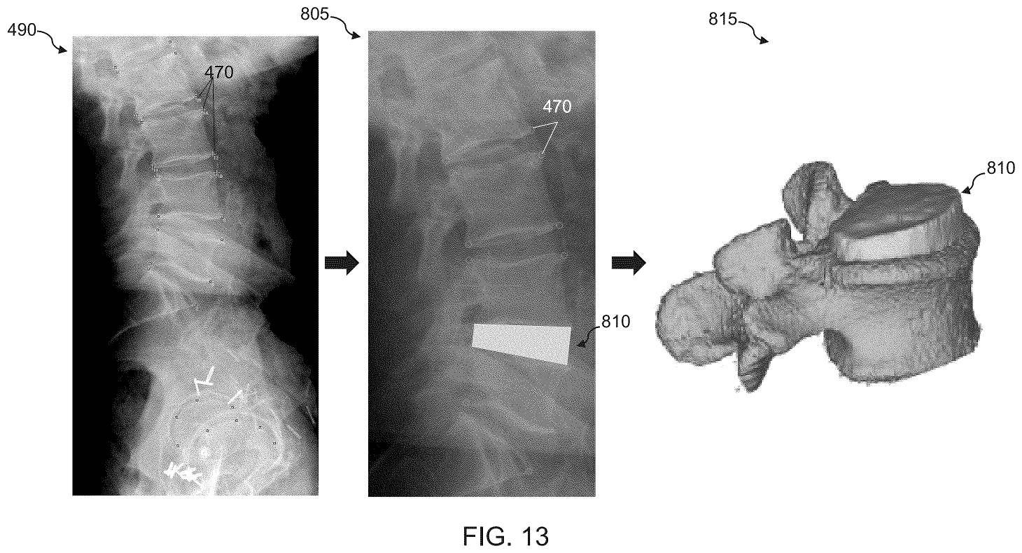

With reference to FIGS. 8C and 13, the step of calculating one or more selected surgical measurements (SSM) from the one or more virtual models of the target anatomy to create a data set 115 may comprise the calculation or determination of disc height. The one or more operative images 490 may comprise patient-specific indicia or markers 470. Indicia or markers 470 may further include tangent lines, reference lines and/or points or angles. The intervertebral disc 810 can be identified within one or more virtual models 805, 815 comprising indicia or markers 470. The intervertebral disc height or disc height may be defined within a 2D virtual model 805 as the area of the intervertebral space or when divided by the length of the endplate as the average disc height. Furthermore, the intervertebral disc height or disc height may also be defined within a 3D virtual model 815 as the volume of the intervertebral disc space or when divided by the area of the endplate as the average disc height. See E. Kamaric et al., “Novel Approach to Measure Intervertebral Disc Height,” Spine Experts Group, 2^{nd} Annual Meeting, Podium Presentation, Proceedings pg. 31, Budapest, Hungary (Dec. 9-11, 2004).

Once the software application computes and/or manipulates the one or more images to output processed virtual models and selected surgical measurements (SSM), the software may create one or more data set files that require storage. The one or more data set files may comprise one or more virtual models (e.g., processed images) and/or quantitative data. The one or more data set files may comprise an image management module and/or a data set results module. In one embodiment, the software application may generate a CSV file after the one or more operative images are image processed via the image processing module (e.g. virtual models). The one or more virtual models may comprise annotated SSMs. The one or more virtual models may comprise 2D or 3D models.

In another embodiment, the software application may further comprise an image management module. The image management module comprises steps to manage the acquired information and encompasses various techniques for annotations, compression, storage, retrieval, and communication of image data as shown in FIG. 5M. The management may further comprise downloading and/or sharing of image data. There are several standards and technologies developed to address various aspects of image management. For example, the medical imaging technology picture archiving and communication system (PACS) provides economical storage and access to images from multiple modalities and the digital imaging and communication medicine (DICOM) standard is used for storing and transmitting medical images. The image data may include raw images, CT scout images, surgeon modified images, and/or analyzed/annotated images (e.g., virtual models). The image data may further comprise 2D and/or 3D images.

In another embodiment, the software application may further comprise a data results module. The data management module comprises steps to manage the acquired quantitative data and encompasses various techniques for annotations, compression, storage, retrieval, and communication of quantitative data as shown in FIG. 5M. Management of data may further include downloading and/or sharing at least one or more quantitative measurements. The quantitative data may include projected surgical measurement data. The projected surgical measurement (PSM) or selected surgical measurement (SSM) data may include the different measurements highlighted in FIG. 8C. The PSM or SSM data includes one or more of pelvic incidence (PI), pelvic tilt (PT), sacral slope (SS), L1 pelvic angle (L1PA), sagittal vertical axis (SVA), center of rotation (COR), lumbar lordosis (LL), segmental lordosis, regional lordosis, wedge angle vertebral body, wedge angle disc, osteotomy sagittal angle, osteotomy transverse trajectory, osteotomy coronal trajectory, convergence angle or transverse pedicle angle, anterior-posterior (A/P) distance, neutral alignment, disc height, implant sizing and/or any combination thereof.

Intraoperative Method

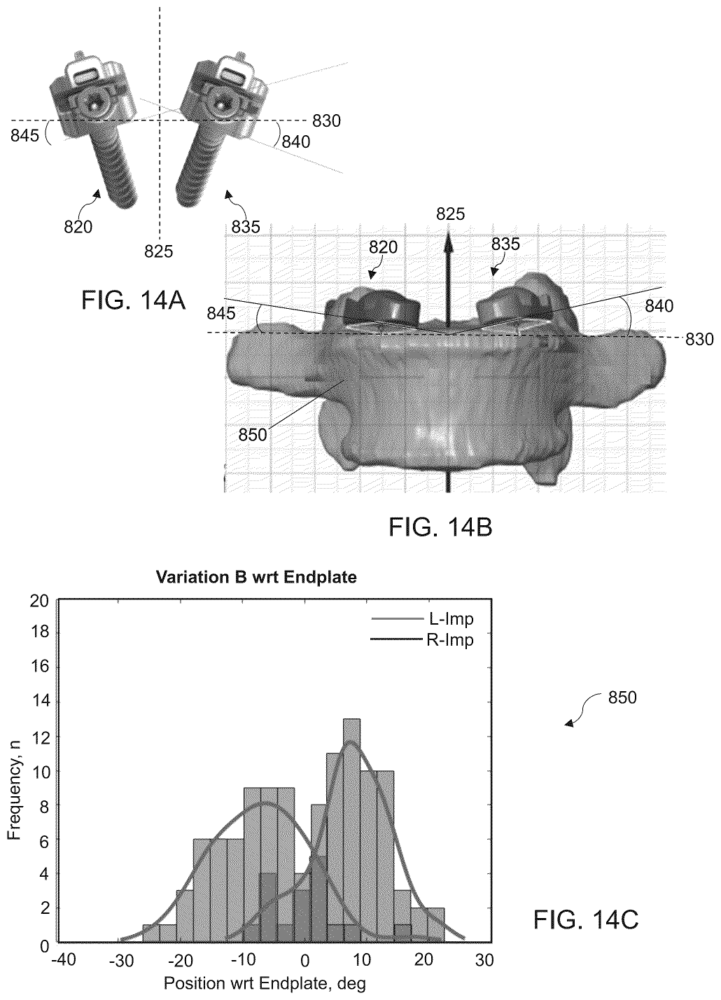

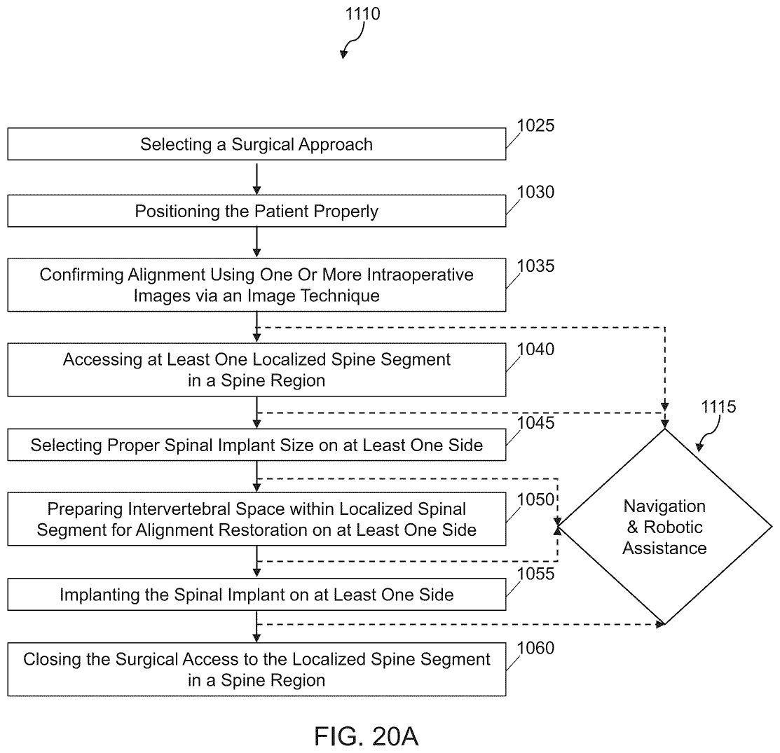

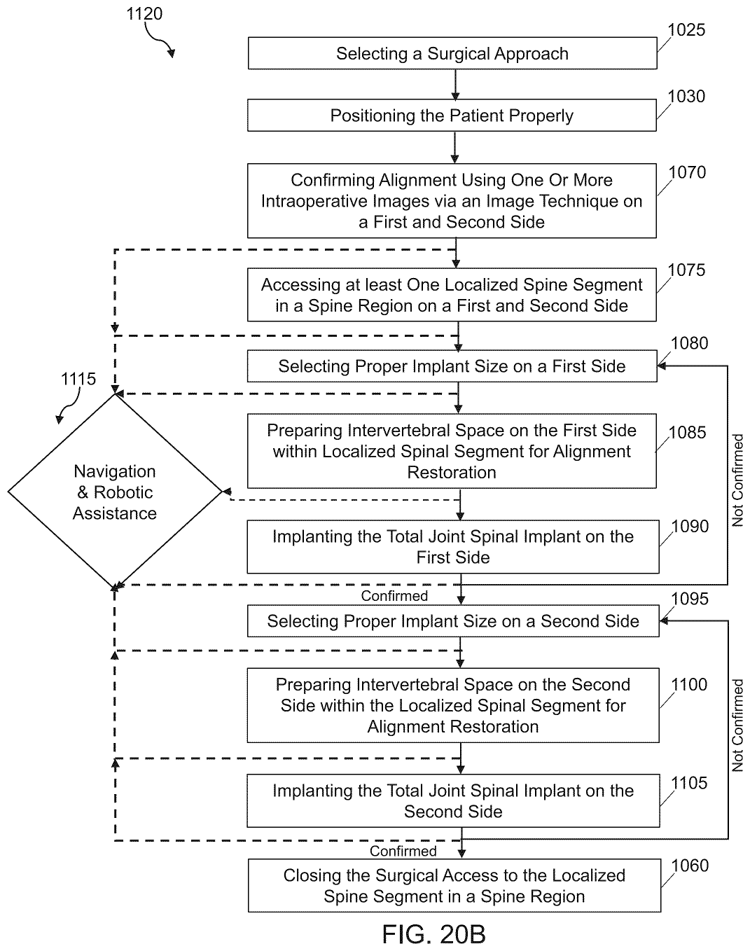

The total joint replacement method to restore alignment and motion may further comprise the execution of a traditional operative protocol or intraoperative method. The traditional method may be performed manually by a surgeon. However, manual surgery may provide some disadvantages which may affect the accuracy of different steps or methods during the operative surgery. Alternatively, the surgeon may desirably select navigation assisted spine surgery and/or robotic assisted spine surgery to improve the accuracy of various operative steps by providing a greater degree of control with orientation assistance in different anatomical situations to provide highly precise positioning or trajectory data, allows precise implant positioning in a more accurate and reproducible position(s), greater visualization, and/or improve patient outcomes (e.g., shorter hospitalizations, reduced pain and discomfort, faster recovery time, reduced blood loss and transfusions, minimal scarring, etc.). Navigation assisted and/or robotic assisted spine surgery may improve the accuracy of different operative steps, including surgical access, optimal or enhanced selection of spinal implant, precise bony preparation and/or precise implant positioning as shown in FIGS. 14A-14C, 15A-15B, 16A-16C, 17A-17C, 20A-20B and/or 21A-21E.

With reference to FIGS. 14A-14C, navigation assisted and/or robotic assisted spine surgery may improve the accuracy of bony preparation. Bony preparation comprises the step of preparing intervertebral space on the second side within localized spinal segment for alignment restoration on at least one side, a first side and/or a second side. The step of preparing intervertebral space on the second side within localized spinal segment for alignment restoration on at least one side, a first side and/or a second side may comprise creating one or more osteotomy angles to improve a patient's spinal alignment thought the correction of imbalanced sagittal and/or coronal vertebra or vertebral segments. The osteotomy angles may include a sagittal osteotomy with a desired sagittal osteotomy angle and/or a coronal osteotomy with a desired osteotomy angle.

In one embodiment, the bony preparation may include one or more coronal osteotomy angles 840, 845. The one or more coronal osteotomy angles 840, 845 is defined as the one or more implants 820,835 that are non-planar with respect to a prepared endplate surface 830 of a vertebral body and/or an endplate surface of a vertebral body. The endplate surface 830 may be generally planar or planar. Also, the coronal osteotomy angles 840, 845 is defined as the one or more implants 820,835 are non-planar with respect to a prepared endplate surface 830 of a vertebral body.

Alternatively, the bony preparation may include a vertebral body 850 with an endplate surface 830 and a vertical axis 825. The vertebral body 850 may comprise a first implant 835 with a first coronal osteotomy angle 840 and a second implant 820 with a second coronal osteotomy angle 845. The first coronal angle 840 may comprise a different angle than the second coronal osteotomy angle 845. The first coronal osteotomy angle 840 may comprise a same angle than the second coronal osteotomy angle 845. The one or more coronal osteotomy angles 840, 845, the first coronal osteotomy angle 840, and/or the second coronal osteotomy angle 845 may comprise a positive, negative or neutral angle. FIG. 14C illustrates a graph of the distribution of the one or more coronal osteotomy angles 840, 845 of the one or more implants 820, 835. Navigation assisted and/or robotic assisted spine surgery may improve the accuracy and/or precision of the distribution of the one or more coronal osteotomy angles 840, 845 (e.g., lowering the delta or difference between the one or more coronal osteotomy angles 840, 845).

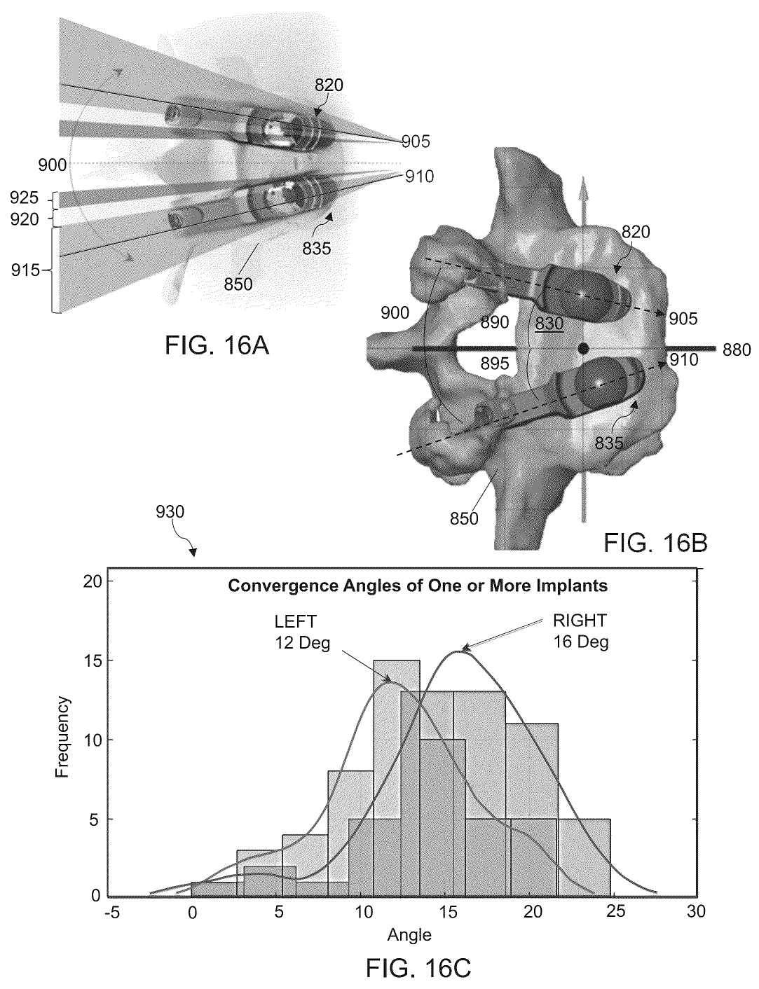

With reference to FIGS. 15A-15B, 16A-16C and/or 17A-17C, navigation assisted and/or robotic assisted spine surgery may improve the accuracy of implant positioning. Implant positioning may be affected by different measurements and/or bony preparations, including one or more of the following: (1) COR (see also FIGS. 11C-11D); (2) A/P distance that the anterior most portion of the one or more implants from the anterior surface of the upper and/or lower vertebral body (see also FIG. 110); (3) convergence angle or transverse pedicle angle, and/or any combination thereof (see also FIGS. 12A-12F and 16A-16C) (4) sagittal osteotomy angle (see FIG. 11I); (5) coronal osteotomy angle (see FIGS. 15A-15C); and/or (6) neutral alignment.

In one embodiment, implant positioning may include one or more anterior-posterior (A/P) distances 860,865 and/or one or more center of rotations (CORs) 870,875. The one or more A/P distances 860, 865 is defined as at least one implant 820 that is placed forward at a distance from the contralateral implant 835. The one or more A/P distances 860, 865 may also be defined as the one or more distances from the proposed anterior surfaces 855,860 of each of the one or more implants 820,835. The one or more center of rotations (CORs) 870,875 is defined as approximately 35-45% anterior from the posterior, caudal endplate of a cranial vertebral body or a vertebral body and/or an average of 40% from the posterior, caudal endplate of a vertebral body. The placement of the COR 870,875 of one or more implants 820,835 may affect the A/P distances 860,865 of the one or more implants 820,835. For example, if the surgeon desires to position the one or more implants 820,835 with respect to COR 870, 875 of the one or more implants 820,835, the A/P distances 860,865 may vary. However, if the surgeon desires to position the one or more implants 820,835 with respect to A/P distances 860,865 (e.g., having equal A/P distances between one or more implants 820, 835), such placement may affect the COR 870,875 of the one or more implants 820,835.

The desired implant positioning may include a vertebral body 850 with an endplate surface 830, anterior vertebral body surfaces 855,860 and/or a A/P axis 880. The vertebral body 850 may comprise a first implant 835 with a first COR 875 and a first A/P distance 865 and a second implant 820 with a second COR 870 and a second A/P distance 860. The first COR 875 may comprise a same COR compared to the second COR 870. The first COR 875 may comprise a different COR compared to the second COR 870. The first A/P distance 865 may comprise a same distance compared to the second A/P distance 860. The first A/P distance 865 may comprise a different distance compared to the second A/P distance 860. FIG. 15B illustrates a graph 885 of the distribution of the one or more implant positions with respect to COR 870, 875 of the one or more implants 820, 835. Navigation assisted and/or robotic assisted spine surgery may improve the accuracy and/or precision of the distribution of the one or more implant positions with respect to COR 870, 875, the one or more implant positions with respect to A/P distances 860, 865, and/or the one or more implant positions with respect to a balance between AP distances 860, 865 and COR 870, 875.

In another embodiment, implant positioning may include one or more convergence angles or transverse pedicle angles 890, 895, 900. The one or more convergence angles and/or transverse pedicle angles 890, 895, 900 may be defined as the total angle 900 between the one or more implants 820,835, which the one or more implants 820, 835 follows the mid axis of the pedicles 905, 910 and/or the total angle 900 between the mid axis of the pedicles 905,910. Alternatively, the one or more convergence angles and/or transverse pedicle angles 890, 895, 900 may be defined as an angle 890, 895 between the A/P axis 880 and the mid axis of the pedicles 905, 910 and/or an angle 890, 895 between the A/P axis 880 and the mid axis of the one or more implants 820,835 that follows or substantially follows the mid axis of the pedicles 905, 910.

The desired implant positioning comprising convergence angles 890, 895, 900 may include a vertebral body 850 with an endplate surface 830, mid axis of the pedicles 905,910 and/or a A/P axis 880. The vertebral body 850 may comprise a first implant 820 with a first convergence angle 890, a first mid-axis of a pedicle 905 and a total convergence angle 900 and/or a second implant 835 with a second convergence angle 895, a second mid-axis of a pedicle 910 and the total convergence angle 900. The first convergence angle 890 may follow, match or substantially match the first mid-axis of the pedicle 905. The second convergence angle 895 may follow, match or substantially match the second mid-axis of the pedicle 910. The first convergence angle 890 may comprise a different angle compared to the second convergence angle 895. The first convergence angle 890 may comprise a same angle compared to the second convergence angle 895.

The substantially match the first and/or second mid-axis of the pedicles 905, 910 may include an angle range 915 that is acceptable for implant positioning. The substantially match may include a 10-15% deviation from the mid-axis of the pedicles 905, 910. The convergence angles 890, 895 may further include a secondary set of angles 920 that may be acceptable for implant positioning, but may affect the overall operation and success of the restoration of motion. The convergence angles 890,895 may further include a tertiary set of angles 925 that may not likely be acceptable for implant positioning. FIG. 16C illustrates a graph 930 of the distribution of the one or more implant positions with respect to convergence angles 890, 895, 900 of the one or more implants 820, 835. Navigation assisted and/or robotic assisted spine surgery may improve the accuracy and/or precision of the distribution of the one or more implant positions with respect to convergence angles 890, 895, 900—it may precisely compute the mid-axis of the pedicles 905, 910 and/or the one or more implants 820, 835 that follows, matches or substantially matches the mid-axis of the pedicles 905, 910 and/or to decrease the delta or the difference of the convergence angles 890,895 between the one or more implants 820, 835.

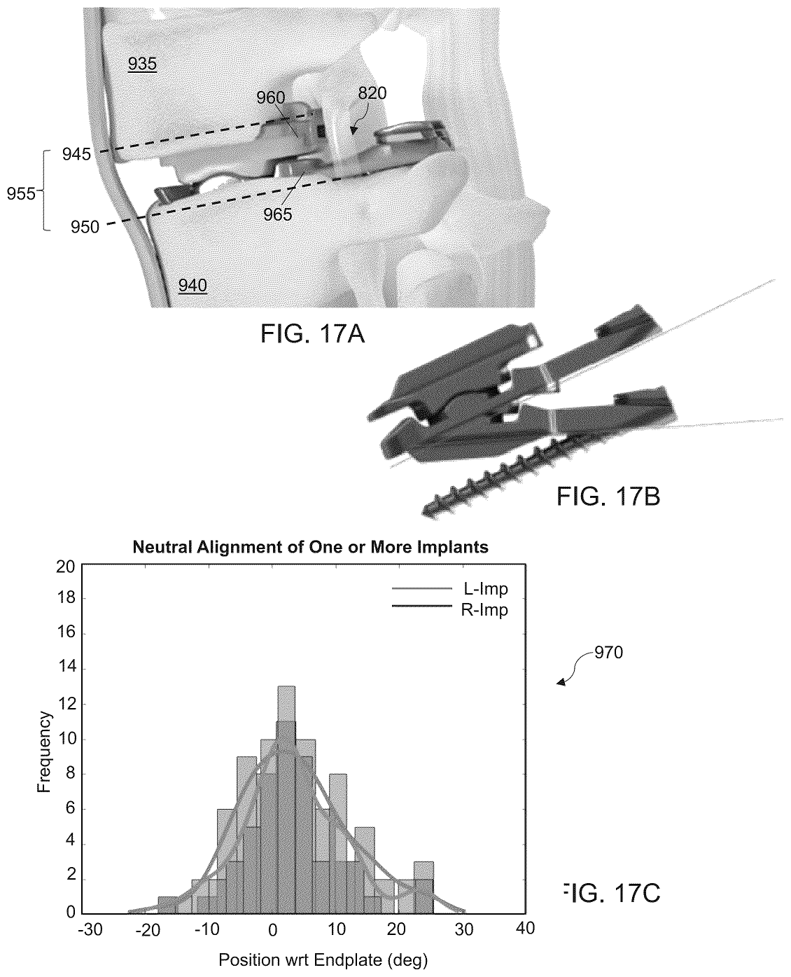

In another embodiment, implant positioning may include neutral alignment. Neutral alignment is defined as the lower endplate surface 945 of the upper vertebral body 935 is parallel or substantially parallel 955 to the upper endplate surface 950 of the lower vertebral body 940. Neutral alignment may also be defined as the superior element 960 of one or more implants 820, 835 is parallel or substantially parallel 955 to the inferior element 965 of the one or more implants 820, 835. Neutral alignment or neutral sagittal alignment is important for correcting spinal alignment and motion restoration via maximizing one or more implants 820, 835 range of motion. As previously discussed above, a sagittal osteotomy angles may be used to create parallel or substantially parallel 955 endplates 945, 950 for a neutral insertion of the one or more implants 820, 835. Such neutral alignment is necessary to prevent an anterior biased tilt or trajectory of the one or more implants 820, 835 as shown in FIG. 17B.

The desired implant positioning comprising neutral alignment may include a vertebral body 850 with a first endplate surface 945 of an upper vertebral body 935, a second plate surface 950 of a lower vertebral body 940, and/or one or more implants 820, 835. The one or more implants are positioned between the first endplate surface 945 and the second endplate surface 950. The first endplate surface 945 and/or the superior element 960 of the one or more implants 820, 835 is parallel or substantially parallel 955 to the second endplate surface 945 and/or the inferior element 965. FIG. 17C illustrates a graph 970 of the distribution of the one or more implant positions with respect to neutral alignment of the one or more implants 820, 835. Navigation assisted and/or robotic assisted spine surgery may improve the accuracy and/or precision of the one or more implant positions with respect to neutral alignment—it may precisely compute the one or more sagittal osteotomy angles for the lower vertebral body 940 to create an endplate surface 950 which the upper vertebral body 935 can be prepared to create another endplate surface 945 that is parallel 955 to each other. The precise computation may narrow or improve the delta or difference of neutral alignment between each of the one or more implants 820, 835.

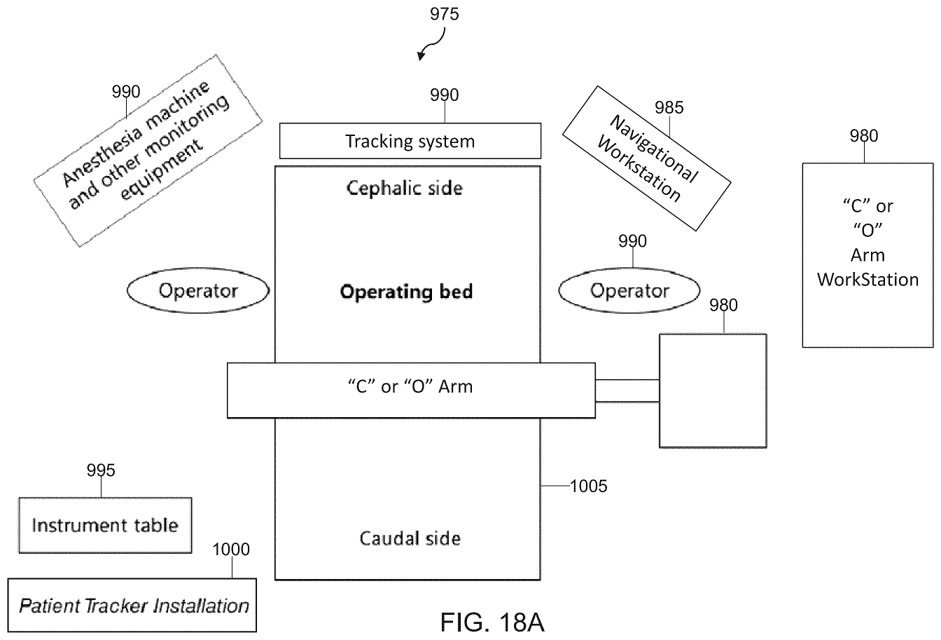

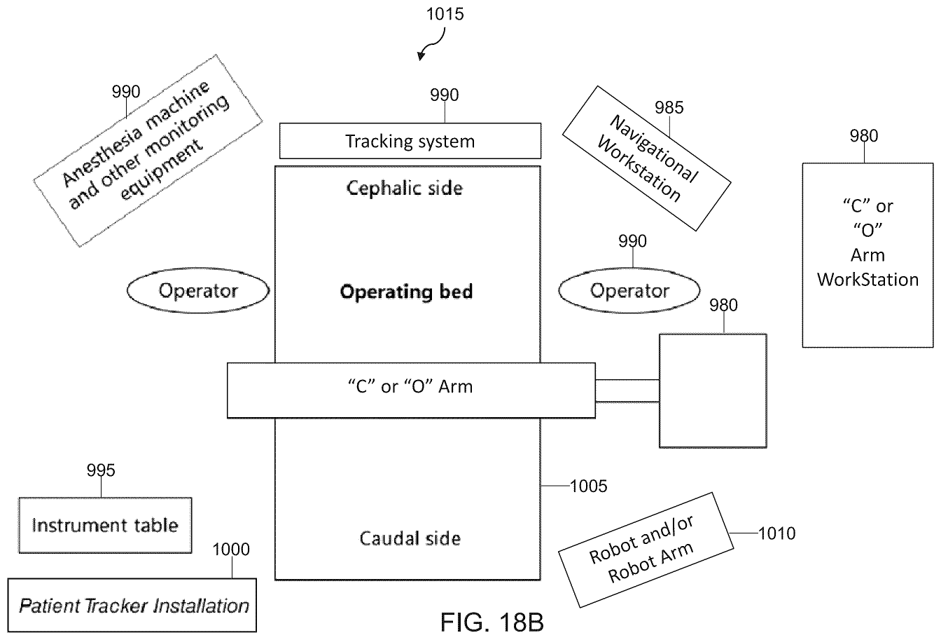

With reference to FIGS. 18A, a top view of one embodiment of a navigational system set-up 975 is shown. The navigational system set-up 975 comprises a navigational system, the navigational system includes a navigational workstation 985, a referencing system 990 and/or an imaging unit 980 using an imaging technique. The navigational system set-up 975 may include other traditional items that include anesthesia monitoring equipment 990, an instrument table 995 to hold sterilized instruments and implants, a patient tracking system table 1000, and an operating bed 1005.

The imaging unit 980 may to a separate imaging machine workstation 980 and/or it may communicate directly with navigational workstation 985. The navigational workstation 985 and/or the imaging unit workstation 980 includes image acquisition processing software (e.g., navigation software), a memory, a processing unit (e.g., computer), a display, and/or a transceiver to engage with the bilateral transfer of data. The imaging unit 980 is used to acquire high-resolution images of a region of interest and/or one or more targeted spinal segment(s). The images are obtained preoperatively, intraoperatively, and/or post-operatively, which the allows the surgeon to manually navigate upon these processed images to improve accuracy of access, bony preparation and implant positioning. The imaging unit 980 may comprise fluoroscopy, computerized tomography (CT), magnetic resonance imaging (MRI) and/or ultrasound machines.

Any type of navigational system may be used. The navigational system may comprise an Airo Mobile Intraoperative CT-based Spinal Navigation system (Brainlab, Munich, Germany). The Airo Mobile uses a mobile circular scanner attached to the operating table that allows for full 360 degree imaging and a scanning stereotactic camera for instrument registration. The navigational system may further comprise a Stealth Station Spine Surgery Imaging and Surgical Navigation with O-arm system (Medtronic, Minneapolis, MN). The Stealth Station Imaging and Surgical Navigation uses similar technology to the Airo, but opens at 90 degrees to allow for mobilization around the patient. The navigational system may further comprise a Ziehm Vision FD Vario 3D with NaviPort Integratiosystem (Ziehm Imaging, Orlando, FL). The Ziehm Vision FD obtains images via a 190-degree rotation with a C-arm around the patient. The navigational system may further comprise a Stryker Spinal Navigation with SpineMask Tracker and SpineMap Software system (Stryker, Kalamazoo, MI). The Stryker Navigation system uses a rectangle of trackers applied directly on the patient with an adhesive glue for referencing. The navigational system may further comprise a 7D Surgical Spine Navigation system (7D Surgical, Toronto, Ontario, Canada). The 7D Surgical Navigation System allows for radiation-free intraoperative navigation with Flash imaging, which uses high-intensity light and an overhead camera to image the surgical field and align with preoperative CT.

The referencing system 990 comprises a dynamic reference frame/array (DRA), light emitting diodes, and a tracking system. The dynamic reference array (DRA) is usually attached to fixed anatomical landmarks, such as the spinous process. One or more preoperative images may be acquired along with the fixed DRA to synchronize between the virtual navigated images and anatomical landmarks. The accuracy of the navigation depends on the stable fixation of this DRA, and, therefore, it must be left undisturbed throughout the surgery. When there is no adequate fixation point within the surgical field as in revision surgery, hypoplastic or immature bone, the iliac crest can be used by inserting a pin into it. In cervical spine surgery, where the distance from the iliac crest is more, the reference array could be fixed to the Mayfield frame.

The DRA further comprises features and/or provisions for attaching three or more spheres known as light-emitting diodes (LED). These LEDs emit light, which is tracked by an electro-optical camera and are known as active arrays. Specialized surgical instruments are used, which also have LEDs attached to them and are called passive arrays as they reflect the infrared rays emitted from the camera and gives the surgeon a real-time tracking of the exact location of these instruments over the surgical field. The 3D orientation between these active and passive LEDs, thus facilitates navigation.

Also, there are a variety of tracking systems to visualize the instrumentation. Various tracking systems are available that include optical, mechanical, acoustic or electromagnetic systems. Optical tracking systems are the most frequently used due to superiority in terms of accuracy. They use infrared camera devices to actively track the light emitted or reflected from the LEDs, which are attached to the dynamic reference frame/array DRA and surgical instruments. One such tracker includes the NavLock Tracker (Medtronic, Inc., Minneapolis, MN) that is a compatible accessory to the StealthStation System. The NavLock Tracker is available in four (4) different geometries. The trackers calculate the location and provide real-time three-dimensional (3D) positional data of the handheld surgical instruments in relation to the surgical field. This requires the “line of sight” maintenance between the LEDs and cameras at all times. Hand movements or excess operating personnel across this line of sight might hinder the navigation process. Electromagnetic systems allow an unobstructed view between transmitter and receiver. However, significant interference of the images can occur due to metal artifacts and electromagnetic fields originating from other commonly used operating room devices such as cautery and electrocardiogram monitoring equipment.

The navigation assisted spinal methods may require one or more manual operation and/or steps from the surgeon. The surgeon will continue to coordinate any methods, including access, approach, bony preparation and/or implant positioning by coordinating his own operations with the pre-processed, high-resolution images on the navigation system screen. The overall benefits include accurate access, surgical approach, bony preparation, implant positioning, minimal radiation exposure to the surgical team, and/or reduction of surgeon fatigue and surgical duration.

Alternatively, the surgeon may desirably decide to use robotic assisted spine surgery to improve the accuracy of surgical access, selection of spinal implant, bony preparation and/or implant positioning. Robotic assisted spine surgery can be used to further accurately restore spinal alignment and range of motion. Robotic assisted spine surgery requires the use of a navigational system as described herein. The robotic system mainly uses preoperative and intraoperative images for surgical planning, proving accurate positioning of surgical tools or implants through robotic arm movement and rigid guidance, assisting the surgeon to complete surgical operations. The robotic intraoperative surgical processes may comprise a plurality of broad steps, including: (1) surgical planning, with the surgeon carrying out the surgical planning and selecting the suitable implants based on the patient images using the navigational software; (2) spatial registration, obtaining the spatial coordinates of the surgical trajectories via software algorithms and tools; (3) trajectory positioning, with the robotic arm providing autonomous movement by holding the surgical instruments to the desired position according to the spatial coordinates of the planned surgical trajectory; and/or (4) assisted surgery, with the surgeon performing the surgical operation under the guidance of the robotic arm.

With reference to FIG. 18B, a top view of one embodiment of a robotic system set-up 1015 is shown. The robotic system set-up 1015 comprises a navigational system, the navigational system includes a navigational workstation 985, a referencing system 990, an imaging unit 980 using an imaging technique and/or a robot or a robotic arm 1010. The navigational system set-up 975 may include other traditional items that include anesthesia monitoring equipment 990, an instrument table 995 to hold sterilized instruments and implants, a patient tracking system table 1000, and an operating bed 1005. As described herein, the navigational system comprises a workstation. The navigational workstation 985 includes image acquisition processing software (e.g., navigation software), memory, a processing unit (e.g., computer), a display and/or a transceiver. The robotic arm 1010 is a mechanism comprising two or more degrees of freedom (DOF), a certain degree of autonomy, and can perform a predetermined task according to planned human instructions.

The robots or robotic arms 1015 comprise different functions, the functions include supervisory-controlled robot, telesurgical robot, and/or shared control robot. Supervisory controlled robots allow the surgeon to plan the operation in its entirety pre-operatively; the robot then performs the operation under close supervision by the surgeon. Telesurgical robots allow the surgeon to directly control the robot and its instruments throughout the entire procedure from a remote location. Finally, most spinal surgery robots are shared-control robots that simultaneously allow both the surgeon and robot the ability to control instruments and motions.

Any type of robotic system may be used. The robotic systems may comprise a Mazor SpineAssist, Mazor Renaissance, and/or Mazor X (Medtronic, Inc., Minneapolis, MN). The Mazor SpineAssist is a shared-control robot that offers navigation superior to traditional intraoperative navigational systems. The Mazor Renaissance contains upgraded software and hardware improvements from the SpineAssist. The Mazor X includes a linear optic camera that allows the robot to perform a volumetric assessment of the work environment to self-detect its location and provide collision avoidance intraoperatively. The robotic systems may further comprise a ROSA Spine (Zimmer Biomet Robotics, Montpellier, France). The ROSA is a free-standing robotic arm and navigation arm. The robotic systems may further comprise a Da Vinci Surgical System (Intuitive Surgical, Sunnyvale, CA). The Da Vinci uses a telesurgical function and operates from a remote telesurgical booth equipped with 3D vision screens, allowing the robot to be an extension of the surgeon's arm. The robotic systems may further comprise an Excelsius GPS (Globus Medical, Inc., Audubon, PA). The Excelsius features real-time imaging, automatic compensation for patient movement, and constant feedback. The robotic system may further comprise a Pulse System (NuVasive, Inc., San Diego, CA). The Pulse System an open imaging platform integrated with Siemens' 3D mobile C-arm, Cios Spine; Cirq Spine System and Loop Imaging Robot (BrainLab AG, Munich, Germany). The robotic system may further comprise a Cuvis Spine System (Curexo, South Korea) and/or a Fusion Robotic System (Integrity Implants, Inc., d/b/a Accelus, Palm Beach Gardens, FL). Both the Cuvis and the Fusion are 3D imaging navigation and robotic targeting systems for spine surgery.

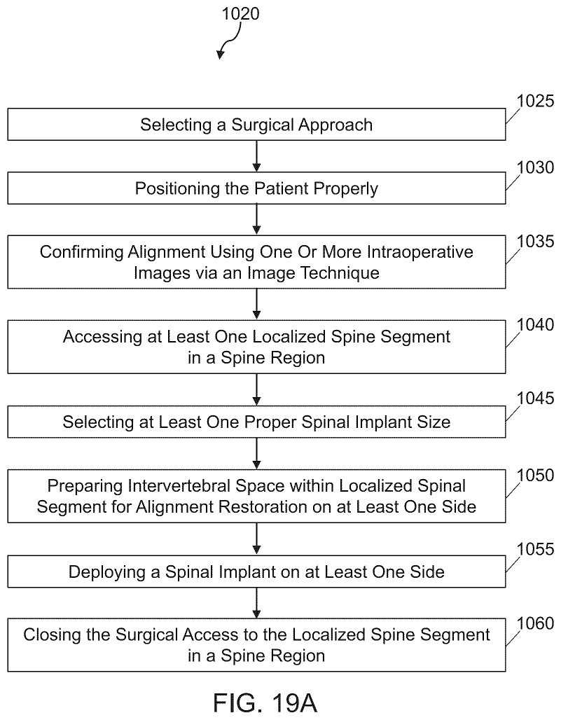

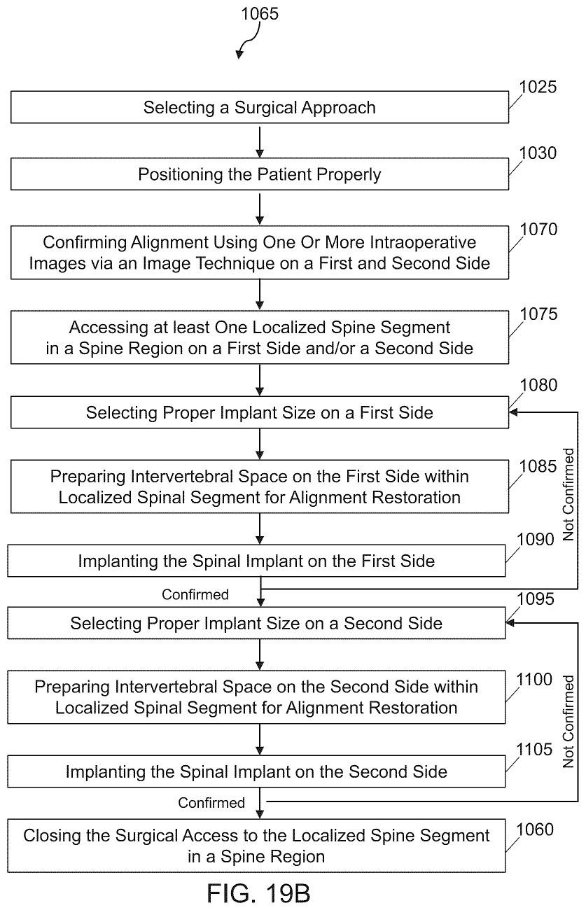

With reference to FIG. 19A, a flowchart illustrating one embodiment of a standard intraoperative or operative protocol 1020 is shown. The standard operative protocol 1020 may be desired by the surgeon, which one or more steps of the surgery is completed manually. The standard operative protocol 1020 comprises the steps of: selecting a surgical approach 1025; positioning the patient properly on a surgical table 1030; confirming alignment using one or more intraoperative images 1035; accessing the localized spinal segment in a spine region 1040; selecting proper implant size (tensioning soft tissues and trialing) on at least one side 1045; preparing an intervertebral space within the localized spinal segment (prepare endplate upper vertebral body, prepare endplate of lower vertebral body, pedicle osteotomy and keel cuts/channels) on the at least one side 1050; and/or deploying or implanting the total joint spinal implant on the at least one side 1055. The intraoperative method may further comprise the step of removing the implanted total joint spinal implant (not shown). The intraoperative method may further comprise the step of closing the surgical access to the localized spine segment in a spine region 1060. A spine region may comprise a cervical region, a thoracic region, a lumbar region, a sacral region, and/or any combination thereof.

With reference to FIG. 19B, a flowchart illustrating an alternative embodiment of the standard intraoperative or operative method 1065 is shown. The alternative standard operative method 1065 comprises the steps of: selecting a surgical approach 1025; positioning the patient properly on a surgical table 1030; confirming alignment using one or more intraoperative images on a first side and a second side 1070; accessing the localized spinal segment in a spine region on a first side and/or a second side 1075; selecting proper implant size (tensioning soft tissues and trialing) on a first side 1080; preparing an intervertebral space within the localized spinal segment (prepare endplate upper vertebral body, prepare endplate of lower vertebral body, pedicle osteotomy and keel cuts/channels) on a first side 1085; deploying or implanting the proper spinal implant on a first side 1090; selecting a second proper implant size (tensioning soft tissues and trialing) on a second side 1095; preparing an intervertebral space within the localized spinal segment (prepare endplate upper vertebral body, prepare endplate of lower vertebral body, pedicle osteotomy and keel cuts/channels) on a second side 1100; and/or deploying or implanting the proper spinal implant on a second side 1105. The intraoperative method may further comprise the step of removing the implanted total joint spinal implant (not shown). The intraoperative method may further comprise the step of closing the surgical access to the localized spine segment in a spine region 1060. A spine region may comprise a cervical region, a thoracic region, a lumbar region, a sacral region, and/or any combination thereof.

Alternatively, the surgeon may desirably decide to use navigation assisted and/or robotic assisted 1115 spine surgery to improve the accuracy of one or more operative steps 1110, 1120 as shown in as shown in FIGS. 14A-14C, 15A-15B, 16A-16C, 17A-17C, 20A-20B and/or 21A-21E. The one or more operative steps 1110, 1120 may comprise one or more steps of: surgical access (e.g., accessing at least one localized spine segment in a spine region on at least one side 1040, on a first side and/or second side 1075), selection of a proper spinal implant on at least one side 1045, on a first side 1080 and/or a second side 1095), bony preparation (e.g., preparing an intervertebral space on at least one side within a localized spine segment 1050 and or a first side 1085 and/or a second side 1100) and/or implant positioning (e.g., this may include implanting a spinal implant on at least one side 1055, a first side 1090 and/or a second side 1105).

More specifically, navigation and/or robotic assisted 1115 surgery may be inserted within and/or into one or more operative steps 1125, 1200, 1205, 1210. The navigation and/or robotic assisted surgery 1115 may include additional procedures or methods than the standard or traditional operative steps 1020, 1065 to facilitate the identification of equipment within desired spatial coordinates, to confirm spatial coordinates and desired trajectory within one or more targeted vertebral segments; positioning the user to desired trajectory; and correcting the deviated trajectory back to the desired trajectory.

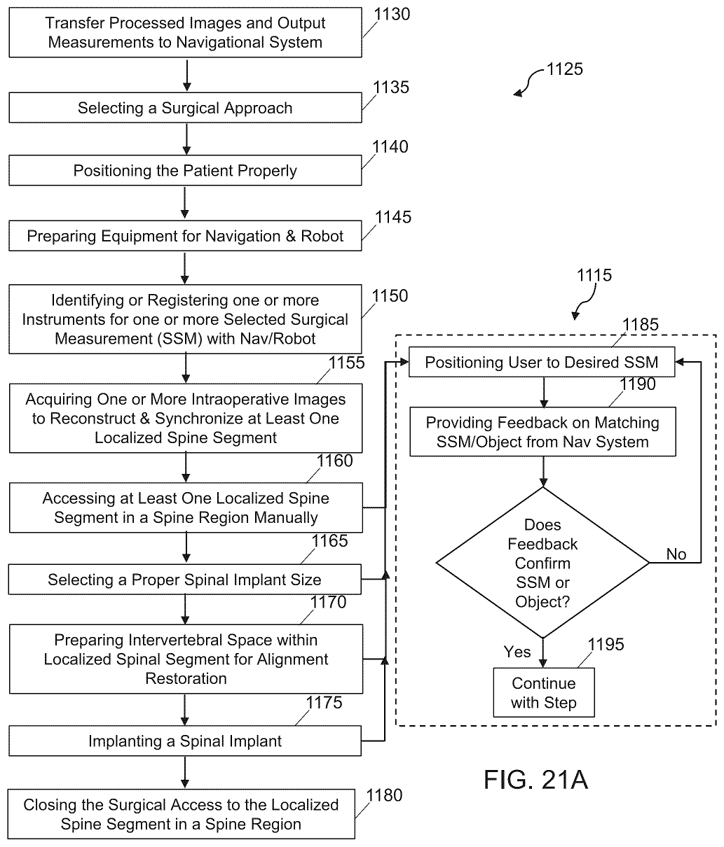

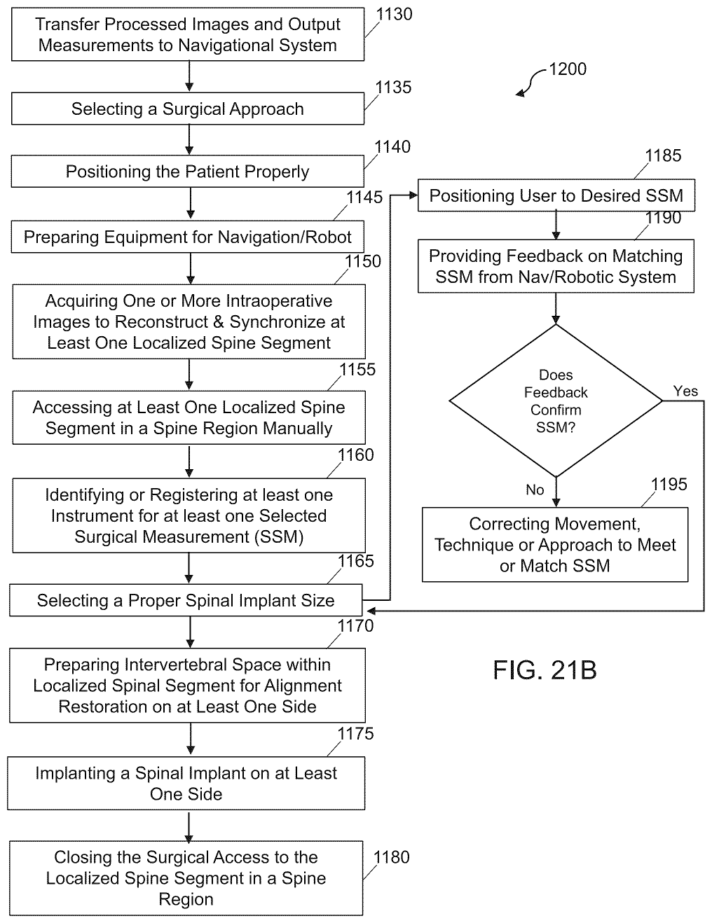

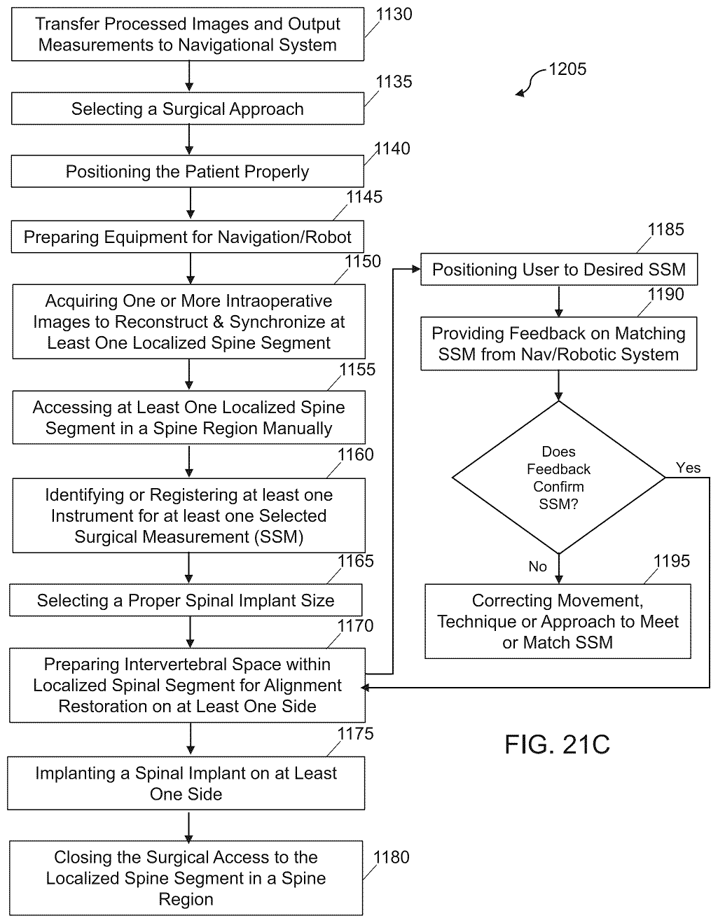

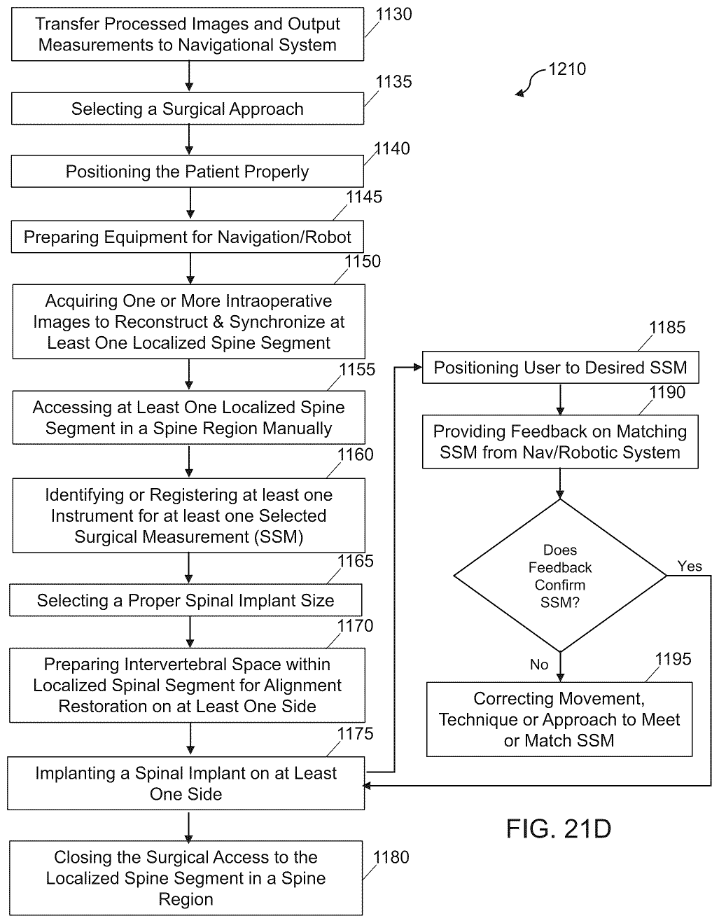

With reference to FIGS. 21A-21D, the flowcharts illustrate different embodiments of one or more operative steps 1125, 1200, 1205, 1210 that can use robotic and/or navigational assistance 1115. The one or more navigational and/or robotic assisted operative steps 1125 may comprise one or more of the following: a method of improving accuracy of selecting a proper implant size by using a surgical navigation and/or robotic system 1200 (see FIG. 21B); a method of improving accuracy of preparing an intravertebral space by using a surgical navigation and/or robotic system 1205 (see FIG. 21C); and/or a method of improving accuracy of implanting a spinal implant by using a surgical navigation and/or robotic system 1210 (see FIG. 21D). The one or more navigational and/or robotic assisted operative steps 1125 may further comprise one or more of the following: confirming alignment using one or more intraoperative images 1035, 1070; accessing at least one localized spine segment in a spine region 1040, 1075.

The one or more navigational and/or robotic assisted operative steps 1125 comprises the steps of: transfer a preoperative plan to a workstation 1130, the preoperative plan comprises selected surgical measurements (SSM) and/or processed images obtained from the preoperative surgical planning software; selecting a surgical approach 1135; positioning the patient properly 1140; preparing the equipment for navigation and/or robotic assisted spine surgery 1145; identifying or registering a surgical instrument for at least one surgical measurement 1150; synchronizing at least one preoperative image with the at least one reconstructed operative image to confirm the at least one surgical measurement from the preoperative plan 1155; accessing at least one localized spine segment in spine region 1160; selecting proper implant size 1165; preparing intervertebral space within a localized spine segment 1170; implanting or deploying a spinal implant on at least one side; implementing navigational and/or robotic assistance 1115 in one or more of the above steps 1115.

The navigation and/or robotic assistance 1115 in one or more of the above one or more steps 1115 includes the steps of: positioning user to desired trajectory and/or object, the desired trajectory and/or object matching or substantially matching one or more SSM 1185; providing feedback to the surgeon to confirm matching or substantially matching the at least one surgical measurement from the preoperative plan and/or correcting deviated trajectory of the user to return to the desired trajectory 1190. The step of providing feedback may comprise visual feedback (using the navigation monitor), an audible feedback, a tactile feedback, and/or any combination thereof. The steps of selecting a surgical approach; positioning the patient properly; preparing the equipment for navigation; acquiring one or more operative images to create a reconstructed image of a localized spinal segment in a spinal region; and/or accessing at least one localized spine segment in spine region comprises completing the technique manually. Alternatively, the completion further comprises navigation assisted or robotic assisted.

In one embodiment, the navigational and/or robotic assisted intraoperative method may comprise the step of transferring data set and/or preoperative plan into an operative workstation 1130. The data set may include computed selected surgical measurements, 2D processed images and/or 3D processed images (see FIG. 8B-8C) from the preoperative surgical planning software as described herein. The data set may be will be used help confirm the operative plan.

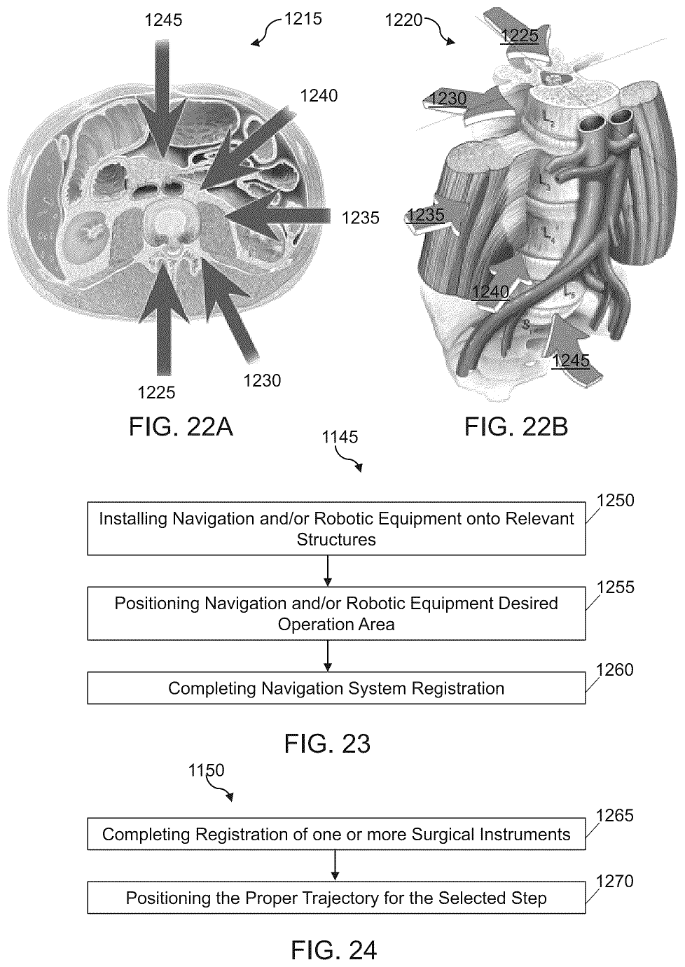

In one embodiment, the navigational and/or robotic assisted intraoperative method may comprise the step of selecting a surgical approach 1135. FIGS. 22A-22B illustrate a top view 1215 of a targeted spine segments (L1-S1) and an isometric view 1220 of the targeted spine segments (L1-S1). The surgical approaches selected may be similar to standard surgical fusion approaches, including posterior lumbar interbody fusion (PLIF) 1225, transforaminal lumbar interbody fusion (TLIF) 1230, minimally invasive transforaminal lumbar interbody fusion (MI-TLIF), lateral lumbar interbody fusion (LLIF) 1235, oblique lumbar interbody fusion/anterior to psoas (OLIF/ATP) 1240, anterior lumbar interbody fusion (ALIF) 1245 and/or any combination thereof. In one preferred embodiment, the surgical approach may comprise a PLIF 1225 or a TLIF 1230.

In another embodiment, the robotic assisted and/or navigation assisted intraoperative method may comprise the step of positioning the patient properly on a surgical table 1140. The step of positioning the patient properly 1140 comprises the steps of: positioning the patient onto the surgical table; and confirming alignment by acquiring one or more intraoperative images using at least one imaging technique. Patient positioning is important to ensure proper sagittal alignment, mobility and implantation of the spinal implant. The patient is initially positioned in a prone position in a neutral alignment with legs extended on an Andrews or Jackson table with bolsters. The patients' prone position on the table should match or substantially match the patient's standing sagittal position. The abdomen should remain free with no pressure on the bladder and with hip pads under the anterior superior iliac spine. This position allows the patient's spine and anatomy to move to a neutral alignment after paralytics and/or posterior bony elements are removed during the decompression step.

With reference to FIG. 23, the flowchart illustrates one embodiment of a robotic assisted and/or navigation assisted intraoperative method that may comprise the step of preparing equipment for navigation and/or robotic assistance 1145. The step of preparing equipment for navigation and/or robotic assistance 1145 includes the steps of: installing navigation and/or robotic equipment onto relevant parties and/or structures 1250; positioning navigation and/or robotic equipment into desired operation area 1255. The step may further include completing navigation system registration 1260.

The step of installing navigation and/or robotic equipment onto relevant parties and/or structures 1250 ensures that all the navigation and/or robotic systems' architecture is available. The navigation system comprises a workstation and a referencing system. The workstation includes an image acquisition/processing software and a computer (e.g., a processing unit and memory). A robotic system comprises robotic arm, a navigation system and a referencing system. The referencing system may include a DRA, LEDs and/or a tracking system or equipment. A portion of the tracking system will be attached or coupled to a portion of a patient's anatomy, table and/or the robot. A portion of the DRA may be disposed and/or coupled onto the portion of the tracking system. Such rigid coupling maintains the relationship between the patient and the DRA during the registration process and/or intraoperative surgical procedure. Furthermore, a probe or a guide may be coupled to the robot or robotic arm. The probe or a guide is a surgical tool that facilitates positioning the trajectory for the surgeon and/or robot.

The step of positioning navigation and/or robotic equipment into desired operation area 1255 may include covering the robot probe, and/or guide with sterile covers. The registrations markers should be further installed and positioned into the operation area, so the registration markers are within the fluoroscopic or image field. Any other set-up for the referencing system or robotic equipment may also be performed to standards known in the art.

The step completing navigation system registration 1260 is a process in which surgeons tell the computer where the bone, targeted anatomy, instruments and/or patient is in the space by identifying the anatomical landmarks for the computer to create proper spatial coordinates. Therefore, workstation computes a relationship between the one or more images (Xray, CT, MRI or patient's 3D bone model) with the patient's anatomy at the operative site. The step of completing navigation system registration 1260 comprises the steps of: registering of one or more navigation tools to navigation system; and/or registering of a patient's targeted anatomy to navigation system. The step of completing navigation system registration 1260 further comprises the steps of: identifying or registering one or more surgical instruments to navigation system 1150; and/or acquiring one or more images using an imaging technique to finalize registration process.

The step of registering of one or more navigation tools, robotic tools and/or robotic units to navigation system may utilize steps or methods known in the art. In one embodiment, a robotic unit has been formally positioned, the robot may proceed with automatic registration and tracking of the robot arm and/or unit. The position information obtained by the -navigation system can be directly mapped to the robotic arm, which provides great convenience for automatic registration and tracking of the robot arm.

The step of registering of a patient's targeted anatomy to a navigation system may include one or more different available anatomical registration techniques. The anatomical registration techniques include tracker pins or fiducials technique, skin surface technique, imaging technique and/or bony landmarks technique. The tracker pin or fiducial technique completes registration by placement of tracker pins or fiducial markers at the target bones during 2D/3D image acquisition. At least four fiducials are required. Registration is completed after images are acquired or the fiducial markers are identified at the surgery.

The second technique, the skin surface-matching technique, is completed by moving a registration tool, e.g., a laser pointer, probe and/or guide, over the surface of the skin. The registration tool collects a plurality of data points for registration. The third method, the imaging technique uses 2D-3D imaging registration. One or more images are obtained intraoperatively are matched automatically with preoperative CT images after manual image adjustment. The fourth technique, the bony landmarks technique, uses bone surfaces for registration. The registration tool should move between defined landmarks on the skull surface. These landmarks must be defined on the virtual patient during the presurgical planning process. These fiducials may include anatomic landmarks (i.e., suture lines or bone prominences or concavities, teeth etc.). If necessary, the registration tool is passed through the skin so it may be placed directly onto the bone surface.

The step of acquiring one or more images using an imaging technique to finalize registration process requires the collection or acquisition of one or more images to confirm proper spatial coordinates of patient's targeted anatomy. One or more images are acquired and transferred to the navigation computer or workstation. The navigation computer or workstation may reconstruct or transform acquired one or more images into sagittal, coronal and/or axial views. The navigation or workstation identifies the DRAs and/or the reference system to facilitate the registration process. The navigation computer and/or workstation correlates the one or more transformed or reconstructed images to the patient's anatomy. Then, the navigation computer and/or workstation synchronizes the one or more intraoperative images with the one or more preoperative images. Accurate registration is confirmed by the surgeon after verifying that the registration tool end correlates to a similar location on the navigation computer and/or workstation. A mismatch should prompt repetition of the step of navigation system registration 1260 process.

With reference to FIG. 24, the flowchart illustrates one embodiment of the step of identifying or registering one or more surgical instruments 1150. The step of identifying or registering one or more surgical instruments 1150 may comprise the step of: registering one or more surgical instruments to the navigation system 1265. The step of identifying or registering one or more surgical instruments 1150 may further comprise the step of: positioning the one or more users and/or surgical instruments to the desired or first trajectory for at least one intraoperative step 1270.

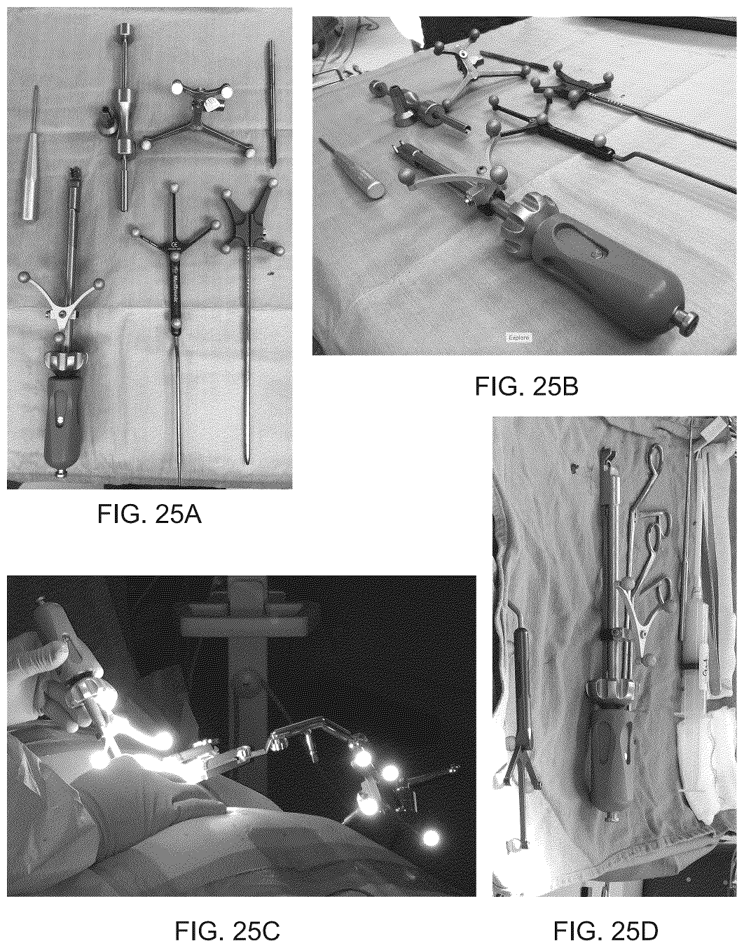

The step of registering one or more surgical instruments to the navigation system 1265 comprises coupling at least one DRA onto one or more surgical instruments. The one or more surgical instruments were pre-registered, templated and/or pre-identified within the navigation system during the preoperative procedure. Alternatively, the one or more surgical instruments can be registered, templated and/or identified during the operative procedure. The one or more surgical instruments are designed and configured to receive at least one DRA, marker and/or fiducial as shown in FIGS. 25A-25D. The navigation system and/or robotic system should facilitate a spatial relationship of the one or more surgical instruments to the target bone and relative to the operating field. Furthermore, the one or more surgical instruments may be projected onto one or more operative images, one or more reconstructed images and/or one or more synchronized images.

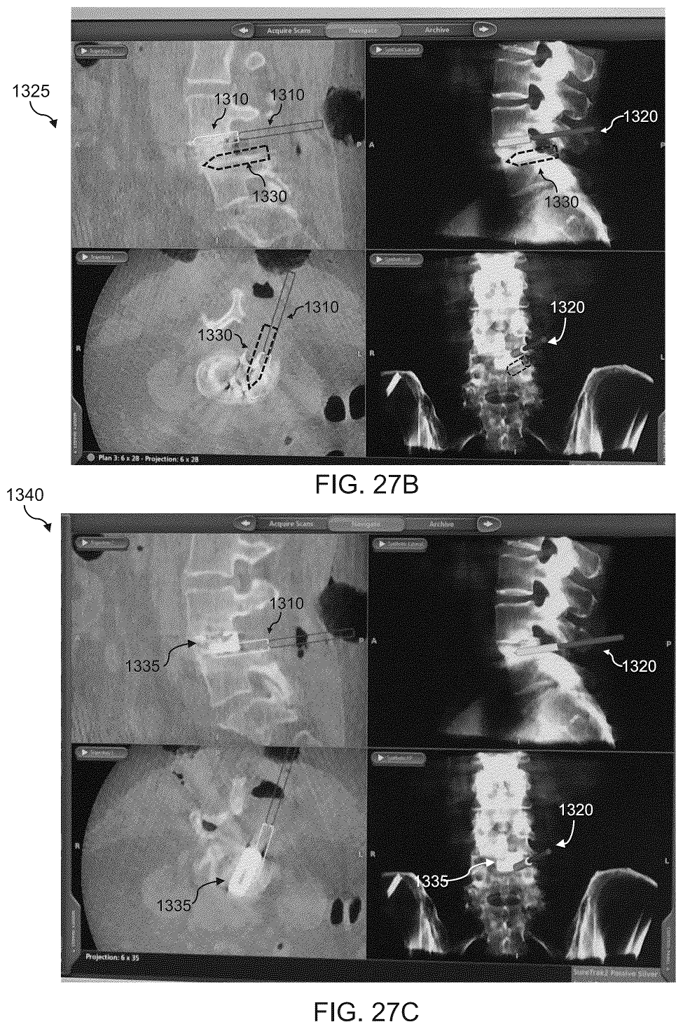

Accordingly, the step of positioning the one or more users and/or surgical instruments to the desired or first trajectory for the at least one intraoperative step 1270. The at least one operative step may comprise a plurality of intraoperative or operative steps. The at least one operative step may comprise the entire or all the intraoperative steps. Once all tools, surgical instruments and patient are registered, the navigation system may transmit spatial coordinates of the one or more surgical instruments relative to the patient's targeted anatomy for positioning of a first trajectory or a desired trajectory. One or more objects matching or substantially matching the first or desired trajectory may be projected onto the display. Such trajectory positioning may allow the navigation system and/or robotic system by holding one or more surgical instruments and/or a registration tool (i.e., a probe, a guide and/or any other tool) to the desired position according to the spatial coordinates of the surgical trajectory for the desired intraoperative step. The trajectory may match or substantially match one or more projected surgical measurements (PSM) and/or selected surgical measurements (SSM). The trajectory may be displayed and/or superimposed onto the workstation as one or more virtual objects. The one or more virtual objects may comprise quantitative measurements (e.g., SSMs), a virtual trajectory path, a virtual implant, and/or any combination thereof as shown in FIGS. 27A-27B.

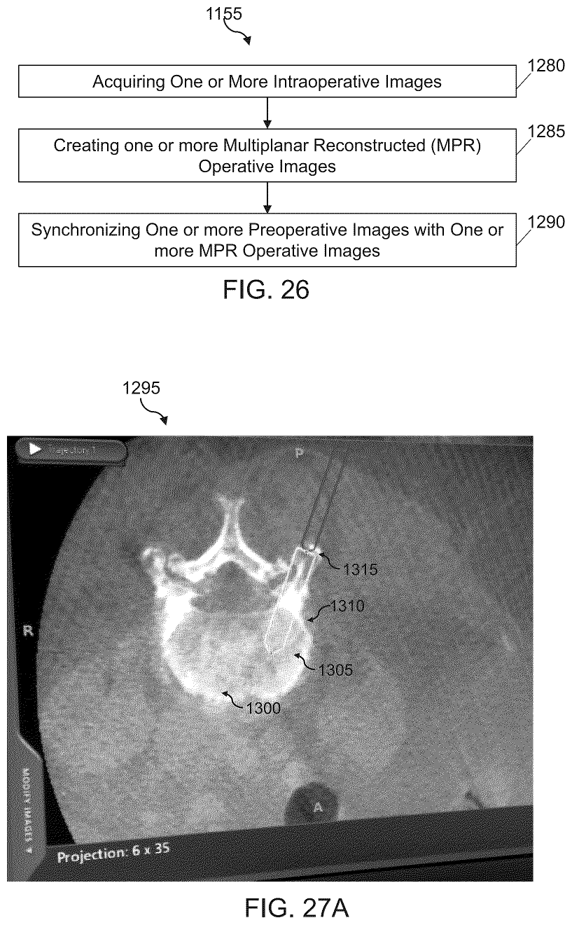

With reference to FIG. 26, the flowchart illustrates one embodiment of the robotic assisted and/or navigation assisted intraoperative method that comprises the step of acquiring one or more operative images to reconstruct the localized spine segment 1155. The step of synchronizing one or more intraoperative images with the one or more preoperative images to reconstruct the localized spine segment 1155 comprises the steps of: acquiring one or more intraoperative images in a plurality of planes using an imaging technique 1280; creating one or more multiplanar reconstructed (MPR) images 1285; and/or synchronizing one or more preoperative images with the one or more operative MPR images 1290. The step of synchronizing one or more intraoperative images with the one or more preoperative images to reconstruct the localized spine segment 1155 may further comprise the step of: positioning the one or more users and/or surgical instruments to the desired or first trajectory for at least one intraoperative step 1270.

The step of acquiring one or more intraoperative images in a plurality of planes using an imaging technique 1280 are obtained in one or more anatomical planes of the targeted anatomy of a patient. The imaging techniques include an MRI, a radiograph, a CT scan, an ultrasound, and/or any combination thereof. The acquisition of the one or more intraoperative images may be acquired using a specialized grid, which allows for easier synchronization of the one or more preoperative images with the one or more multiplanar reconstructed (MPR) images. The one or more intraoperative images in the one or more anatomical planes are used to create one or more MPR images 1285. The one or more MPR images may include 2D and/or 3D reconstructed intraoperative images of a patient's targeted anatomy.

The step of synchronizing one or more preoperative images with the one or more operative MPR images 1290 comprises the steps of: superimposing the one or more preoperative images onto the one or more operative MPR images. The step of synchronizing one or more preoperative images with the one or more operative MPR images 1290 may further comprise the step of: superimposing one or more selected surgical measurements (SSM) or projected surgical measurement (PSM) and/or one or more objects onto the one or more preoperative images, one or more operative images, one or more MPR images and/or one or more synchronized images.

The step of superimposing the one or more preoperative images onto the one or more operative MPR images comprises the step of acquiring the one or more preoperative images from the preoperative surgical plan as described herein. The one or more preoperative images may include original or raw one or more preoperative images, the one or more modified preoperative images and/or the one or more processed preoperative images or processed virtual models. The one or more processed preoperative images or processed virtual models are images of a targeted anatomy that are enhanced using the preoperative surgical planning software. The one or more processed preoperative images or processed preoperative virtual models are uploaded, transmitted and/or displayed to a workstation to be superimposed onto one or more operative images. The one or more operative images comprise one or more MPR images. The workstation should synchronize the one or more processed preoperative images or processed preoperative virtual models with the one or more operative MPR images to create one or more synchronized images. The synchronization process confirms that the anatomic landmarks of the targeted anatomy of the preoperative and/or operative images correlates to the one or more operative MPR images to ensure of the desired targeted anatomy is reached. The navigation workstation confirms these anatomical landmarks by matching multiple reference points on each vertebral body.

With reference to FIGS. 27A-27C, the one or more operative images or MPR images 1295, 1325, 1340 depict one embodiment of the step of superimposing one or more selected surgical measurements (SSM) or projected surgical measurement (PSM) and/or one or more objects into the one or more preoperative images, one or more operative images, MPR images and/or synchronized images. The one or more objects may include a text annotation (not shown), a trajectory path 1310, a virtual surgical instrument 1320, a spinal implant 1335 and/or any combination thereof. The trajectory path 1310 may comprise the trajectory of a surgical instrument, surgical approach and/or one or more spinal implants. The trajectory path of an approach may include a first end 1305 and a second end 1315. The first end 1305 may delineate the anterior most position that the user, surgeon and/or robot may proceed. The second end 1315 may delineate the posterior most position that the surgeon and/or robot may be positioned.

In one embodiment, a first one or more objects are superimposed onto one or more operative images and/or MPR images and a second one or more objects are superimposed onto one or more operative images and/or MPR images. The first one or more objects may comprise a desired or first trajectory path 1310 to a targeted anatomical location. The second one or more objects may comprise a non-desired, deviated or second trajectory path 1330 to the targeted anatomical location. The second trajectory path 1330 is different than the first trajectory path 1310. The workstation provides feedback to correct the user (e.g., robot or surgeon) from the second trajectory path back to or return to the first trajectory path 1310.

In another embodiment, a first one or more objects are superimposed onto one or more operative images and/or MPR images and a second one or more objects are superimposed onto one or more operative images and/or MPR images. The first one or more objects may comprise a desired or first one or more spinal implants 1335 at a targeted anatomical location. The one or more implants 1335 provide visual support of the trajectory path. The second one or more objects may comprise a virtual tool 1320 that is deviating from the targeted anatomical location and/or does not follow or substantially follow the trajectory path of the one or more implants 1335. The trajectory path of the virtual tool 1320 is different than the trajectory path of the one or more implants 1335. The workstation provides feedback to correct the user (e.g., robot or surgeon) from the second trajectory path of the virtual tool 1320 back to or return to the first trajectory path of the one or more implants 1335.

In another embodiment, step of acquiring one or more intraoperative images to reconstruct the localized spine segment 1155 may further comprise the step of conducting real-time operative planning. The step of real-time operative planning comprises the steps of: acquiring one or more intraoperative images in a plurality of planes; and creating one or more multiplanar reconstructed (MPR) images. The step of conducting real-time operative planning may further comprise the step of superimposing one or more objects into the one or more preoperative images, one or more operative images and/or MPR images as described herein. Real-time operative planning allows the surgeon to have an option to omit the one or more preoperative images from the preoperative plan to create a new operative plan for the surgical approach and/or provide a trajectory for the surgeon and/or robot. The surgeon must perform the step of confirming an anatomical location to initiate a starting point. The confirmation of a starting point may be done by two techniques. The first technique includes direct visualization, which should confirm an adequate starting point based on the anatomical landmarks the surgeon would recognize for freehand techniques. The second technique includes confirming bony anatomy with the navigation. This can be done by using a passive planar navigation probe and placing it on an anatomical bony landmark, such as the spinous process. The navigation system should confirm that the probe position on the landmark corresponds to the probe position seen on the navigation screen. If there is a mismatch, then the surgeon needs to consider re-registration of the reference frame.

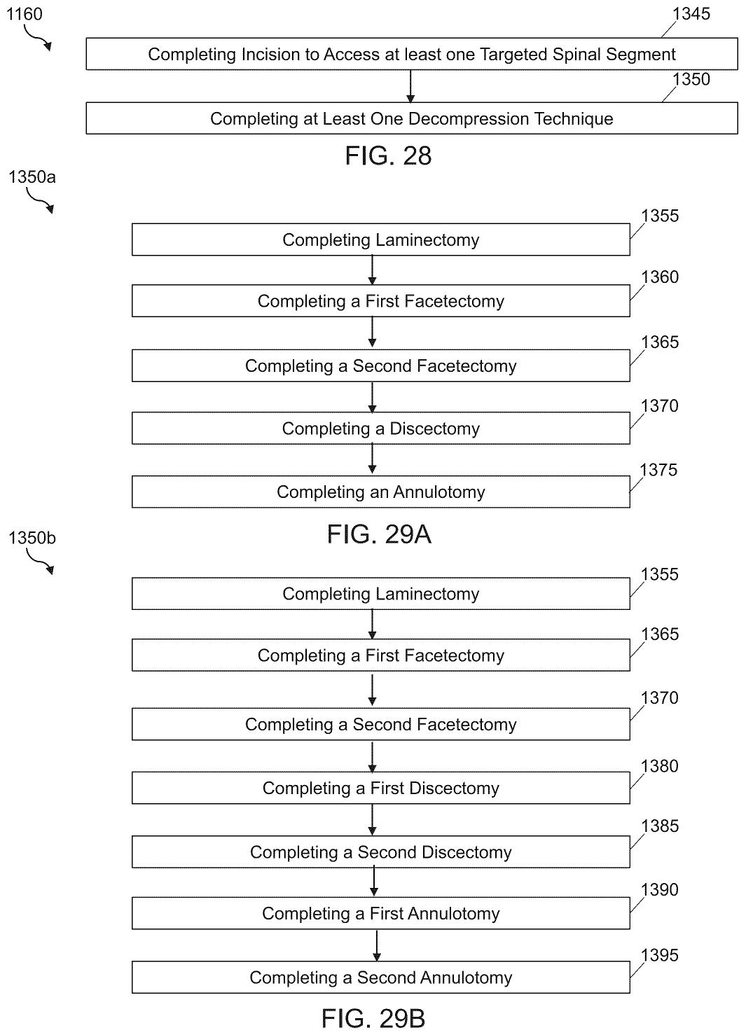

With reference to FIG. 28, the navigation assisted and/or robotic assisted intraoperative method may comprise the step of: accessing a localized spine segment within a spine region 1160. The step of accessing the localized spinal segment in a spine region 1160 may comprise the steps of: completing the incision to access a targeted anatomical location 1345; and/or completing at least one decompression technique 1350, 1350a, 1350b. The targeted anatomical location may comprise one or more vertebral segments within the spinal region.

In one embodiment, the step of completing the incision to access at least one targeted anatomical location 1345 may be performed according to standard surgical techniques (manually), navigation assisted and/or robotic assisted. The step of completing the incision to access at least one targeted spinal segment within a spinal region 1345 may include localizing the incision at, near and/or proximate to the at least one localized spinal segment in a desired spine region. The spine region may include a thoracic region, a cervical region, lumbar region, sacral region and/or any combination thereof. The at least one spine segment comprises one or more localized spinal segments that may include one or more cervical vertebral segments (C1-C8), thoracic vertebral segments (T1-T12), lumbar vertebral segments (L1-L5), and/or sacral vertebral segment (S1-S5).

The incision to access the targeted spinal segment in a spine region may comprise a standard midline incision with a subperiosteal exposure that extends laterally to include the medial edge of transverse process. Utilize intraoperative imaging with an imaging technique to confirm the proper index level and/or targeted spinal segment. The surgeon may expose the entirety of the facet joint and lamina at the targeted spinal segment within a spine region. Alternatively, the surgeon may expose the entirety of the facet joint, lamina and transverse process. The surgeon may choose to not dissect the transverse process of the L5 vertebra because it may be necessary to maintain the integrity of the iliolumbar ligament. Disruption of the iliolumbar ligament can weaken the transverse process. This is especially important for patients with spondylolisthesis at L5-S1. Furthermore, the surgeon may desirably avoid violating the cranial facet capsule of the facet joint and to avoid transverse process fracture, especially at L5. The surgeon should attempt to achieve hemostasis.

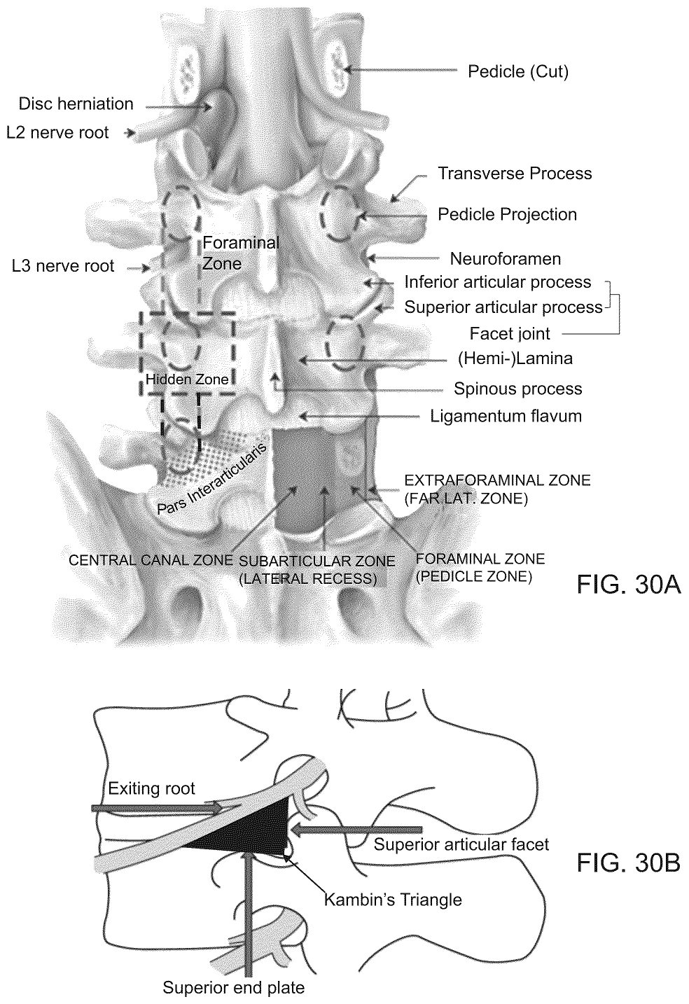

With reference to FIGS. 29A-29B, the step of completing at least one decompression technique 1350, 1350a, 1350b may comprise one or more decompression techniques to achieve pedicle-to-pedicle exposure of the foramen and exiting nerve root and/or exposure of the “hidden zone” and/or Kambin's triangle on at least one side at the at least one targeted spinal segment as shown in FIGS. 30A-30B. FIGS. 30A-30B illustrates the one embodiment of the “hidden zone” and Kambin's Triangle. The hidden zone may be bordered by the subarticular (lateral recess) a zone, the foraminal (pedicle) zone and the extraforaminal (far lateral) zone. Within these zones lies the “hidden zone.” Kambin's Triangle may provide access to the “hidden zone.”

The one or more decompression techniques 1350, 1350a, 1350b may include one or more of the following: a laminectomy, a laminotomy, a foraminotomy, a laminoplasty, a discectomy, an annulotomy and/or any combination thereof. The one or more decompression techniques may comprise a complete or partial technique. The one or more decompression techniques may be completed on least one side. The one or more decompression techniques may be completed on a first side and a second side.

In one embodiment, the step of completing at least one decompression technique 1350 may comprise a laminectomy and at least one facetectomy. The at least one facetectomy may comprise an inferior and/or superior facetectomy. The at least one facetectomy may comprise a complete/full or partial facetectomy. The laminectomy may include a standard laminectomy, a Gill laminectomy and/or a hemi-Gill laminectomy.

With reference to FIG. 29A, the step of completing at least one decompression technique 1350a may comprise the steps of: completing a laminectomy 1355, a first facetectomy 1360, and a second facetectomy 1365. The step of completing at least one decompression technique 1350a may further comprise the steps of a completing a discectomy 1370 and/or completing an annulotomy 1375. The first or second facetectomy 1360, 1365 may comprise a complete or partial facetectomy. The first facetectomy 1360 may comprise a complete inferior facetectomy, complete superior facetectomy, a partial inferior facetectomy, and/or a partial superior facetectomy. The second facetectomy 1365 may comprise a complete inferior facetectomy, complete superior facetectomy, a partial inferior facetectomy, and/or a partial superior facetectomy. In another embodiment, the step of completing at least one decompression technique 1350a may comprise the steps of completing a laminectomy 1355, a first facetectomy 1360 that includes a complete inferior facetectomy, completing a second facetectomy 1365 that includes a partial superior facetectomy. The step of completing at least one decompression technique may further comprise a completing a discectomy 1370 and/or completing an annulotomy 1375.

With reference to FIG. 29B, the step of completing at least one decompression technique 1350b may comprise the steps of: completing a laminectomy 1355, completing a first facetectomy 1360 on a first side, and completing a second facetectomy on a second side 1365. The step of completing at least one decompression technique 1350b may further comprise the steps of: completing a first discectomy one a first side 1370, completing a second discectomy on a second side; completing a first annulotomy on a first side 1390; and or completing a second annulotomy on a second side 1390. The first or second facetectomy 1360, 1365 may comprise a complete or partial facetectomy. The first facetectomy 1360 may comprise a complete inferior facetectomy, complete superior facetectomy, a partial inferior facetectomy, and/or a partial superior facetectomy. The second facetectomy 1365 may comprise a complete inferior facetectomy, complete superior facetectomy, a partial inferior facetectomy, and/or a partial superior facetectomy. In another embodiment, the step of completing at least one decompression technique 1350b may comprise the steps of completing a laminectomy 1355, a first facetectomy 1360 that includes a complete inferior facetectomy and a partial superior facetectomy on a first side; completing a second facetectomy 1365 that includes a complete inferior facetectomy and partial superior facetectomy on a second side. The step of completing at least one decompression technique 1350b may further comprise a completing a first discectomy on a first side 1380; completing a second discectomy on a second side 1385; completing a first annulotomy on a first side 1390; and/or completing a second annulotomy on a second side 1395. The first side and/or second side comprises the right or left sides of a patient and/or the pedicles.

In one embodiment, the step of completing a laminectomy 1355 and completing a facetectomy 1360, 1365 may be performed to standard surgery techniques, navigation assisted techniques and/or robotic assisted techniques. In another embodiment, the step of completing a laminectomy 1355 and completing a facetectomy 1360, 1365 may be performed to standard surgery techniques with supplemental considerations. The supplemental considerations may include confirming flushness and/or the location of the nerve root. The surgeon may confirm that the medial portion of the superior articular process should be flush with the medial wall of the inferior pedicle. Also, the surgeon may remove the top portion of the superior articular process to be flush with the inferior pedicle.

The surgeon may avoid violating the cranial facet capsule of the facet joint. The supplemental considerations may further include having the surgeon confirm the visualization and/or location of the nerve root. The exiting nerve root should be exposed and visualized from the hidden zone to the far lateral zone. Any tethering tissue from the lateral pedicle to the exiting nerve root should be released to allow gentle superior-lateral retraction of the exiting nerve root. The surgeon may desirably want to avoid dissecting iliolumbar ligaments to avoid weakening of the transverse process. The surgeon should utilize gentle distraction of the nerve root. The veins near the lateral tethering ligaments may cause bleeding and may be controlled with hemostatic agents or bipolar forceps. Furthermore, the surgeon should release the intraforaminal ligaments to allow for complete decompression of the traversing and exiting nerve roots. If the surgeon identifies osteophytes, the surgeon may engage in the operation to remove any compressive osteophytes using standard instruments known in the art.

In one embodiment, the step of completing a discectomy 1370, a first discectomy 1380, and/or a second discectomy 1385 may be performed to standard surgery techniques, navigation assisted techniques and/or robotic assisted techniques. In another embodiment, the step of completing a discectomy 1370, a first discectomy 1380, and/or a second discectomy 1385 may be performed to standard surgery techniques with supplemental considerations. The supplemental considerations may include the assurance of nerve damage. The surgeon should protect the exiting nerve root and the lateral thecal sack by retracting both for protection of the neural elements prior to initiation of the discectomy. The supplemental considerations may further include a discectomy and a rectangular annulotomy on a first side and/or a second side (e.g., a bilateral annulotomy). On a first side, the surgeon may incise the disc and complete a rectangular annulotomy from paracentral to far lateral zone. The discectomy should remove endplate cartilage, nucleus pulposus, while preserving the lateral annulus and anterior longitudinal ligament (ALL). On a second side, the surgeon may incise the disc and complete a rectangular annulotomy from paracentral to far medial zone. The same procedure may be completed bilaterally (e.g., on a first side and/or second side) to allow for bilateral transforaminal approach with complete discectomy. The surgeon should ensure that the entire nucleus pulposus is removed to minimize risk for future disc herniation and to allow adequate space for implantation of the prosthesis.

The supplemental considerations may further include mobilizing the intervertebral disc space of the spinal segment. The surgeon may consider utilizing an osteotome or a thin osteotome. The osteotome may be inserted medial to the pedicle preliminarily mobilize the disc space. The surgeon may desirably want to avoid disruption of the endplates at the index level. The supplemental considerations may further include evaluation of patients with prior surgery. In the cases of patients with prior surgery at the index level, significant epidural scaring can make the decompression and/or disc preparation more challenging. The risk of incidental durotomy is increased with patients who have had prior surgery. The surgeon should pay careful attention to the location of prior laminotomy/laminectomy so the surgeon is familiar with the altered anatomy in these cases. Additionally, the principle of identifying previously unoperated anatomy and working from a virgin area to a previously operated area for the decompression should be followed.

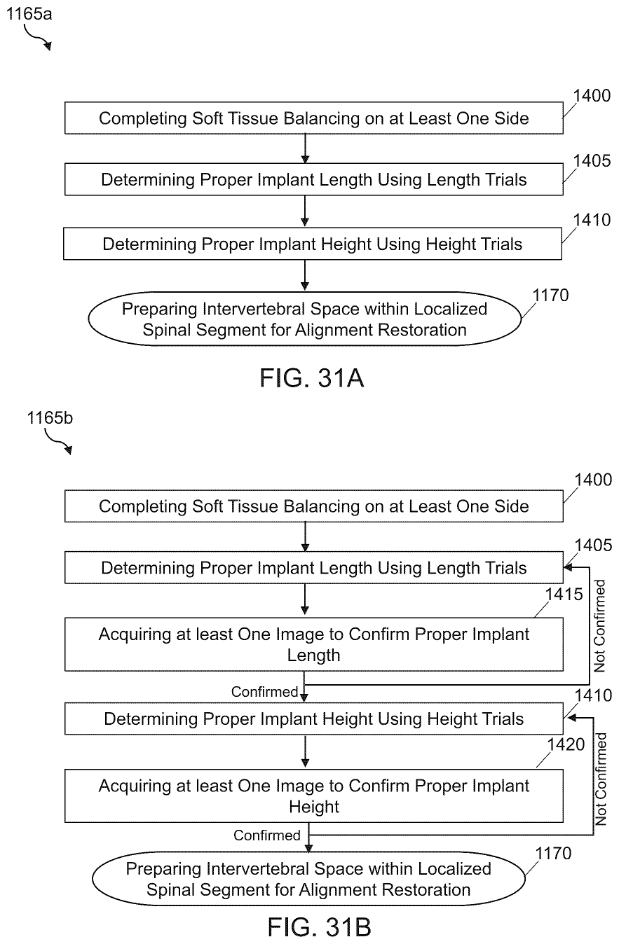

With reference to FIG. 31A, the navigation assisted and/or robotic assisted intraoperative method may comprise the step of selecting the proper spinal implant size 1165, 1165a. The step of selecting the proper implant size 1165, 1165a, 1165b may comprise the steps of: balancing soft tissue on at least one side 1400; determining the proper implant length using implant length trial tools 1405; and determining the proper implant height using implant height trial tools 1410. The step of the step of selecting the proper spinal implant size may be performed and/or completed to standard manual techniques, navigation-assisted and/or robotic-assisted techniques.

With reference to FIG. 31B, the step of selecting the proper implant size 1165, 1165b may further comprise the steps of: balancing soft tissue on the at least one side 1400; determining the proper implant length using implant length trial tools one the at least one side 1405; confirming approximated implant length by acquiring one or more images using at least one imaging technique 1415; determining the proper implant height using implant length trial tools on the at least one side 1410; confirming approximated implant height by acquiring one or more images using at least one imaging technique 1420. The step of selecting the proper implant size may further comprise the step of matching the convergence of the implant trial height and/or implant trial length tools to match the axis of the pedicle at the treated spinal unit or spinal segment (e.g., matching the transverse pedicle angle at the treated spinal unit or spinal segment) on a first side and/or a second side.

In one embodiment, the step selecting a proper implant size 1165, 1165a, 1165b may comprise the step of balancing soft tissue by mobilizing the intravertebral space on at least one side 1400 and/or a first balancing of a first soft tissue by mobilizing the intervertebral space and a second balancing of a second soft tissue (not shown). The surgeon may begin intervertebral space mobilization with the smallest provided distractor to increase intervertebral height at least one side. Alternatively, the surgeon may begin intervertebral space mobilization with the smallest provided distractor on a first side and/or a second side. The surgeon should sequentially introduce other distractors with increased width and/or height into the intervertebral space to allow tensioning of the soft tissues on at least one side, a first side and/or a second side. Alternatively, the surgeon should sequentially introduce other distractors with increased width and/or height into the intervertebral space to allow a first balancing or tensioning of the soft tissues on a first side and a second tensioning or balancing a second side. The first tensioning should be the same tension and/or substantially the same as the second tensioning. The first tensioning should be a different tension than the second tensioning.

The surgeon should be aware that soft tissue responses vary depending on the severity and duration of the patient's illness so the amount of time to allow for soft tissue relaxation between insertion of larger distractors may differ. The surgeon should prevent excessive distraction of the intervertebral space and endplate fracture or compression. The proper device height will vary by patient and should primarily be estimated based on soft tissue tension.

In another embodiment, the step selecting a proper implant size 1165, 1165a, 1165b may comprise the step of determining the proper implant length using implant length trial tools 1405, 1405a, 1405b may comprise the steps of: preparing a length trial tool; introducing at least one length trial on at least one side; verifying the center of rotation (COR); and/or confirming proper implant length by acquiring at least one image using at least one imaging technique. The step of determining the proper implant length using implant length trial tools 1405, 1405a, 1405b, 1405c may further comprise superimposing one or more objects onto one or more operative images; positioning user to desired trajectory, first trajectory and/or first one or more objects; generating a second trajectory and/or a second one or more objects; correcting user to return to first trajectory and/or first one or more objects after confirming that second trajectory and/or second one or more objects is different than the first trajectory and/or first one or more objects.

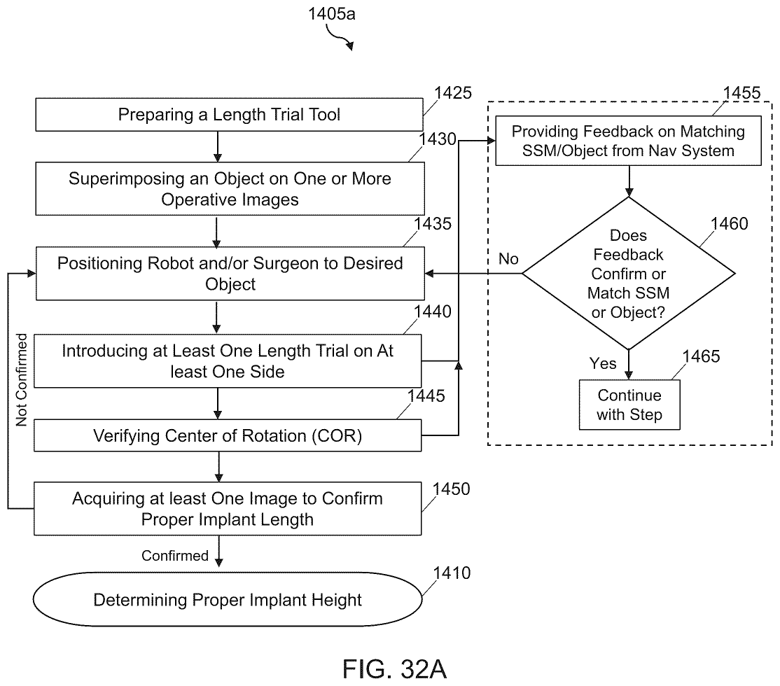

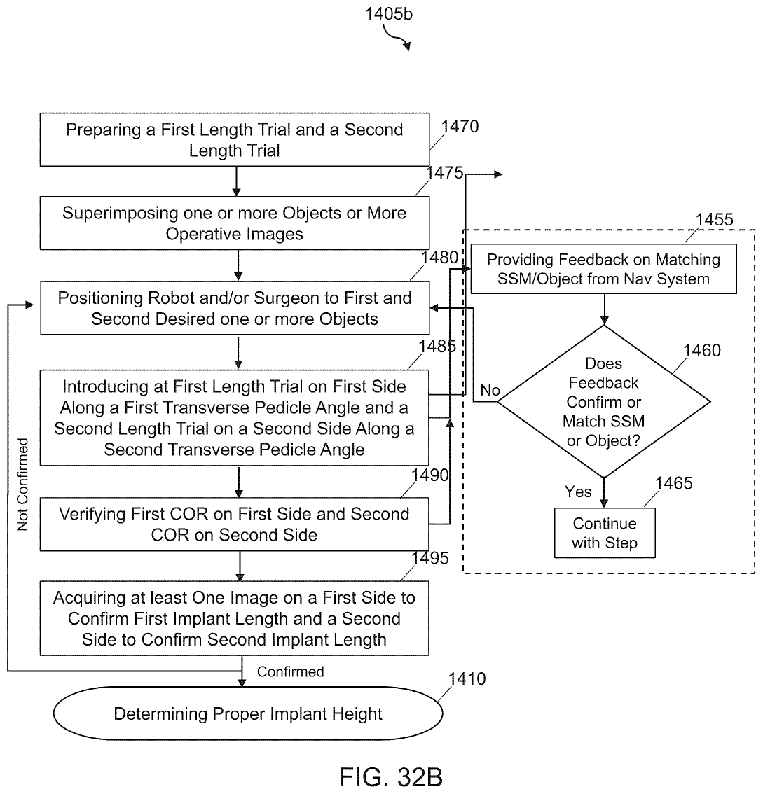

With reference to FIG. 32A, the step of determining the proper implant length using implant length trial tools 1405, 1405a may comprise the steps of: preparing a length trial tool 1425; superimposing an one or more objects onto one or more operative images 1430; positioning user (e.g., robot and/or surgeon) to desired one or more objects 1435; introducing at least one length trial on at least one side 1440; providing feedback on matching or substantially matching one or more desired objects 1455, 1460; verifying the center of rotation (COR) 1445; providing feedback on matching or substantially matching the desired one or more objects 1455; and/or confirming proper implant length by acquiring at least one image using at least one imaging technique 1455. The step of determining the proper implant length using implant length trial tools 1405, 1405a may further comprise the steps of: generating a second one or more objects that can be superimposed onto the one or more operative images and/or continuing with the step of determining the proper implant length using length trial tools 1465, 1465a. The one or more operative images comprises MPR images and/or synchronized images. The step of introducing at least one length trial on at least one side 1440 may follow, match and/or substantially match the transverse pedicle angle.

With reference to FIG. 32B, the step of determining the proper implant length using implant length trial tools 1405, 1405b comprises the steps of: preparing a first and a second length trial tool 1470; superimposing a first one or more objects on a first side and a second one or more objects on a second side onto one or more operative images 1475; positioning user (e.g., robot and/or surgeon) to a first desired one or more objects on a first side and a second desired one or more objects on a second side 1480; introducing the first length trial on a first side that follows, matches or substantially matches the first transverse pedicle angle and the second length trial on a second side that follows, matches or substantially matches the second transverse pedicle angle 1485; providing feedback on matching or substantially matching a first and second desired one or more objects 1455; verifying the first center of rotation (COR) on a first side and a second COR on a second side 1490; providing feedback on matching or substantially matching a first and second one or more objects 1455; and/or confirming proper implant length on a first side and a second side by acquiring at least one image using at least one imaging technique 1495. The step of determining the proper implant length using implant length trial tools 1405, 1405b may further comprise the steps of: generating a third one or more objects on a first side that can be superimposed onto the one or more operative images and a fourth one or more objects on a second side; and/or continuing with the step of determining the proper implant length using length trial tools 1465, 1465b. The one or more operative images comprises MPR images and/or synchronized images.

The first length trial may be the same length as the second length trial. The first length trial may be a different length than the second length trial. Accordingly, first transverse pedicle angle may comprise the same angle as the second transverse pedicle angle. The first transverse pedicle angle may comprise a different angle than the second transverse pedicle angle. The first desired one or more objects may comprise a same object as the second desired one or more objects. The first desired one or more objects may comprise a different object as the second desired one or more objects. The first desired one or more objects may be different or the same as the third one or more objects. The second desired one or more objects may be different or the same as the fourth one or more objects.

The one or more objects, second one or more objects, third one or more objects and/or fourth one or more objects may comprise a text annotation (not shown), a trajectory path 1310, a virtual surgical instrument 1320, a spinal implant 1335 and/or any combination thereof as shown in FIGS. 27A-27C. The trajectory path 1310 may comprise the trajectory of a surgical instrument, surgical approach and/or one or more spinal implants. The text annotation may comprise one or more SSMs. In one embodiment, the one or more objects, the first one or more objects, the second one or more objects, the third one or more objects and/or the fourth one or more objects comprises text annotations that include one or more SSMs, one or more virtual trajectory paths 1310, and/or one or more virtual tools 1320. The PSMs or SSMs include a convergence angle or transverse pedicle angle and a center of rotation (COR). The SSM may further include a force value. The one or more virtual trajectory paths 1310 and/or virtual tools 1320 should follow, match or substantially match the convergence angle or transverse pedicle angle trajectory path.

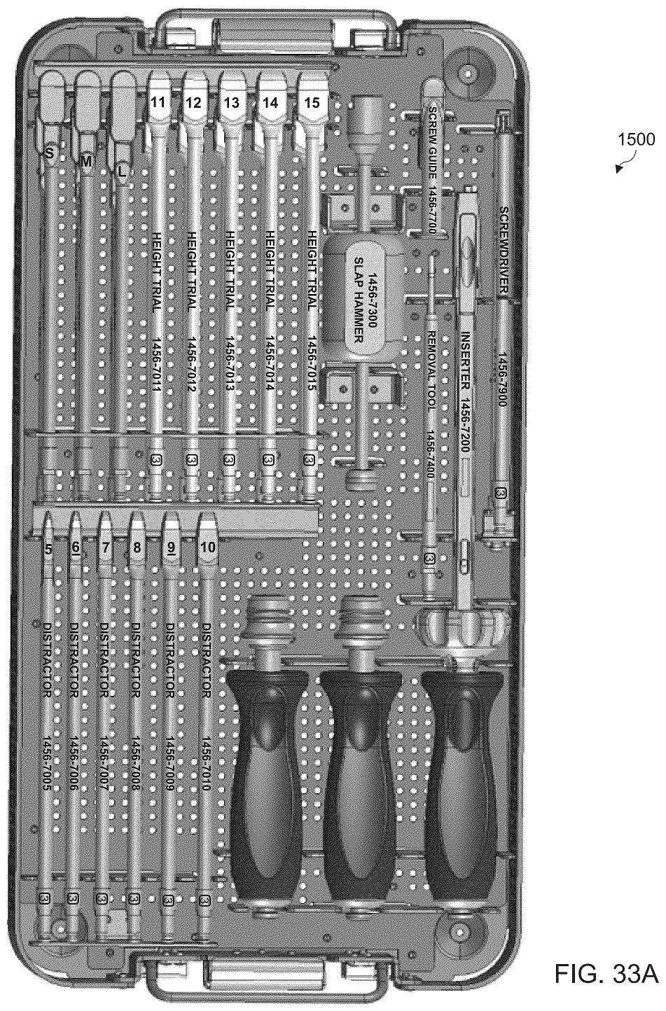

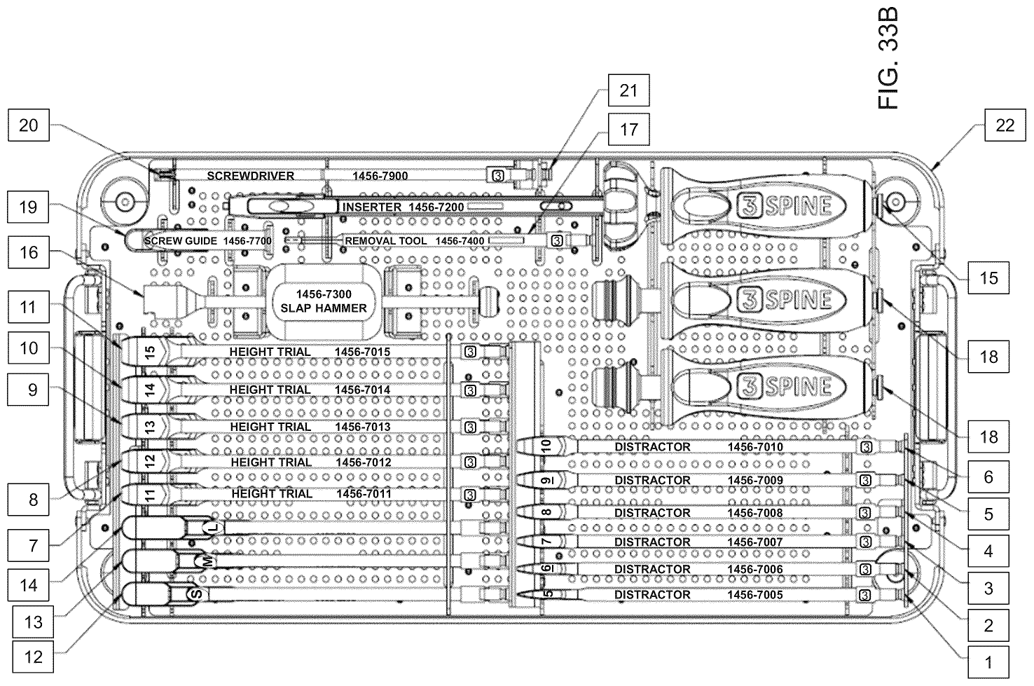

In one embodiment, the step of determining the proper implant length using implant length trial tools 1405, 1405a, 1405b may comprise the step of preparing a length trial tool on at least one side 1425 and/or the step of preparing a first length trial tool for a first side and/or a second length trial tool for a second side 1470. The surgeon may require the use of sequential length trials 1505 of different lengths—small (or short), medium and large (or long). The surgeon may confirm that the surgical kit 1500 may contain at least three length trials 1505. The instrumentation kit 1500 may comprise a variety of available tools for use throughout the procedure as shown in FIGS. 33A-33B and Table 2 below.

TABLE 2
Surgical Tool Kit

| No. | Tool Description |
|---|---|
| 1 | Distractor, 5 |
| 2 | Distractor, 6 |
| 3 | Distractor, 7 |
| 4 | Distractor, 8 |
| 5 | Distractor, 9 |
| 6 | Distractor, 10 |
| 7 | Height Trial, 11 |
| 8 | Height Trial, 12 |
| 9 | Height Trial, 13 |
| 10 | Height Trial, 14 |
| 11 | Height Trial, 15 |
| 12 | Length Trial, Short |
| 13 | Length Trial, Medium |
| 14 | Length Trial, Long |
| 15 | Inserter |
| 16 | Slap Hammer |
| 17 | Removal Tool |
| 18 | Hudson Handle |
| 19 | Screw Guide |
| 20 | Screwdriver |
| 21 | Removal Driver |
| 22 | Base |
| 23 | Lid |
| 24 | Powered Reciprocator |

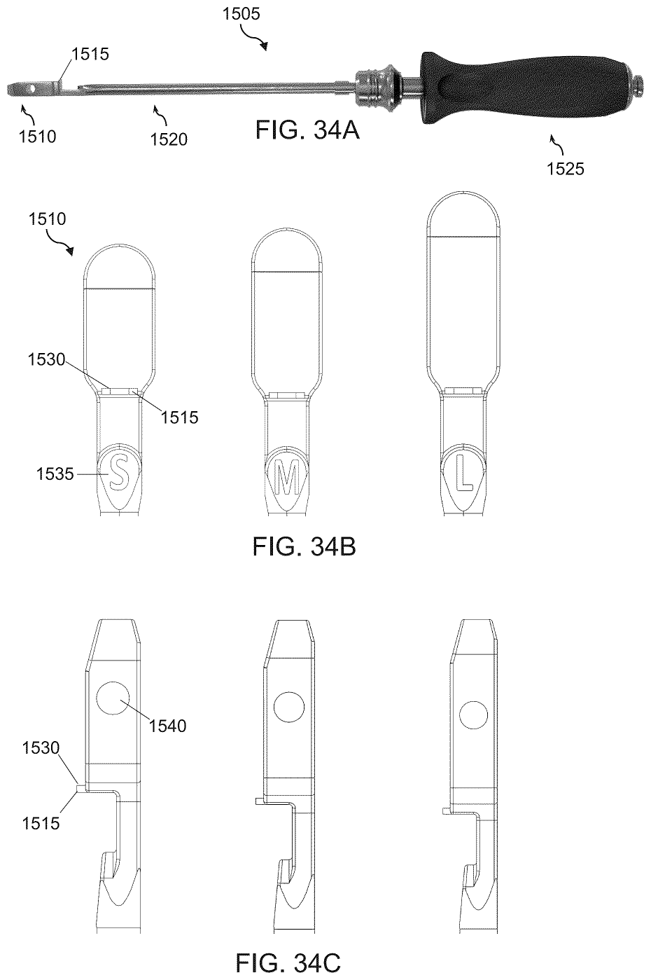

The at least three length trials 1505 match and/or approximates of the implant as shown in FIGS. 34A-34C. The at least three length trials comprise a short or small (25 mm), a medium (29 mm), and a long or large (32 mm). Each of the length trials 1505 comprise a length trial body 1510, a shaft 1520, and a handle 1525. Each of the length trials 1505 further comprises a size indicator 1535 and a center or rotation (COR) indicator 1540. The length trial body 1510 comprises a substantially rounded rectangle shape. The length trial body 1510 further comprises a positive stop or tab 1515 at the posterior end of the length trial body 1510. The positive stop or tab 1515 extends upwardly from a top surface of the length trial body 1510. The positive stop or tab 1515 extends upwardly and perpendicularly from a top surface of the length trial body. The positive stop or tab 1515 extends upwardly and obliquely from a top surface of the length trial body. The positive stop or tab 1515 includes an anterior facing surface 1530. Accordingly, the surgeon may attach the length trial tool 1505 to a standard handle or Hudson handle 1525 and/or a robotic arm for easier manual manipulation.

In one embodiment, the step of determining the proper implant length using implant length trial tools using trial tools 1405, 1405a, 1405b, the step preparing a length trial tool on at least one side 1425, and/or the step of preparing a first and a second length trial tool on first and/or second sides 1470 may further comprise the step of re-identifying and/or re-registering surgical instruments within the navigation system and/or robot. The surgical instruments may have been previously tracked during the preoperative procedure (e.g., a baseline registration) and during the start of the operative procedure the step of preparing equipment for navigational and/or robotic assistance 1145, and may not require further reregistration. The surgeon or staff may be able to couple a portion of a reference system and/or robotic arm to the previously identified surgical instrument and begin immediate use of the instrument. Alternatively, the surgeon or staff may couple a portion of a reference system and/or couple to a robotic arm to the previously identified instrument (e.g., the baseline registration during the preoperative method), and may require intraoperative confirmation of spatial relationship or coordinates between the patient and the pre and intraoperative images.

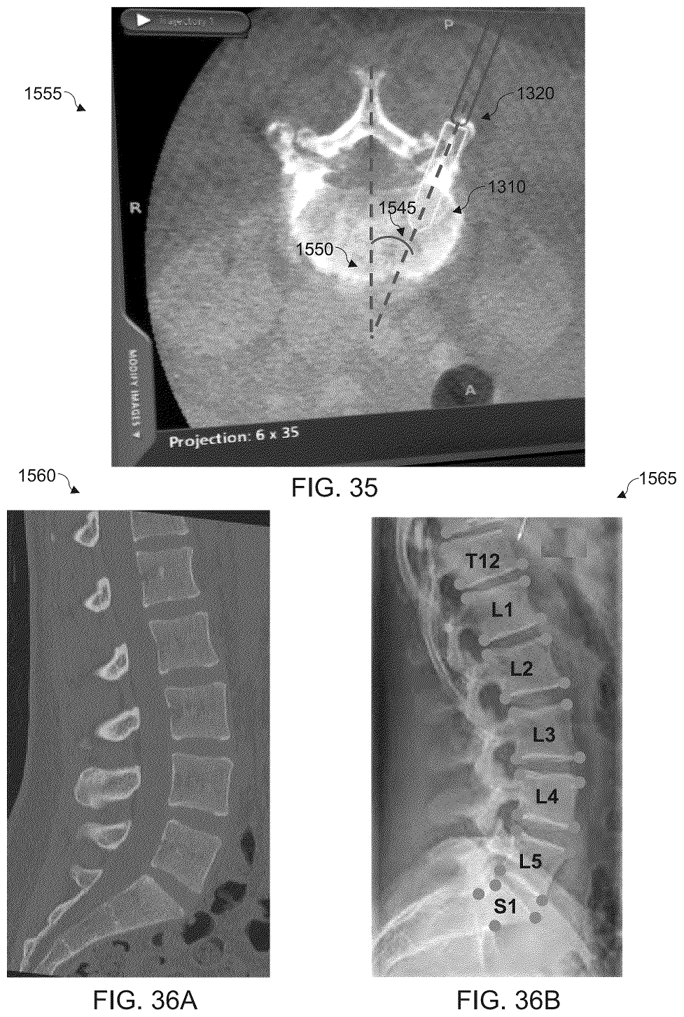

With reference to FIGS. 35 and/or 36A-36D, the different images 1555, 1560, 1565, 1570, 1575 depict the step of superimposing one or more objects onto one or more operative images for the at least one side 1430 and/or the step of superimposing a first one or more objects for a first side and/or a second one or more objects for a second side onto one or more operative images 1475. The operative images may comprise a one or more preoperative images, one or more operative images, one or more synchronized operative images and/or one or more MPR images. The object may comprise an annotated text 1545, 1580, surgical measurement or projected surgical measurement (PSM) 1550, 1585, a trajectory path 1310, a virtual surgical instrument 1320, a virtual spinal implant 1335 and/or any combination thereof. The trajectory path 1310 may comprise the trajectory of a surgical instrument, a surgical approach, one or more spinal implants and/or an SSM. The PSMs or SSMs include a convergence angle, A/P distance and a center of rotation (COR). The SSM may further include a force value. The one or more virtual trajectory paths 1310 and/or virtual tools 1320 should follow, match or substantially match the convergence angle or transverse pedicle angle trajectory path.

Accordingly, the step of determining the proper spinal implant length 1405, 1405a, 1405b may further comprise the step of positioning the user (e.g., surgeon and/or robot) to the desired one or more objects for the at least one intraoperative step on the at least one side 1435 and/or the step of positioning the user to a first one or more objects for a first side and a second one or more objects for a second side for the at least one operative step 1480. The navigation system may transmit spatial coordinates of the one or more surgical instruments relative to the targeted anatomy for trajectory positioning. The user (e.g., surgeon and/or robotic system) may hold the one or more surgical instruments and/or a registration tool (i.e., a probe, a guide and/or any other tool) to the desired position according to the spatial coordinates of the surgical trajectory for the desired intraoperative step. The trajectory may match or substantially match one or more projected surgical measurements (PSM). The one or more objects comprise one or more PSMs, a virtual surgical instrument, a virtual spinal implant, and/or any combination thereof.

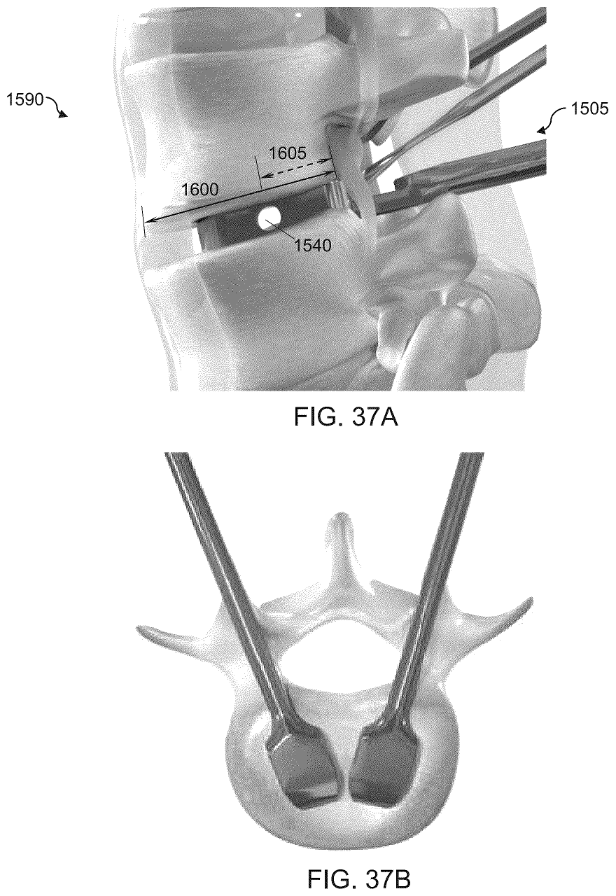

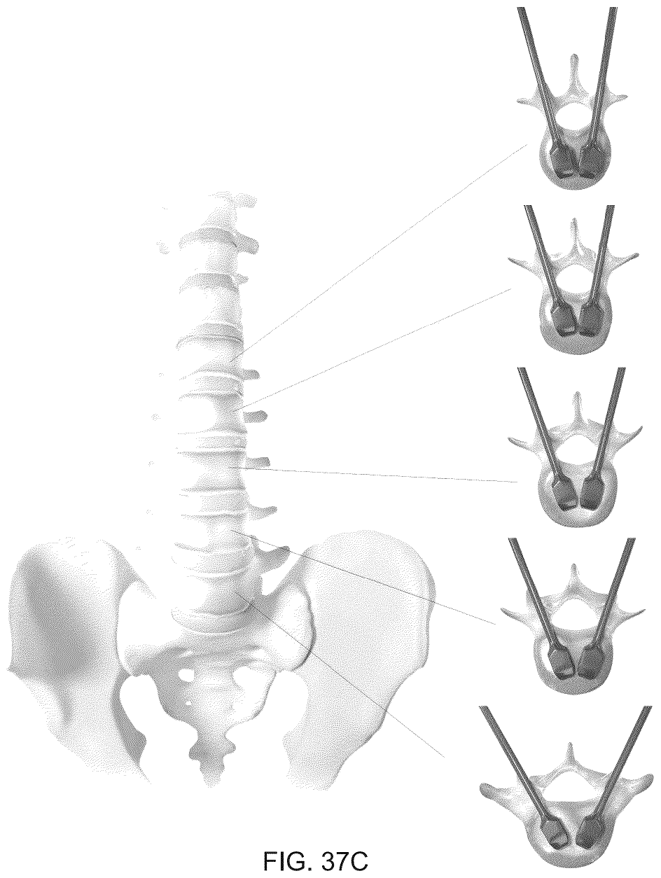

In another embodiment, the step of determining the proper implant length 1405, 1405a, 1405b may comprise the step of introducing at least one length trial on at least one side 1440 and/or the step of introducing a first length trial on the first side and/or a second length trial on a second side 1485. The step of introducing at least one length trial on at least one side, a first side and/or a second side 1440, 1485 includes the steps of: inserting a length trial into the targeted intervertebral disc space; translating the at least one length trial within the targeted intervertebral disc space until reaching the positive stop tab. Alternatively, step of determining the proper implant length on at least one side, a first side and/or a second side 1440, 1485 includes the steps of: inserting the length trial into the targeted intervertebral disc space; and translating the at least one length trial on at least one side that matches or substantially matches the transverse pedicle angle (or convergence angle) as shown in FIGS. 35 and 37B. Substantially matches may comprise at least up to a +/−10 percent deviation from the transverse pedicle angle central axis.

The step of inserting a length trial into the targeted intervertebral space should follow a custom desired trajectory path. The custom desired trajectory path may be a path of least resistance and/or along and/or substantially along the transverse pedicle angle or convergence angle between a distracted disc space. Should the surgeon follow along, match or substantially match the transverse pedicle angle, such alignment, convergence and/or positioning (may also be referred to as a “toe-in angle”) would help facilitate stability and resist shear forces. The orientation of pedicles, known as the transverse pedicle angles, vary across each spine segment within a spine region as shown in FIGS. 37B-37C. The convergence angles or transverse pedicle angles further varies by patient and by each spine segment within a spine region. Alternatively, the surgeon may not follow, match or substantially match the transverse pedicle angle. Furthermore, the step of inserting a length trial into the targeted intervertebral space may comprise insertion on at least one side, a first side and/or a second side.

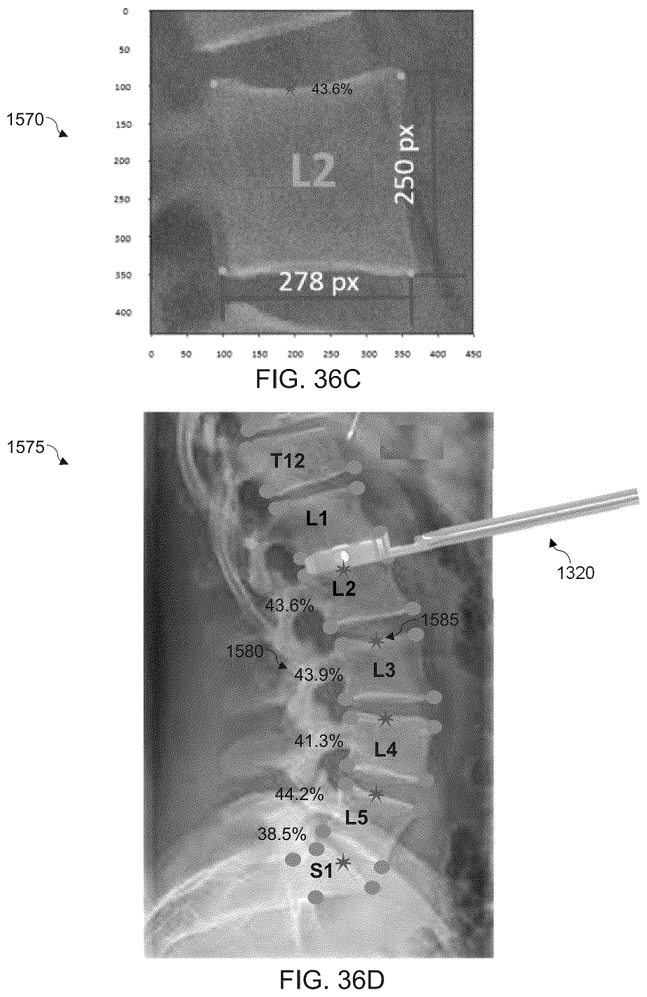

With reference to FIGS. 36D and 37A, the sagittal view images of one or more spinal segments 1575, 1590 illustrate the translation of at least one length trial virtual tool 1320 within the targeted intervertebral space. The at least one length trial, a first length trial and/or a second length trial should inserted into the distracted intervertebral disc space toward the anterior direction of the distracted intervertebral space following one or more superimposed objects, a custom path and/or the transverse pedicle angle until the surgeon contacts a portion of the anterior facing surface 1530 of a positive stop tab 1515 on the at least one length trial 1505 onto a portion of the posterior surface of vertebral body and/or a portion of the apophyseal ring. The one or more superimposed objects may comprise one or more annotated texts including one or more selected surgical measurements (SSM) 1545, 1580, a virtual trajectory path 1310, and/or a virtual instrument or tool 132, an indicator 1585, and/or any combination thereof. The virtual trajectory 1310, the indicator, and/or annotated text 1545, 1580 may match or substantially match one or more PSMs or SSMs. The surgeon should note that prior to inserting a trial tool, the surgeon should ensure that the exiting nerve root is gently retracted laterally, and the thecal sack should be retracted medially to protect the neural elements if one or both retractions are necessary.

Alternatively, the step of determining the proper spinal implant length 1405, 1405a, 1405b comprises the step of confirming or verifying the center of rotation (COR) on at least one side 1445 and/or the step of confirming a first COR on a first side and a second COR on a second side 1490 as shown in FIGS. 36A-36D and 37A. The surgeon may confirm through one or more techniques. The first technique allows the surgeon to calculate manually. The surgeon can obtain the total anterior to posterior length of a vertebral body, Ltot, 1600. The surgeon may calculate the COR is positioned at least an average of 40% anterior from the posterior end of the caudal endplate of the vertebral body, Lcor, 1605. A second technique allows the surgeon to use visual indicators 1540 on the length trial 1505. The surgeon should translate the length trial 1505 along a custom path until the surgeon reaches the positive stop tab 1515. The tactile feedback from the positive stop tab 1515 will provide the surgeon tactile confirmation and the visual indicator 1540 and/or radiopaque marker on the length trial will provide a visual confirmation through an imaging technique. The positioning of the radiolucent marker and/or the visual indicator 1540 on the length trial 1505 relative to the vertebral body is intended to approximate the implant's center of rotation (COR). The confirmation may be completed subjectively by looking at the radiopaque marker or the visual indicator 1540 and approximating its position is at least 40% anterior from the posterior end of the caudal endplate of the cranial vertebral body using at least one image.

A third technique may incorporate the navigation system and/or the preoperative virtual models from the surgical planning software by computing quantitative measurements, indicators and/or trajectory paths. The computerized quantitative measurements may be obtained during the preoperative procedure with the preoperative software application as shown in FIGS. 35 and 36A-36D. The preoperative software will approximate the COR based on processed and/or analyzed preoperative images and will be different at each spine segment within each spine region. The computer analysis of the COR will be superimposed on the one or more intraoperative images or one or more MPR images and/or in or more synchronized images. The user would insert at least one length trial 1505 into an intervertebral space following a desired path and translate the at least length trial 1505 until the positive stop tab 1515 is reached creating an excessive force. The radiopaque marker and/or indicator 1540 should be aligned with the one or more superimposed objects.

In another embodiment, the step of determining the proper implant length 1405, 1405a, 1405b may comprise the step of providing feedback on matching or substantially matching one or more objects that were superimposed on the one or more images 1455 and/or the step of providing feedback on matching or substantially matching a first one or more objects on a first side and a second one or more objects on a second side 1455. The feedback may comprise visual feedback (using the navigation monitor or workstation display), audible feedback, tactile feedback, and/or any combination thereof. The feedback may include visual and/or audible feedback to correct or reposition the surgeon's trajectory and/or robot's trajectory to appropriately match or substantially match the one or more objects and/or projected surgical measurement. The one or more objects may include the SSM (selected surgical measurement), a virtual trajectory, a virtual tool, a virtual spinal implant, and/or any combination thereof. The SSM may include a convergence angle or transverse pedicle angle, and a COR. The virtual trajectory may match or substantially match the convergence angle or transverse pedicle angle.

In one embodiment, the step of providing feedback 1455 may further comprise the steps of: generating a non-desired trajectory path and/or non-desired one or more objects; comparing the desired trajectory path to the non-desired trajectory path 1460; correcting user to return to desired trajectory path if the non-desired trajectory path is different than the desired trajectory path 1435, 1480. In another embodiment, the step of providing feedback 1455 may further comprise the steps of: generating a second trajectory path and/or non-desired second one or more objects; comparing the first trajectory path to the second trajectory path 1460; correcting user to return to first trajectory path if the second trajectory path is different than the first trajectory path 1435, 1480. The step of providing feedback 1455 may comprise at least one side, a first side and/or a second side.

In another embodiment, the step of determining the proper implant length 1405, 1405a, 1405b may comprise the step of confirming proper or approximated spinal implant length by acquiring at least one image using at least one imaging technique for the at least one side 1450 and the step of confirming a first spinal implant length on a first side and a second spinal implant length on a second side 1495. The at least one image technique may comprise an MRI, a radiograph, a CT scan, an ultrasound, and/or any combination thereof. The at least one imaging technique may further comprise 2D or 3D images. While the one or more length trials are in position within the disc space, the surgeon may confirm one or more of the following: the proper tensioning of the soft tissues; the proper implant length; the proper center of rotation (COR) of the implant; the proper convergence angle (e.g., transverse pedicle angle or toe-in angle); the length trials do not contact a portion of the ALL or remaining annulus and/or any combination thereof. Should the proper and/or approximated spinal implant length is unable to be confirmed, the surgeon will use the subsequent length trial tool, and repeat the steps beginning from the step of assembling a length trial tool (using the new length trial).

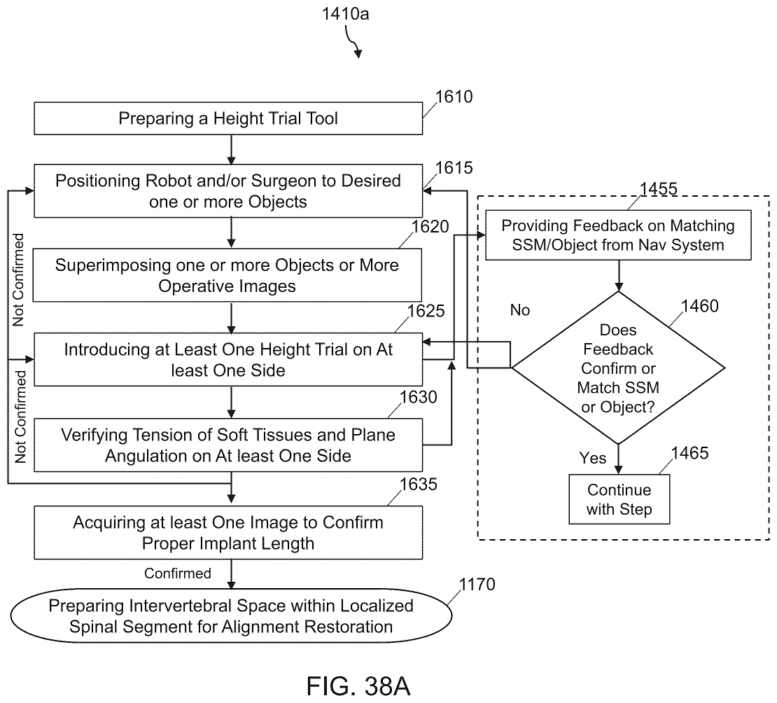

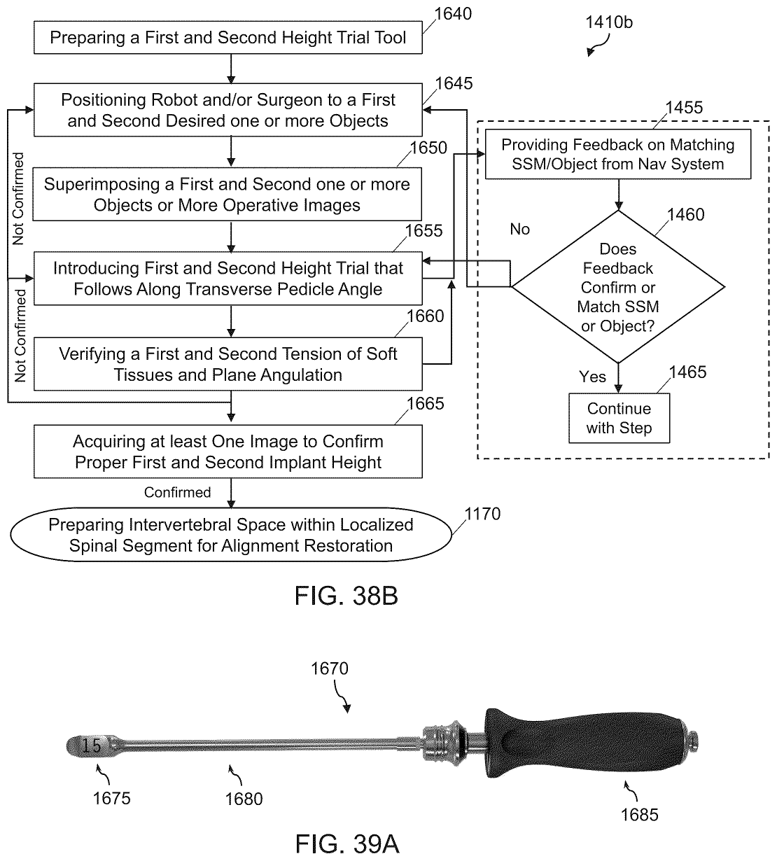

With reference to FIGS. 38A, the step of selecting the proper spinal implant size 1165, 1165a, 1165b comprises the step of determining the proper spinal implant height 1410, 1410a using navigation and/or robotic assistance. The step of determining the proper or approximated spinal implant height 1410, 1410a comprises the steps of: preparing at least one height trial tool on at least one side 1610; superimposing one or more objects onto one or more operative images for at least one side 1620; positioning robot and/or surgeon to desired one or more objects on the at least one side 1615; introducing at least one height trial that follows along and/or substantially follows along a custom path on the at least one side 1625; verifying soft tissue tension and intervertebral space angulation on the at least one side 1630; providing feedback on matching or substantially matching the desired one or more objects on the at least one side 1455; and confirming proper or approximate spinal implant height on the at least one side by acquiring at least one image using at least one imaging technique 1635. The custom path may comprise a convergence angle and/or transverse pedicle angle.

With reference to FIG. 38B, the step of selecting the proper spinal implant size 1165, 1165a, 1165b comprises the step of determining the proper spinal implant height for a first and second side 1410, 1410b. The step of determining the proper or approximated spinal implant height for the first and second side 1410a, 1410b comprises the steps of: preparing a first height trial tool on a first side and second height trial tool on a second side 1640; superimposing a first one or more objects onto a first one or more operative images for a first side and a second one or more objects onto a second one or more operative images for a second side 1650; positioning a user (e.g., surgeon or robot) to desired first one or more objects on the first side and positioning the robot and/or surgeon to desired second one or more objects on the second side 1645; introducing a first height trial tool that follows a first custom path on a first side and a second height trial tool that follows and/or substantially follows along a second custom path on a second side 1655; providing feedback on matching or substantially matching the desired first one or more objects on a first side and the second one or more objects on a second side 1455; verifying a first soft tissue tension and first intervertebral space angulation on the first side and a second soft tissue tension and second intervertebral space angulation on the second side 1660; and confirming proper or approximate first spinal implant height on the first side and a second spinal implant on a second side by acquiring at least one image using at least one imaging technique on a first side and a second side 1665.

The first height trial may comprise a same height as the second height trial. The first height trial may comprise a different height as the second height trial. The first soft tissue tension may comprise a same tension as the second soft tissue tension. The first soft tissue tension may comprise a different tension as the second soft tissue tension. The first custom path may comprise the same path as the second custom path. The first custom path may comprise a different custom path as the second custom path. The first transverse pedicle angle may comprise a same angle and the second transverse pedicle angle. The first transverse pedicle angle may comprise a different angle as the second transverse pedicle angle. The first one or more objects may comprise a same object as the second one or more objects. The first one or more objects may comprise a different object as the second one or more objects. The first spinal implant may comprise a same implant than the second spinal implant. The first spinal implant may comprise a different implant than the second spinal implant. The first one or more operative images may comprise a same one or more images than the second one or more operative images. The first one or more operative images may comprise a different one or more images than the second one or more operative images.

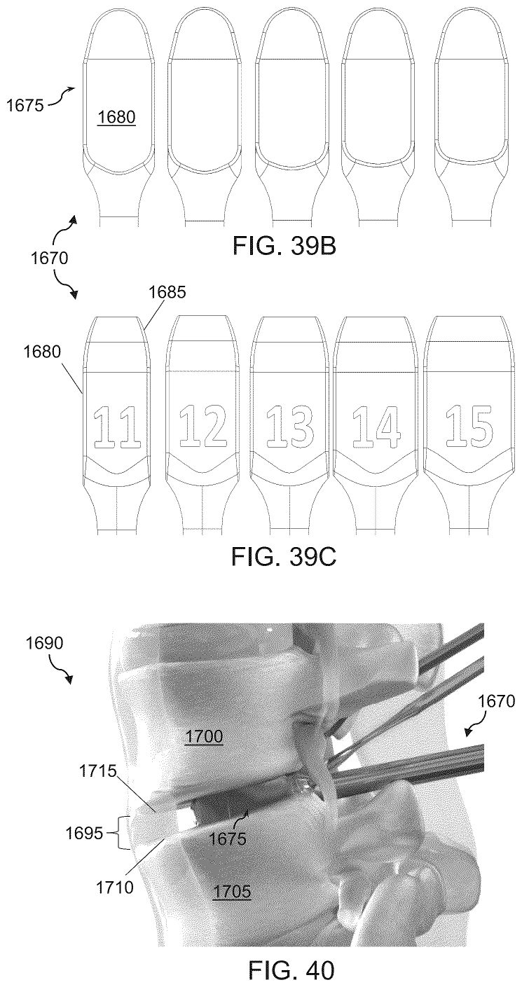

The step of selecting the proper implant height 1410, 1410a, 1410b comprises the step of preparing a height trial tool on at least one side 1610 and/or the step of preparing a first height trial tool on a first side and a second height trial tool on a second side 1640. The step of preparing a first height trial tool 1610, 1640 comprises the step of attaching the height trial 1670 to a handle or robot. The surgeon may confirm that the surgical instruments within the kit 1500 contains at least three length trials 1670 as shown in FIGS. 33A-33B. The at least three height trials or more that may match and/or approximates the height of the implant. In one exemplary embodiment, the height trials 1670 comprise heights of 11 mm, 12 mm, a 13 mm, a 14 mm and a 15 mm as shown in FIGS. 39A-39C and 40. Each height trial 1670 comprises a height trial body 1675, a shaft 1680. The height trial 1670 may further comprise a handle 1685. The height trial body 1675 comprises a first contacting surface 1680 and a second contacting surface 1685. Accordingly, the surgeon or user may attach the at least one height trial, the first height trial and/or a second height trial 1670 to a standard handle known in the art for easier manual manipulation and/or the surgeon may attach the height trial to a custom handle, e.g., the Hudson handle, for easier manual manipulation. Alternatively, the surgeon and/or the user may attach the at least one height trial, the first height trial and/or the second height trial 1670 to a robot.

The step of preparing a height trial tool, a first height trial tool and/or a second height trial tool 1610, 1640 may further comprise the step of re-identifying and/or re-registering surgical instruments within the navigation system and/or robot. The surgical instruments may have been previously tracked during the preoperative procedure (e.g., a baseline registration), and may not require further reregistration. The surgeon or staff may be able to couple a portion of a reference system and/or robotic arm to the previously identified surgical instrument and begin immediate use of the instrument. Alternatively, the surgeon or staff may couple a portion of a reference system and/or couple to a robotic arm to the previously identified instrument (e.g., the baseline registration during the preoperative method), and may require intraoperative confirmation of spatial relationship or coordinates between the patient and the pre and intraoperative images. As disclosed herein, each of the relevant surgical instruments may be adapted and configured to receive a portion of a reference frame, a fiducial, and/or marker.

With reference to FIGS. 35 and/or 36A-36D, the different images 1555, 1560, 1565, 1570, 1575 depict the step of superimposing one or more objects onto one or more operative images 1620 and/or the step of superimposing a first one or more objects and/or a second one or more objects onto one or more operative images 1650. The operative images may comprise a one or more preoperative images, one or more operative images, one or more synchronized operative images and/or one or more MPR images. The object may comprise an annotated text 1545, 1580, surgical measurement or projected surgical measurement (PSM) 1550, 1585, a trajectory path 1310, a virtual surgical instrument 1320, a virtual spinal implant 1335 and/or any combination thereof. The trajectory path 1310 may comprise the trajectory of a surgical instrument, a surgical approach, one or more spinal implants and/or an SSM. The PSMs or SSMs include a convergence angle and A/P distance. The SSM may further include a force value and/or a resistance value. The one or more virtual trajectory paths 1310 and/or virtual tools 1320 should follow, match or substantially match the convergence angle or transverse pedicle angle trajectory path.

The step of determining the proper spinal implant height 1410, 1410a, 1410b may comprises the step of positioning the user (e.g., surgeon or robot) to the desired one or more objects for at least one intraoperative step for the at least one side 1615 and the step of positioning the user to a first one or more objects on a first side and a second one or more objects on a second side for at least one operative step 1645. The navigation system may display the one or more objects, a first one or more objects and/or a second one or more objects. The spatial coordinates may be transmitted to the robot and/or the proper referencing system to confirm the one or more surgical instruments, a first surgical instrument and/or a second surgical instrument is positioned to desired trajectory or the one or more objects relative to the targeted anatomy. The user comprising a surgeon and/or a robotic system may hold the one or more surgical instruments and/or a registration tool (i.e., a probe, a guide and/or any other tool) to the desired position according to the spatial coordinates of the surgical trajectory for the desired intraoperative step. The trajectory may match or substantially match one or more projected surgical measurements (PSM). The one or more objects comprise one or more PSMs, a virtual surgical instrument, a virtual spinal implant, and/or any combination thereof.

In another embodiment, the step of determining the proper implant height 1410, 1410a, 1410b comprises the step of introducing at least one height trial on at least one side 1625 and/or the step of introducing a first height trial on a first side and/or a second height trial on a second side 1655. The step of introducing at least one height trial on at least one side, the first side and/or second side 1625, 1655 includes the steps of: inserting at least one height trial into the targeted intervertebral disc space following or substantially following a path or custom path; translating the at least one height trial within the targeted intervertebral disc space following and/or substantially following along a custom path. The at least one height trial comprises a single height trial. The at least one height trial may comprise a first height trial and a second height trial. The custom path or path comprises matches or substantially matches the transverse pedicle angle (or convergence angle). Substantially matches may comprise at least up to a +/−10 percent deviation from the transverse pedicle angle central axis.

The step of translating the height trial 1670 along a custom desired path may comprise a custom path selected during preprocedural surgical planning, from the surgical planning software and/or real-time operative planning. The desired path and/or custom path may further comprise a path that follows along, match or substantially match the transverse pedicle angle or convergence angle located within the intervertebral space 1695. Such path alignment, convergence and/or positioning (may also be referred to as a “toe-in angle”) would help facilitate stability and resist shear forces. The orientation of pedicles, known as the transverse pedicle angles, vary across each patient and each spine segment within a spine region. As shown in FIG. 37C, the pedicles converge lateral to medial. Alternatively, the surgeon may not have the height trial to match or substantially match the transverse pedicle angle.

With reference to FIG. 40, a sagittal view of a targeted vertebral segment 1690 within a spine region depicts the height trial tool 1670 within the intervertebral space 1695. The height trial, a first height trial and/or a second height trial 1670 should be translated within the targeted intervertebral space 1690 along a custom path and pushed or slid toward the anterior direction of the distracted intervertebral space 1695. The first contacting surface 1680 should contact a first endplate 1715 of a first vertebral body 1700 and the second contacting surface 1685 should contact a second endplate 1710 of a second vertebral body 1705. The surgeon or user will push or translate until at least a portion of the height trial body 1675 and/or the entire height trial body is disposed within the intervertebral space 1695. Alternatively, the surgeon and/or user will push or translate until at least the posterior end or second end of the height trial body 1675 aligns with the posterior surface of a first vertebral body 1700 and/or second vertebral body 1705.

The step of determining the proper implant height 1410, 1410a, 1410b comprises the step of verifying a tension of the soft tissues and the plane angulation on at least one side 1630 and/or the verifying a first tension of the soft tissues and a first plane angulation on first side and a second tension of the soft tissues and a second plane angulation on a second side 1660. The tension of the soft tissues includes verifying that the soft tissues are not over tensioned or excessively tensioned. The user and/or surgeon may verify using visual indicators on the one or more operative images and/or one or more MPR images. Alternatively, the user and/or surgeon may verify through a measurement, the measurement includes a force value, a resistance value and/or a compression value. An accepted resistance or force value may be set, then while translating the height trial tool 1670 one or more force values may be collected and compared to the accepted for value. If the second force value exceeds the first force value, the navigation and/or robotic system may prompt the user and/or surgeon that the intervertebral space is not properly tensioned and requires further space distraction. Thus, returning the step of introducing at least one height trial tool, a first height trial, and/or a second height trial 1625, 1655. Also, the plane angulation comprises verifying the angle of the inferior endplate of the superior vertebral body relative to the superior endplate of the inferior vertebral body. The angle comprises a coronal angle, a sagittal angle, and/or a convergence angle. The angles may be visually determined by confirming on the one or more operative images, and/or computed by the navigational software.

In another embodiment, the step of determining the proper implant length 1405, 1405a, 1405b may comprise the step of providing feedback on matching or substantially matching one or more objects that were superimposed on the one or more images 1455 and/or the step of providing feedback on matching or substantially matching a first one or more objects on a first side and a second one or more objects on a second side 1455. The feedback may comprise visual feedback (using the navigation monitor or workstation display), audible feedback, tactile feedback, and/or any combination thereof. The feedback may include visual and/or audible feedback to correct or reposition the surgeon's trajectory and/or robot's trajectory to appropriately match or substantially match the one or more objects and/or projected surgical measurement. The one or more objects may include the SSM (selected surgical measurement), a virtual trajectory, a virtual tool, a virtual spinal implant, and/or any combination thereof. The SSM may include a convergence angle or transverse pedicle angle, and a COR. The virtual trajectory may match or substantially match the convergence angle or transverse pedicle angle.

In one embodiment, the step of providing feedback 1455 may further comprise the steps of: generating a non-desired trajectory path and/or non-desired one or more objects; comparing the desired trajectory path to the non-desired trajectory path 1460; correcting user to return to desired trajectory path if the non-desired trajectory path is different than the desired trajectory path 1435, 1480. In another embodiment, the step of providing feedback 1455 may further comprise the steps of: generating a second trajectory path and/or non-desired second one or more objects; comparing the first trajectory path to the second trajectory path 1460; correcting user to return to first trajectory path if the second trajectory path is different than the first trajectory path 1435, 1480. The step of providing feedback 1455 may comprise at least one side, a first side and/or a second side.

The step of determining the proper implant height 1410, 1410a, 1410b comprises the step of confirming proper or approximate spinal implant height on the at least one side 1635 and/or the step of confirming first spinal implant height on a first side and/or a second spinal implant height on a second side 1665. The surgeon may desirably acquire at least on image using at least one imaging technique to confirm proper implant height on at least one side, a first side and/or a second side. The at least one image technique may comprise an MRI, a radiograph, a CT scan, an ultrasound, and/or any combination thereof.

While the at least one height trial, a first height trial and/or a second height trial 1670 are in position within the intervertebral disc space 1695, the surgeon may confirm one or more of the following: the proper tensioning of the soft tissues; the proper or approximate implant height; the proper plane angulation of the upper vertebral body relative to the lower vertebral body; the proper convergence angle (e.g., transverse pedicle angle or toe-in angle); and/or whether the height trials 1670 do not contact a portion of the ALL or remaining annulus and/or any combination thereof. The surgeon may mark the medial edge of the height trial 1670 for a reference line or a guideline for the step of preparing the intervertebral disc space 1695 (e.g., for marking the medial edge of the rasps for the osteotomy). Should the proper and/or approximated spinal implant height is unable to be confirmed, the surgeon will use the subsequent height trial tool, and repeat the steps beginning from the step of preparing a height trial tool (using the new height trial) 1640. In another embodiment, the step of determining the proper spinal implant length and the step of determining the proper implant height may be reversed.

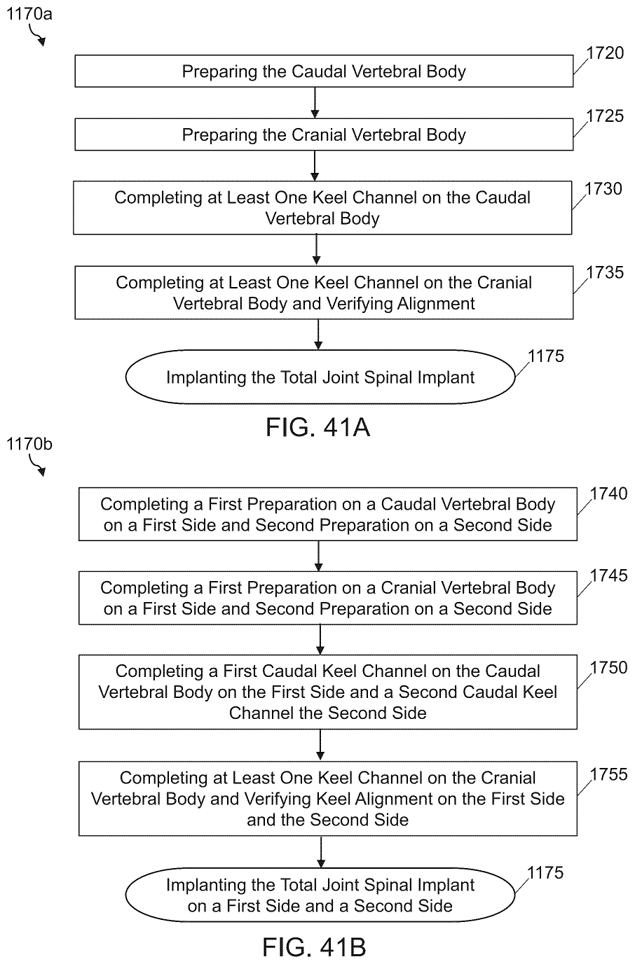

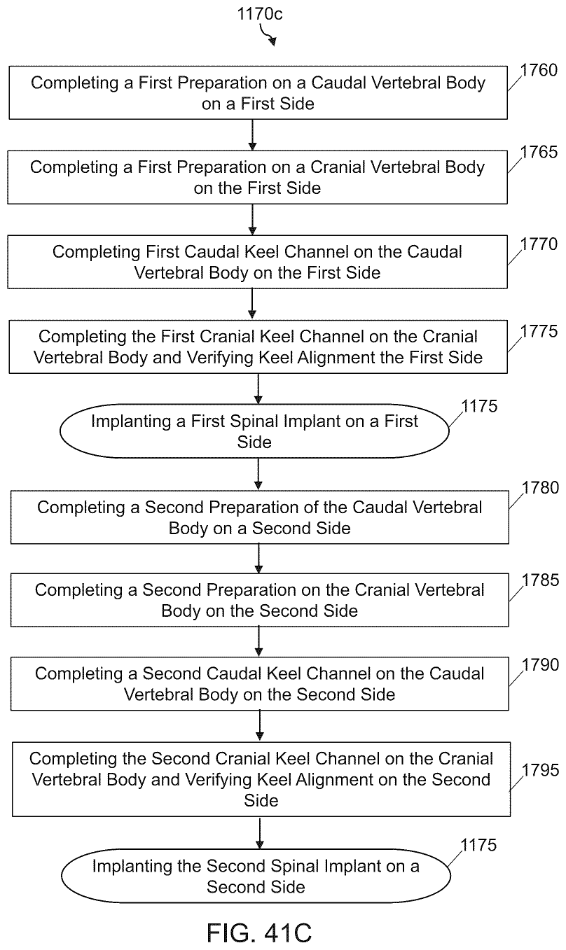

With reference to FIG. 41A, the navigation assisted or robotic assisted intraoperative procedure or intraoperative method 1125 comprises the step of preparing the intervertebral space within a localized spinal segment for alignment and motion restoration 1170, 1170a. The step of preparing the intervertebral space within a localized spinal segment for alignment and motion restoration on at least one side 1170, 1170a comprises the steps of: preparing a caudal vertebral body on the at least one side 1720; preparing a cranial vertebral body on at least one side 1725; completing at least one caudal keel channel on the caudal vertebral body on at least one side 1730; and completing at least one cranial keel channel on the cranial vertebral body on the at least one side 1735.

With reference to FIG. 411B, the robotic assisted and/or navigation assisted intraoperative procedure or intraoperative method 1125 comprises the step of preparing the intervertebral space within a localized spinal segment for alignment and motion restoration for a first side and/or a second side 1170, 1170b. The step of preparing the intervertebral space within a localized spinal segment for alignment and motion restoration on a first side and a second side 1170, 1170b comprises the step of: completing a first preparation on the caudal vertebral body on a first side and a second preparation on the caudal vertebral body on a second side 1740; completing a first preparation on a cranial vertebral body on a first side and a second preparation on the cranial vertebral body on a second side 1745; completing first caudal keel channel on the caudal vertebral body on a first side and a second caudal keel channel on the caudal vertebral body on a second side 1750; and completing a first cranial keel channel on the cranial vertebral body on the first side and verifying a first keel alignment and a second cranial keel channel on the cranial vertebral body on the second side and verifying a second keel alignment 1755.

With reference to FIG. 41C, the navigation assisted and/or robotic assisted intraoperative procedure or intraoperative method 1125 comprises the step of preparing the intervertebral space within a localized spinal segment for alignment and motion restoration 1170, 1170c. The step of preparing the intervertebral space within a localized spinal segment for alignment and motion restoration on a first side and a second side 1170, 1170c comprises the step of: completing a first preparation on the caudal vertebral body on a first side 1760; completing a first preparation on a cranial vertebral body on a first side 1765; completing first caudal keel channel on the caudal vertebral body on a first side 1770; and completing a first cranial keel channel on the cranial vertebral body on the first side and verifying a first keel alignment 1770; implanting a first spinal implant on a first side 1175; completing a second preparation on the caudal vertebral body on a second side 1780; completing a second preparation on a cranial vertebral body on a second side 1785; completing a second caudal keel channel on the caudal vertebral body on a second side 1790; and completing a second cranial keel channel on the cranial vertebral body on the second side and verifying a second keel alignment 1795; implanting a second spinal implant on a second side 1175.

The first preparation on the caudal vertebral body on the first side may be the same as the second preparation on the caudal vertebral body on the second side. The first preparation on the caudal vertebral body on the first side may be different than the second preparation on the caudal vertebral body on the second side. The first preparation on the cranial vertebral body on the first side may be the same as the second preparation on the cranial vertebral body on the second side. The first preparation on the cranial vertebral body on the first side may be different than the second preparation on the cranial vertebral body on the second side. The first spinal implant may be the same size as the second spinal implant. The first spinal implant may be a different size than the second spinal implant. The first keel alignment may comprise a same keel alignment as the second keel alignment. The first keel alignment may comprise a different keel alignment as the second keel alignment.

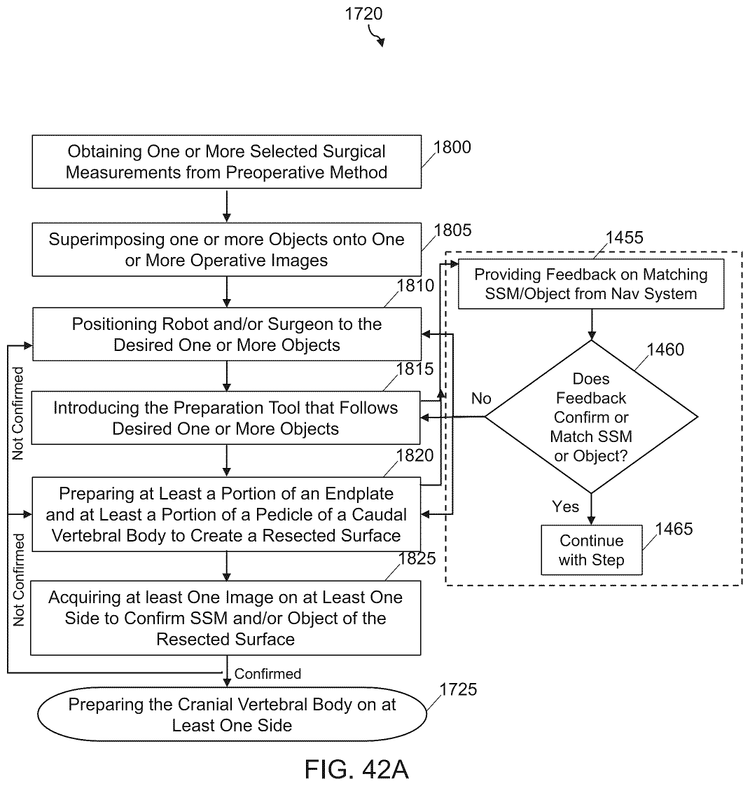

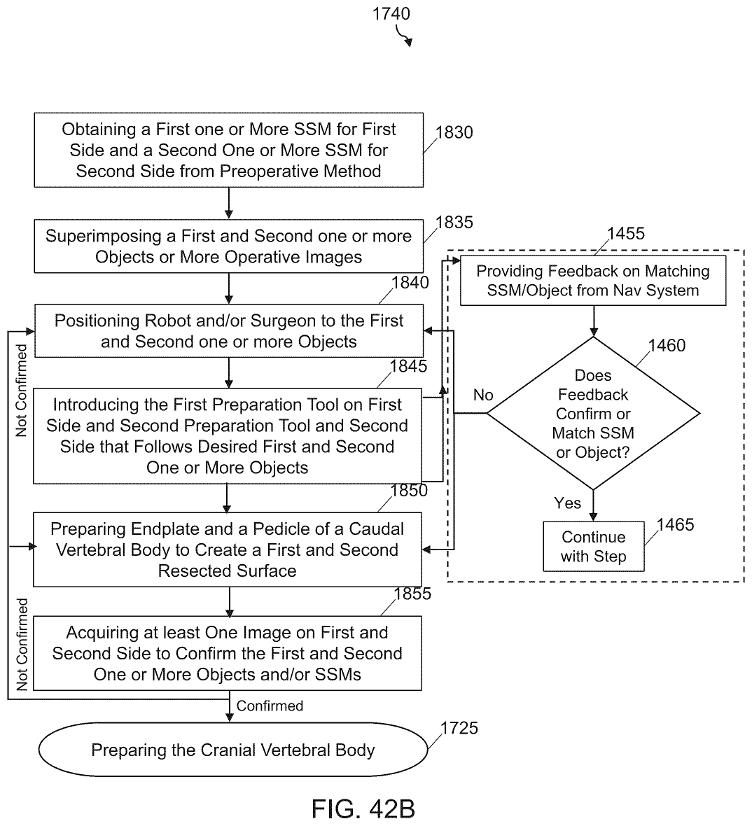

With reference to FIG. 42A, the step of preparing an intervertebral space within a localized spinal segment for alignment and motion restoration 1170, 1170a, 1170b, 1170c comprises the step of preparing a caudal vertebral body on at least one side 1720, 1760, 1780. The step of preparing a caudal vertebral body 1720, 1760, 1780 comprises the steps of: obtaining one or more selected surgical measurements (SSM) or PSM for optimal alignment from the preoperative method or procedure or surgical plan for the at least one side 1800; superimposing one or more objects onto one or more operative images for at least one side 1805; positioning robot and/or surgeon to the desired one or more objects on the at least one side 1810; introducing the preparation tool on at least one side that follows a custom path and/or the one or more objects 1815; providing feedback on matching or substantially matching the one or more objects on the one or more operative images 1455; preparing a portion of an endplate and a pedicle on a caudal vertebral body on at least one side to create resected surface 1820; providing feedback on matching or substantially matching the one or more objects on the one or more operative images 1455; and confirming the SSM and/or one or more objects by acquiring at least one image using at least one imaging technique 1825. The SSM comprises a sagittal osteotomy angle, a coronal osteotomy angle, a convergence angle or transverse pedicle angle, an A/P distance, implant size height, and/or implant size length.

With reference to FIG. 42B, the step of preparing an intervertebral space within a localized spinal segment for alignment and motion restoration 1170, 1170a, 1170b, 1170c comprises the step of completing a first preparation on a caudal vertebral body on a first side and a second preparation on the caudal vertebral body on a second side second side 1740. The step of step of completing a first preparation on a caudal vertebral body on a first side and a second preparation on the caudal vertebral body on a second side second side 1740 comprises the steps of: obtaining a first SSM for a first side and a second SSM for a second side for optimal alignment (e.g., sagittal and/or coronal alignment) from the preoperative method or procedure or surgical plan 1830; superimposing a first one or more objects onto a first one or more operative images of a first side and a second one or more objects onto a second one or more operative images of a second side 1835; positioning a user (e.g., robot and/or surgeon) to the desired first one or more objects on the first side and a second one or more objects on the second side 1840; introducing a first preparation tool on a first side that follows a first one or more objects and a second preparation tool on a second side that follows one or more objects 1845; providing feedback on matching or substantially matching the first and/or second one or more objects on the one or more operative images 1455; completing a first preparation on a portion of an endplate and a pedicle on a caudal vertebral body on a first side to create a first resected surface that matches or substantially matches the first one or more objects and/or first SSM for optimal alignment and a second preparation on a portion of an endplate and a pedicle on the caudal vertebral body on a second side to create a second resected surface that matches or substantially matches the second SSM or second one or more objects for optimal alignment 1850; providing feedback on matching or substantially matching the one or more objects on the one or more operative images 1455; and confirming the first and second one or more objects by acquiring at least one image using at least one imaging technique 1855.

The first preparation on the caudal vertebral body on the first side may be the same as the second preparation on the caudal vertebral body on the second side. The first preparation on the caudal vertebral body on the first side may be different than the second preparation on the caudal vertebral body on the second side. The first spinal implant may be the same size as the second spinal implant. The first spinal implant may be a different size than the second spinal implant. The first one or more SSM and/or first one or more objects may comprise the same object as the second one or more SSM and/or second one or more objects. The first one or more SSM or first one or more objects may comprise a different object than the second one or more SSM and/or second one or more objects. The first transverse pedicle angle may comprise a same transverse angle as the second transverse pedicle angle. The first transverse pedicle angle may comprise a different transverse angle than the second transverse pedicle angle. The first optimal alignment may be the same or different compared to the second optimal alignment.

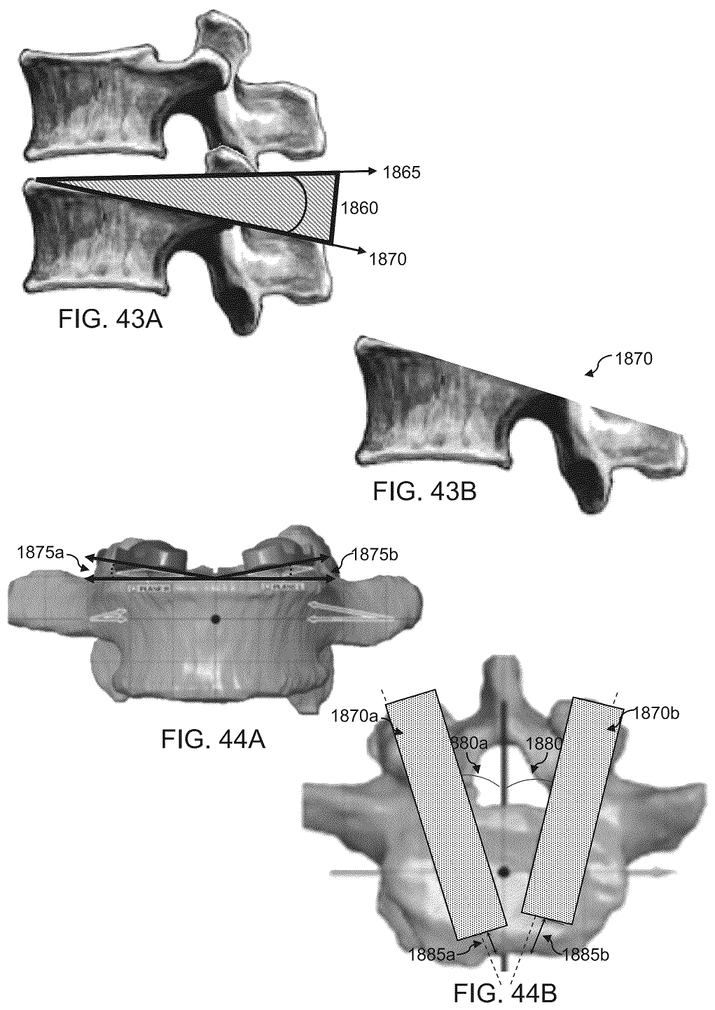

The step of preparing the caudal vertebral body 1720, 1740, 1760, 1780 comprises the step of obtaining one or more SSM or PSM, a first one or more SSM or PSM and/or a second SSM or PSM for optimal alignment for the one or more preoperative methods or procedures or preoperative surgical plan 1800, 1830 as shown in FIGS. 43A-43B and 44A-44B. The one or more SSM or PSM, first SSM, and/or a second SSM may comprise a sagittal osteotomy angle or trajectory 1860 (e.g., sagittal angle of correction), a coronal osteotomy angle or trajectory 1875a, 187b (e.g., coronal angle of correction), convergence angle or transverse pedicle angle 1880a, 1880b; and/or A/P distance 1885a, 1885b. The sagittal osteotomy angle or trajectory comprises an angle of 0 degrees to 40 degrees. The coronal osteotomy angle or trajectory comprises an angle of 0 degrees to 40 degrees. The transverse pedicle angle may comprise 0 degrees to 45 degrees.

The sagittal osteotomy angle 1860, the coronal osteotomy angle 1875a, 1875b, the transverse pedicle angle 1880a, 1880b and/or the A/P distance 1885a, 1885b may be different in different spine segments within different spine regions. A first coronal osteotomy angle 1875a may comprise a different angle than the second coronal osteotomy angle 1875b. A first coronal osteotomy angle 1875a may comprise a same angle than the second coronal osteotomy angle 1875b. A first sagittal osteotomy angle 1860 may comprise a different angle than the second sagittal osteotomy angle 1860. A first sagittal osteotomy angle 1860 may comprise a same angle than the second sagittal osteotomy angle 1860. A first A/P distance 1885a may comprise the same distance as the second A/P distance 1885b. The first A/P distance 1885a may comprise a different distance as the second A/P distance 1885b. The one or more SSM or PSM may be transformed into one or more objects that are superimposed onto the one or more preoperative images, one or more operative images, one or more synchronized image, one or more MPR images.

With reference to FIGS. 35 and/or 36A-36D, the different images 1555, 1560, 1565, 1570, 1575 depict the step of superimposing one or more objects onto one or more operative images on the at least one side 1805 and/or the step of superimposing a first one or more objects and/or a second one or more objects onto one or more operative images 1835. The operative images may comprise a one or more preoperative images, one or more operative images, one or more synchronized operative images and/or one or more MPR images. The object may comprise an annotated text 1545, 1580, surgical measurement or projected surgical measurement (PSM) 1550, 1585, a trajectory path 1310, a virtual surgical instrument 1320, a virtual spinal implant 1335 and/or any combination thereof. The trajectory path 1310 may comprise the trajectory of a surgical instrument, a surgical approach, one or more spinal implants and/or an SSM. The PSMs or SSMs include a sagittal osteotomy angle, a coronal osteotomy angle convergence angle and A/P distance. The SSM may further include a force value and/or a resistance value. The one or more virtual trajectory paths 1310 and/or virtual tools 1320 should follow, match or substantially match the convergence angle or transverse pedicle angle trajectory path.

In another embodiment, the step of preparing the caudal vertebral body 1720, 1740, 1760, 1780 comprises the step of positioning the user, surgeon and/or robot to the desired one or more objects on the at least one side 1810 and/or the step of positioning the user to a first one or more objects or trajectory on a first side and the second one or more objects or trajectory on a second side 1840. The navigation system may display the one or more objects, a first one or more objects and/or a second one or more objects. The spatial coordinates may be transmitted to the robot and/or the proper referencing system to confirm the one or more surgical instruments, a first surgical instrument and/or a second surgical instrument is positioned to desired trajectory or the one or more objects relative to the targeted anatomy. The user comprising a surgeon and/or a robotic system may hold the one or more surgical instruments and/or a registration tool (i.e., a probe, a guide and/or any other tool) to the desired position according to the spatial coordinates of the surgical trajectory for the desired intraoperative step. The trajectory may match or substantially match one or more projected surgical measurements (PSM). The one or more objects comprise one or more PSMs, a virtual surgical instrument, a virtual spinal implant, and/or any combination thereof.

In another embodiment, the step of preparing the caudal vertebral body 1720, 1740, 1760, 1780 comprises the step of introducing a preparation tool on at least one side 1815 and/or the step of introducing a first preparation tool on a first side and a second preparation tool on a second side 1845. The step of introducing a preparation tool on at least one side, a first side and/or a second side 1815, 1845 comprises the steps of: assembling the preparation tool, a first preparation tool and/or a second preparation tool; distracting the intervertebral disc space of a localized spine segment within a spine region; and inserting the preparation tool, the first preparation tool and/or a second preparation tool into the localized intervertebral space. The step of introducing a preparation tool on at least one side, a first side and/or a second side may further comprise the step of re-registering and/or re-identifying the preparation tool.

In another embodiment, the step of introducing a preparation tool, a first preparation tool and/or a second preparation tool on at least one side, a first side and/or a second side 1815, 1845 comprises the step of assembling the preparation tool, a first preparation tool and/or a second preparation tool. The step of assembling or preparing the preparation tool, a first preparation tool and/or a second preparation tool comprises attaching the preparation tool to a handle system or a robot. The preparation tool comprises a rasp, the rasp includes a long, short and/or flat rasp. The handle system comprises a manual handle, and/or a powered handle system. The surgeon may prepare the endplates and/or pedicles manually using manual handle and/or using a powered system under navigational and/or robotic assistance.

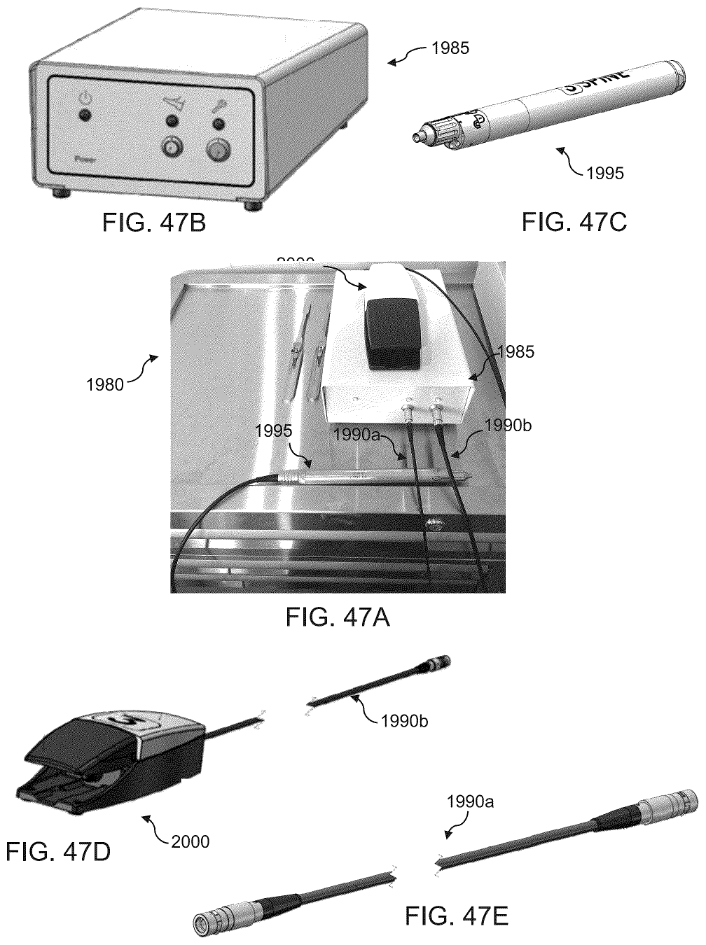

In one embodiment, the surgeon may use a powered handle system 1980. The powered handle system 1980 comprises a powered reciprocating control box 1985, a powered handle or handpiece 1995, a footswitch pedal 2000, one or more cable connectors 1990a, 1990b as shown in FIGS. 47A-47E. The rasp may comprise a long or short and flat rasp. To use the powered system, the surgeon should connect the cable connectors to the control box and cord into an outlet allowing the powered system to activate. The surgeon may subsequently insert a rasp into an unlocked powered handpiece and twist the locking collar on the powered handpiece to lock the rasp into the handpiece. The surgeon may test the powered system by stepping on the footswitch pedal. The pressure on the footswitch determines the variable speed to activate and/or reciprocate the rasp.

In another embodiment, the step of introducing a preparation tool, a first preparation tool and/or a second preparation tool on at least one side, a first side and/or a second side 1815, 1845 comprises the step of re-identifying and/or re-registering surgical instruments within the navigation system and/or robot. The surgical instruments may have been previously tracked during the preoperative procedure (e.g., a baseline registration), and may not require further reregistration. The surgeon or staff may be able to couple a portion of a reference system and/or robotic arm to the previously identified surgical instrument, and begin immediate use of the instrument. Alternatively, the surgeon or staff may couple a portion of a reference system and/or couple to a robotic arm to the previously identified instrument (e.g., the baseline registration during the preoperative method), and may require intraoperative confirmation of spatial relationship or coordinates between the patient and the pre and intraoperative images. As disclosed herein, each of the surgical instruments, including the powered handpiece of the powered system may be adapted and configured to receive a portion of a reference system including at least one tracker, at least one probe, one or more fiducials and/or one or more LEDs. The handpiece may comprise an indentation or channel along the length of the handpiece, between the first end and/or second end of the handpiece. The portion of the reference system may align with the indentation and/or channel.

In another embodiment, the step of introducing a preparation tool, a first preparation tool and/or a second preparation tool on at least one side, a first side and/or a second side 1815, 1845 comprises the step of distracting the intervertebral disc space of a localized spine segment within a spine region. This step requires the surgeon to distract the interverbal disc space on at least one side, a first side and/or a second side. The surgeon should over distract the at least one side, a first side and/or a second side by using a height trial. The height trial should comprise at least 1 mm larger or taller than the height trial determined at the step of determining the proper implant height. Maintaining the at least one side, a first side and/or a second side using a height trial that is at least 1 mm greater than determined implant height from the previous step will provide extra tension on the tissues and increase the intervertebral disc space for easier endplate preparation and the completion of the osteotomy of the pedicles.

In another embodiment, the step of introducing a preparation tool, a first preparation tool and/or a second preparation tool on at least one side, a first side and/or a second side 1815, 1845 comprises the step of inserting the preparation tool into the localized intervertebral space of at least one side and/or inserting a first preparation tool into the localized intervertebral space of a first side and a second preparation tool into the localized intervertebral space on a second side that follows or substantially follows a custom path. This step may require the surgeon to introduce or insert the inactivated powered rasp into the distracted intervertebral disc space on the native caudal vertebral body into the at least one side, a first side and/or a second side along a desired path and/or desired one or more objects, a first one or more objects and/or a second one or more objects. The desired path may be a custom path chosen by the surgeon. The desired path, one or more objects, a first one or more objects and/or a second one or more objects may comprise a path that follows along, match or substantially match the pedicle transverse angle (or pedicle axis) on the at least one side, a first side and/or a second side within a distracted intervertebral disc space. The rasp comprises a long, flat rasp and/or a short, flat rasp. The surgeon should confirm placement and/or positioning of the preparation tools by acquiring at least one image with at least one imaging technique on the native caudal endplate surface and/or pedicle surface. The surgeon may refer or obtain the pre-operative measurements, the preoperative measurements include the optimal sagittal and/or coronal angle of correction.

In another embodiment, the step of preparing the caudal vertebral body 1720, 1740, 1760, 1780 comprises the step of completing at least one preparation along a custom path on at the least one side to create at least one resected surface 1820 and/or the step of completing a first preparation along the first custom path on the first side to create a first resected surface and a second preparation along a second custom path on the second side to create a second resected surface 1850. The preparation comprises the step of completing a resection of the endplate and osteotomy of a pedicle within the disc space of a localized spine segment and/or the vertebral bodies. The step of completing an osteotomy and/or resection within a disc space and/or vertebral body of a localized spine segment is a corrective osteotomy to restore sagittal and/or coronal balance, along with maximizing implant range of motion or motion restoration. The osteotomy and/or resection will result in the creation of one or more parallel resected surfaces or endplates for neutral insertion of the spinal implant. The degree of osteotomy, the osteotomy sagittal angle (e.g., the sagittal angle of correction (AOC) and the osteotomy coronal angle (e.g., the coronal angle of correction) that is necessary is generally the angle to create parallel cranial and caudal prepared endplates at the index vertebral segment level and as determined in preoperative (or intraoperative if using navigation) planning and confirmed intraoperatively. The custom path, first custom path and/or a second custom path comprises matching or substantially matching the convergence angle and/or transverse pedicle angle.

With reference to FIGS. 43A-43B and 44A-44B, the different vertebral body segments depict one embodiment of the preparation of the superior endplate and the pedicle of the caudal vertebral body. The one or more SSM or PSM, first SSM, and/or a second SSM may comprise a sagittal osteotomy angle or trajectory 1860 (e.g., sagittal angle of correction), a coronal osteotomy angle or trajectory 1875a, 187b (e.g., coronal angle of correction), convergence angle or transverse pedicle angle 1880a, 1880b; and/or A/P distance 1885a, 1885b. The sagittal osteotomy angle or trajectory comprises an angle of 0 degrees to 40 degrees. The coronal osteotomy angle or trajectory comprises an angle of 0 degrees to 40 degrees. The transverse pedicle angle may comprise 0 degrees to 45 degrees.

The sagittal osteotomy angle 1860, the coronal osteotomy angle 1875a, 1875b, the transverse pedicle angle 1880a, 1880b and/or the A/P distance 1885a, 1885b may be different in different spine segments within different spine regions. A first coronal osteotomy angle 1875a may comprise a different angle than the second coronal osteotomy angle 1875b. A first coronal osteotomy angle 1875a may comprise a same angle than the second coronal osteotomy angle 1875b. A first sagittal osteotomy angle 1860 may comprise a different angle than the second sagittal osteotomy angle 1860. A first sagittal osteotomy angle 1860 may comprise a same angle than the second sagittal osteotomy angle 1860. A first A/P distance 1885a may comprise the same distance as the second A/P distance 1885b. The first A/P distance 1885a may comprise a different distance as the second A/P distance 1885b. The one or more SSM or PSM may be transformed into one or more objects that are superimposed onto the one or more preoperative images, one or more operative images, one or more synchronized image, one or more MPR images.

The surgeon may activate the powered rasp (see FIGS. 47A-47E) that is disposed onto the native endplate surface 1865 by pressing on the footswitch—a light pressure equals a slow reciprocating speed, and a hard pressure equals a faster reciprocating speed. The surgeon prepares at least a portion of the native superior endplate 1865 and the pedicle on the caudal vertebral body by utilizing the flat reciprocating rasp at a desired speed until the posterior aspect is flush with the vertebral body and a slight bleeding is observed to facilitate bony ingrowth to the spinal implant after deployment to create at least one resected surface, first resected surface, and/or a second resected surface 1870, 1870a, 1870b on at least one side, a first side and/or a second side. Alternatively, the surgeon may prepare at least a portion of the native superior endplate 1865 on the caudal vertebral body following along a custom path. The custom path may match or substantially match the pedicle transverse angle (e.g., the pedicle axis or convergence angle) utilizing the long, flat rasp as a desired speed until the posterior aspect is flush with the vertebral body and a slight bleeding is observed to facilitate bony ingrowth to the spinal implant after deployment to create at least one resected surface, a first resected surface and/or a second resected surface 1870, 1870a, 1870b. The transverse pedicle angle may include 0 degrees to 45 degrees. Accordingly, the surgeon may prepare at least a portion of the superior endplate of the caudal vertebral body to comprise a first resected shape, a first resected length, a first resected width and a first resected depth.

The at least one resected surface, a first resected surface and/or a second resected surface 1870, 1870a, 1870b may comprise a flat, planar plane or surface and/or be parallel to the native endplate surface. The at least one resected surface, a first resected surface and/or a second resected surface 1870, 1870a, 1870b may comprise an osteotomy angle, the angles may comprise 0 degrees to 40 degrees. The angles may comprise a sagittal angle, a coronal angle, a transverse pedicle angle (e.g., a convergence angle), and/or any combination thereof. The angles of the at least one resected surface, a first resected surface and/or a second resected surface 1870, 1870a, 1870b may be angled relative to the native endplate plane 1865. The at least one resected surface, a first resected surface and/or a second resected surface 1870, 1870a, 1870b may be below and parallel relative to the native endplate plane 1865. The at least one resected surface, a first resected surface and/or a second resected surface 1870, 1870a, 1870b may be below and angled relative to the native endplate plane 1865. The first resected surface may comprise a flat and angled surface.

Alternatively, the angle of the at least one resected surface, a first resected surface and/or a second resected surface 1870, 1870a, 1870b may match or substantially match the sagittal and/or coronal osteotomy angle. The angle of the at least one resected surface, a first resected surface and/or a second resected surface 1870, 1870a, 1870b on at least one side, a first side and/or a second side may match or substantially match the transverse pedicle angle. The at least one resected surface, a first resected surface and/or a second resected surface 1870, 1870a, 1870b may comprise a resected shape, first resected length and first resected width may match or substantially match the shape, length and width of the spinal implant.

The at least one resected surface, a first resected surface and/or a second resected surface 1870, 1870a, 1870b on at least one side, a first side and/or a second side may extend from a portion of the superior endplate to a portion of the pedicle on the caudal vertebral body. The at least one resected surface, a first resected surface and/or a second resected surface 1870, 1870a, 1870b on at least one side, a first side and/or a second side may comprise resected shape, a resected length, a resected width and a resected depth. The resected surface length and/or the resected width may match at least a portion of the spinal implant length and/or width. The resected surface may be positioned or created at an angle. The angle of the resected surface may match or substantially match the pre-determined SSM or PSM, one or more objects, and/or an AOC from pre-procedure step. The angle may comprise an osteotomy sagittal angle, an osteotomy coronal angle, a transverse pedicle angle (e.g., convergence angle), and/or any combination thereof.

In another embodiment, the step of preparing the caudal vertebral body on at least one side, a first side and/or a second side 1720, 1740, 1760, 1780 may comprise the step of providing feedback on matching or substantially matching one or more objects that were superimposed on the one or more images 1455 and/or the step of providing feedback on matching or substantially matching a first one or more objects on a first side and a second one or more objects on a second side 1455. The feedback may comprise visual feedback (using the navigation monitor or workstation display), audible feedback, tactile feedback, and/or any combination thereof. The feedback may include visual and/or audible feedback to correct or reposition the surgeon's trajectory and/or robot's trajectory to appropriately match or substantially match the one or more objects and/or projected surgical measurement. The one or more objects may include the SSM (selected surgical measurement), a virtual trajectory, a virtual tool, a virtual spinal implant, and/or any combination thereof. The SSM may include a convergence angle or transverse pedicle angle, and a COR. The virtual trajectory may match or substantially match the convergence angle or transverse pedicle angle.

In one embodiment, the step of providing feedback 1455 may further comprise the steps of: generating a non-desired trajectory path and/or non-desired one or more objects; comparing the desired trajectory path to the non-desired trajectory path 1460; correcting user to return to desired trajectory path if the non-desired trajectory path is different than the desired trajectory path 1435, 1480. In another embodiment, the step of providing feedback 1455 may further comprise the steps of: generating a second trajectory path and/or non-desired second one or more objects; comparing the first trajectory path to the second trajectory path 1460; correcting user to return to first trajectory path if the second trajectory path is different than the first trajectory path 1435, 1480. The step of providing feedback 1455 may comprise at least one side, a first side and/or a second side.

Furthermore, step of preparing the caudal vertebral body on the at least one side, first side and/or second side 1720, 1740, 1760, 1780 may include the step of acquiring at least image using an imaging technique on at least one side, a first side and/or a second side 1825, 1855 to confirm the one or more objects, a first one or more objects and/or a second one or more objects of the resected surface, the first resected surface and/or the second resected surface on at least one side, a first side and/or a second side 1870, 1870a, 1870b by acquiring at least one image using at least one imaging technique. The surgeon may verify that the angles of the resected surface, the first resected surface and/or second resected surface by comparing it to the preoperative surgical plan and/or the at least one or more objects, a first one or more objects and/or second one or more objects displayed or superimposed onto the workstation. The at least one image may be performed by using at least one imaging technique. The imaging techniques may comprise 2D or 3D images. The imaging techniques may comprise an MRI, a radiograph, a CT scan, an ultrasound, and/or any combination thereof. The preparation of the endplate surface and pedicle surface of caudal vertebral body on the contralateral side may be performed by repeating the above steps.

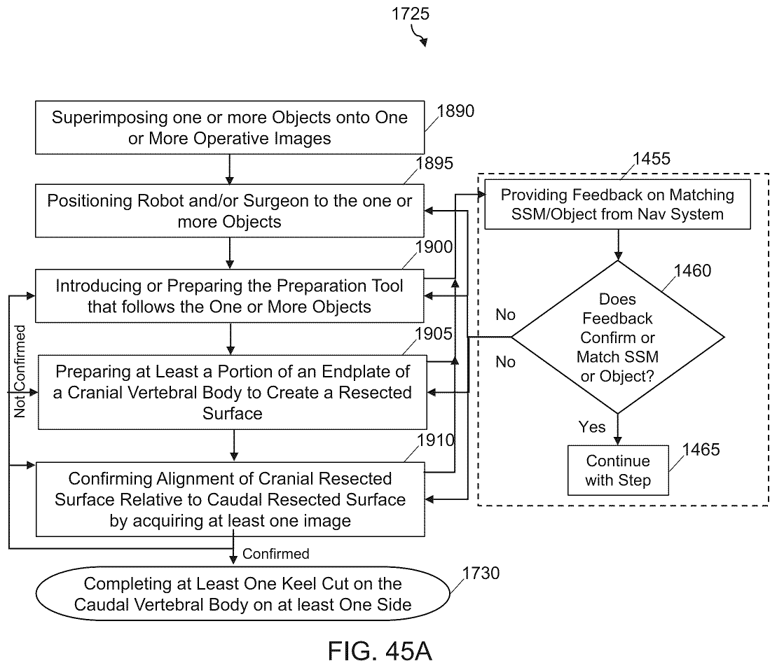

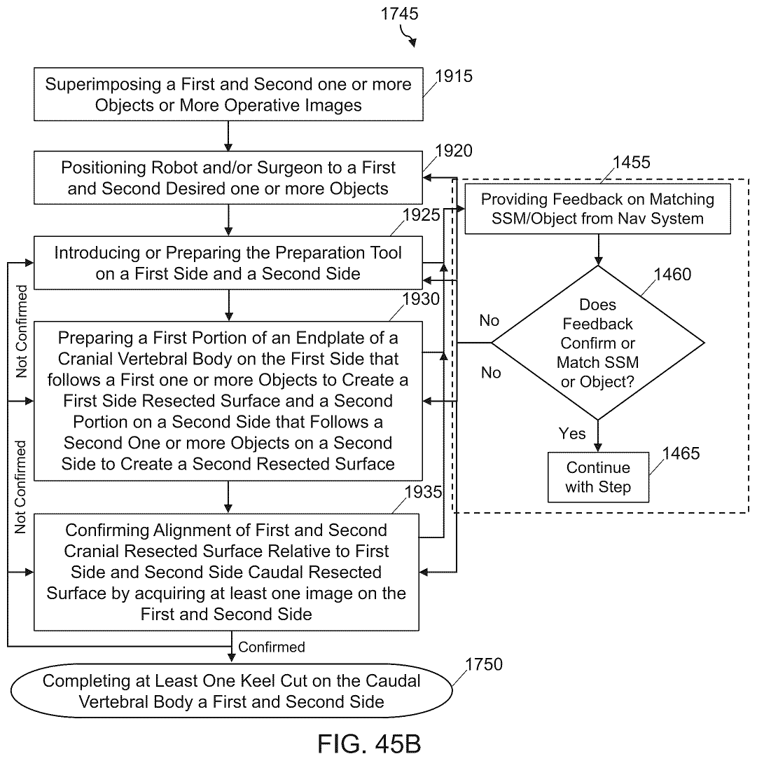

With reference to FIG. 45A, the step of preparing an intervertebral space within a localized spinal segment for alignment and motion restoration 1170, 1170a, 1170b, 1170c comprises the step of preparing a cranial vertebral body on at least one side 1725, 1765, 1785. The step of preparing a cranial vertebral body on at least one side 1725, 1765, 1785 comprises the steps of: superimposing a one or more objects onto one or more operative images on at least one side 1890; positioning a user, robot and/or surgeon to the one or more objects or one or more trajectories 1895; introducing a preparation tool within an intervertebral disc space on at least one side that follows the one or more objects and/or a custom path 1900; preparing a portion of an endplate on a cranial vertebral body on at least one side to create resected or prepared surface 1905; confirming the one or more objects and/or SSM/PSM on the at least one side by acquiring at least one image using at least one imaging technique 1910; providing feedback to correct the introducing step, the preparing step and the confirming alignment step 1455. The step of preparing a cranial vertebral body on at least one side 1725, 1765, 1785 may further comprise the steps of obtaining one or more SSM or PSM from the preoperative method and/or software application for optimal alignment. The one or more objects may match or substantially match the SSM/PSM, the SSM comprises sagittal angle, a resection depth, a transverse pedicle angle or convergence angle. The custom path or trajectory path matches or substantially matches the transverse pedicle angle or convergence angle.

With reference to FIG. 45B, the step of preparing an intervertebral space within a localized spinal segment for alignment and motion restoration 1170, 1170a, 1170b, 1170c comprises the step of a first preparation on a cranial vertebral body on a first side and a second preparation of the cranial vertebral body on a second side 1745. The step of the first preparation on a cranial vertebral body on a first side and a second preparation of the cranial vertebral body on a second side 1745 comprises the steps of: superimposing a first one or more objects onto a first one or more operative images on a first side and a second one or more objects onto a second one or more operative images on a second side 1915; positioning robot and/or surgeon to the first one or more objects on a first side and a second one or more objects on a second side 1920; introducing a first preparation tool on a first side that follows a first path or trajectory and a second preparation tool on a second side that follows a second path or trajectory 1925; completing a first preparation on a portion of an endplate on a cranial vertebral body on a first side to create a first resected or prepared surface and a second preparation on a portion of an endplate and a pedicle on the caudal vertebral body on a second side to create a second resected or prepared surface 1930, and/or confirming the first and second one or more objects by acquiring at least one image using at least one imaging technique 1935; providing feedback to correct the introducing step, the preparing step and the confirming alignment step 1455.

The step of preparing a cranial vertebral body on a first side and/or a second side 1745 may further comprise the steps of obtaining one or more SSM or PSM from the preoperative method and/or software application for optimal alignment. The one or more objects may match or substantially match the SSM/PSM, the SSM comprises sagittal angle, a resection depth, a transverse pedicle angle or convergence angle. The custom path or trajectory path matches or substantially matches the transverse pedicle angle or convergence angle.

The first preparation on the cranial vertebral body on the first side may be the same as the second preparation on the cranial vertebral body on the second side. The first preparation on the cranial vertebral body on the first side may be different than the second preparation on the cranial vertebral body on the second side. The first angle of correction may comprise the same angle as the second angle of correction. The first angle of correction may comprise a different angle than the second angle of correction. The first transverse pedicle angle may comprise a same transverse angle as the second transverse pedicle angle. The first transverse pedicle angle may comprise a different transverse angle than the second transverse pedicle angle. The first and/or second transverse pedicle angle may comprise an angle of 0 to 45 degrees. The first one or more objects may comprise a same object as the second one or more objects. The first one or more objects may comprise a different one or more objects than the second one or more objects. The first one or more SSM or PSM may comprise a same or different measurement than the second one or more SSM or PSM. The first custom path or trajectory may be the same or different compared to the second custom path or trajectory.

In one embodiment, the step of preparing the cranial vertebral body on at least one side, a first side and/or a second side 1725, 1745 1765, 1785 comprises the step of obtaining one or more SSM or PSM, a first one or more SSM or PSM and/or a second SSM or PSM for optimal alignment (e.g., sagittal and/or coronal alignment) from the preoperative method or procedure or software application. The one or more SSM or PSM, the first one or more SSM or PSM, and/or a second one or more SSM or PSM may comprise a resection depth, a sagittal osteotomy angle or trajectory (e.g., sagittal angle of correction), a coronal osteotomy angle or trajectory (e.g., coronal angle of correction), a transverse pedicle angle or convergence angle, an A/P distance, and/or neutral alignment. The sagittal osteotomy angle or trajectory comprises an angle of 0 degrees to 40 degrees. The coronal osteotomy angle or trajectory comprises an angle of 0 degrees to 40 degrees. The transverse pedicle angle may comprise 0 degrees to 45 degrees. The sagittal osteotomy angle, the coronal osteotomy angle, the transverse pedicle angle and/or the A/P distance may be different in different spine segments within different spine regions. A first coronal osteotomy angle may comprise a different angle than the second coronal osteotomy angle. A first coronal osteotomy angle may comprise a same angle than the second coronal osteotomy angle. A first sagittal osteotomy angle may comprise a different angle than the second sagittal osteotomy angle. A first sagittal osteotomy angle may comprise a same angle than the second sagittal osteotomy angle. The resection depth may comprise 0 to 10 mm.

With reference to FIGS. 35 and/or 36A-36D, the different images 1555, 1560, 1565, 1570, 1575 depict the step of superimposing one or more objects onto one or more operative images 1890 and/or the step of superimposing a first one or more objects and/or a second one or more objects onto one or more operative images 1915. The operative images may comprise a one or more preoperative images, one or more operative images, one or more synchronized operative images and/or one or more MPR images. The object may comprise an annotated text 1545, 1580, surgical measurement or projected surgical measurement (PSM) 1550, 1585, a trajectory path 1310, a virtual surgical instrument 1320, a virtual spinal implant 1335 and/or any combination thereof. The trajectory path 1310 may comprise the trajectory of a surgical instrument, a surgical approach, one or more spinal implants and/or an SSM. The PSMs or SSMs include a resection depth, a sagittal osteotomy angle, a coronal osteotomy angle, convergence angle, A/P distance and/or neutral alignment. The SSM may further include a force value and/or a resistance value. The one or more virtual trajectory paths 1310 and/or virtual tools 1320 should follow, match or substantially match the convergence angle or transverse pedicle angle trajectory path. The trajectory path may match or substantially match one or more SSM or PSM, a first one or more SSM and/or a second one or more SSM or PSM. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance and/or neutral alignment.

In another embodiment, the step of preparing the cranial vertebral body 1725, 1745, 1765, 1785 comprises the step of positioning the user, surgeon and/or robot to the desired one or more objects on the at least one side 1895 and/or the step of positioning the user to a first one or more objects or trajectory on a first side and the second one or more objects or trajectory on a second side 1920. The navigation system may display the one or more objects, a first one or more objects and/or a second one or more objects. The spatial coordinates may be transmitted to the robot and/or the proper referencing system to confirm the one or more surgical instruments, a first surgical instrument and/or a second surgical instrument is positioned to desired trajectory or the one or more objects relative to the targeted anatomy. The user comprising a surgeon and/or a robotic system may hold the one or more surgical instruments and/or a registration tool (i.e., a probe, a guide and/or any other tool) to the desired position according to the spatial coordinates of the surgical trajectory for the desired intraoperative step. The trajectory may match or substantially match one or more projected surgical measurements (PSM). The one or more objects comprise one or more PSMs, a virtual surgical instrument, a virtual spinal implant, and/or any combination thereof. In one exemplary embodiment, the one or more objects, the first one or more objects and/or the second one or more objects comprises a one or more text annotations and one or more trajectory paths. The text annotations include PSM or SSM, the SSM includes a resection depth, an osteotomy sagittal angle (e.g., a sagittal angle of correction), an osteotomy coronal angle (e.g., a coronal angle of correction), a transverse pedicle angle or convergence angle, A/P distance and/or neutral alignment.

In another embodiment, the step of preparing the cranial vertebral body 1725, 1745, 1765, 1785 comprises the step of introducing a preparation tool along a path or trajectory on at least one side 1900 and/or the step of introducing a first preparation tool along a first custom path or trajectory on a first side and a second preparation tool along a second custom path or trajectory on a second side 1925. The step of introducing a preparation tool on at least one side, a first side and/or a second side 1900, 1925 comprises the steps of assembling one or more preparation tools on at least one side, a first side and/or a second side; distracting the intervertebral disc space of a localized spine segment within a spine region; and inserting the one or more preparation tools into the localized intervertebral space on at least one side, a first side and/or a second side.

In another embodiment, the step of introducing a preparation tool, a first preparation tool and/or a second preparation tool on at least one side, a first side and/or a second side 1900, 1925 comprises the step of re-identifying and/or re-registering surgical instruments within the navigation system and/or robot. The surgical instruments may have been previously tracked during the preoperative procedure (e.g., a baseline registration), and may not require further reregistration. The surgeon or staff may be able to couple a portion of a reference system and/or robotic arm to the previously identified surgical instrument and begin immediate use of the instrument. Alternatively, the surgeon or staff may couple a portion of a reference system and/or couple to a robotic arm to the previously identified instrument (e.g., the baseline registration during the preoperative method), and may require intraoperative confirmation of spatial relationship or coordinates between the patient and the pre and intraoperative images. As disclosed herein, each of the surgical instruments, including the powered handpiece of the powered system may be adapted and configured to receive a portion of a reference system including at least one tracker, at least one probe, one or more fiducials and/or one or more LEDs. The handpiece may comprise an indentation or channel along the length of the handpiece, between the first end and/or second end of the handpiece. The portion of the reference system may align with the indentation and/or channel.

In another embodiment, the step of introducing a preparation tool on at least one side, a first side and/or a second side 1900, 1925 comprises the step of assembling the preparation tool, a first preparation tool and/or a second preparation tool. The preparation tool comprises a rasp, a manual handle, a powered handle system and/or any combination thereof. The surgeon may prepare the endplates and/or pedicles manually and/or using a powered system. In one embodiment, the surgeon may use a powered system. The surgeon may use a powered handle system 1980. The powered handle system 1980 comprises a powered reciprocating control box 1985, a powered handle or handpiece 1995, a footswitch pedal 2000, one or more cable connectors 1990a, 1990b as shown in FIGS. 47A-47E. The rasp may comprise a long or short and flat rasp. To use the powered system, the surgeon should connect the connector cables to the control box and cord into an outlet allowing the powered system to activate. The surgeon may subsequently insert a rasp into an unlocked powered handpiece and twist the locking collar on the handpiece to lock the rasp into the handpiece. The surgeon may test the powered system by stepping on the footswitch. The pressure on the footswitch determines the variable speed to activate and/or reciprocate the rasp.

In another embodiment, the step of introducing a preparation tool, a first preparation tool and/or a second preparation tool on at least one side, a first side and/or a second side 1815, 1845 comprises the step of distracting the intervertebral disc space of a localized spine segment within a spine region. This step requires the surgeon to distract the interverbal disc space on at least one side, a first side and/or a second side. The surgeon and/or the robot should over distract the at least one side, a first side and/or a second side by using a height trial. The height trial should comprise at least 1 mm larger or taller than the height trial determined at the step of determining the proper implant height. Maintaining the at least one side, a first side and/or a second side using a height trial that is at least 1 mm greater than determined implant height from the previous step will provide extra tension on the tissues and increase the intervertebral disc space for easier endplate preparation and the completion of the osteotomy of the pedicles. This distraction should be maintained when transitioning from the step of preparing the caudal vertebral body to the step of preparing the cranial vertebral body.

In another embodiment, the step of introducing a preparation tool on at least one side, a first side and/or a second side 1815, 1845 comprises the step of inserting the preparation tool into the localized intervertebral space on at least one side, a first side and/or a second side. This step may require the surgeon and/or robot to introduce or insert the inactivated powered rasp into the distracted intervertebral disc space on the native cranial vertebral body into the at least one side, a first side and/or a second side along a desired path, the one or more objects and/or the one or more SSMs or PSMs. The desired path and/or the one or more objects may be a custom path or virtual trajectory. Alternatively, the desired path and/or one or more objects may comprise a path or trajectory that follows along, match or substantially match the pedicle transverse angle (or pedicle axis) on the at least one side, a first side and/or a second side within a distracted intervertebral disc space. The rasp comprises a long, flat rasp and/or a short, flat rasp. The surgeon should confirm placement and/or positioning of the preparation tool by acquiring at least one image with at least one imaging technique on the native cranial endplate surface.

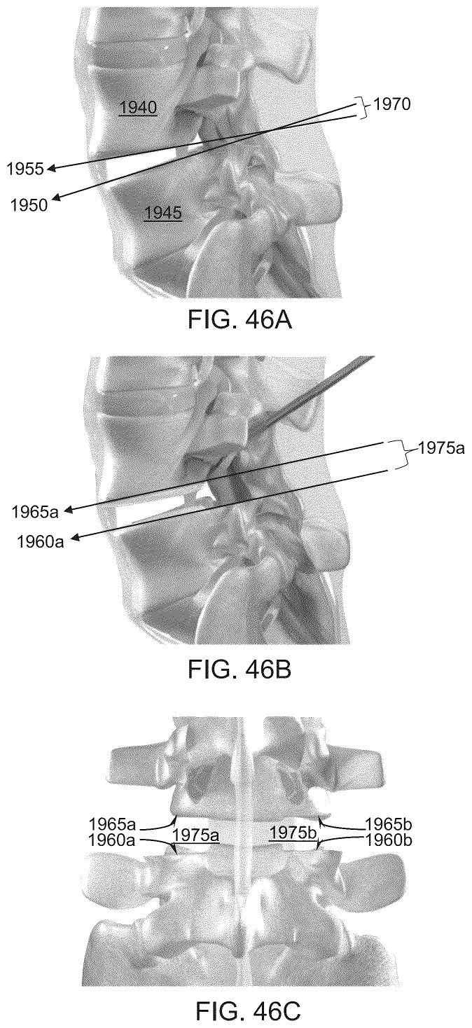

FIGS. 46A-46C depicts one embodiment of the preparation of the caudal vertebral body 1820, 1850 and of the cranial vertebral body 1905, 1930 to achieve parallel or substantially parallel resected or prepared surfaces for optimal alignment and range of motion. The native endplate surfaces of the cranial vertebral body 1940 and the native endplate surface 1950 of the caudal vertebral body 1945 illustrate that the intervertebral space 1970 is not parallel. The user, surgeon and/or robot uses the previously obtained one or more selected surgical measurements to prepare the native caudal endplate surface 1950 and the native cranial endplate surface 1955. The spatial coordinates are transmitted by the workstation to position the user and/or surgeon, and the user and/or surgeon may activate the powered rasp that is disposed onto the native endplate surface by pressing on the footswitch—a light pressure equals a slow reciprocating speed, and a hard pressure equals a faster reciprocating speed (refer to FIGS. 47A-47E). The surgeon prepares at least a portion of the native inferior endplate surface 1955 on the cranial vertebral body by utilizing the flat reciprocating rasp at a desired speed until a slight bleeding and a particular depth and/or angle is observed to facilitate bony ingrowth to the spinal implant after deployment to create a resected or prepared surface, a first resected or prepared surface and/or a second resected or prepared surface 1965a, 1965b.

Alternatively, the surgeon may prepare at least a portion of the native inferior endplate surface 1955 on the cranial vertebral body 1945 following along, matching or substantially matching the one or more objects, the one or more SSM or PSM, and/or a custom path. The custom path, one or more objects, and/or one or more SSMs comprises a pedicle transverse angle (e.g., the pedicle mid axis or convergence angle). The transverse pedicle angle may include 0 degrees to 45 degrees. Accordingly, the surgeon may prepare at least a portion of the native inferior endplate surface 1955 of the cranial vertebral body to comprise a portion of at least one resected surface, a first resected surface, and/or a second resected surface 1965a, 1965b. Alternatively, the surgeon may prepare the entirety of the native cranial surface 1955 to create an entire at least one resected surface, a first rested surface and/or a second resected surface. The resected surface comprises a resected or prepared shape, a first resected or prepared length, a first resected or prepared width and a first resected or prepared depth.

The step of preparing the cranial vertebral body on the at least one side, first side and/or second side 1725, 1745, 1765, 1785 may include the step of acquiring at least image using an imaging technique to confirm the one or more SSMs on at least one side, a first side and/or a second side 1910, 1935. The SSMs comprises a coronal osteotomy angle, a sagittal osteotomy angle, transverse pedicle angle or convergence angle, resection depth, A/P distance and/or neutral alignment. The surgeon may acquire at least one image using at least one imaging technique on at least one side, a first side and/or a second side. The surgeon may further verify SSMs by comparing it to the SSMs measured during the pre-operative planning procedures for restoration or substantial restoration of a patient's coronal and/or sagittal alignment. The at least one image may be performed by using at least one imaging technique. The imaging techniques may comprise 2D or 3D images. The imaging techniques may comprise an MRI, a radiograph, a CT scan, an ultrasound, and/or any combination thereof. The preparation of the endplate surface of the cranial vertebral body on the contralateral side may be performed by repeating the above steps.

In another embodiment, the step of preparing the cranial vertebral body on the at least one side, first side and/or second side 1725, 1745, 1765, 1785 may comprise the step of providing feedback on matching or substantially matching one or more objects that were superimposed on the one or more images 1455 and/or the step of providing feedback on matching or substantially matching a first one or more objects on a first side and a second one or more objects on a second side 1455. The feedback may comprise visual feedback (using the navigation monitor or workstation display), audible feedback, tactile feedback, and/or any combination thereof. The feedback may include visual and/or audible feedback to correct or reposition the surgeon's trajectory and/or robot's trajectory to appropriately match or substantially match the one or more objects and/or projected surgical measurement. The one or more objects may include the SSM (selected surgical measurement), a virtual trajectory, a virtual tool, a virtual spinal implant, and/or any combination thereof. The SSM may include a convergence angle or transverse pedicle angle, A/P distance, neutral alignment, coronal osteotomy angle and/or sagittal osteotomy angle.

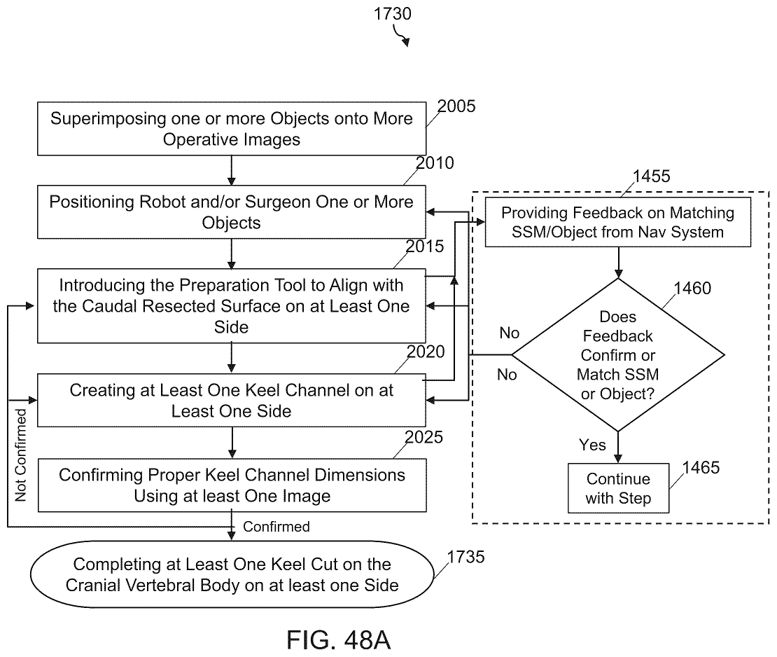

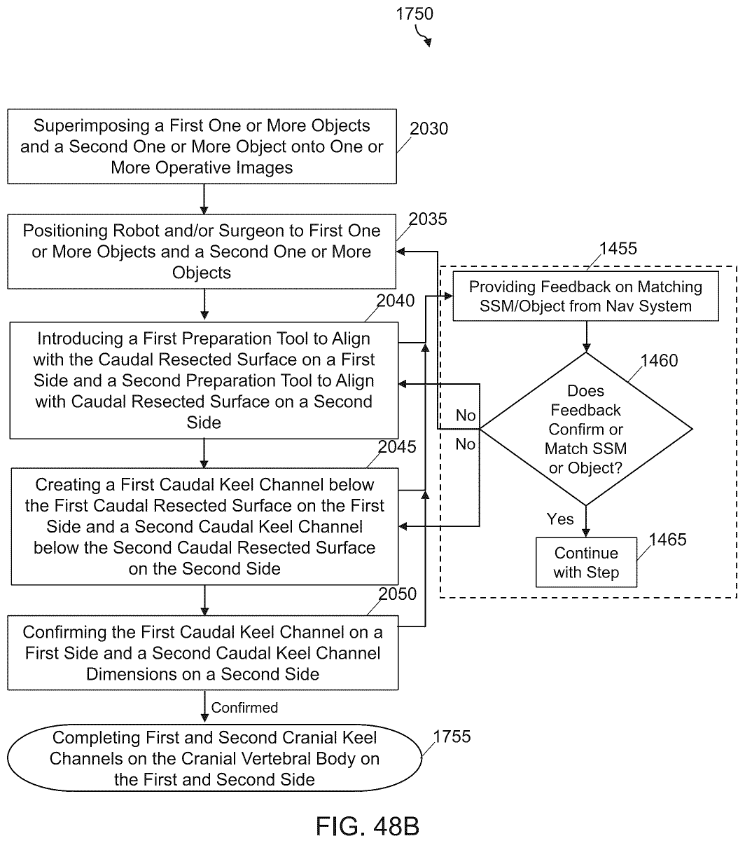

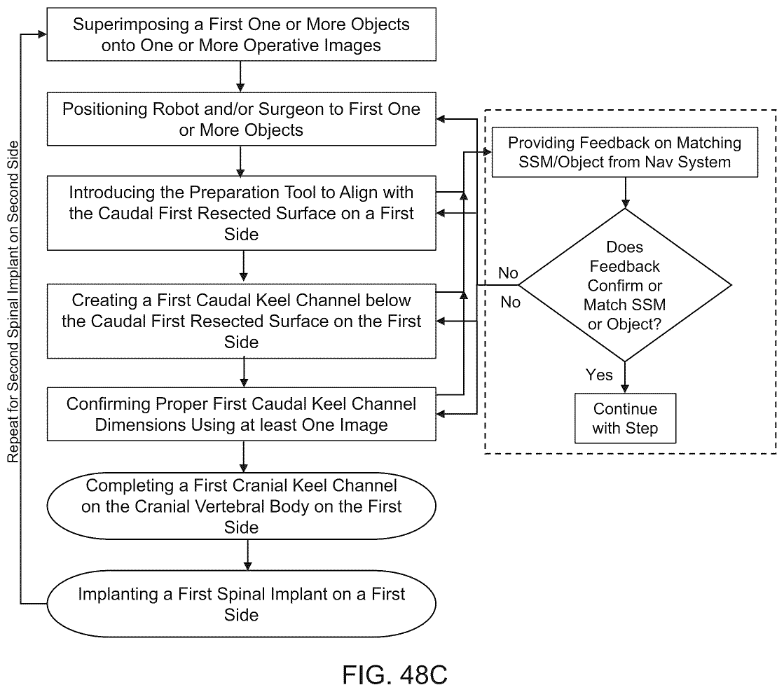

With reference to FIG. 48A, the step of preparing an intervertebral space within a localized spinal segment for alignment and motion restoration 1170, 1170a, 1170b, 1170c comprises the step of completing at least one keel channel on the caudal vertebral body on least a one side 1730. The step of completing at least one caudal keel channel on the caudal vertebral body on at least one side 1730 comprises the steps of: superimposing a one or more objects onto one or more operative images onto at least one side 2005; positioning user, robot and/or surgeon to the one or more objects and/or one or more trajectories 2010; introducing the preparation tool into prepared intervertebral disc space on the at least one side 2015; creating at least one keel channel on the resected surface of the caudal vertebral body that follows along the one or more objects and/or the one or more trajectories 2020; confirming the at least one keel channel placement or positioning by acquiring at least one image using at least one imaging technique on the at least one side 2025; and/or providing feedback on matching or substantially matching the one or more objects 1455. The step of completing at least one caudal keel channel on the caudal vertebral body may further comprise the step of obtaining one or more SSM or PSM for optimal alignment. The one or more objects and/or one or more trajectories may comprise following, matching or substantially matching the convergence angle or the transverse pedicle angle.

With reference to FIG. 48B, the step of preparing an intervertebral space within a localized spinal segment for alignment and motion restoration 1170, 1170a, 1170b, 1170c comprises the step of completing a first caudal keel channel on a first and a second caudal keel channel on a second side 1750. The step of completing a first caudal keel channel on a first and a second caudal keel channel on a second side 1750 comprises the steps of: superimposing a first one or more objects onto a first one or more operative images on a first side and a second one or more objects onto a second one or more images on a second side 2030; positioning a user, a robot and/or surgeon to the first one or more objects on a first side and a second one or more objects on a second side 2035; introducing a first preparation tool onto a first resected or prepared caudal surface within an intervertebral disc space on a first side and a second preparation tool onto a second resected or prepared caudal surface within the prepared intervertebral disc space on a second side 2040; creating at first caudal keel channel on the caudal vertebral body on the first side and a second caudal keel channel on the caudal vertebral body on the second side 2045; confirming the first keel channel placement or positioning on a first side and the second keel channel placement or positioning on a second side by acquiring at least one image using at least one imaging technique 2050; and/or providing feedback on matching or substantially matching the first and/or second one or more objects 1455. The step of completing at least one caudal keel channel on the caudal vertebral body may further comprise the step of obtaining a first one or more SSM or PSM for a first side and a second one or more SSM for a second side for optimal alignment.

With reference to FIG. 48C, the step of preparing an intervertebral space within a localized spinal segment for alignment and motion restoration comprises the step of completing a first keel channel on the caudal vertebral body on a first side. The step of completing a first caudal keel channel on the caudal vertebral body on the first side comprises the steps of: superimposing a one or more objects onto a first one or more operative images on a first side; positioning robot and/or surgeon to the first one or more objects; introducing the first preparation tool into the prepared intervertebral disc space; creating a first caudal keel channel below the first prepared or resected surface on the caudal vertebral body; confirming the at least one caudal keel channel placement or positioning by acquiring at least one image using at least one imaging technique; and/or providing feedback on matching or substantially matching the first one or more objects. With this embodiment, the surgeon should proceed with all the remaining steps of preparing the intervertebral space within a localized spinal segment for alignment and motion restoration for a first side prior to completing the steps for a second side. The remaining steps include step of preparing an intervertebral space within a localized spinal segment for alignment and motion restoration for a first side; implanting a first spinal implant on a first side; preparing an intervertebral space within a localized spinal segment for alignment and motion restoration for a second side; and implanting a second spinal implant on a second side. The step of completing a first caudal keel channel on the caudal vertebral body on the first side comprises the steps of: repeating all steps to implant a second spinal implant on a second side. FIG. 48A comprises similar steps, and may be adapted to optionally repeat the steps for an additional side or a second side.

The first keel channel may comprise the same dimensions as the second keel channel. The first keel channel may comprise different dimension as the second keel channel. The first transverse pedicle angle of the first keel channel may comprise the same angle as the second transverse pedicle angle of the second keel channel. The first spinal implant may comprise the same selected size as the second spinal implant. The first spinal implant may comprise a different selected size as the second spinal implant. The selected sizes may include different lengths or heights. The first one or more objects may comprise a same object as the second one or more objects. The first one or more objects may comprise a different object as the second on or more objects. The first trajectory may comprise a same and/or different trajectory than the second trajectory. The first SSM or PSM may comprise a different measurement than the second PSM or SSM. The first SSM or PSM may comprise a same measurement than the second PSM or SSM.

In one embodiment, the step of completing at least one caudal keel channel on the caudal vertebral body on at least one side, a first side and/or a second side 1730, 1750, 1770, 1790 comprises the step of obtaining one or more SSM or PSM, a first one or more SSM or PSM and/or a second SSM or PSM for optimal alignment (e.g., sagittal and/or coronal alignment) from the preoperative method or procedure or software application. The one or more SSM or PSM, the first one or more SSM or PSM, and/or a second one or more SSM or PSM may comprise a sagittal osteotomy angle or trajectory (e.g., sagittal angle of correction), a coronal osteotomy angle or trajectory (e.g., coronal angle of correction), a transverse pedicle angle or convergence angle, an A/P distance, keel alignment, and/or neutral alignment. The sagittal osteotomy angle or trajectory comprises an angle of 0 degrees to 40 degrees. The coronal osteotomy angle or trajectory comprises an angle of 0 degrees to 40 degrees. The transverse pedicle angle may comprise 0 degrees to 45 degrees. The sagittal osteotomy angle, the coronal osteotomy angle, the transverse pedicle angle and/or the A/P distance may be different in different spine segments within different spine regions. A first coronal osteotomy angle may comprise a different angle than the second coronal osteotomy angle. A first coronal osteotomy angle may comprise a same angle than the second coronal osteotomy angle. A first sagittal osteotomy angle may comprise a different angle than the second sagittal osteotomy angle. A first sagittal osteotomy angle may comprise a same angle than the second sagittal osteotomy angle.

In one embodiment, the step of completing at least one caudal keel channel, a first caudal keel channel and/or a second caudal keel channel on the caudal vertebral body on at least one side, a first side and/or a second side 1730, 1750, 1770, 1790 comprises the step of superimposing one or more objects, a first one or more objects and/or a second one or more objects onto one or more operative images 2005, 2030. Alternatively, the step of superimposing one or more objects onto one or more operative images further comprises the step of transforming the preoperative SSM or PSM into one or more objects, a first one or more objects and/or a second one or more objects onto one or more operative images. The operative images may comprise a one or more preoperative images, one or more operative images, one or more synchronized operative images and/or one or more MPR images.

The one or more objects, the first one or more objects, and/or a second one or more objects may comprise a surgical measurement or projected surgical measurement (PSM), a trajectory path, a virtual surgical instrument, a virtual spinal implant and/or any combination thereof. The trajectory path may comprise the trajectory of a surgical instrument, surgical approach and/or one or more spinal implants. The trajectory path, surgical instrument and/or spinal implant may include a first end and a second end. The trajectory path may match or substantially match one or more SSM or PSM, a first one or more SSM and/or a second one or more SSM or PSM. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance, keel alignment and/or neutral alignment.

In one embodiment, the step of completing at least one caudal keel channel, a first caudal keel channel and/or a second caudal keel channel on the caudal vertebral body on at least one side, a first side and/or a second side 1730, 1750, 1770, 1790 comprises the step of positioning the user, surgeon and/or robot to the desired one or more objects, a first one or more objects and/or a second one or more objects for the selected intraoperative step and/or the entire intraoperative steps 2010, 2035. The navigation system may display one or more objects, the first one or more objects and/or the second one or more objects. The navigation system may further transmit spatial coordinates for the targeted anatomy to create a surgical trajectory, a first surgical trajectory, a second surgical trajectory and projecting one or more objects, the first one or more objects and the second one or more objects that matches the surgical trajectory.

The surgeon and/or robotic system may hold the one or more surgical instruments and/or a registration tool (i.e., a probe, a guide and/or any other tool) to the desired position and/or one or more objects according to the spatial coordinates of the surgical trajectory for the desired intraoperative step. The one or more objects, the first one or more objects and/or a second one or more objects may match or substantially match one or more projected surgical measurements (PSM). The one or more objects comprise one or more PSMs, a virtual surgical instrument, a virtual spinal implant, and/or any combination thereof. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance, keel alignment, and/or neutral alignment.

In one embodiment, the step of completing at least one caudal keel channel, a first caudal keel channel and/or a second caudal keel channel on the caudal vertebral body on at least one side, a first side and/or a second side 1730, 1750, 1770, 1790 may comprise the step introducing a preparation tool, a first preparation tool and/or a second preparation tool on at least one side, a first side and/or a second side 2015, 2040. The step of introducing a preparation tool, a first preparation tool and/or a second preparation tool on at least one side, a first side and/or a second side 2015, 2040 comprises the steps of: assembling the keel tools; and inserting the keel tool into the prepared interverbal disc space.

The step of introducing a preparation tool, a first preparation tool and/or a second preparation tool on at least one side, a first side and/or a second side 2015, 2040 comprises the steps of registering or re-identification of one or more surgical instruments for the navigation system and/or robotic system. The surgical instruments may have been previously tracked during the preoperative procedure (e.g., a baseline registration), and may not require further reregistration. The surgeon or staff may be able to couple a portion of a reference system and/or robotic arm to the previously identified surgical instrument and begin immediate use of the instrument. Alternatively, the surgeon or staff may couple a portion of a reference system and/or couple to a robotic arm to the previously identified instrument (e.g., the baseline registration during the preoperative method), and may require intraoperative confirmation of spatial relationship or coordinates between the patient and the pre and intraoperative images. As disclosed herein, each of the surgical instruments, including the powered handpiece of the powered system may be adapted and configured to receive a portion of a reference system including at least one tracker, at least one probe, one or more fiducials and/or one or more LEDs. The handpiece may comprise an indentation or channel along the length of the handpiece, between the first end and/or second end of the handpiece. The portion of the reference system may align with the indentation and/or channel.

In another embodiment, the step introducing a preparation tool, a first preparation tool and/or a second preparation tool on at least one side, a first side and/or a second side 2015, 2040 comprises the step of assemble the at least one preparation tool, a first preparation tool and/or a second preparation tools. The tools may comprise a rasp, a manual handle, and/or a powered system. The surgeon may create the caudal keel channels using a manual and/or a powered system. In one embodiment, the surgeon may use a powered system as shown in FIGS. 47A-47E. The powered system comprises a powered control box 1985, a footswitch 2000, a powered handpiece 1995, and/or one or more connection cables 1990a, 1990b. The rasp may comprise a long or short keel rasp. To use the powered system, the surgeon should connect the connecting cables into the control box and cord into an outlet allowing the powered system to activate. The surgeon may subsequently insert a rasp into an unlocked handpiece and twist the locking collar on the handpiece to lock the rasp into the handpiece. The surgeon may test the powered system by stepping on the footswitch. The pressure on the footswitch determines the variable speed to activate and/or reciprocate the rasp.

In one embodiment, the step of completing at least one caudal keel channel, a first caudal keel channel and/or a second caudal keel channel on the caudal vertebral body on at least one side, a first side and/or a second side 1730, 1750, 1770, 1790 may comprise the steps of inserting the at least one keel tool, a first keel tool and/or a second keel tool onto at least one resected caudal surface, a first resected caudal surface, and/or a second caudal keel surface within the prepared interverbal disc space. The user, surgeon and/or robot may align the keel rasp onto the resected surface, the first resected surface and/or the second resected surface of the caudal vertebral body. The surgeon may continue to translate, slide or introduce the inactivated powered rasp to the at least one side, a first side and/or a second side along the desired resected surface, a custom path, one or more objects and/or one or more SSM or PSM to ensure that the proper path or trajectory is followed. Alternatively, the surgeon and/or robot may introduce the inactivated powered rasp following along the pedicle transverse angle (or pedicle mid axis or convergence angle) on the at least one side within an intervertebral disc space. The rasp comprises a long or short keel rasp. Furthermore, the surgeon and/or robot may desirably retract the exiting nerve root and lateral thecal sack to protect the neural elements during the use of the rasp.

In one embodiment, the step of completing at least one caudal keel channel on the caudal vertebral body on at least one side, a first side and/or a second side 1730, 1750, 1770, 1790 comprises the step of creating at least one keel channel on at least one side 2020 and/or the step of creating a first keel channel on a first side and/or a second keel channel on a second side 2045. The surgeon should realign the keel rasp to the posterior end of the caudal vertebral body and activate the powered rasp by pressing on the footswitch—a light pressure equals a slow reciprocating speed, and a hard pressure equals a faster reciprocating speed. The surgeon prepares and/or creates a keel channel on a least a portion of the resected surface on the caudal vertebral at a desired speed on at least one side, a first side and/or a second side. Alternatively, the surgeon may create the keel channel on the caudal vertebral body following along, matching or substantially matching the pedicle transverse angle (e.g., the pedicle axis) to create a keel channel on at least one side, a first side and/or a second side.

The keel rasp comprises a long or short keel rasp. The keel channel comprises a keel channel depth, a width and a length. The keel channel depth on the caudal vertebral body extends from a top surface of the caudal resected surface downwards towards the inferior direction. The keel channel length of the caudal vertebral body further extends from the posterior end of the caudal vertebral body towards the anterior surface of the caudal vertebral body. The keel channel may be positioned centrally on the caudal resected surface and/or following along the transverse mid axis of the caudal resected surface. The keel channel depth, width and length matches or substantially matches a portion of the spinal implant keel length, width and/or depth. The keel channel width matches or substantially matches the widest portion of the spinal implant keel width.

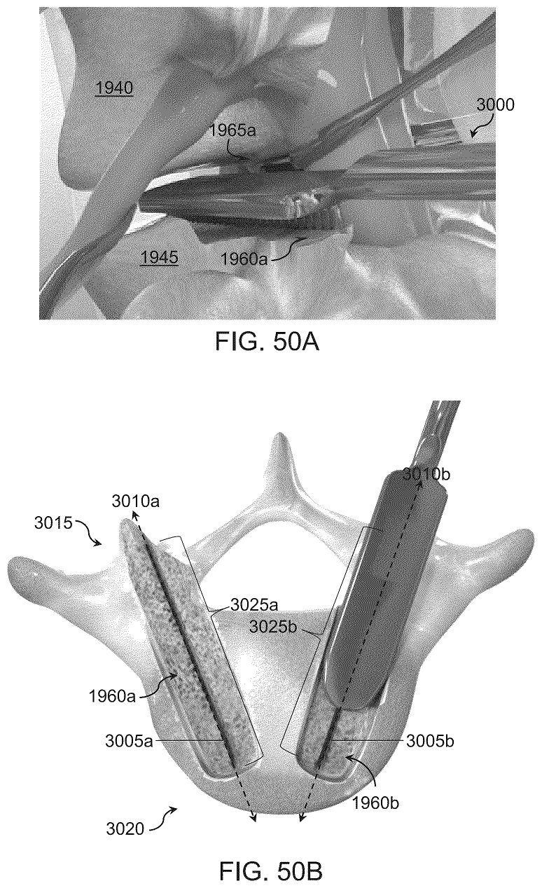

In another embodiment, the surgeon prepares and/or creates a first keel channel on a least a portion of the first resected surface on the caudal vertebral body on a first side following a first custom path or trajectory and a second keel channel on the second resected surface on the caudal vertebral body on a second side following a second custom path or trajectory as shown in FIGS. 50A-50B. Alternatively, the surgeon may create the first keel channel on the caudal vertebral body on a first side following along, matching or substantially matching a first pedicle transverse angle (e.g., the pedicle axis or convergence angles) and a second keel channel on the caudal vertebral body on a second side following along, matching or substantially matching a second pedicle transverse angle.

FIGS. 50A-50B illustrate a first caudal keel channel 3005a on a first caudal resected surface 3005a disposed on a first side of the caudal vertebral body 1945 and a second caudal keel channel 3005b disposed on a second caudal resected surface 3005b on the second side 3005b on the caudal vertebral body 1945. The first and second keel channels 3005a, 3005b comprises a keel channel depth, a width and a length. The first and second keel channel depth on the caudal vertebral body 1945 extends from first and second top resected surface 1960a, 1960b downwards towards the inferior direction. The first and second keel channel length 3025a, 3025b of the caudal vertebral body 1945 further extends from the posterior end 3015 of the caudal vertebral body towards the anterior end 3020 of the caudal vertebral body 1945. The first and/or second keel channel length 3025a, 3025b matches or substantially matches the caudal resected surface length. The first and/or second keel channel length 3025a, 3025b is less than the resected surface length. Each of the first and second keel channels depth, width and length matches or substantially matches each of the spinal implant keel length, width and/or depth. The first and second keel channel width matches or substantially matches the spinal implant keel width. The first and second keel channel depth of the caudal vertebral body matches or substantially matches the spinal implant keel depth.

In another embodiment, the step of completing at least one caudal keel channel on the caudal vertebral body on at least one side, a first side and/or a second side 1730, 1750 comprises the step of confirming at least one keel channel on a first side 2025 and/or the step of confirming a first caudal keel channel on a first side of a caudal vertebral body and a second caudal keel channel on the second side on the caudal vertebral body by acquiring at least one or more images. The surgeon may confirm the at least one caudal keel channel, a first caudal keel channel and/or a second caudal keel channel dimensions, the alignment and/or the trajectory or custom path. The at least one caudal keel channel, a first caudal keel channel and/or a second caudal keel channel dimensions comprises a length, a width and/or a depth. The alignment comprises confirmation that the keel channel follows or substantially is positioned along the transverse mid axis of the caudal resected surface and/or centrally located between the width of the resected surface. The custom path or trajectory path comprises confirmation of the at least one caudal keel channel, the first caudal keel channel and/or the second caudal keel channel follows, matches or substantially matches the convergence angle (or transverse pedicle angle). One or more operative images are acquired using at least one imaging technique. The imaging techniques may comprise 2D or 3D images. The imaging techniques may comprise an MRI, a radiograph, a CT scan, an ultrasound, and/or any combination thereof.

In another embodiment, the step of completing at least one caudal keel channel, a first caudal keel channel and/or a second caudal keel channel on the caudal vertebral body on at least one side, a first side and/or a second side 1730, 1750, 1770, 1790 may comprise the step of providing feedback on matching or substantially matching one or more objects that were superimposed on the one or more images 1455 and/or the step of providing feedback on matching or substantially matching a first one or more objects on a first side and a second one or more objects on a second side 1455. The feedback may comprise visual feedback (using the navigation monitor or workstation display), audible feedback, tactile feedback, and/or any combination thereof. The feedback may include visual and/or audible feedback to correct or reposition the surgeon's trajectory and/or robot's trajectory to properly match or substantially match the one or more objects and/or projected surgical measurement. The one or more objects may include the SSM (selected surgical measurement), a virtual trajectory, a virtual tool, a virtual spinal implant, and/or any combination thereof. The SSM may include a keel alignment, keel dimensions, convergence angle or transverse pedicle angle, A/P distance, neutral alignment, coronal osteotomy angle and/or sagittal osteotomy angle.

In one embodiment, the step of providing feedback 1455 may further comprise the steps of: generating a non-desired trajectory path and/or non-desired one or more objects; comparing the desired trajectory path to the non-desired trajectory path 1460; correcting user to return to desired trajectory path if the non-desired trajectory path is different than the desired trajectory path 2010, 2035, 2040, 2045. In another embodiment, the step of providing feedback 1455 may further comprise the steps of: generating a second trajectory path and/or non-desired second one or more objects; comparing the first trajectory path to the second trajectory path 1460; correcting user to return to first trajectory path if the second trajectory path is different than the first trajectory path 2010, 2035, 2040, 2045. The step of providing feedback 1455 may comprise at least one side, a first side and/or a second side.

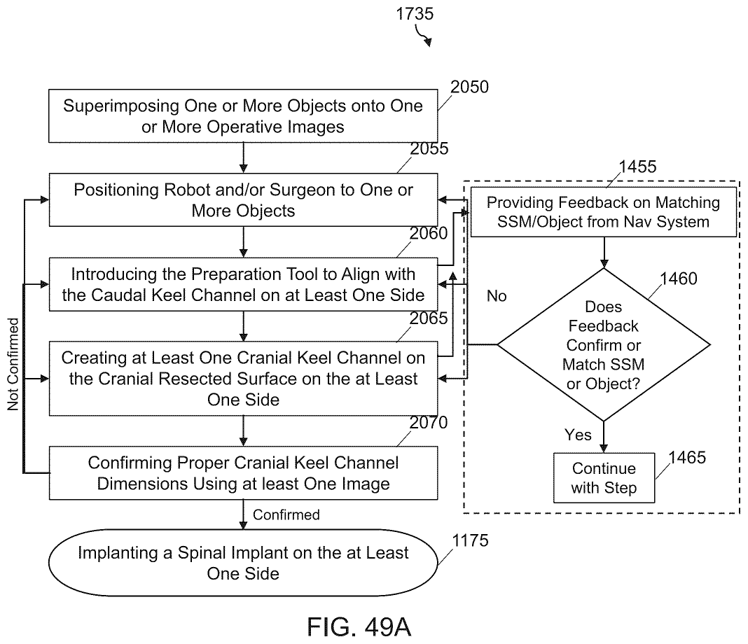

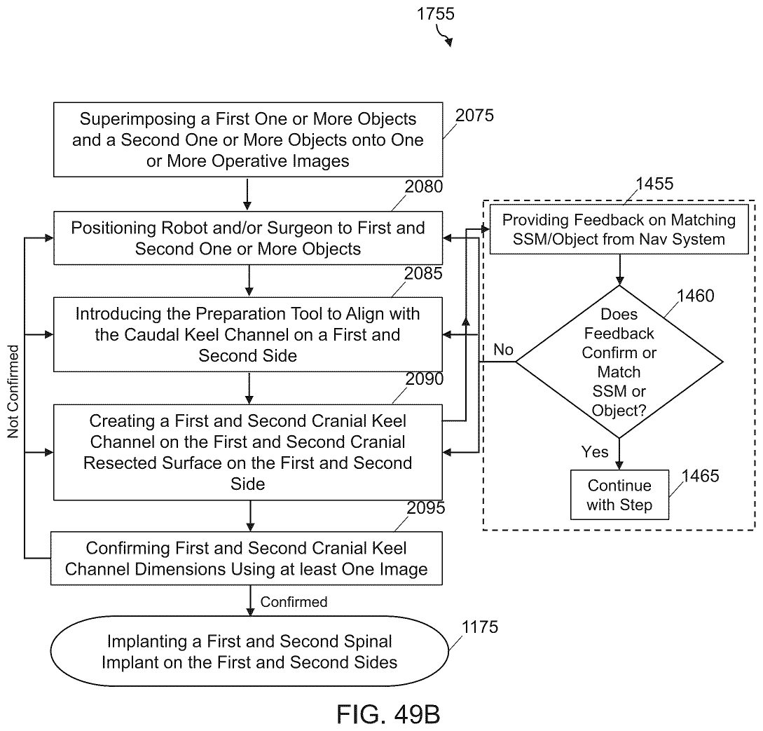

With reference to FIG. 49A, the step of preparing an intervertebral space within a localized spinal segment for alignment and motion restoration 1170, 1170a, 1170b, 1170c comprises the step of completing at least one keel channel on the cranial vertebral body on least a one side 1735, 1775, 1795. The step of completing at least one keel channel on the cranial vertebral body on at least one side 1730, 1775, 1795 comprises the steps of: superimposing one or more objects onto one or more images on the at least one side 2050; positioning a user, robot and/or surgeon to the one or more objects and/or trajectory path on at least one side 2055; introducing the preparation tool into prepared intervertebral disc space to align with the caudal keel channel on the at least one side 2060; creating at least one cranial keel channel on the cranial vertebral body that follows the one or more objects on the at least one side 2065; confirming the at least one cranial keel channel placement or positioning by acquiring at least one image using at least one imaging technique 2070; providing feedback on matching or substantially matching the one or more objects 1455. The one or more objects comprises an SSM or PSM, a virtual trajectory, a virtual tool, a virtual spinal implant. The virtual trajectory may match or substantially match the SSM or PSM calculated from the preoperative planning and/or preoperative software application. The custom path or trajectory path may follow, match and/or substantially match the transverse pedicle angle or convergence angle.

With reference to FIG. 49B, the step of preparing an intervertebral space within a localized spinal segment for alignment and motion restoration 1170, 1170a, 1170b, 1170c comprises the step of completing a first cranial keel channel on a cranial vertebral body on a first side and second cranial keel channel on the cranial vertebral body on a second side 1755. The step of completing a first cranial keel channel on a cranial vertebral body on a first side and second cranial keel channel on the cranial vertebral body on a second side 1755 comprises the steps of: superimposing a first one or more objects onto one or more operative images on a first side and a second one or more objects onto the one or more images on a second side 2075; positioning a user, robot and/or surgeon to the first one or more objects and/or trajectory path on a first side and a second one or more objects and/or trajectory path on a second side 2080; introducing a first preparation tool into the prepared intervertebral disc space that aligns with the first caudal keel channel on a first side and a second preparation tool into the prepared intervertebral disc space that aligns with the second caudal keel channel on a second side 2085; creating at first keel channel on the cranial vertebral body on the first side and a second keel channel on the cranial vertebral body on the second side 2090; confirming the first and second cranial keel channel by acquiring at least one image using at least one imaging technique on a first and second side 2095; providing feedback on matching or substantially matching the first and/or second one or more objects 1455. The one or more objects comprises an SSM or PSM, a virtual trajectory, a virtual tool, a virtual spinal implant. The virtual trajectory may match or substantially match the SSM or PSM calculated from the preoperative planning and/or preoperative software application. The custom path or trajectory path may follow, match and/or substantially match the transverse pedicle angle or convergence angle.

In another embodiment, the step of preparing an intervertebral space within a localized spinal segment for alignment and motion restoration 1170, 1170a, 1170b, 1170c comprises the step of completing a first cranial keel channel on the cranial vertebral body on a first side. The step of completing a first cranial keel channel on the cranial vertebral body on the first side comprises the steps of: superimposing a first one or more objects onto a first one or more operative images on a first side; positioning robot and/or surgeon to the first one or more objects on a first side; introducing the first preparation tool into the prepared intervertebral disc space that aligns with the first caudal keel channel on a first side; creating a first cranial keel channel above the first prepared or resected surface on the cranial vertebral body; confirming the at least one cranial keel channel placement or positioning by acquiring at least one image using at least one imaging technique; providing feedback on matching or substantially matching the first one or more objects on a second side.

The first keel channel comprises following along, matching or substantially matching the transverse pedicle angle. The transverse pedicle angle comprises an angle of 0 degrees to 45 degrees. With this embodiment, the surgeon should proceed with all the remaining steps of preparing the intervertebral space within a localized spinal segment for alignment and motion restoration for a first side prior to completing the steps for a second side. The remaining steps include step of preparing an intervertebral space within a localized spinal segment for alignment and motion restoration for a first side; implanting a first spinal implant on a first side; preparing an intervertebral space within a localized spinal segment for alignment and motion restoration for a second side; and implanting a second spinal implant on a second side. In one embodiment, the step of preparing a first cranial keel channel on the cranial vertebral body on a first side comprises the step of repeating all the above steps for a second cranial keel channel on the cranial vertebral body on a second side.

The first cranial keel channel may comprise the same dimensions as the second cranial keel channel. The first cranial keel channel may comprise different dimensions as the second cranial keel channel. The first transverse pedicle angle of the first cranial keel channel may comprise the same angle as the second transverse pedicle angle of the second cranial keel channel. The first spinal implant may comprise the same selected size as the second spinal implant. The first spinal implant may comprise a different selected size as the second spinal implant. The selected sizes may include different lengths or heights.

In one embodiment, the step of completing at least one cranial keel channel, a first cranial keel channel and/or a second cranial keel channel on the cranial vertebral body on at least one side, a first side and/or a second side 1735, 1755, 1775, 1795 comprises the step of superimposing one or more objects, a first one or more objects and/or a second one or more objects onto one or more operative images 2050, 2075. Alternatively, the step of superimposing one or more objects onto one or more operative images further comprises the step of transforming the preoperative SSM or PSM into one or more objects, a first one or more objects and/or a second one or more objects onto one or more operative images. The operative images may comprise a one or more preoperative images, one or more operative images, one or more synchronized operative images and/or one or more MPR images.

The one or more objects, the first one or more objects, and/or a second one or more objects may comprise a surgical measurement or projected surgical measurement (PSM), a trajectory path, a virtual surgical instrument, a virtual spinal implant and/or any combination thereof. The trajectory path may comprise the trajectory of a surgical instrument, surgical approach and/or one or more spinal implants. The trajectory path may match or substantially match one or more SSM or PSM, a first one or more SSM and/or a second one or more SSM or PSM. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance, keel alignment and/or neutral alignment.

In one embodiment, the step of completing at least one cranial keel channel, a first cranial keel channel and/or a second cranial keel channel on the cranial vertebral body on at least one side, a first side and/or a second side 1735, 1755, 1775, 1795 comprises the step of positioning the user, surgeon and/or robot to the desired one or more objects, a first one or more objects and/or a second one or more objects onto one or more images for the selected intraoperative step and/or the entire intraoperative steps 2055, 2080. The navigation system may display one or more objects, the first one or more objects and/or the second one or more objects. The navigation system may further transmit spatial coordinates for the targeted anatomy for one or more objects, the first one or more objects and the second one or more objects. The surgeon and/or robotic system may hold the one or more surgical instruments and/or a registration tool (i.e., a probe, a guide and/or any other tool) to the desired position and/or one or more objects according to the spatial coordinates of the surgical trajectory for the desired intraoperative step. The one or more objects, the first one or more objects and/or a second one or more objects may match or substantially match one or more projected surgical measurements (PSM). The one or more objects comprise one or more PSMs, a virtual surgical instrument, a virtual spinal implant, and/or any combination thereof. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance, keel alignment, and/or neutral alignment.

In another embodiment, the step of completing at least one cranial keel channel, a first cranial keel channel and/or a second cranial keel channel on the cranial vertebral body on at least one side, a first side and/or a second side 1735, 1755, 1775, 1795 comprises the step of introducing the at least one preparation tool, a first preparation tool, and/or a second preparation tool into the prepared intervertebral disc space that aligns with the at least one caudal keel channel on at least one side, a first side and/or a second side 2060, 2085. The step of introducing the at least one preparation tool, a first preparation tool, and/or a second preparation tool into the prepared intervertebral disc space that aligns with the at least one caudal keel channel on at least one side, a first side and/or a second side 2060, 2085 comprises the steps of: assembling the at least one preparation tool, a first preparation tool and/or a second preparation tool; and inserting a portion of the at least one preparation tool, a first preparation tool and/or a second preparation tool into the at least one caudal keel channel, a first caudal keel channel and/or second caudal keel channel to align the at least one cranial keel channel, a first cranial keel channel and/or a second cranial keel channel.

In one embodiment, the surgeon may subsequently prepare and/or assemble the proper preparation tools. The tools may comprise a keel rasp, a manual handle, and/or a powered system. The tools may further comprise an AO modular connector and/or a keel alignment guide 3030. In one embodiment, the surgeon may use a powered system, a keel alignment guide and a keel rasp. The powered system comprises a powered reciprocating system 1980 as shown in FIGS. 47A-47E. The rasp may comprise a long or short keel rasp. To assemble the powered reciprocating system, the surgeon should connect the cables and cord into an outlet allowing the powered reciprocating system to activate. The surgeon may subsequently insert an AO modular connector portion of the short keel rasp into an unlocked powered handpiece and/or insert one end with the AO modular connector into the unlocked powered handpiece. Twist the locking collar on the handpiece to lock the rasp into the powered handpiece.

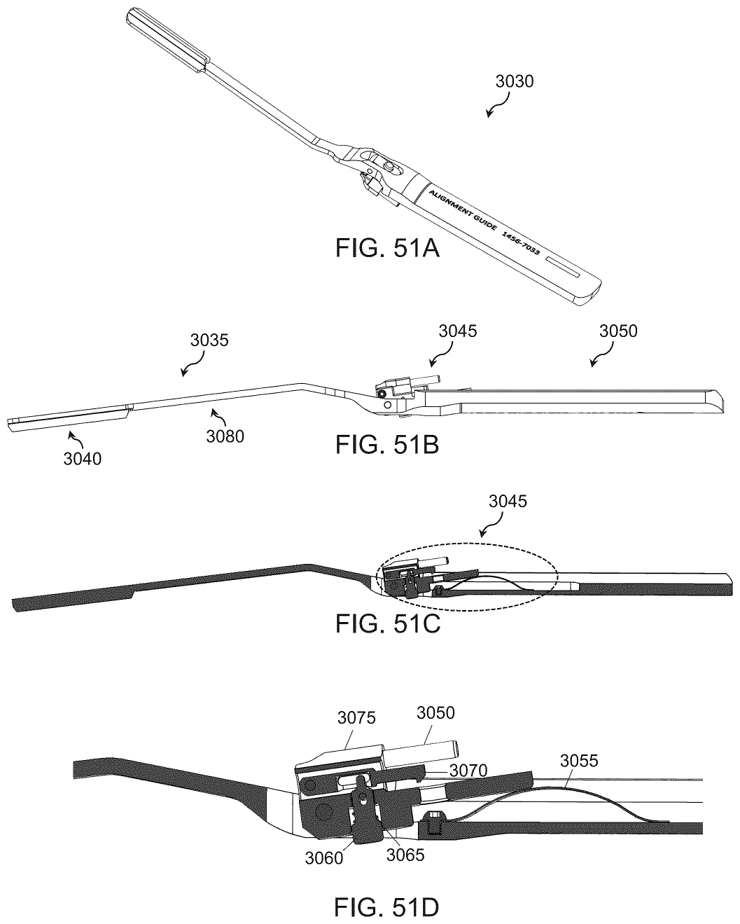

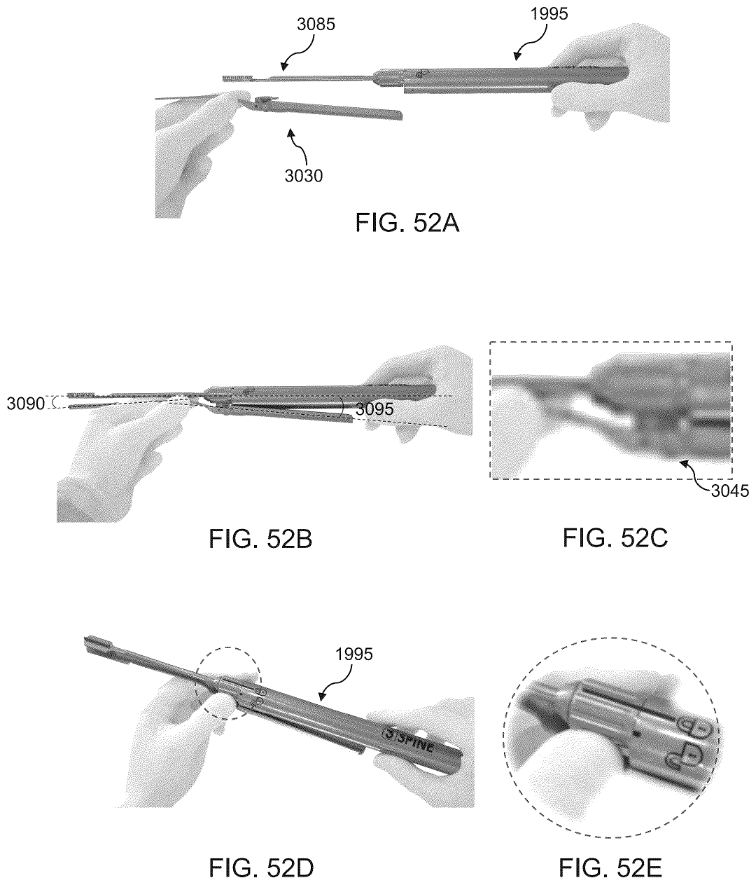

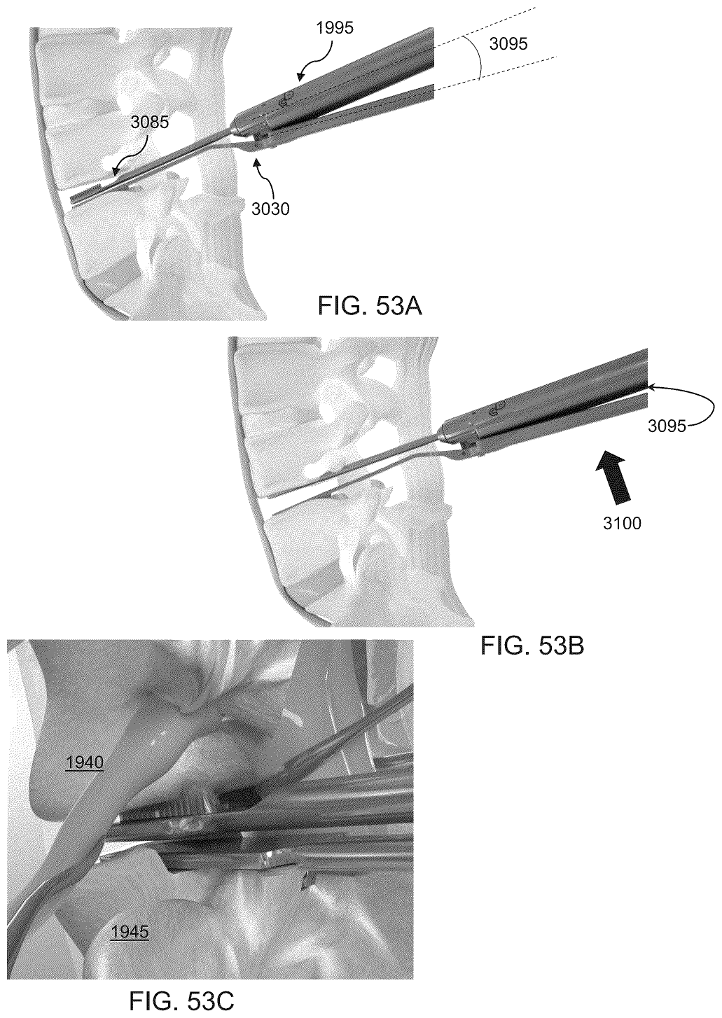

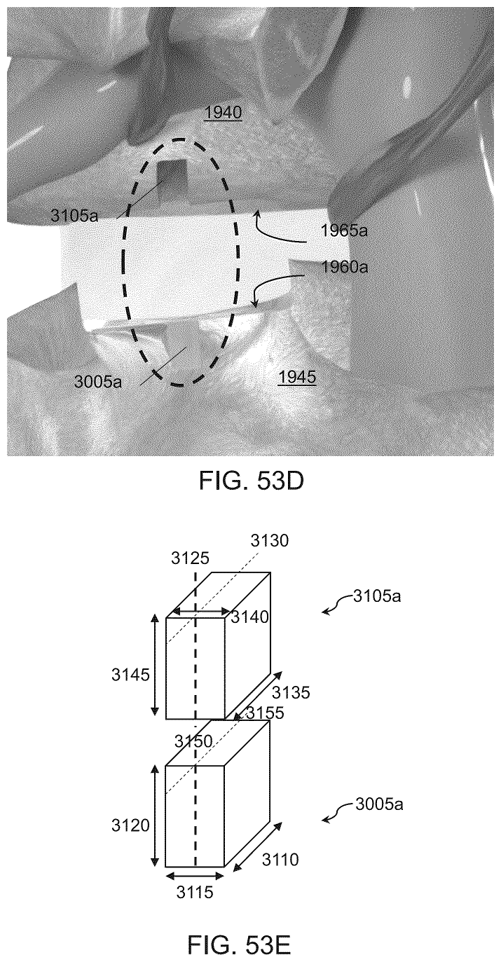

The surgeon may also assemble the alignment guide, if necessary, as shown in FIGS. 51A-51D and 52A-52E. The alignment guide 3030 comprises a keel member 3035, a connection mechanism 3045, and a handle 3050. The keel member 3035 comprises a shaft 3080 and a keel base 3040. The connection mechanism 3045 comprises a base 3075, one or more dowel pines 3050, a latch 3070, a leaf spring 3055, a compression spring 3065, and a button 3060. The alignment guide 3030 connects to the powered handpiece 1995 of the powered reciprocating system 1980. More specifically, the surgeon should align the one or more dowels pins 3050 of the alignment guide 3030 into the alignment pin openings (not shown) of the powered handpiece 1995 until the latch 3070 engages. A portion of the powered handpiece 1995 may contact a portion the handle 3050 of the alignment guide 3030 to create a first angle or distance 3090 at a first end and a second angle or distance 3095 at the second end at a first or neutral position. The first angle or distance 3090 may comprise a range of 0 to 5 degrees. The first angle or distance 3090 is less than the second angle or distance 3095. At least a portion of the keel member 3035 of the alignment guide 3030 may contact the keel rasp 3085 coupled to the powered handpiece 1995 at the first or neutral position (see FIGS. 52B and 53A). The first and second angles 3090, 3095 are angles of the handle 3050 relative to the powered handpiece 1995. If readjustment of the alignment guide is necessary, push the button 3060 on the bottom of the alignment guide 3030 and slide distally until the desired location is reached. The surgeon may test the powered system by stepping on the footswitch prior to inserting the tool assembly into the intervertebral space. The pressure on the footswitch determines the variable speed to activate and/or reciprocate the rasp.

In another embodiment, the step of introducing the at least one preparation tool, a first preparation tool, and/or a second preparation tool into the prepared intervertebral disc space that aligns with the at least one caudal keel channel on at least one side, a first caudal keel channel on a first side and/or a caudal keel channel on a second side comprises the step of inserting a portion of the at least one preparation tool, a first preparation tool and/or a second preparation tool into the at least one caudal keel channel, a first caudal keel channel and/or second caudal keel channel to align the at least one cranial keel channel, a first cranial keel channel and/or a second cranial keel channel.

The step of inserting a portion of the at least one preparation tool, a first preparation tool and/or a second preparation tool into the at least one caudal keel channel, a first caudal keel channel and/or second caudal keel channel to align the at least one cranial keel channel, a first cranial keel channel and/or a second cranial keel channel comprises the step of retracting the exiting nerve root and the thecal sac using retractors to protect the neural elements. This step may further comprise aligning the keel member 3035 to the posterior end of the caudal keel channel 3005a. The surgeon and/or user may insert or introduce a portion of the preparation tool assembly into a portion of the caudal keel channel 3005 on the one the at least one side, a first side and/or a second side without pressing, squeezing or compressing 3100 the handle 3050 of the alignment guide 3030 relative to the powered handpiece 1995 and/or toward the powered handle piece 1995 as shown in FIGS. 53A-53D.

Alternatively, surgeon may align and insert or introduce a portion of the alignment guide 3030 rasp into the at least one caudal keel channel, the first caudal keel channel and/or a second caudal keel channel 3005a within the prepared intravertebral disc space that follows, matches and/or substantially matches the pedicle transverse angle (or pedicle axis) on the at least one side without pressing, squeezing or compressing 3100 the handle 3050 of the alignment guide 3030 relative to the powered handpiece 1995 as shown in FIG. 53A. The surgeon should avoid contacting the resected surface 1965a of the cranial (or superior) vertebral body 1940 by accidentally pressing, squeezing or compressing 3100 the handle 3050 of the alignment guide 3030 relative to the powered handpiece 1995. The squeezing or compressing action 3100 will cause the keel member 3035 and/or the keel base 3040 to lift superiorly toward the prepared cranial resected surface 1965a and contact at least a portion of the cranial resected surface 1965a. The keel member 3035 and/or the keel base 3040 should be spaced apart from the cranial resected surface 1965a of the cranial (or superior) vertebral body 1940. The spacing may include a distance of at least 1 mm. The surgeon should continue to translate the preparation tool assembly toward the anteriorly within the at least one caudal keel channels until the alignment guide stops translating to confirm fit and/or any resistance. The surgeon may confirm placement, alignment and/or positioning of the tools by acquiring at least one image with at least one imaging technique.

The step of completing at least one cranial keel channel, a first cranial keel channel and/or a second cranial keel channel on the cranial vertebral body on at least one side, a first side and/or a second side 1735, 1755, 1775, 1795 comprises the step of creating at least one cranial keel channel along a custom path or trajectory path on at least one side 2065 and/or the step of creating first cranial keel channel along a first custom path or trajectory path on a first side and a second cranial keel channel along a second custom path or trajectory path on a second side 2090. The surgeon and/or user may activate the powered rasp by pressing on the footswitch—a light pressure equals a slow reciprocating speed, and a hard pressure equals a faster reciprocating speed. The surgeon and/or user may compress 3100 the handle 3050 of the alignment guide 3030 toward the powered handpiece 1995 to lift the keel member 3035 and/or the keel base 3040 of the alignment guide 3030 superiorly towards at least a portion of the cranial resected or prepared surface 1965a until it contacts at least a portion of the at least one resected or prepared surface, a first resected or prepared surface and/or a second resected or prepared surface 1965a of the cranial vertebral body 1940 on at least one side, a first side and/or a second side as shown in FIG. 53B.

The compression 3100 and the contact with at least a portion of the cranial resected surface 1965a will prepare and/or create at least one cranial keel channel, a first cranial keel channel and/or a second cranial keel channel 3105a, 3105b on a least a portion of the cranial resected surface 1965a on the cranial vertebral body 1940. The keel rasp 3085 may stamp or inscribe as shown in FIG. 53C-53D. The surgeon and/or may desirably remove the alignment guide 3030 from the powered handpiece 1995 to complete the remaining portion of the at least one cranial keel channel 3105a on at least one side, a first side and/or a second side. The surgeon and/or user may slide or translate the preparation tool assembly from posterior direction to the anterior direction and/or the anterior to the posterior direction.

The at least one cranial keel channel, a first cranial keel channel and/or a second cranial keel channel 3105a, 3105b on the cranial vertebral body 1940 extends from the cranial resected surface 1965a towards the superior direction. The at least one cranial keel channel, a first cranial keel channel and/or a second cranial keel channel 3105a, 3105b comprises a cranial keel channel depth 3145, a width 3140 and a length 3135. The cranial keel channel depth 3145, width 3140 and length 3135 matches or substantially matches a portion of the spinal implant keel length, width and/or depth. The cranial keel channel width 3145 matches or substantially matches the widest portion of the spinal implant keel width. The cranial keel channel length 3135 may match or substantially match the cranial resected surface length. The cranial keel channel length 3135 may be less than the cranial resected surface length. Once the keel channel has been created, the surgeon may remove the alignment guide and/or the keel rasp from the prepared intervertebral space.

The step of completing at least one cranial keel channel, a first cranial keel channel and/or a second cranial keel channel on the cranial vertebral body on at least one side, a first side and/or a second side 1735, 1755, 1775, 1795 comprises the step of confirming cranial keel channel placement, alignment and/or positioning on the at least one side 2070 and/or the step of confirming a first cranial keel channel placement and alignment on a first side and a second cranial keel channel placement and alignment on a second side by acquiring at least one image with at least one imaging technique 2095. The placement, alignment and/or positioning of the at least one cranial keel channel includes longitudinal alignment along 3130, 3155 and/or vertical alignment 3125, 3150 of the at least one caudal keel channel 3005a on the at least one caudal vertebral body 1945 relative to the at least one cranial keel channel 3105a on the at least one cranial vertebral body 1940. Alternatively, the placement, alignment and/or positioning includes longitudinal alignment along 3130, 3155 and/or vertical alignment 3125, 3150 of the first caudal keel channel 3005a on the at least one caudal vertebral body 1945 relative to the first cranial keel channel 3105a on the at least one cranial vertebral body 1940 and the second caudal keel channel 3005b on the at least one caudal vertebral body 1945 relative to the second cranial keel channel 3105b on the at least one cranial vertebral body.

In one embodiment, the at least one side comprises at least one caudal keel channel 3005a and at least one cranial keel channel 3105a. The at least one caudal keel channel 3005a comprises a caudal longitudinal axis 3150 and the at least one cranial keel channel 3105a comprises a cranial longitudinal axis 3130. The cranial longitudinal axis 3130 and the caudal longitudinal axis 3155 are parallel and/or substantially parallel. In another embodiment, the at least one caudal keel channel 3005a comprises a caudal vertical axis 3150 and the at least one cranial keel channel 3105a comprises a cranial vertical axis 3125. The cranial vertical axis 3125 and the caudal vertical axis 3150 are co-axial and/or substantially co-axial. In another embodiment, the at least a portion of the cranial longitudinal axis 3130 and at least a portion of the caudal longitudinal axis 3155 are parallel and/or substantially parallel, and/or a least a portion of the cranial vertical axis 3125 and the caudal vertical axis 3150 are co-axial and/or substantially co-axial.

In another embodiment, the first side comprises first caudal keel channel 3005a and a first cranial keel channel 3105a, and the second side comprises a second caudal keel channel 3005b and a second cranial keel channel 3105b. The first caudal keel channel 3005a comprises a first caudal longitudinal axis 3150 and the second caudal keel channel 3005b comprises a second caudal longitudinal axis. The first cranial keel channel 3105a comprises a first cranial longitudinal axis 3130 and the second cranial keel channel 3105b comprises a second cranial longitudinal axis. The first cranial longitudinal axis 3130 and the first caudal longitudinal axis 3155 are parallel and/or substantially parallel on the first side and the second cranial longitudinal axis and the second caudal longitudinal axis are parallel and/or substantially parallel on the second side. In another embodiment, the first caudal keel channel 3005a comprises a first caudal vertical axis 3150 and the first cranial keel channel 3105a comprises a first cranial vertical axis 3125, and the second caudal keel channel 3005b comprises a second caudal vertical axis and the second cranial keel channel 3105b comprises a second cranial vertical axis. The first cranial vertical axis 3125 and the first caudal vertical axis 3150 are co-axial and/or substantially co-axial on a first side and the second cranial vertical axis and the second caudal vertical axis are co-axial and/or substantially co-axial on a second side.

In another embodiment, the first side includes a first caudal keel channel and a first cranial keel channel as shown in FIGS. 53D-53E. The first caudal keel channel comprises a first caudal longitudinal axis and the first cranial keel channel comprises a cranial longitudinal axis. The first cranial longitudinal axis and the first caudal longitudinal axis are parallel and/or substantially parallel. In another embodiment, the first caudal keel channel comprises a first caudal vertical axis and the first cranial keel channel comprises a first cranial vertical axis. The first cranial vertical axis and the first caudal vertical axis are co-axial and/or substantially co-axial. In another embodiment, the first cranial longitudinal axis and the first caudal longitudinal axis are parallel and/or substantially parallel and the first cranial vertical axis and the first caudal vertical axis are co-axial and/or substantially co-axial.

Surgical Deployment Technique—Implanting a Spinal Implant

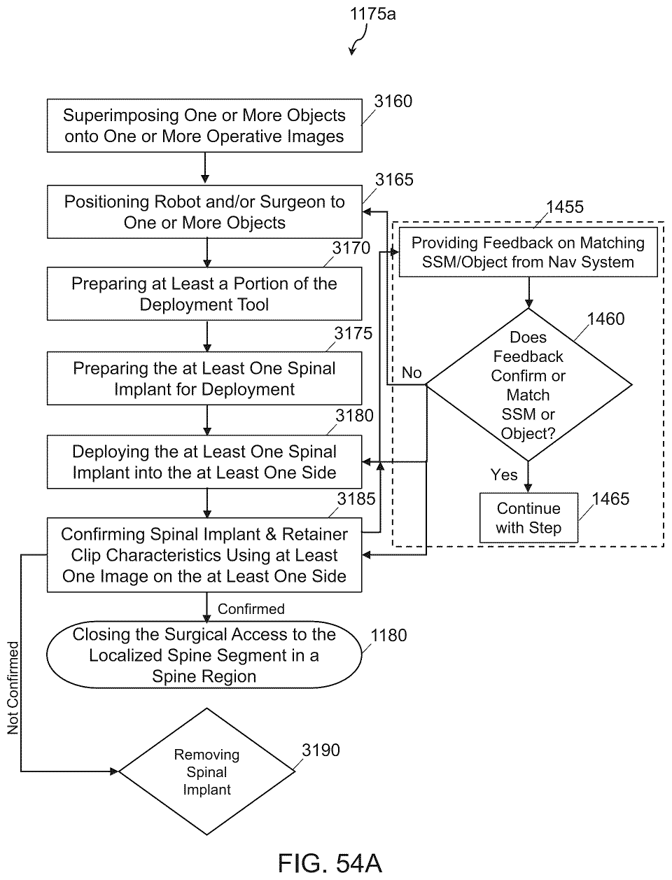

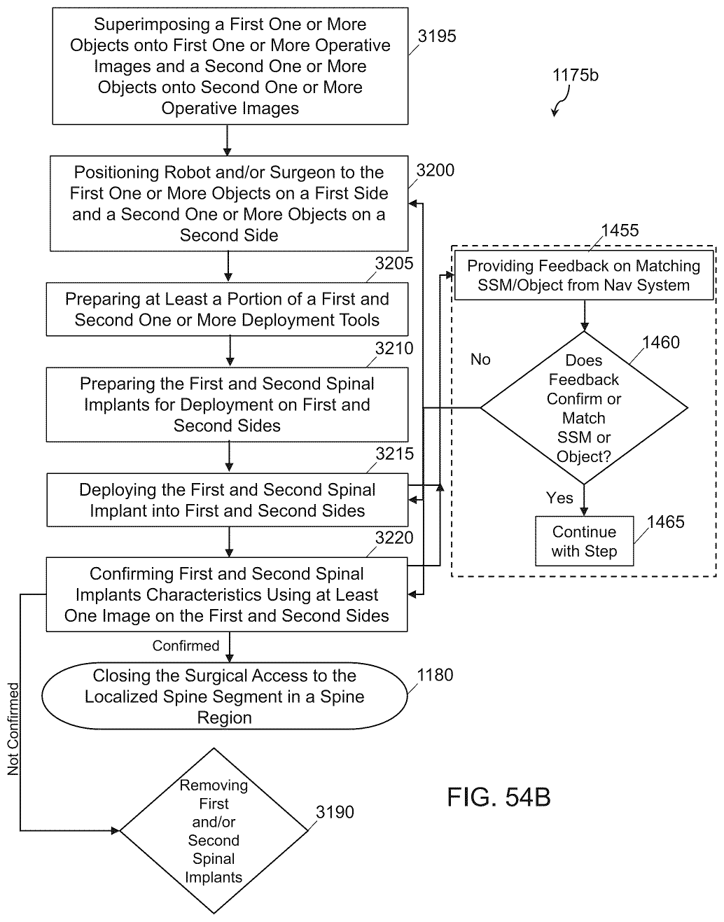

With reference to FIG. 54A, the navigation assisted and/or robotic assisted intraoperative procedure 1125 may comprise the step of implanting a spinal implant within a prepared intervertebral space on at least one side 1175, 1175a. The step of implanting at least one spinal implant, a first spinal implant and/or a second spinal implant on at least one side, a first side and/or a second side 1175, 1175a comprises the steps of: superimposing one or more objects onto one or more images on the at least one side 3160; positioning a user, robot and/or surgeon to the one or more objects and/or trajectory path on at least one side 3165; Preparing at least a portion of at least one deployment tool 3170; preparing the at least one spinal implant onto the at least one preparation tool for deployment 3175; deploying the at least one spinal implant into the prepared intravertebral space on the at least one side 3180; confirming at least one spinal implant characteristics using at least one image with at least one imaging technique on the at least one side 3185; providing feedback on matching or substantially matching the one or more objects on the at least one side 1455. If the first spinal implant characteristic fails confirmation, the surgeon may complete the step of removing the first spinal implant on the at least one side 3190. If the first spinal implant characteristics confirmation is successful, the surgeon should proceed with the steps of preparing the intervertebral space within a localized spinal segment for alignment and motion restoration 1170, 1170a, 1170b, 1170c for a second side. The remaining steps include preparing an intervertebral space within a localized spinal segment for alignment and motion restoration 1170, 1170a, 1170b, 1170c for a second side; and implanting a second spinal implant on a second side 1175, 1175a, 1175b.

With reference to FIG. 54B, the navigation and/or robotic assisted intraoperative procedure 1125 may comprise the step of implanting a first spinal implant within the prepared intervertebral space in a first side and a second spinal implant within the prepared intervertebral space on a second side 1175, 1175b. the step of implanting a first spinal implant within the prepared intervertebral space in a first side and a second spinal implant within the prepared intervertebral space on a second side 1175, 1175b comprises the steps of: superimposing a first one or more objects onto one or more images on a first side and a second one or more objects onto one or more images on a second side 3195; positioning a user, robot and/or surgeon to the first one or more objects or first trajectory path on the first side and the second one or more objects or second trajectory path on the second side 3200; preparing at least a portion of a first and second deployment tools 3205; preparing a first and second spinal implant for deployment 3210; deploying the first spinal implant to the first prepared intravertebral space in the first side and the second spinal implant into the second intravertebral space in the second side 3215; confirming the first and second spinal implant characteristics using at least one image with at least one imaging technique on a first side and a second side 3220; providing feedback on matching or substantially matching the first and/or second one or more objects 1455. If the first and/or second spinal implant characteristics fail confirmation, the surgeon may complete the step of removing the first and/or second spinal implant 3190.

The first spinal implant may comprise the same selected size as the second spinal implant. The first spinal implant may comprise a different selected size as the second spinal implant. The first prepared intervertebral space may comprise the same vertebral localized segment as the second prepared intervertebral space. The first intervertebral space may comprise a different localized segment as the second prepared intervertebral space. The at least one spinal implant, the first spinal implant and/or a second spinal implant characteristics may comprise confirmation of aligned cranial and caudal keel channels, the angle of convergence or transverse pedicle angle, the implant positioning, the proper intervertebral space preparation, the proper implant sizing, and/or any combination thereof. The first one or more objects are the same objects as the second one or more objects. The first one or more objects are different than the second one or more objects. The first trajectory path is the same trajectory as the second trajectory path. The first trajectory path is a different trajectory as the second trajectory path.

The step of implanting at least one spinal implant, a first implant and/or a second implant within a prepared intervertebral space on at least one side, a first side and/or a second side 1175, 1175a, 1175b comprises the step of superimposing one or more objects, a first one or more objects and/or a second one or more objects onto one or more operative images 3160, 3195. Alternatively, the step of superimposing one or more objects, a first one or more objects and/or a second one or more objects onto one or more operative images 3160, 3195 further comprises the step of transforming the preoperative SSM or PSM into one or more objects, a first one or more objects and/or a second one or more objects onto one or more operative images. The operative images may comprise a one or more preoperative images, one or more operative images, one or more synchronized operative images and/or one or more MPR images.

The one or more objects, the first one or more objects, and/or a second one or more objects may comprise a surgical measurement or projected surgical measurement (PSM), a trajectory path, a virtual surgical instrument, a virtual spinal implant and/or any combination thereof. The trajectory path may comprise the trajectory of a surgical instrument, surgical approach and/or one or more spinal implants. The trajectory path may match or substantially match one or more SSM or PSM, a first one or more SSM and/or a second one or more SSM or PSM. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance, keel alignment and/or neutral alignment.

The step of implanting at least one spinal implant, a first implant and/or a second implant within a prepared intervertebral space on at least one side, a first side and/or a second side 1175, 1175a, 1175b comprises the step of positioning the user, surgeon and/or robot to the desired one or more objects, a first one or more objects and/or a second one or more objects on at least one side, a first side and/or a second side 3165, 3200 onto one or more images for the selected intraoperative step and/or the entire intraoperative steps. The navigation system may display one or more objects, the first one or more objects and/or the second one or more objects. The navigation system may further transmit spatial coordinates for the targeted anatomy for one or more objects, the first one or more objects and the second one or more objects.

The surgeon and/or robotic system may position the one or more surgical instruments and/or a registration tool (i.e., a probe, a guide and/or any other tool) to the one or more objects, first one or more objects and/or second one or more objects, trajectory path or custom path according to the spatial coordinates of the surgical trajectory for the desired intraoperative step. The one or more objects, the first one or more objects and/or a second one or more objects may match or substantially match one or more projected surgical measurements (PSM). The one or more objects comprise one or more PSMs, a virtual surgical instrument, a virtual spinal implant, and/or any combination thereof. In one exemplary embodiment, the one or more objects, the first one or more objects and/or the second one or more objects comprises a PSM or SSM. The PSM or SSM includes an osteotomy sagittal angle (e.g., a sagittal angle of correction), an osteotomy coronal angle (e.g., a coronal angle of correction), a transverse pedicle angle or convergence angle, A/P distance, keel alignment, and/or neutral alignment.

The step of implanting at least one spinal implant, a first implant and/or a second implant within a prepared intervertebral space on at least one side, a first side and/or a second side 1175, 1175a, 1175b comprises the steps of: preparing at least a portion of at least one deployment tool for the at least one side 3170 and/or the steps of preparing at least a portion of a first deployment tool for a first side and at least a portion of a second deployment tool for the second side 3205. The step of preparing at least a portion of the at least one deployment tool, a first deployment tool and/or a second deployment tool for the at least one side, first side and/or second side 3170, 3205 comprises the steps of: acquiring a portion of the at least one deployment tool, a first deployment tool and/or a second deployment tool parts; and assembling at least a portion of the at least one deployment tool, a first deployment tool and/or a second deployment tool.

The step of acquiring a portion of the at least one deployment tool, a first deployment tool and/or a second deployment tool parts includes confirming one or more of the deployment tool parts are available to assemble into a deployment tool assembly. With reference to FIGS. 55A-55C, the at least one deployment tool, a first deployment tool and/or second deployment tool 3225 assembly comprises a handle subassembly 3230, a shaft subassembly 3250 and a draw bar 3270. The handle subassembly 3230 comprises a locking knob 3245, a handle 3240, and an endcap 3234. The handle subassembly 3230 further comprises a handle core 3285. The shaft subassembly 3250 comprises a shaft 3260, a grasping tip 3265, a dowel pin 3255. The shaft may be adapted and configured to receive a portion of a reference system, the reference system including a probe, a fiducial, an LED, a marker and/or any combination thereof. The draw bar 3270 comprises an opening 3275, the opening 3275 is sized and configured to receive a portion of the dowel pin 3255. The locking knob comprises a locking channel 3280.

The user and/or surgeon may dispose and/or couple the draw bar 3270 onto the shaft subassembly 3250. The draw bar 3270 comprises a first end, a second end and an opening 3275. The shaft subassembly 3250 comprises a first end, a second end. The first end of the shaft subassembly 3250 comprises a grasping tip 3265, the grasping tip 3265 includes a hook. The user and/or surgeon may align the opening 3275 on the draw bar 3270 to the dowel pin 3255 that extends from the shaft subassembly 3250. The first end of the draw bar 3270 may flush or below the first end of the shaft subassembly 3250. The locking knob 3245 may be secured onto the shaft subassembly 3250 and the draw bar 3270 by turning clockwise. The surgeon and/or user should ensure that the second end of the draw bar 3270 may be disposed within the locking knob 3245 to secure the draw bar 3270 and prevent premature or unwarranted translation. The locking knob 3245 that is secured over the draw bar 3270 and the shaft subassembly 3250 should be coupled and/or secured to the handle subassembly 3230.

The step of implanting at least one spinal implant, a first implant and/or a second implant within a prepared intervertebral space on at least one side, a first side and/or a second side 1175, 1175a, 1175b comprises the step of preparing the at least one spinal implant for deployment on the at least one side 3175 and the step of preparing a first spinal implant for deployment on a first side and a second spinal implant for deployment on a second side 3210. The step of preparing at least one spinal implant, a first spinal implant and/or a second spinal implant on at least one side, a first side and/or a second side 3175, 3210 comprises the steps of: selecting at least one pre-determined and/or proper sized at least one spinal implant, a first implant and/or a second implant; and assembling the at least one spinal implant, a first implant and/or a second implant; and securing the at least one spinal implant, a first implant and/or a second implant to the at least one deployment tool, a first deployment tool and/or a second deployment tool.

The step of selecting at least one predetermined spinal implant includes a selection of the implant that was pre-determined during the step of selecting a proper spinal implant size on at least one side. Alternatively, the selection of the proper spinal implant may further comprise a first spinal implant and a second implant that was predetermined during the step of selecting a proper first spinal implant on a first side and a second spinal implant on a second side. As discussed herein, the surgeon engaged in tissue balancing, length trialing and height trialing on at least one side a spinal region to determine the proper size (e.g., length and height) of the spinal implant, a first spinal implant and/or a second spinal implant that should be deployed into the prepared intervertebral space. The surgeon may acquire the proper size from 15 different available sizes.

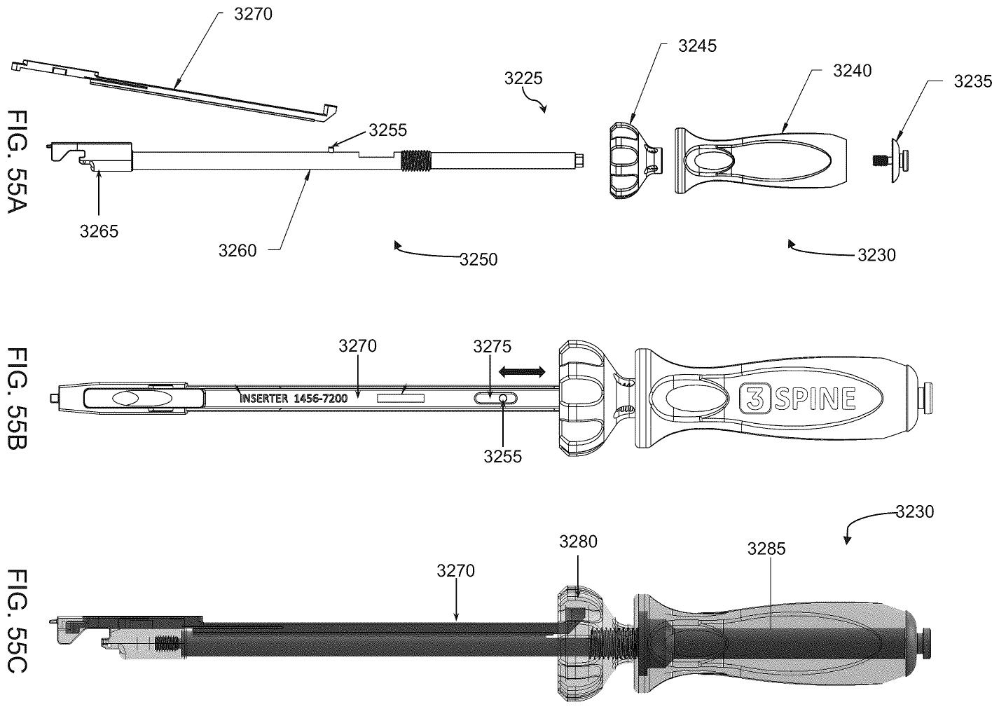

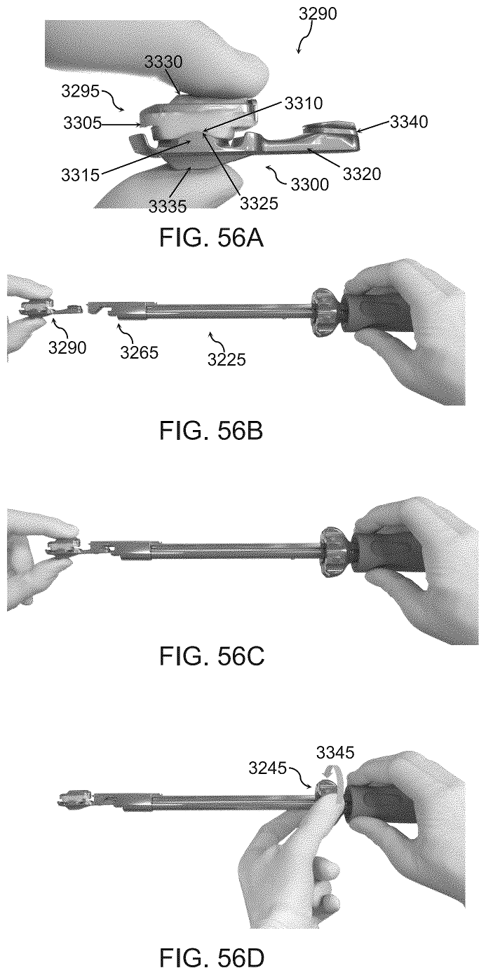

With reference to FIG. 56A, the surgeon may complete the step of assembling at least one spinal implant, a first spinal implant and/or a second spinal implant 3290 by assembling the selected and/or predetermined superior component 3295 and inferior component 3300. In one embodiment, the at least one spinal implant, a first spinal implant and/or a second spinal implant 3290 may comprise a superior component 3295, an inferior component 3300, and a fixation screw (not shown). In another embodiment, the at least one spinal implant, a first spinal implant and/or a second spinal implant may further comprise a retainer ring (not shown). The superior component 3295 comprises a superior articulation component 3305 and a superior keel 3330, the superior articulation component 3305 comprises a socket surface 3310. The socket surface 3310 comprising a concave shape.

The inferior component 3300 comprises an inferior articulation component 3315, an inferior keel 3335 and a bridge 3320. The inferior articulation component 3315 comprises a ball surface 3325. The ball surface 3325 comprising a convex or hemi-spherical shape. The socket surface 3310 of the superior component 3295 contacts and engages the ball surface 3325 of the inferior component 3300 to create a polyaxial articulation joint. The polyaxial articulation joint is movable in different orientations, including flexion, extension, rotation, lateral flexion; contralateral flexion; and/or any combination thereof. The fixation screw is disposed through the posterior end of the bridge 3320 to be secured to inferior or caudal vertebral body. The fixation screw is disposed at an angle. The angle may match or substantially match the sagittal pedicle angle. The bridge 3320 further comprises a channel 3340, the channel 3340 is sized and configured to receive a retainer ring.

The superior component 3295 further comprises a superior base and a superior keel 3330. The superior articulation component 3305 is disposed onto the superior base and/or the superior articulation component 3305 is coupled to the superior base. The superior base comprises a first material and the superior articulation component 3305 comprises a second material. The first material may comprise the same material as the second material. The first material may comprise a different material than the second material. The materials may comprise a polymer, a metal and/or a ceramic. The polymer may comprise thermoplastics or thermosets. The polymer may further comprise cross-linked polymers. The polymer may comprise polyethylene (PE), high density polyethylene (HDPE), ultra-high molecular weight polyethylene (UHMWPE), and/or any combination thereof. The polymers may further comprise being cross-linked one or more times. The polymers may further comprise antioxidant doped or impregnated polymers. The antioxidants may include Vitamin E or Vitamin C. The metals may comprise stainless steel, titanium, titanium alloys, cobalt chrome, cobalt chrome alloys, and/or any combination thereof.

The inferior component 3300 further comprises an inferior base and an inferior keel 3335. The inferior articulation component 3315 is disposed onto the inferior base and/or the inferior articulation component 3315 is coupled to the inferior base. The bridge 3320 is coupled to the posterior end of the inferior base and extends posteriorly and/or extends in the posterior direction. The inferior base comprises a third material. Each of the first material, second material and/or third material may comprise the same material. Each of the first material, second material and/or the third material may comprise a different material. The materials may comprise a polymer, a metal and/or a ceramic. The polymer may comprise thermoplastics or thermosets. The polymer may further comprise cross-linked polymers. The polymer may comprise polyethylene (PE), high density polyethylene (HDPE), ultra-high molecular weight polyethylene (UHMWPE), and/or any combination thereof. The polymers may further comprise being cross-linked one or more times. The polymers may further comprise antioxidant doped or impregnated polymers. The antioxidants may include Vitamin E or Vitamin C. The metals may comprise stainless steel, titanium, titanium alloys, cobalt chrome, cobalt chrome alloys, and/or any combination thereof.

At least a portion of the bridge 3320, the inferior base and/or the superior base may comprise a coating. Alternatively, the entirety of the bridge 3320, inferior base and/or superior base may comprise a coating. The coatings may include inorganic coatings or organic coatings. The coatings may further include a metal coating, a polymer coating, a composite coating (ceramic-ceramic, polymer-ceramic, metal-ceramic, metal-metal, polymer-metal, etc.), a ceramic coating, an anti-microbial coating, a growth factor coating, a protein coating, a peptide coating, an anti-coagulant coating, an antioxidant coating and/or any combination thereof. The antioxidant coatings may comprise naturally occurring or synthetic compounds. The natural occurring compounds comprises Vitamin E and Vitamin C (tocotrienols and tocopherols, in general), phenolic compounds and carotenoids. Synthetic antioxidant compounds include a-lipoic acid, N-acetyl cysteine, melatonin, gallic acid, captopril, taurine, catechin, and quercetin, and/or any combination thereof. The coatings can be impregnated, applied and/or deposited using a variety of coating techniques. These techniques include sintered coating, electrophoretic coating, electrochemical, plasma spray, laser deposition, flame spray, biomimetic deposition and wet methods such as sol-gel-based spin- and -dip or spray-coating deposition have been used most often for coating implants.

The metal coatings may comprise titanium, titanium alloys, cobalt-chrome alloys, platinum and stainless steel, and/or any combination thereof. More specifically, the metal coating includes titanium and/or cobalt-chrome molybdenum (CoCrMo). The polymer coatings may include thermoplastic or thermoset polymers. The polymers may further include carbon fiber, polyether ether ketone (PEEK), polyethylene (PE), ultra-high molecular weight polyethylene (UHMWPE), polycarbonate (PC), polypropylene (PP) and/or any combination thereof. The ceramic coatings may include alumina ceramics, Zirconia (ZrO2) ceramics, Calcium phosphate or hydroxyapatite (Ca10(PO46(OH)2) ceramics, titanium dioxide (TiO2), silica (SiO2), Zinc Oxide (ZnO) and/or any combination thereof.

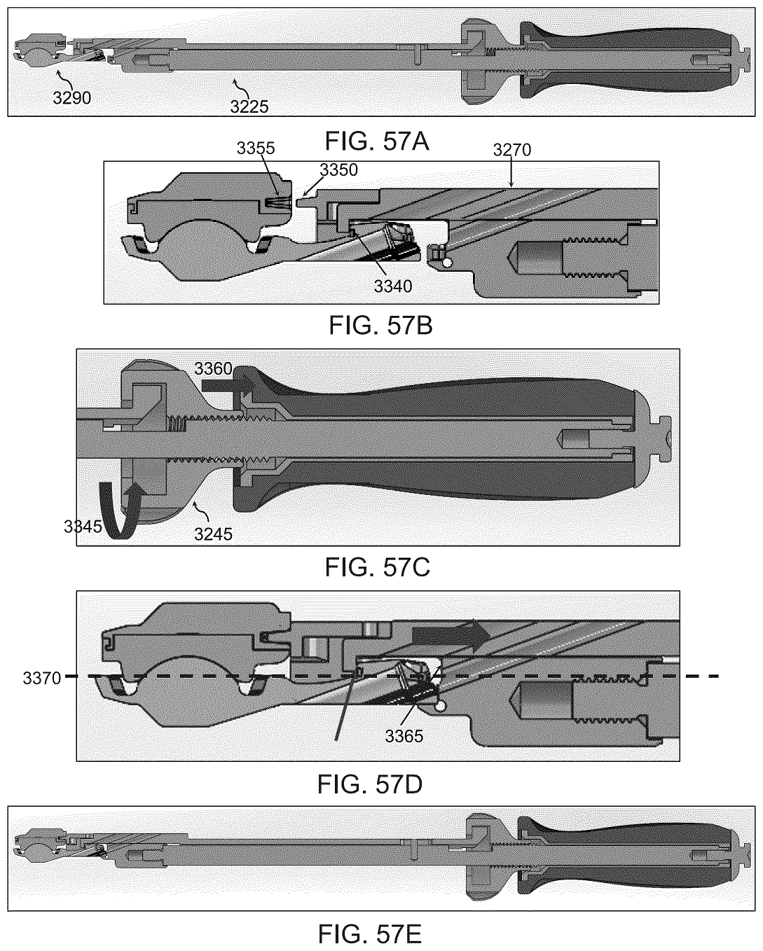

With reference FIGS. 56B-56D and FIGS. 57A-57E, the surgeon may complete the step of securing the at least one spinal implant, a first spinal implant and/or a second spinal implant 3290 to the at least one deployment tool, a first deployment tool and/or a second deployment tool 3225. The surgeon and/or user can secure the posterior end or second end of the spinal implant 3290 into the hook of the grasping tip 3265 disposed on the first end of the deployment tool 3225. More specifically, the surgeon and/or user can insert the hook of the grasping tip 3265 to contact or engage with the retainer clip channel 3340 on the posterior end of the bridge 3320 of the spinal implant 3290. The locking knob 3245 should be rotated 3345 (e.g., clockwise or counterclockwise) to translate 3360 the at least one spinal implant 3290 posteriorly until at least a portion of the at least one spinal implant 3290 contacts and/or engages with the stop wall 3365 of the grasping tip 3265 and the protrusion 3350 on the draw bar 3270 and/or the grasping tip 3265 is inserted into the protrusion socket or recess. The protrusion socket or recess 3355 is sized and configured to receive at least a portion of the protrusion 3350 of the draw bar 3270 and/or the grasping tip 3265. The surgeon and/or user should have some force feedback on the locking knob 3245 to indicate that contact has been made against the stop wall 3365 of the grasping tip 3265. The at least one spinal implant, a first spinal implant and/or a second spinal implant 3290 should be in a neutral position and/or aligned with the longitudinal axis 3370 of the deployment tool 3225. The at least one spinal implant, a first spinal implant and/or a second spinal implant 3290 should be in a neutral position and/or parallel with the longitudinal axis 3370 of the deployment tool 3225.

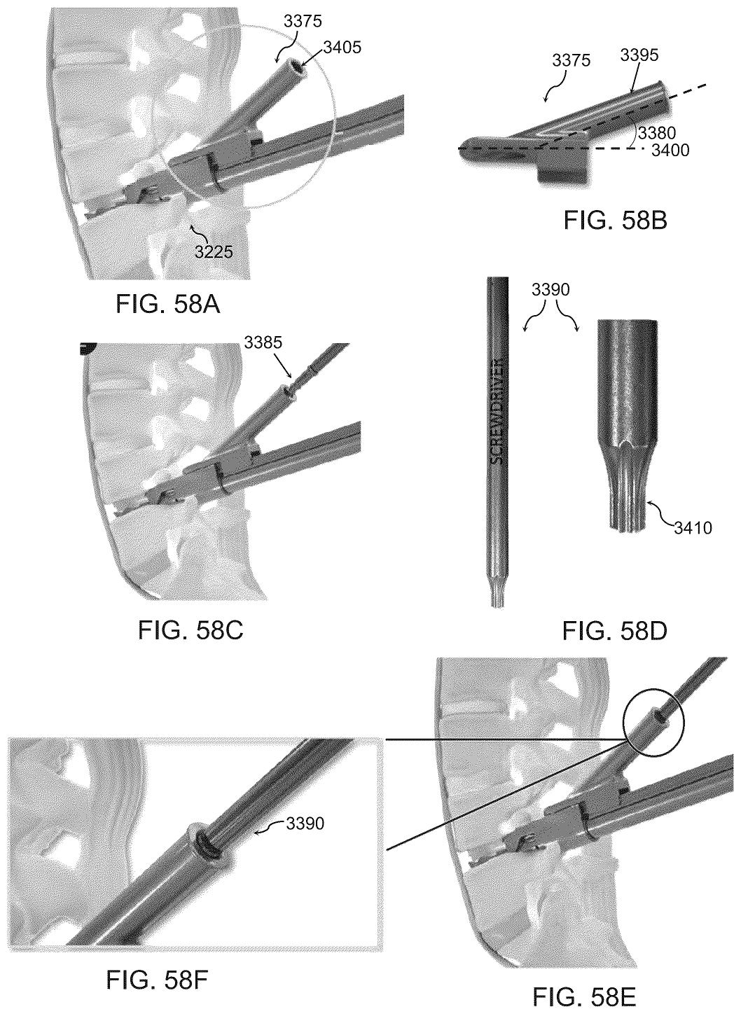

The step of implanting at least one spinal implant, a first implant and/or a second implant within a prepared intervertebral space on at least one side, a first side and/or a second side 1175, 1175a, 1175b comprises the steps of: deploying at least one spinal implant into at least one side 3180 and/or deploying a first spinal implant into a first side and a second spinal implant into a second side 3215. With reference to FIGS. 58A-58F, the sagittal isometric views of the images depict one embodiment of the step of deploying the at least one spinal implant, a first spinal implant, and/or a second spinal implant 3290 into the at least one side, a first side and/or a second side 3180, 3215. The step of deploying the at least one spinal implant, a first spinal implant and/or a second spinal implant 3290 into the at least one side, a first side and/or a second side 3180, 3215 comprises the steps of: aligning the each of the superior keel and/or inferior keels on the spinal implant to each of the caudal and/or cranial keel channels on at least one side; securing fixation screw into the caudal or inferior vertebral body into the at least one side; and releasing or disengaging the deployment tool on the at least one side.

With reference to FIG. 58A, the surgeon may complete the step of aligning each of the superior keels 3330 and/or inferior keels 3335 on the spinal implant 3290 to each of the caudal and/or cranial keel channels on at least one side, a first side and/or a second side. The surgeon should align the superior keel 3330 of the at least one spinal implant, a first implant and/or a second implant 3290 with the cranial keel channel 3105a, 3105b and the inferior keel 3335 of the at least one spinal implant, a first spinal implant and/or a second spinal implant with the caudal keel channel 3005a, 3005b. Translate and/or push the deployment tool 3225 forward until the superior keel 3330 and/or the inferior keel 3335 of the at least one spinal implant, first spinal implant and/or second spinal implant contacts and/or engages with at least one anterior end or anterior surface of the caudal keel channels 3005a, 3005b and/or cranial keel channels 3105a, 3105b. The pushing force should be a minimal force. The surgeon and/or user should be aware to not over distract prepared intravertebral disc space that what was originally pre-determined during the step of selecting a proper spinal implant size 1165.

With reference to 58B-58D, the surgeon and/or user may complete the step of securing the fixation screw 3385 to the caudal or inferior vertebral body. The surgeon and/or user may desirably attach a screw guide 3375 near and/or proximate to the first end of the deployment tool 3225. The screw guide 3375 will be positioned and/or disposed onto a portion of the grasping tip 3265, the draw bar 3270 and/or a portion to the shaft subassembly 3250 and secured. Alternatively, the screw guide 3375 may be attached prior to the step of aligning each of the superior keel 3330 and/or inferior keel 3335 to each of the caudal keel channel 3005a, 3005b and/or cranial keel channels 3105a, 3105b. The guide barrel or the guide tube 3395 of the screw guide 3375 is positioned obliquely and/or at an angle 3380 relative the longitudinal axis 3400 of the body of the screw guide 3375. Accordingly, the longitudinal axis 3400 of the guide barrel or the guide tube 3395 is at an angle relative to the longitudinal axis 3400 of the body of the screw guide 3375.

The surgeon and/or user may desirably insert the fixation screw 3385 into the bore (e.g., inner diameter) 3405 of the guide barrel or guide tube 3395 of the screw guide 3375. A deployment screwdriver 3390 may be utilized to secure the fixation screw 3385 into the caudal vertebral body. The deployment screwdriver 3390 may be powered and/or manual. The deployment screwdriver 3390 is coupled to a handle (e.g., Hudson handle not shown) for manual installation. The surgeon and/or user will insert the drive tip or tip 3410 into a portion of the drive recess of the fixation screw 3385. The surgeon and/or user may begin to rotate the deployment screwdriver 3390 clockwise to perforate the cortex using the fixation screw tip until the surgeon receives feedback and/or torque feedback (e.g., tightening). The fixation screw 3385 comprises a self-tapping tip. The surgeon should not over-rotate, over-tighten or over-torque the fixation screw 3385 into the caudal vertebral body—it may cause changes to positioning, alignment, and/or prepared bone interface.

With reference to FIG. 58E-58F, the surgeon and/or user may complete the step of disengaging, disconnecting and/or releasing the at least one spinal implant, a first spinal implant and/or a second spinal implant 3290 deployed into the at least one side, a first side and/or a second side. Once the fixation screw 3385 is secured into the caudal vertebral body, the deployment screwdriver 3390 may be removed. The surgeon and/or user may slightly release the deployment tool 3225 securing force from the at least one spinal implant, a first spinal implant and/or a second spinal implant 3290 by turning and/or rotating the locking knob 3245 (e.g., counterclockwise or clockwise) of the deployment tool 3225. The deployment screwdriver 3390 should be rotated (e.g., counterclockwise or clockwise) until the reference line on the deployment screwdriver 3390 is visible above a top surface of the guide barrel and/or the guide tube 3395 of the screw guide 3375. Disconnect and/or disengage the deployment screwdriver 3390 from the at least one spinal implant, a first spinal implant and/or a second spinal implant 3290 and remove from the prepared intravertebral space.

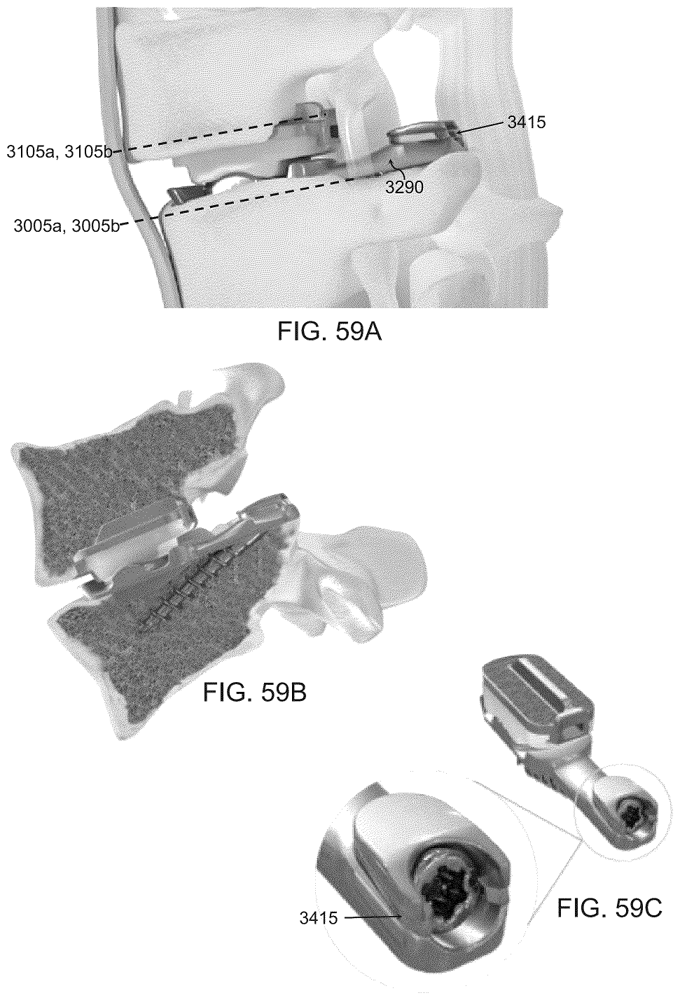

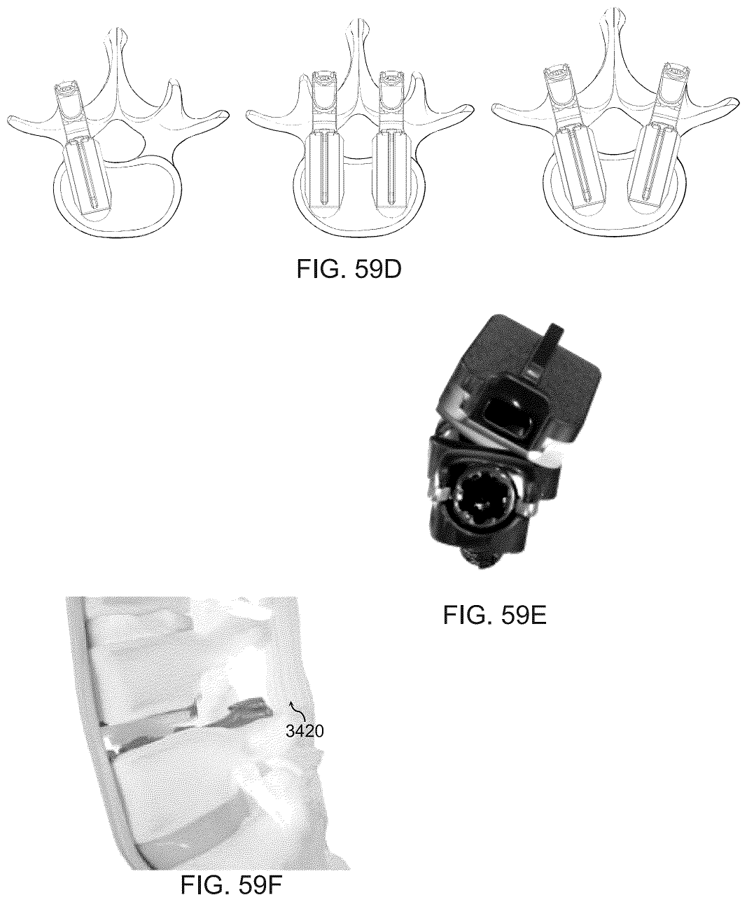

The step of implanting at least one spinal implant, a first implant and/or a second implant within a prepared intervertebral space on at least one side, a first side and/or a second side 1175, 1175a, 1175b comprises the step of confirming at least spinal implant characteristics on the at least one side 3185 and/or the step of confirming a first spinal implant characteristics on the first side and a second spinal implant characteristics on a second side 3220. With reference to FIGS. 59A-59C, the surgeon should confirm the one or more spinal implant characteristics of the at least one spinal implant, a first spinal implant and/or a second spinal implant using at least one image with at least one imaging technique on at least one side, the first side, and/or the second side.

The one or more spinal implant characteristics, a first one or more spinal implant characteristics and/or a second one or more spinal implant characteristics as shown in FIGS. 59A-59F. The one or more spinal implant characteristics may comprise proper or improper neutral alignment, the neutral alignment includes the cranial resected or prepared surface 3105a, 3105b is parallel or substantially parallel to the caudal resected surface 3005a, 3005b for optimal spinal implant function (see FIG. 59A). The one or more spinal implant characteristics may further comprise proper or improper confirming the sagittal osteotomy angle (see FIG. 59B) and/or coronal osteotomy angle. The one or more spinal implant characteristics may further comprise proper retainer clip 3415 positioning. The positioning of the retainer clip 3415 should be properly seated into the retainer clip channel 3340. Unseated portions of the retainer clip 3415 may facilitate premature backing out the fixation screw 3385 and/or clotting issues (see FIG. 59C).

The one or more spinal implant characteristics may further comprise the toe-in angle or convergence angle or transverse pedicle angle (see FIG. 59D). The one or more spinal implant characteristics may further comprise proper caudal-to-cranial keel channel alignment (see FIG. 59E). The caudal keel channel and the cranial keel channel should be parallel in the longitudinal axis and/or co-axial in the vertical axis and/or in the same plane. If not aligned, new keel channels may be warranted. The one or more spinal implant characteristics may further comprise proper or improper implant sizing. Confirmation of implant sizing includes confirming the proper length and height of the spinal implant with one or more operative images. If the length of the spinal implant is pressing into the ALL or annulus 3420 as shown in FIG. 59F, a smaller implant length may be indicated. If excessive force (i.e., more than hand force or soft tapping with a mallet is required for implantation), a larger osteotomy or a shorter implant height may be indicated. If the implant is loose on the ipsilateral or contralateral side after implantation, a larger height may be indicated. Confirm the new implant size with one or more operative images (see FIG. 59F). The one or more spinal implant characteristics may further comprise proper or improper tensioning of the soft tissues (ensure to not over tension) when the spinal implant is deployed. Substantially may comprise within ten percent of parallel alignment and/or measurement.

Surgical Removal Technique

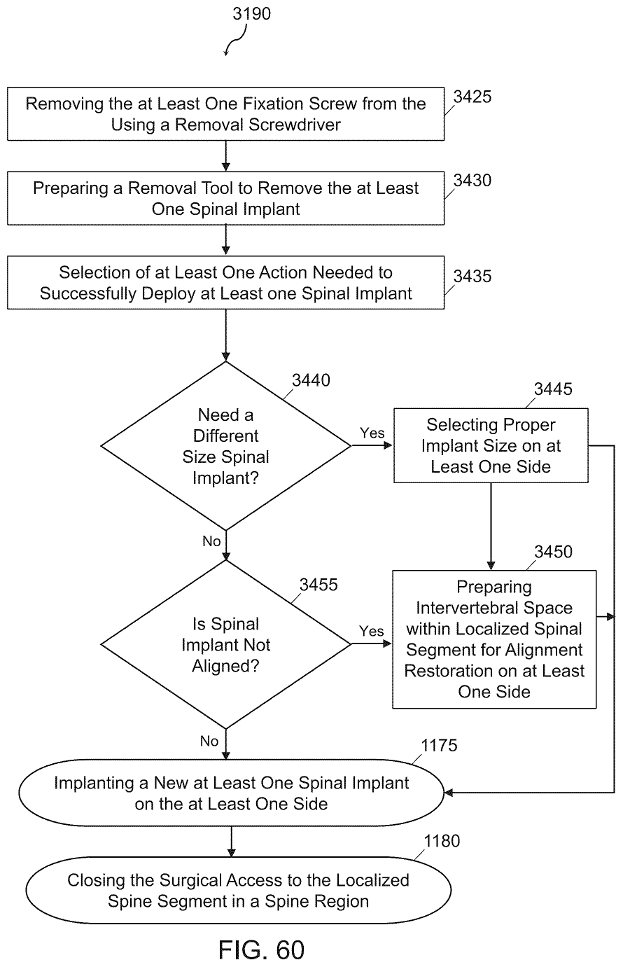

With reference to FIG. 60, the navigation assisted and/or robotic assisted preoperative procedure 1125 may comprise the step of removing at least spinal implant from the at least one side 3190 and/or removing a first spinal implant on a first side and a second spinal implant on a second side. The step of removing at least one spinal implant, a first spinal implant and/or a second spinal implant may be necessary if one or more of the spinal implant characteristics, including the tensioning, alignment, positioning, and/or placement of the at least one spinal implant deployed within the prepared intervertebral space within a spine region is unable to be confirmed as meeting surgeon and implant requirements and/or the preoperative surgical plan.

The step of removing at least one spinal implant, a first spinal implant and/or a second spinal implant the at least one side, a first side and/or a second side comprises the steps of: removing the at least one fixation screw from the at least one spinal implant 3425; removing the at least one spinal implant using at least one removal tool 3430; selecting at least one action needed to successfully deploy at least one spinal implant 3435; and implanting a new or subsequent at least one spinal implant 1175. The selection of at least one action 3535 comprises the actions: does a different sized spinal implant required 3440; if yes, the reselecting a new proper spinal implant size is required 3445; and re-preparing the intervertebral (IV) space is required 3450 and/or is the spinal implant not aligned 3455—if yes, then re-preparing the intervertebral space is required 3450. However, if the selection to actions is “no,” the surgeon and/or user may proceed with completing the step of implanting a spinal implant 1175, 1175a, 1175b, 1175c and closing the surgical access to the localized spine segment 1180. “Not aligned” comprises improper neutral alignment, improper keel alignment, improper sagittal osteotomy angle, and/or improper coronal osteotomy angle.

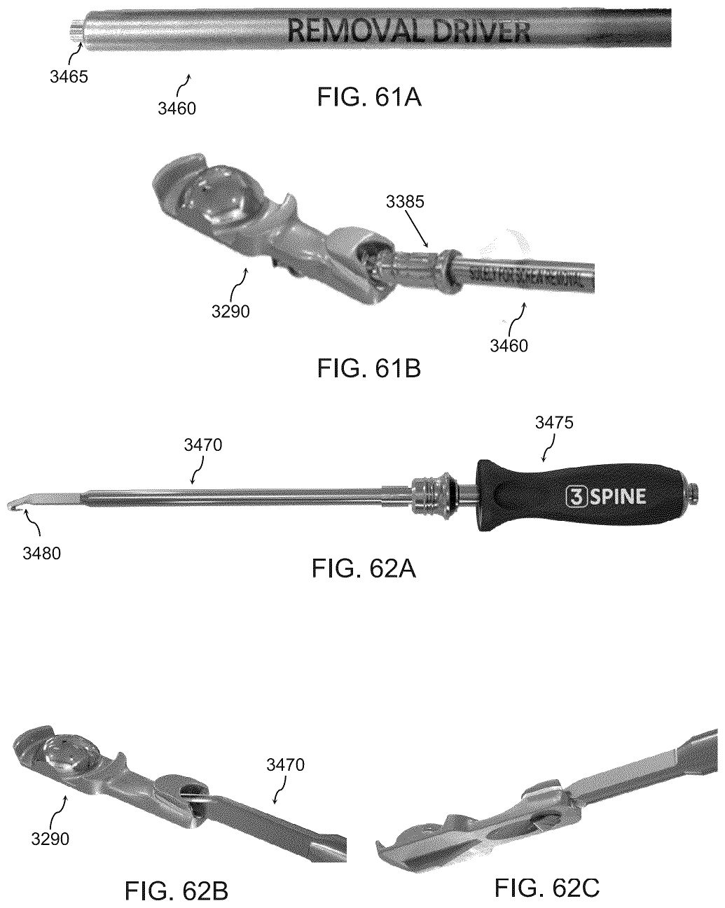

With reference to FIG. 61A-61B, the surgeon and/or user may complete the step of removing the at least one fixation screw 3385 from the at least one spinal implant, a first spinal implant, and/or a second spinal implant 3290 on the at least one side, a first side and/or a second side. A removal screwdriver 3460 may be utilized to disengage and remove the fixation screw 3385 from the caudal vertebral body. The removal screwdriver 3460 may be powered and/or manual. The removal screwdriver 3460 is coupled to a handle (e.g., Hudson handle) for manual installation. The surgeon and/or user will insert the drive tip or tip 3465 into a portion of the drive recess of the fixation screw 3385. The edge, rim and/or shoulder of the removal screwdriver 3460 will force the retainer clip 3415 to be movable from the fixed position or closed position, which a portion of the retainer clip 3415 contacts or engages a surface on the fixation screw 3385 to prevent premature backing out, to a unfixed position or an open position, the at least a portion of retainer clip 3415 expanding to allow the removal driver 3460 to disengage the fixation screw 3385 from the portion of the retainer clip 3415. The surgeon and/or user may begin to rotate the deployment screwdriver 3460 counterclockwise until the surgeon receives feedback and/or torque feedback (e.g., loosening) and continue rotating until the fixation screw 3385 is removed from the at least one spinal implant 3290.

With reference to FIGS. 62A-62C, the step of removing the at least one spinal implant, a first spinal implant and/or a second spinal implant from the at least one side, a first side and/or a second side 3190 comprises the step of assembling a removal tool 3470. The removal tool 3470 may be utilized to grasp a portion of the at least one spinal implant, a first spinal implant and/or a second spinal implant 3290 and remove the at least one spinal implant, the first spinal implant and/or the second spinal implant from the prepared intervertebral space within a spine region. The removal tool 3470 may be powered and/or manual. The removal tool 3470 is coupled to a handle 3475 (e.g., Hudson handle) for manual installation. The removal tool 3470 comprises a first end and a second end. The second end may include the handle 3475. The first end includes a hook 3480.

The surgeon and/or user may insert the first end of the removal tool 3470 into the prepared intervertebral space. The surgeon and/or user should locate the fixation screw bore disposed on the bridge 3320 of the inferior component 3300 of the spinal implant 3290. The hook 3480 disposed in the first end of the removal tool 3470 grasps is inserted into the screw bore of the bridge 3320 of the inferior component 3300 of the spinal implant 3290. The removal tool 3470 should translate posteriorly until at least a portion of the hook 3480 on the first end of the removal tool 3470 contacts or engages with a portion of an inner diameter of the screw bore. Once the removal tool 3470 is secured, the surgeon and/or user may begin translating or pulling the at least one spinal implant, a first spinal implant and/or a second spinal implant 3290 on the at least one side, the first side and/or the second side to remove the superior component 3295 and inferior component 3300 of the spinal implant 3290. After spinal implant 3290 is retracted from the prepared interverbal space, the spinal implant 3290 may be discarded. Ensure both pieces, superior component 3295 and/or inferior components 3300 of the spinal implant 3290 were removed.

In another embodiment, the surgeon may complete the step of selecting at least one action needed to successfully deploy at least one spinal implant on the at least one side. The selecting of one action may comprise re-selecting a different sized spinal implant or re-preparing the Intervertebral space within a localized spine segment for alignment restoration on at least one side. The action of re-selecting of a different sized spinal implant may be necessary if excessive friction during or excessive tension was noted during deployment of the at least one spinal implant. The surgeon should repeat one or more steps disclosed herein, described as implanting at least a spinal implant on the at least one side. For example, if too much tension and/or too much friction was observed, it may be necessary to decrease the height of the implant.

### INCORPORATION BY REFERENCE

The entire disclosure of each of the publications, patent documents, and other references referred to herein is incorporated herein by reference in its entirety for all purposes to the same extent as if each individual source were individually denoted as being incorporated by reference.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus intended to include all changes that come within the meaning and range of equivalency of the descriptions provided herein.

Many of the aspects and advantages of the present invention may be more clearly understood and appreciated by reference to the accompanying drawings. The accompanying drawings are incorporated herein and form a part of the specification, illustrating embodiments of the present invention and together with the description, disclose the principles of the invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the disclosure herein. What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

I/We claim:

1. A computer-assisted surgery system for performing a surgical procedure at a target area on a patient's anatomy, said computer-assisted surgery system comprising:
a robotic device having a plurality of joints and a plurality of actuators; and
a computer in communication with said robotic device and configured to command said robotic device to operate, said computer including a non-transitory computer-readable medium with instructions stored thereon, that when executed by a processor, perform the steps comprising:
accessing one or more preoperative images of a targeted anatomy of a patient; generating a first data set, the first data set comprises one or more preoperative virtual models of the target anatomy, the one or more preoperative virtual models including identification of anatomical landmarks;
calculating one or more selected surgical measurements from the one or more preoperative virtual models of the target anatomy to create a second data set; and
creating a preoperative plan by analyzing the one or more preoperative virtual models and the one or more selected surgical measurements;
wherein said robotic device is configured to be controlled by said computer.

2. The computer-assisted surgery system of claim 1, wherein said computer is further configured to receive a preoperative image dataset of the patient's anatomy taken with an imaging modality and register the preoperative image dataset to the patient's anatomy.

3. The computer-assisted surgery system of claim 2, wherein said computer is further configured to receive a second intraoperative image dataset of the patient's anatomy and register the preoperative image dataset and the second intraoperative image dataset to the patient's anatomy.

4. The computer-assisted surgery system of claim 1, wherein said computer is further configured to define the target area relative to a virtual representation of the patient's anatomy and register the virtual representation of the patient's anatomy to the patient's anatomy.

5. The computer-assisted surgery system of claim 1, further comprising a camera and an array of trackable markers attachable to the patient's anatomy.

6. The computer-assisted surgery system of claim 1, wherein said robotic device comprises a robotic arm configured to move said guide in at least two degrees of freedom.

7. The method of claim 1, further comprising displaying a virtual representation of the preoperative plan on a display.

8. A non-transitory computer-readable medium with instructions stored thereon, that when executed by a processor, perform the steps comprising:
accessing one or more preoperative images of a targeted anatomy of a patient;
generating a first data set, the first data set comprises one or more preoperative virtual models of the target anatomy, the one or more preoperative virtual models including identification of anatomical landmarks;
calculating one or more selected surgical measurements from the one or more preoperative virtual models of the target anatomy to create a second data set; and
creating a preoperative plan by analyzing the one or more preoperative virtual models and the one or more selected surgical measurements.

9. The non-transitory computer-readable medium with instructions of claim 8, wherein the steps further comprises the steps of:
storing the first and second data set in a storage device; and
displaying the first or second data set via a display medium;

10. The non-transitory computer-readable medium with instructions of claim 8, wherein the target anatomy comprises a spine segment of a spine in a spine region.

11. The non-transitory computer-readable medium with instructions of claim 8, wherein target anatomy comprises a first spine segment in a first spine region and a second spine segment in a second spine region.

12. The non-transitory computer-readable medium with instructions of claim 11, wherein the first spine region is different than the second spine region.

13. The non-transitory computer-readable medium with instructions of claim 11, wherein the spine region comprises a member of the group consisting of a cervical region, a thoracic region, a lumbar region and a sacral region.

14. The non-transitory computer-readable medium with instructions of claim 8, wherein the one or more preoperative images comprises a member of the group consisting of one or more raw preoperative images and one or more modified preoperative images.

15. The non-transitory computer-readable medium with instructions of claim 8, wherein the one or more virtual model comprises a member of the group consisting of at least one 2D virtual model and at least one 3D virtual model.

16. The non-transitory computer-readable medium with instructions of claim 8, wherein the first data set comprises image data and the second data set comprises numerical data.

17. The non-transitory computer-readable medium with instructions of claim 16, wherein the numerical data comprises one or more selected surgical measurements.

18. The non-transitory computer-readable medium with instructions of claim 36, wherein the storage device comprises a cloud-based storage medium.

19. The non-transitory computer-readable medium with instructions of claim 9, wherein the display medium comprises a workstation, the workstation includes a navigation workstation.
